(19) United States
(12) Patent Application Publication
(10) Pub. No.: US 2010/0291142 A1
(43) Pub. Date: Nov. 18, 2010

(54) Bacterial Artificial Chromosome Containing Feline Herpes Virus Type 1 Genome and Uses Thereof

(76) Inventors: Roger Maes, Okemos, MI (US); Tai Shih-Han, Frederick, MD (US); Niikura Masahiro, Burnaby (CA); Cheng Hans, Haslett, MI (US); Kruger John, Okemos, MI (US)

Correspondence Address: MEDLEN & CARROLL, LLP, 101 HOWARD STREET, SUITE 350, SAN FRANCISCO, CA, 94105, US

(21) Appl. No.: 12/682,969

(22) PCT Filed: Oct. 17, 2008
(86) PCT No.: PCT/US08/11914

§ 371 Date: Jul. 30, 2010

Related U.S. Application Data
(60) Provisional application No. 60/999,605, filed on Oct. 18, 2007.

(51) Int. Cl. A61K 39/12; C12N 15/63; C12N 5/07; C12N 15/79; C12N 7/00; C12N 7/02; G01N 33/566; A61P 37/04; A61P 31/22

(52) U.S. Cl. 424/205.1; 435/320.1; 435/325; 435/235.1; 435/239; 424/229.1; 436/501

(57) ABSTRACT

The present invention relates to recombinant feline herpes virus type 1 (FHV-1) nucleic acids and proteins. In particular the present invention provides compositions comprising the full length FHV-1 genome or portions thereof, and infectious FHV-1 virions produced therefrom. The FHV-1 compositions are suitable for use in inducing an immune response in inoculated subjects and for use in identifying agents that attenuate FHV-1 infection.

### FIELD OF THE INVENTION

The present invention relates to recombinant feline herpes virus type 1 (FHV-1) nucleic acids and proteins. In particular the present invention provides compositions comprising the full length FHV-1 genome or portions thereof, and infectious FHV-1 virions produced therefrom. The FHV-1 compositions are suitable for use in inducing an immune response in inoculated subjects and for use in identifying agents that attenuate FHV-1 infection.

### BACKGROUND OF THE INVENTION

Feline herpesvirus type 1 (FHV-1), a member of the Varicellovirus genus of the Alphaherpesvirinae subfamily, is a significant viral pathogen of Felidae. With its worldwide distribution, FHV-1 not only accounts for approximately 50-75% of all diagnosed viral upper respiratory infections in cats, but is also an important cause of ocular lesions in cats. The transmission of this virus is primarily via the oronasal route. FHV-1 replicates extensively in the mucosae of the upper respiratory tract, resulting in high fever, depression, anorexia, sneezing, conjunctivitis, keratitis, and ocular and nasal discharge. The mortality is higher in neonatal cats, since the virus tends to generalize in this age group.

Exposure of pregnant queens can lead to abortion, though infection with FHV-1 is not a common cause of abortion in cats. Like herpesvirus infections in other species, the acute phase of the disease is followed by lifelong latency. During the latent stage, viral DNA persists in neural tissues, mainly the sensory ganglia, but infectious virus is not produced. Different biological stresses, or the administration of corticosteroids, can induce the necessary biochemical stimuli in latently infected cells that lead to renewed production of infectious virus, which then travels to the periphery and is a potential source of viral transmission. In addition to the disease caused directly by its infection, FHV-1 is also a contributor to feline chronic rhinosinusitis (Johnson et al., Vet. Microbiol. 108(3-4):225-233, 2005).

A number of commercial modified live or inactivated virus vaccines are currently available for the control of FHV-1 infection. Unfortunately, the immunity induced by existing commercial vaccines cannot totally protect cats from field virus infection and, as a consequence, from field virus latency (Gaskell and Povey, Res Vet Sci, 27:167-174, 1979; Harbour et al., Vet Rec, 128:77-80, 1991; Tham and Studdert, Vet Rec, 120:321-326, 1987; Weigler et al., Arch Virol, 142:2389-2400, 1997; and Yokoyama et al., Arch Virol, 141:481-494, 1996). Further, previous work has been done with constructs consisting of a raccoonpox vector in which either the gB or gD gene of FHV-1 was inserted (U.S. Pat. No. 6,010,703). Both gB and gD are very immunogenic proteins. In addition, the raccoonpox vector replicates very well in feline cells, but does not cause any clinical signs. The immune response induced by vaccination with raccoonpox-gB or raccoonpox-gD can be differentiated from immunity resulting from field virus exposure by glycoprotein-specific immunologic assays, according to the DIVA principle. Thus what is needed in the art are improved FHV-1 formulations for inducing broadly active immune responses that recognize multiple FHV-1 isolates, prevent infections with field virus, and the resulting field virus latency.

### SUMMARY OF THE INVENTION

The present invention relates to recombinant feline herpes virus type 1 (FHV-1) nucleic acids and proteins. In particular the present invention provides compositions comprising the full length FHV-1 genome or portions thereof, and infectious FHV-1 virions produced therefrom. The FHV-1 compositions are suitable for use in inducing an immune response in inoculated subjects and for use in identifying agents that attenuate FHV-1 infection.

The present invention provides bacterial artificial chromosomes (BACs) comprising a feline herpes virus type I (FHV-1) genome. In some embodiments, the BAC further comprises loxP sites flanking the FHV-1 genome. In some preferred embodiments, the FHV-1 genome comprises one or more of an unique long (UL) region, an unique short (Us) region, an inverted repeat short (IRs) region and a terminal repeat short (TRs) region. In some embodiments, the FHV-1 genome comprises a polynucleotide with the nucleotide sequence set forth as SEQ ID NO:1, or a polynucleotide at least 100 kb in length that hybridizes under highly stringent conditions to the nucleotide sequence set forth in SEQ ID NO:1. In additional embodiments, the FHV-1 genome further comprises a polynucleotide with the nucleotide sequence set forth as SEQ ID NO:2, or a polynucleotide at least 5 kb in length that hybridizes under highly stringent conditions to the nucleotide sequence set forth in SEQ ID NO:2. In additional embodiments, the FHV-1 genome further comprises a polynucleotide with the nucleotide sequence set forth as SEQ ID NO:3, or a polynucleotide at least 5 kb in length that hybridizes under highly stringent conditions to the nucleotide sequence set forth in SEQ ID NO:3, as shown in FIG. 5. In some preferred embodiments, the BAC further comprises a marker for selection in eukaryotic cells (e.g., green fluorescent protein, luciferase, beta-galactosidase, xanthine phosphoribosyltransferase, etc.). In some embodiments, the FHV-1 genome has a deletion in one or more FHV-1 genes selected from the group consisting of gG gene, gD gene, gI gene, gE gene, and gC gene, and/or any other non-essential FHV-1 gene. Also provided by the present invention are isolated polynucleotides comprising a feline herpes virus type 1 (FHV-1) genome, or a portion thereof as defined above. In some preferred embodiments the FHV-1 genome comprises one or more of an unique long (UL) region, an unique short (Us) region, an inverted repeat short (IRs) region, and a terminal repeat short (TRs) region. Moreover, the present invention provides isolated polynucleotides consisting of from one to sixty two open reading frames (ORFs) of a unique long (UL) region of an FHV-1 genome. In some embodiments the present invention provides isolated polynucleotides consisting of from one to sixty-four open reading frames (ORFs) of a unique long (UL) region of an FHV-1 genome. In some embodiments, the isolated polynucleotide consists of one, two, three, four, five, six, seven, eight, nine or ten ORFs of said unique long (UL) region. In other embodiments, the isolated polynucleotide consists of one, two, three, four, five, six, seven, eight, nine or up to seventy-eight ORFs and any combinations thereof of said unique long (U_{L}) region. In additional embodiments, the present invention provides a prokaryotic host cell (e.g., E. coli) transformed with a FHV-1 BAC. In further embodiments, the present invention provides a eukaryotic host cell (e.g., CRFK) transfected with a FHV-1 BAC. In still further embodiments, the present invention provides a host cell co-transfected with a FHV-1 BAC, and a polynucleotide encoding Cre recombinase in operable combination with a promoter.

Additionally, the present invention provides methods for producing feline herpes virus type 1 (FHV-1), comprising providing: i) a cell line permissive for FHV-1 infection, ii) a bacterial artificial chromosome (BAC) comprising a FHV-1 genome flanked by loxP sites, and iii) an expression vector comprising a polynucleotide encoding Cre recombinase, in operable combination with a promoter; contacting the cell line with the BAC and the expression vector to produced a transfected cell line; and culturing the transfected cell line under conditions suitable for production of FHV-1. In some embodiments, the methods further comprise step d) purifying the FHV-1. Furthermore, the present invention provides kits for producing feline herpes virus type 1 (FHV-1), comprising: i) a cell line permissive for FHV-1 infection, ii) a bacterial artificial chromosome (BAC) comprising a FHV-1 genome flanked by loxP sites, iii) an expression vector comprising a polynucleotide encoding Cre recombinase, in operable combination with a promoter, and iv) instructions for contacting the cell line with the BAC and the expression vector to produced a transfected cell line, and culturing the transfected cell line under conditions suitable for production of FHV-1. Also provided are compositions (e.g., immunogens or vaccines) comprising FHV-1 produced by the methods or with the kits of the present invention. In some embodiments, the compositions further comprising a pharmaceutically acceptable carrier. In still further embodiments, the present invention provides methods for immunizing a cat against feline herpesvirus 1 (FHV-1), comprising administering to a cat the composition comprising FHV-1 and a pharmaceutically acceptable carrier. In some embodiments, the administering comprises intramuscular or intranasal inoculation. In other embodiments, the administering comprises subcutaneous or intradermal inoculation.

The present invention provides a bacterial artificial chromosome (BAC) comprising a feline herpes virus type I (FHV-1) genome. In some embodiments, the BAC further comprises loxP sites flanking the BAC. In some preferred embodiments, the FHV-1 genome comprises a unique long (U_{L}) region. In other embodiments, the FHV-1 genome further comprises a unique short (Us) region. In further embodiments the FHV-1 genome further comprises one or both of an inverted repeat short (IRs) region and a terminal repeat short (TRs) region. In still further embodiments the FHV-1 genome comprises a polynucleotide with a sequence selected from the group consisting of SEQ ID NO:1, SEQ ID NO:2, SEQ ID NO:3, and SEQ ID NO:4. In additional embodiments the BAC further comprises a marker for selection in eukaryotic cells. In some embodiments a host cell is transformed with the BAC. In other embodiments, a host cell co-transformed with the BAC of claim 1 and a polynucleotide encoding Cre recombinase in operable combination with a promoter.

Additionally, the present invention provides a method for producing feline herpes virus type 1 (FHV-1), comprising, a) providing: i) a cell line permissive for FHV-1 infection, ii) a bacterial artificial chromosome (BAC) flanked by loxP sites comprising a FHV-1 genome, and iii) an expression vector comprising a polynucleotide encoding Cre recombinase, in operable combination with a promoter, b) contacting said cell line with said BAC and said expression vector to produced a transfected cell line; and c) culturing said transfected cell line under conditions so that FHV-1 is produced. In some embodiments the methods further comprise step d) purifying said FHV-1. Also provided are compositions, a composition comprising FHV-1 produced by the method. In some embodiments the composition further comprises a pharmaceutically acceptable carrier. In still further embodiments, the present invention provides for methods of immunizing a cat against feline herpesvirus 1 (FHV-1), comprising administering to a cat the composition of FHV-1 and pharmaceutically acceptable carrier. In some embodiments the administering comprises intramuscular, subcutaneous, intradermal, or intranasal inoculation. The present invention also provides for a kit for producing feline herpes virus type 1 (FHV-1), comprising: a) a cell line permissive for FHV-1 infection; b) a bacterial artificial chromosome (BAC) flanked by loxP sites comprising a FHV-1 genome; an expression vector comprising a polynucleotide encoding Cre recombinase, in operable combination with a promoter; and d) instructions for contacting the cell line with the BAC and the expression vector to produced a transfected cell line, and culturing the transfected cell line under conditions suitable for production of FHV-1. In other embodiments the kit further comprises: i) a first marker for selection, in operable combination with an endonuclease recognition site; ii) a plasmid comprising a homing endonuclease I-SceI, in operable combination with a second marker for selection; iii) an expression vector comprising a polynucleotide encoding Cre recombinase, in operable combination with a promoter; and iv) instructions for contacting the host cell with the BAC, expression vector, and said first marker for selection to produce a first transformed host cell, and growing the first transformed host cell under conditions suitable for selection of a first transformed host cell, and contacting said first transformed host cell with the plasmid to produce a second transformed host cell, and growing said second transformed host cell under conditions suitable for selection of said second transformed host cell.

In other embodiments the present invention provides a method for producing a feline herpes virus type 1 (FHV-1) mutant, comprising: a) providing: i) a host cell permissive for FHV-1 infection; ii) a bacterial artificial chromosome (BAC) flanked by loxP sites comprising a FHV-1 genome; iii) a first marker for selection, in operable combination with an endonuclease recognition site; and b) contacting said host cell with said BAC, and said first marker for selection, and said plasmid to produce a first transformed host cell; c) growing said first transformed host cell under conditions suitable for selection of said first marker for selection wherein said selection includes expression of a protein. In other embodiments the method further comprises d) providing: i) a plasmid comprising a homing endonuclease I-SceI, in operable combination with a second marker for selection; e) contacting said first transformed host cell with said plasmid to produce a second transformed host cell whereby said first marker for selection is deleted from said second transformed host cell; and f) growing said second transformed host cell under conditions suitable for selection of said second marker for selection. In still further embodiments the method provides the first marker for selection recombines with a target gene or portion thereof whereby said target gene or portion thereof is replaced by said first marker for selection. In still further embodiments, the method provides the target gene is selected from the group consisting of: gG gene, gI gene, gC gene, and gE gene. In still further embodiments, the method further comprises step d) purifying said first transformed host cell. In other embodiments the method further comprises step g) purifying said second transformed host cell. In still further embodiments the method further comprises step h) repeating steps b) through g) to produce a feline herpes virus type 1 (FHV-1) double mutant.

Also provided are compositions, a composition comprising FHV-1 produced by the method of producing a FHV-1 double mutant. In some embodiments, the composition further comprising a pharmaceutically acceptable carrier. In further methods, a method for immunizing a cat against feline herpesvirus 1 (FHV-1), comprising administering to a cat the composition of a FHV-1 double mutant. In some embodiments the administering comprises intramuscular, subcutaneous, intradermal, or intranasal inoculation.

The present invention also provides for a method for differentiating between immunity resulting from vaccinations with BAC-derived gene-deleted FHV-1 or field virus, comprising: a) providing; i) a patient suspected of having FHV-1; ii) a biological sample derived from said patient, wherein said sample is serum wherein said serum contains an FHV-1 antibody capable of interacting with a ligand wherein said ligand is an FHV-1 antigen; b) incubating said sample with said ligand under conditions such that said sample binds to said ligand thereby forming a sample-ligand complex; and c) detecting said sample-ligand complex, thereby differentiating between said FHV-1 antibodies generated from vaccination and infection.

In still further compositions, a composition, comprising the BAC, wherein said FHV-1 genome comprises a polypeptide selected from the group consisting of a nucleotide sequence of at least 135 kb in length that hybridizes under high stringency conditions to the nucleotide sequence set forth in SEQ. ID No:4, a nucleotide sequence of at least 100 kb in length that hybridizes under high stringency conditions to the nucleotide sequence set forth in SEQ. ID No:1, a nucleotide sequence of at least 5 kb in length that hybridizes under high stringency conditions to the nucleotide sequence set forth in SEQ. ID No:2, a nucleotide sequence of at least 5 kb in length that hybridizes under high stringency conditions to the nucleotide sequence set forth in SEQ. ID No:3. In some embodiments the composition further comprises a pharmaceutically acceptable carrier. In further methods for immunizing a cat against feline herpesvirus 1 (FHV-1), comprising administering to a cat the composition. In some embodiments the method for administering comprises intramuscular, subcutaneous, intradermal, or intranasal inoculation.

In some embodiments the method for producing a mutant further comprises step a) iv) an expression vector comprising a polynucleotide encoding Cre recombinase, in operable combination with a promoter. In yet further compositions, the composition comprises FHV-1 produced with one mutant. In some further embodiments the composition further comprises a pharmaceutically acceptable carrier. In still other embodiments, a method for immunizing a cat against feline herpesvirus 1 (FHV-1), comprising administering to a cat the composition of FHV-1 single mutant. In other embodiments the method of administering comprises intramuscular, subcutaneous, intradermal, or intranasal inoculation. In still other methods, a method of producing an immunogen, comprising: a) providing; i) a cell line permissive for FHV-1 infection; ii) a bacterial artificial chromosome (BAC) flanked by loxP sites comprising a FHV-1 genome; and b) contacting said cell line with said BAC to produce; and c) culturing said FHV-1 BAC under conditions suitable for production of said immunogen. The method wherein said contacting includes a m.o.i of 0.01. In some embodiments, the method further comprises step d) collecting a supernatant wherein said collection is at 24, 48, and 72 hours post inoculation. In still further embodiments, the method further comprises providing step a) iii) an expression vector comprising a polynucleotide encoding Cre recombinase, in operable combination with a promoter and wherein said contacting further comprises said expression vector.

While specific embodiments are given it is optionally desirable for other things to be used that are known in the state of the art. In some embodiments the methods express proteins and those proteins can be used as further compositions and for immunizing cats. Further embodiments also include use of (and methods for producing) the FHV-1 BAC clone with the BAC removed and portions thereof of any of the FHV-1 genome or expressed proteins for use as compositions and immunizing a cat. While some embodiments comprise use of a pharmaceutically acceptable carrier the use of the carrier is optional and the embodiments can be used alone or in other formulations as known in the art of drug delivery. Further embodiments include methods of production for large-scale batches for manufacturing as known in the art. While specific cells, examples, reagents, methods, and compositions are given they are not meant to be limiting and include other known comparable techniques in the state of the art. Some embodiments comprise deletions to form mutants and include deletions of portions of the targeted gene or sequences. Further embodiments include, but are not limited to, using SEQ ID NO:4 with the BAC sequences removed (i.e. for example, SEQ ID NO:4 lacking BAC sequences).

### DESCRIPTION OF THE DRAWINGS

FIG. 1 provides a schematic of the BAC backbone plasmid utilized during development of the present invention. It contains two recombination arms flanking the sequence of the targeted FHV-1 gene, a green fluorescence protein (GFP) gene, an origin of replication and a chloramphenicol resistance gene, as well as F-factors for single copy maintenance in E. coli. See FIG. 19 for an updated schematic.

FIG. 2 provides a schematic of the process employed for cloning an FHV-1 genome as a BAC, by co-transfection of a BAC backbone plasmid and genomic DNA of the FHV-1 C-27 strain into Crandell-Reese feline kidney (CRFK) cells, followed by selection for homologous recombinants. Recombinant virus particles were harvested from the cell culture medium and plaque purified before DNA extracted from fluorescent viral clones was extracted and transformed into E. coli.

FIG. 3 shows the location in which PCR primers anneal to DNA templates of the wild type virus, the BAC backbone plasmid, and the recombinant virus. Also see FIG. 17A/B for another embodiment.

FIGS. 4A and 4B show the fluorescent antibody staining for FHV-1 of plaques produced by the C-27 strain, while FIGS. 4C and 4D show the staining of plaques produced by the BAC clones, two days after inoculation of CRFK monolayers.

FIG. 5 shows the physical structure of the FHV-1 genome, with shaded boxes indicating sequenced portions. Also see FIG. 22 for a complete structure.

FIG. 6A provides a map of a contig covering the bulk of the FHV-1 UL region, while FIG. 6B provides the nucleic acid sequence of the 105,901 by contig (SEQ ID NO:1) showing locations Contig00003: 114777-117439; Contig00006 (BAC insertion): 105902-114776; and Contig00008: 1-105901.

FIG. 7A provides a map of a contig covering a portion of the FHV-1 IRS/TRS regions, while FIG. 7B provides the nucleic acid sequence of the 5,787 by contig (SEQ ID NO:2) showing Contig00025: 1-5787.

FIG. 8A provides a map of a contig covering the FHV-1 US region and a portion of the IRS/TRS regions, while FIG. 8B provides the nucleic acid sequence of the 15,327 by contig (SEQ ID NO:3) showing locations Contig00001: 5239-5677 (reverse complement); Contig00002: 7087-11972 (reverse complement); Contig00022: 6966-7086 (reverse complement); Contig00023: 1-5238 (reverse complement); and Contig00024: 5678-6725 (reverse complement).

FIG. 9A provides a graph of the growth curves of the wild type C-27 strain and the BAC clone. The viruses were inoculated on CRFK monolayers at an MOI of 0.01, supernatants were subsequently collected and titrated at 0, 6, 24, 48 and 72 hours post inoculation. While 9B shows the growth curve with error bars.

FIG. 10 provides an overview of the recombineering approach to introduce mutations into the FHV-1 BAC.

FIG. 11A provides a diagram showing the process of gC engineering and approximate location of primers (grey arrows) used for PCR assays, while FIG. 11B provides a diagram of gE engineering and approximate location of primers (grey arrows).

FIG. 12A provides a gel of the PCR reactions using primer pairs B (lanes B1-B4) and C (lanes C1-C4), with four FHV-1ΔgCKnR clones as template DNA and parent FHV-1 BAC as negative control (lanes B− and C−). The PCR products were separated in a 1% agarose gel and stained with ethidium bromide while FIG. 12B provides a gel of the PCR reactions using primer pairs E (lanes E (lanes E1-E4) and F (lanes F1-F4), with four FHV-1ΔgEKnR clones as template DNA and parent FHV-1 BAC as negative control (lanes E− and F−). The PCR products were separated in a 1% agarose gel and stained with ethidium bromide. FIG. 12C provides a gel from PCR reactions using primers RM1188 and RM1189, with five FHV-1ΔgC clones (lanes 1-5) while FIG. 12D provides a gel from PCR reactions using primers RM1191 and RM1193, with ten FHV-1ΔgE clones (lanes 1-10).

FIG. 13 provides a chart listing the open reading frames (ORFs) identified for FHV-1.

FIG. 14A and FIG. 14B provide a listing of primers used in construction and verification of the BAC clone and generating the complete sequence of FHV-1 genome and construction of gC-, gE-, and gC-/gE-deletion mutants of FHV-1.

FIG. 15A provides a map of the FHV-1 genome, while 15B provides the nucleic acid sequence of the exemplary embodiment of 147,238 by FHV-1 BAC clone (SEQ. ID. NO:4) showing locations: Contig00003: 1036-3698; Contig00006(BAC insertion): 3699-12573; Contig00008: 12574-118474; Contig00025: 119140-124926; 141451-147237; Contig00001: 130771-131209 (reverse complement); Contig00002: 132604-137489 (reverse complement); Contig00022: 132483-132603 (reverse complement); Contig00023: 125533-130770 (reverse complement); Contig00024: 131210-132257 (reverse complement); gap 1:1-1035; 147238-147238; gap 2: 124927-125532; 140845-141450; and gap 3: 118475-119139.

FIG. 16 provides a phylogenic analysis on the FHV-1 glycoproteins.

FIGS. 17A and B shows the location in which PCR primers anneal to DNA templates of the wild type virus, the BAC backbone plasmid, and the recombinant virus, while 17C shows detection of different components in the recombinant virus Clone #11A1-01.

FIG. 18 provides a listing of the DNA sequence analysis tools.

FIG. 19 provides a schematic of the BAC backbone plasmid utilized during development of the present invention. It contains two recombination arms flanking the sequence of the targeted FHV-1 gene, a green fluorescence protein (EGFP) gene, an origin of replication and a chloramphenicol resistance gene, two loxP sites, as well as F-factors for single copy maintenance in E. coli.

FIGS. 20A and B provide the sequence listings for each amplicon listed in Table 4.

FIG. 21 provides a diagram showing the physical structure of the FHV-1 genome. The locations of SEQ ID:1, 2, 3, and 4; previous Gap 1, 2, and 3 that are now filled; and a listing of corresponding contigs. The figure is not to scale.

FIG. 22 provides the nucleic acid sequence of the exemplary embodiment of 147,238 by FHV-1 BAC clone (SEQ. ID. NO:4).

### DEFINITIONS

To facilitate understanding of the invention, a number of terms are defined below.

The use of the article “a” or “an” is intended to include one or more.

As used herein, terms defined in the singular are intended to include those terms defined in the plural and vice versa.

As used herein, the terms “purified” and “isolated” refer to molecules (polynucleotides or polypeptides) or organisms that are removed or separated from their natural environment. “Substantially purified” molecules or organisms are at least 50% free, preferably at least 75% free, more preferably at least 90% and most preferably at least 95% free from other components with which they are naturally associated.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/l denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42 C when a probe of about 500 nucleotides in length is employed.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies.

The term “adjuvant” as used herein refers to any compound that when injected together with an antigen, non-specifically enhances the immune response to that antigen. Exemplary adjuvants include but are not limited to incomplete Freunds adjuvant (IFA), aluminum-based adjuvants (e.g., AIOH, AIPO4, etc), and Montanide ISA 720. The terms “excipient,” “carrier” and “vehicle” as used herein refer to usually inactive accessory substances into which a pharmaceutical substance (e.g., EHEC cells, vaccine viruses) is suspended. Exemplary carriers include liquid carriers (such as water, saline, culture medium, aqueous dextrose, and glycols) and solid carriers (such as carbohydrates exemplified by starch, glucose, lactose, sucrose, and dextrans, anti-oxidants exemplified by ascorbic acid and glutathione, and hydrolyzed proteins). Moreover, a diluent or aqueous solution capable of use as a pharmaceutical and includes excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The terms “double deletion” and “double mutant” as used herein refers to a mutation in one or more positions of a genomic sequence. The mutation can be a result of a deletion, addition, translocation, inversion, rearrangement or other similar changes in the genome.

The term “transfection” as used herein refers to the introduction of foreign DNA into eukaryotic cells (e.g. CRFK cells). Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, viral infection, biolistics (i.e., particle bombardment) and the like.

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein.

The term “polynucleotide” refers to a molecule comprised of several deoxyribonucleotides or ribonucleotides, and is used interchangeably with oligonucleotide. Typically, oligonucleotide refers to shorter lengths, and polynucleotide refers to longer lengths, of nucleic acid sequences.

The term “marker for selection” as used herein refers to a gene which encodes an enzyme having an activity that confers resistance to an antibiotic (ampicillin, kanamycin, chloramphenicol, zeocin, tetracycline, etc.) drug, or digestion of an indicator such as X-gal, upon the cell in which the marker for selection is expressed, or which confers expression of a trait which can be detected (e.g., luminescence or fluorescence). Examples are beta-galactosidase, green fluorescent protein (GFP), luciferase, xanthine phosphoribosyltransferase etc.).

The terms “restriction,” “restriction digest,” “restriction enzyme digestion,” and “restriction pattern” as used herein refers to a procedure of digesting nucleic acids with restriction enzymes to selectively cleave the nucleic acid sequences into shorter fragments. The short fragments can then be analyzed by gel electrophoresis.

The term “glycoprotein” as used herein refers to any protein with one or more covalently linked oligosaccharide chains. It includes most secreted proteins and most proteins exposed on the outer surface of the plasma membrane.

The term “promoter” as used herein refers to a nucleotide sequence in DNA to which RNA polymerase binds to begin transcription.

The term “host cell” as used herein refers to a cell (e.g. prokaryotic, eukaryotic) capable of harboring a virus, vector, BAC, plasmid, etc.

The term “transformation” as used herein refers to introduction of an inheritable alteration/mutation to prokaryotic cells (e.g. E. coli) from the uptake, incorporation, or expression of foreign DNA. Transformation may be accomplished by many means known in the art. For example, chemically induced, microinjection, protoplast fusion, electroporation, lipofection, viral infection etc. Also see transfection.

The term “monoclonal antibody” as used herein refers to an antibody obtained from a population of substantially homogeneous antibodies, i.e., the individual antibodies comprising the population are identical except for possible naturally-occurring mutations that may be present in minor amounts (Harlow et al., Antibodies: A Laboratory Manual, 1988). Antibodies are contemplated to be from mice, rats, sheep, goat, humans, dogs, cats, horse and any other known sources.

The term “antibody” as used herein refers to an intact antibody or fragments thereof. Antibody fragments comprise a portion of an intact antibody, preferably the antigen binding or variable region of the intact antibody. Examples of antibody fragments include Fab, Fab′, F(ab′).sub.2, and Fv fragments; diabodies; linear antibodies (Zapata et al., Protein Eng. 8(10): 1057-1062, 1995); single-chain antibody molecules; and multi-specific antibodies formed from antibody fragments (Harlow et al., Antibodies: A Laboratory Manual, 1988).

The term “ELISA” as used herein refers to an enzyme linked immunoassay. The test is highly sensitive and can detect and quantitate antigens and antibodies. There are numerous configurations, for performing immunoassays some include use of fluorescent labels, biotin-streptavidin, enzymes, etc. as discussed in Harlow et al., Antibodies: A Laboratory Manual, 1988.

The term “DIVA” (Differentiating Infected from Vaccinated Animals) as used herein refers to a serological method to differentiate immune responses induced by vaccination from those induced by infection.

The term “Recombineering” (recombination-mediated genetic engineering) as used herein refers to a powerful method for fast and efficient manipulation of the BAC clones in E. coli. The method is based on homologous recombination in E. coli using recombination proteins provided from λ phage.

The term “kit” as used herein refers to any delivery system for delivering materials. In the context of recombinant viruses, such delivery systems include systems that allow for the storage, transport, or delivery of recombinant reagents (e.g., vectors, nucleic acid sequences, enzymes, etc. in the appropriate containers) and/or supporting materials (e.g., buffers, written instructions for performing the recombination etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains an BAC. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

The term “ligand” as used herein refers to any molecule that binds to a specific site on a protein or other molecule. For example, antigens, antibodies, hormones, small molecule etc.

The term “expression vector” or “vector” as used herein refers to a recombinant DNA molecule containing a desired coding sequence and appropriate nucleic acid sequences necessary for the expression (i.e., transcription and/or translation) of the operably linked coding sequence in a particular host organism. The most preferred vector as used herein, is the bacterial artificial chromosome vector but other expression vectors are exemplified by, but not limited to, bacterial plasmid, phagemid, shuttle vector, cosmid, virus, chromosome, mitochondrial DNA, plastid DNA, and nucleic acid fragment. Nucleic acid sequences used for expression in prokaryotes include a promoter, optionally an operator sequence, a ribosome binding site and possibly other sequences. Eukaryotic cells are known to utilize promoters, enhancers, and termination and polyadenylation signals.

The term “recombines” or “recombination” as used herein refers to the process in which a strand of genetic material (DNA molecules, RNA molecules, nucleic acids, chromosomes, genes etc.) is broken and the fragments are rejoined in new combinations. It includes an artificial and deliberate recombination of different fragments of DNA from different organisms, such as the insertion of a marker for selection in place of a gene.

The terms “immunity” and “immune response” as used herein, refers to the alteration in the reactivity of an organism's immune system upon exposure to an antigen. The term “immune response” encompasses but is not limited to one or both of the following responses: antibody production (e.g., humoral immunity), and induction of cell-mediated immunity (e.g., cellular immunity including helper T cell and/or cytotoxic T cell responses).

The terms “antigen,” “immunogen,” “antigenic,” “immunogenic,” “antigenically active,” and “immunologically active” as used herein, refer to any substance that is capable of inducing a specific humoral and/or cell-mediated immune response. An immunogen generally contains at least one epitope. Immunogens are exemplified by, but not restricted to molecules which contain a peptide, polysaccharide, nucleic acid sequence, and/or lipid. Complexes of peptides with lipids, polysaccharides, or with nucleic acid sequences are also contemplated, including (without limitation) glycopeptide, lipopeptide, glycolipid, etc. These complexes are particularly useful immunogens where smaller molecules with few epitopes do not stimulate a satisfactory immune response by themselves.

The terms “infection” or “infected” as used herein, refers to a patient in which a pathogen has established itself. Most preferably, the pathogen is feline herpes virus type 1 (FHV-1).

The term “exposure” as used herein, refers to a patient, which has been in contact with a pathogen. Most preferably, the pathogen is feline herpes virus type 1 (FHV-1).

The term “inoculation” or “vaccination” or “immunizing” as used herein, are interchangeable and refer to the introduction of a vaccine into the body for the purpose of inducing immunity.

The terms “patient” or “individual” or “subject” as used herein, are interchangeable and refer to mammals. Most preferably, Felidae.

The term “non-essential gene” as used herein, refers to a gene that can be deleted without affecting the ability for replication in vivo or in vitro.

The term “multiplicity of infection” or “moi”, as used herein, refers to the average number of infectious particles.

### DETAILED DESCRIPTION OF THE INVENTION

The present invention relates to recombinant feline herpes virus type 1 (FHV-1) nucleic acids and proteins. In particular the present invention provides compositions comprising the full length FHV-1 genome or portions thereof, and infectious FHV-1 virions produced therefrom. The FHV-1 compositions are suitable for use in inducing an immune response in inoculated subjects and for use in identifying agents that attenuate FHV-1 infection.

### I. Feline Herpes Virus Type 1 (FHV-1)

As a member of the Alphaherpesvirinae, FHV-1 has a double-stranded DNA genome, approximately 134 kb in size, and more precisely the FHV-1 genome has been determined to be the exemplary embodiment of 135,796 by with an overall G+C content of approximately 45%. The physical structure of the FHV-1 genome is similar to that of other alphaherpesviruses (i.e. varicelloviruses) (Grail et al., Arch Virol, 116:209-220, 1991; and Rota et al., Virol, 154:168-179, 1986). Alphaherpesviruses encode 65-80 open reading frames (Alba et al., Genome Res, 11:43-54, 2001) in a genome having two unique sequence segments, Unique Long (U_{L}) and Unique Short (Us). The Us region is flanked by a pair of identical but inverted sequences, termed Terminal Repeat Short (TRs) and Inverted Repeat Short (IRs). The genome consists of a 105,901 by long U_{L} and a 8,440 by long U_{S} region, with the latter being flanked by inverted IRs and TRs elements of 10,496 by each. Alphaherpesvirus has also been shown to have an associated thymidine kinase gene, which has been shown to have highly divergent proteins (Nunberg et al., J Virol, 63:3240-3249). FHV-1 has been shown to contain 23 virion-associated proteins (Fargeaud et al., Arch Virol, 80:69-82, 1984). Seven glycoproteins have been identified, designated as gB, gC, gD, gE, gG, gH, gI and an then an eighth gL. Further studies have identified at least three more glycoproteins, gM, gN, and gK. Although studies of the functions of some of these glycoproteins have been conducted (reviewed in Maeda et al., J Vet Med Sci, 60:881-888, 1998), it is clear that much more work is needed to define the roles of the FHV-1 glycoproteins in viral pathogenesis and immunity.

Our knowledge about FHV-1 at the molecular level is still limited. In recent years, many herpesvirus genomes of human and veterinary medical importance have been completely sequenced, among them herpes simplex virus 1 (HSV-1) and 2 (HSV-2), varicella-zostervirus (VZV), bovine herpesvirus 1 (BHV-1) and 5 (BHV-5), equine herpesvirus 1 (EHV-1) and 4 (EHV-4), and pseudorabies virus (PRV). However, only a fraction of the FHV-1 genomic sequence is currently available.

### II. Bacterial Artificial Chromosomes (BACs)

BACs are single copy F-factor-based plasmid vectors that can stably hold up to 300 kb of foreign DNA (Shizuya et al., Proc Natl Acad Sci USA, 89:8794-8797, 1992). BACs have several advantages over the other vectors including cloning capacity, stability in E. coli, and the efficiency of manipulation. First, BACs are much more stable than other vectors, because the strict control of the F-factor replicon maintains a single copy of the BAC per bacterial cell. This reduces the risk of otherwise frequent recombination events via repetitive DNA elements present in the DNA inserts (Kim et al., Nucleic Acid Res, 20:1083-1085, 1992). The capacity and stability of BACs enables the cloning of an entire herpesvirus genome into a single plasmid. Secondly, for subsequent functional genetics study, once a viral genome is cloned into a BAC, it can be manipulated within E. coli. Utilizing prokaryotic recombinases, such as recA, recE, recT (Link et al., J Bacteriol, 179:6228-6237, 1997; Horsburgh et al., Gene Ther, 6:922-930, 1999; and Narayanan et al., Gene Ther, 6:442-447, 1999) or the mini-lambda system (Court et al., Gene, 315:63-69, 2003; and Costantino and Court, 100:15748-15753, 2003), site-specific mutations can be introduced, theoretically anywhere in the viral genome. All mutagenesis steps can be controlled, analyzed and the mutants can be stably maintained in E. coli. This is in contrast to methods employing other vectors, where the recombination takes place in eukaryotic cells and the analyses can only start after the virus has been reconstituted and isolated. Unwanted additional changes that may have occurred in the viral genome during growth in eukaryotic cells, such as deletions, rearrangements or illegitimate recombinations, frequently can only be observed after considerable time and effort. Finally, it is safer to work with herpesviruses when the viral genome is primarily maintained as a BAC. These properties have made BACs the vectors of choice for the cloning of eukaryotic genome libraries and large viral genomes. Several herpesvirus genomes of medical and veterinary importance have been cloned in BACs since the first successful report (Messerle et al., Proc Natl Acad Sci USA, 94:14759-14763, 1997), including herpes simplex virus (Stavropoulos and Strathdee, J Virol, 72:7137-7143, 1998; Saeki et al., Hum Gene Ther, 9:2787-2794, 1998; Tanaka et al., J Virol, 77:1382-1391, 2003; and Horsburgh et al., U.S. Pat. No. 6,277,621, 2001), Epstein-Barr virus (Delecluse et al., Proc Natl Acad Sci USA, 95:8245-8250, 1998), human cytomegalovirus (Borst et al., J Virol, 73:8320-8329, 1999; Marchini et al., J Virol, 75:1870-1878, 2001; and Yu et al., J Virol, 76:2316-2328, 2002), psuedorabies virus (Smith and Enquist, J Virol, 73:6405-6414, 1999), equine herpesvirus (Rudolph et al., J Vet Med B Infect Dis Vet Public Health, 49:31-36, 2002), Marek's disease virus (Schumacher et al., J Virol, 74:11088-11098, 2000; Niikura et al., Arch Virol, 151(3):557-49, 2006).

### III. FHV-1 BAC Clones

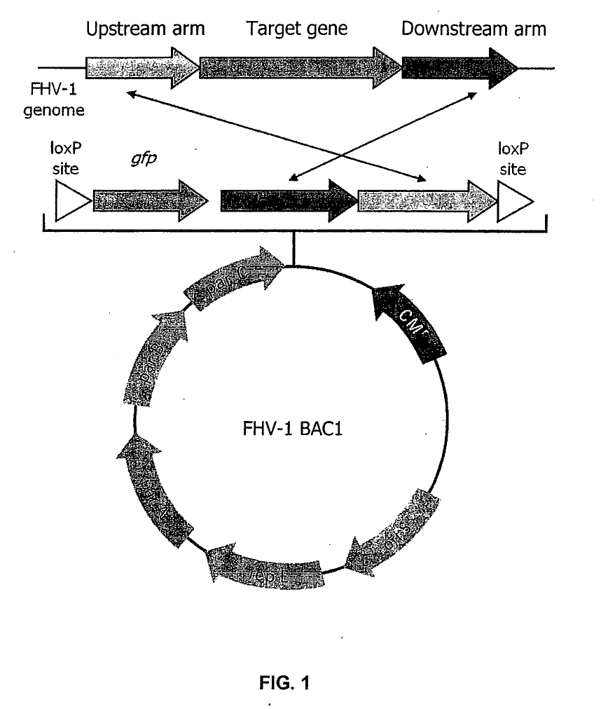

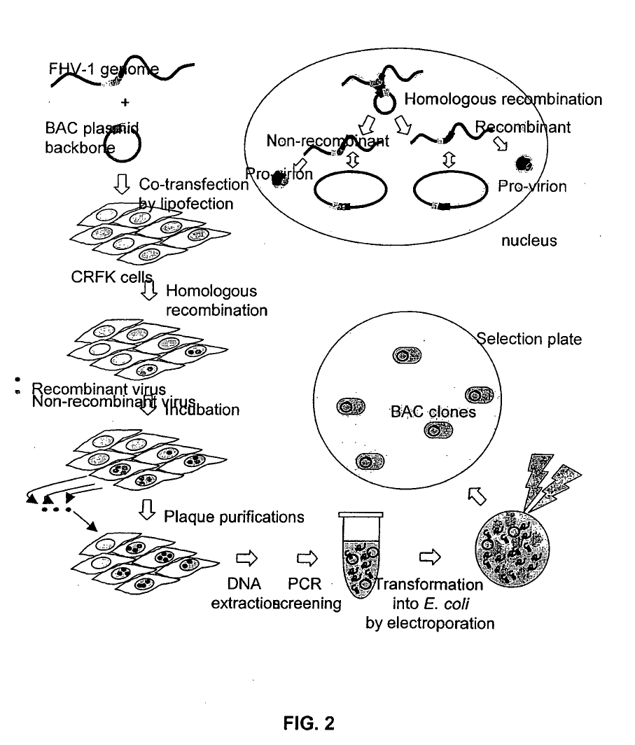

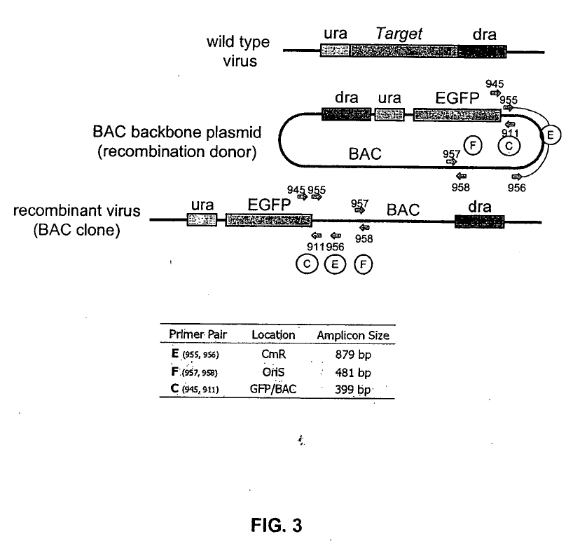

In an exemplary embodiment, a BAC backbone plasmid was constructed consisting of two recombination arms flanking targeted FHV-1 gG sequence, a green fluorescence protein (GFP) gene as the selection marker in eukaryotic cells, an origin of replication (prokaryotic), and a chloramphenicol resistance gene as the selection marker in E. coli, as well as F-factors for single copy maintenance in E. coli, as shown in FIGS. 1-3. The gG gene was selected as the target for BAC insertion because gG homologues have been described in several alphaherpesviruses as a minor non-essential glycoprotein, which does not affect overall immunogenecity (Baranowski et al., Vet Microbiol, 53:91-101, 1996). This BAC donor plasmid was co-transfected with genomic DNA of the FHV-1 C-27 strain into Crandell-Reese feline kidney (CRFK) cells, where homologous recombination takes place. Recombinant virus particles were harvested from the cell culture medium and subjected to a series of plaque purifications. One clone that consistently produced fluorescent, yet morphologically atypical plaques (albeit they are morphologically similar to those produced by the parent strain), has been isolated and designated as Clone #11A1-01. In addition to fluorescence-based screening, a set of PCR assays was developed to detect different components of the FHV-1 BAC clone, and for screening recombinant viral plaques. Clone #11A1-01 showed all of the expected PCR results indicating that the BAC donor plasmid was properly inserted into the FHV-1 genome.

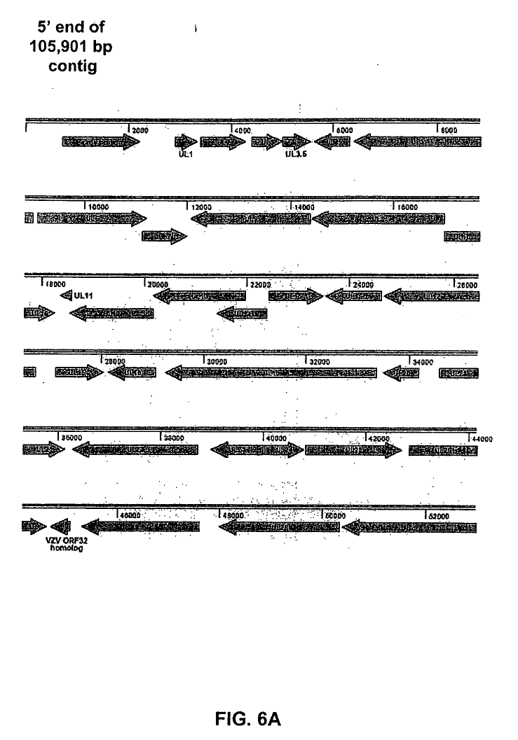

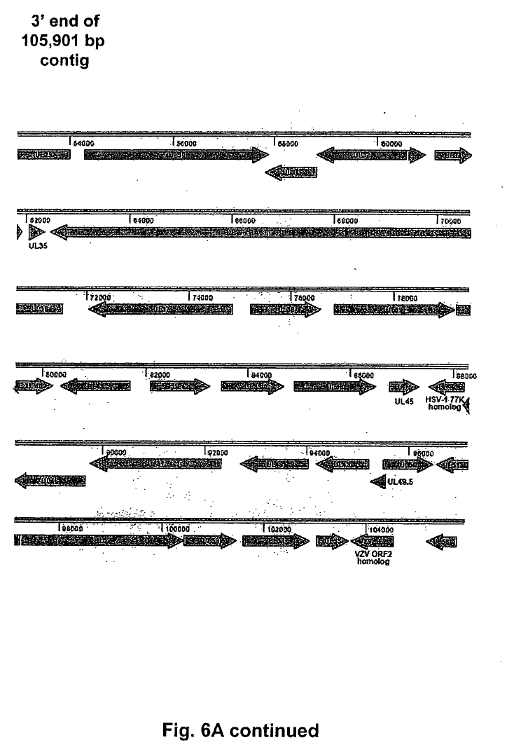

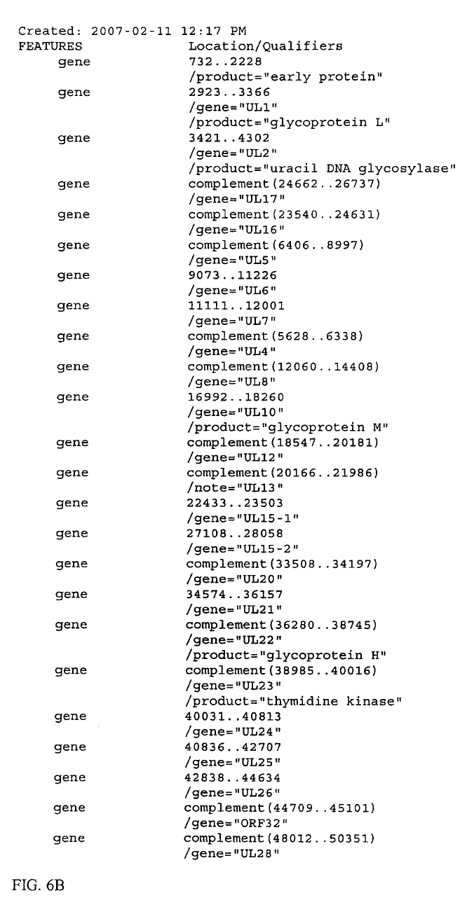

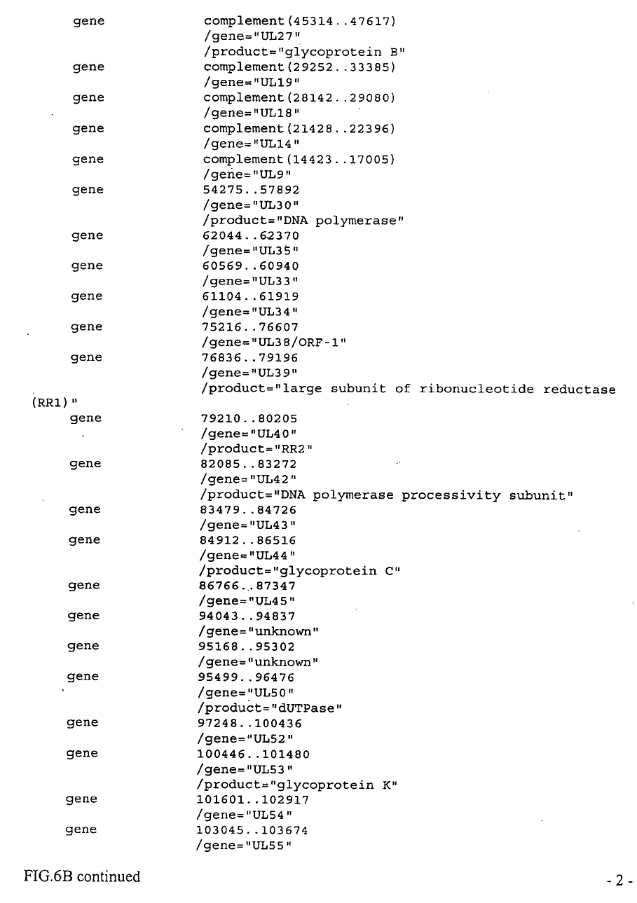

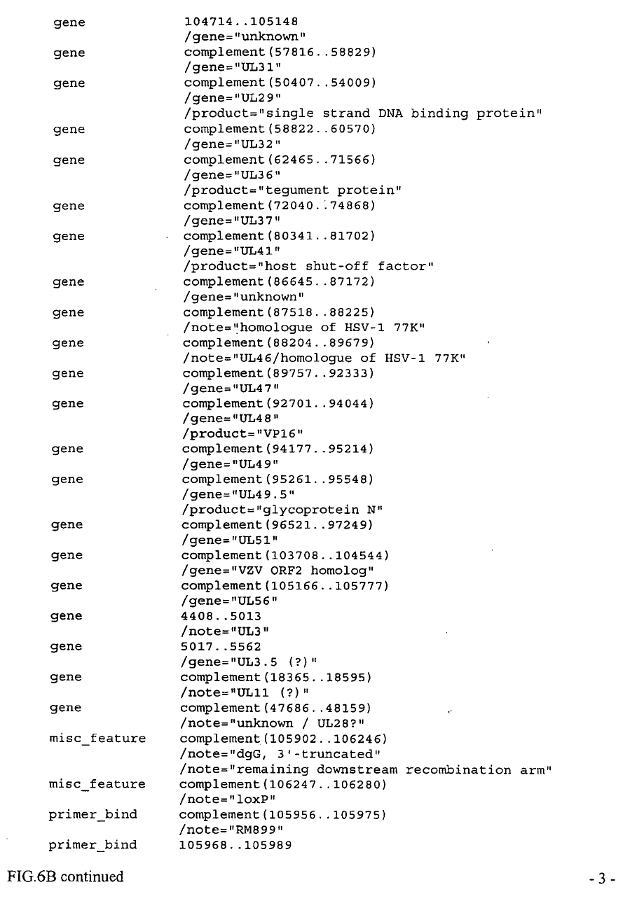

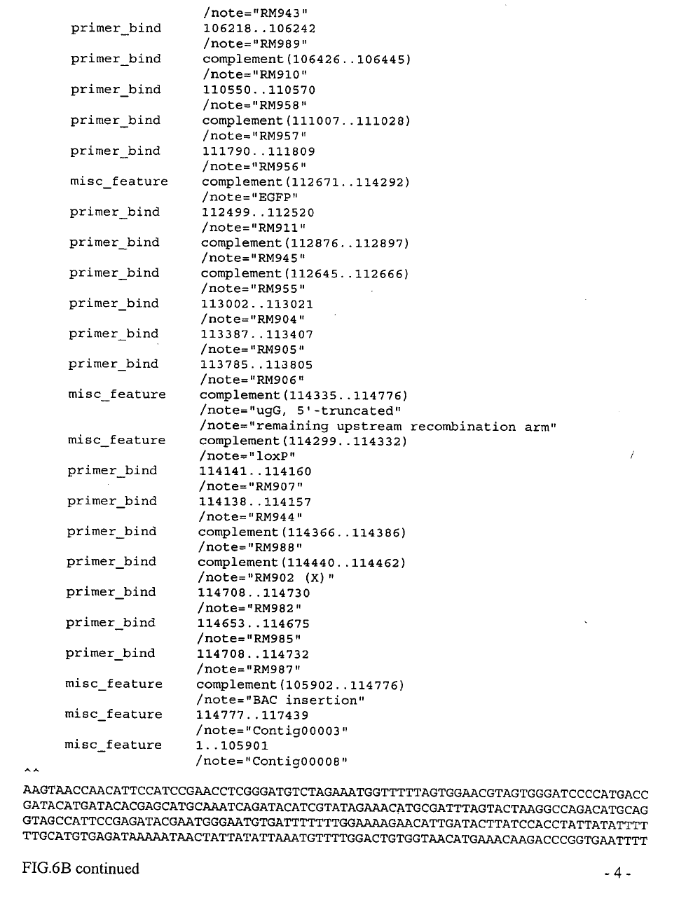

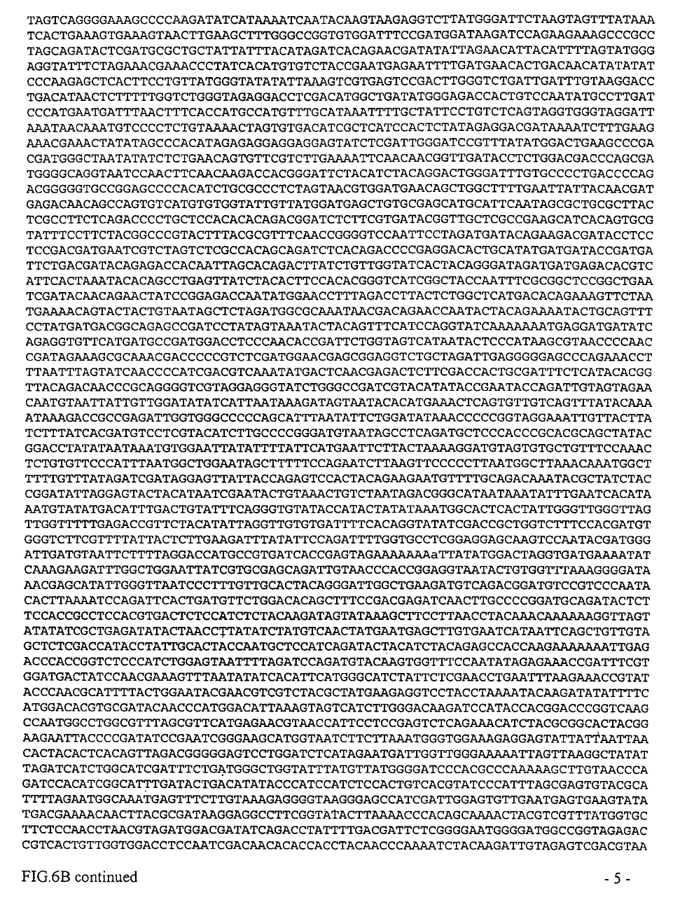

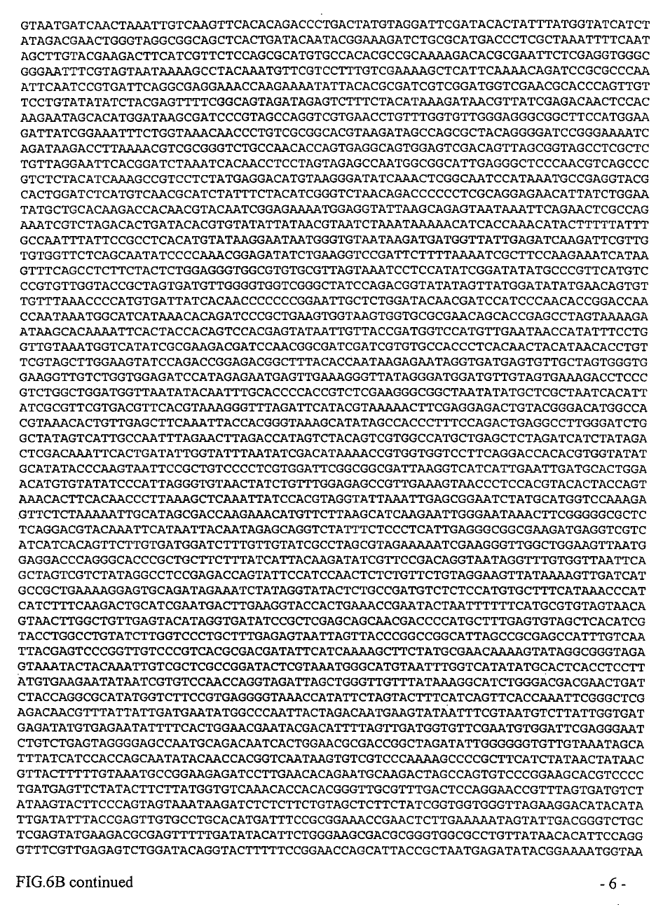

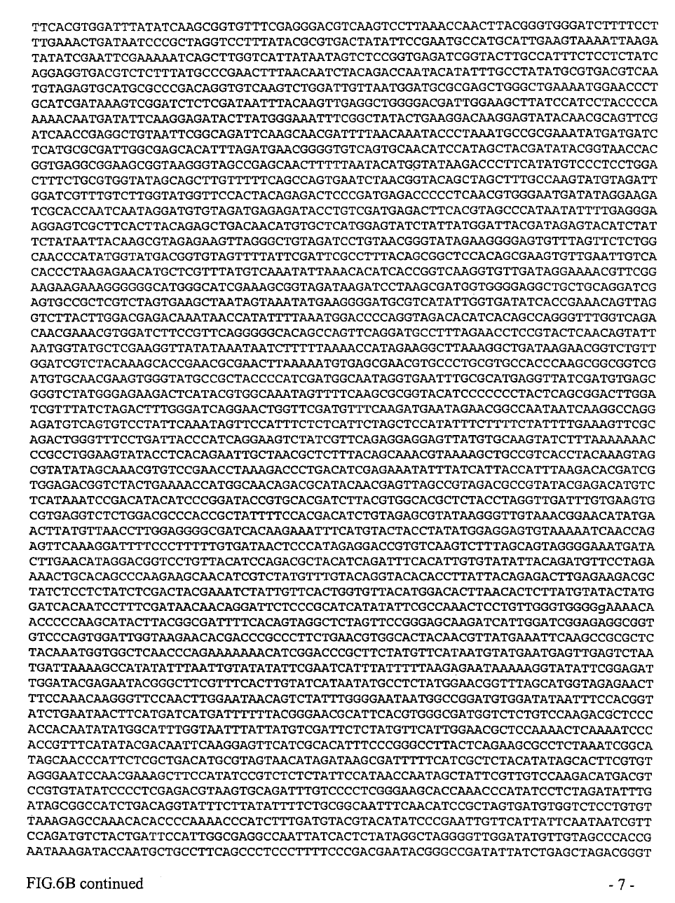

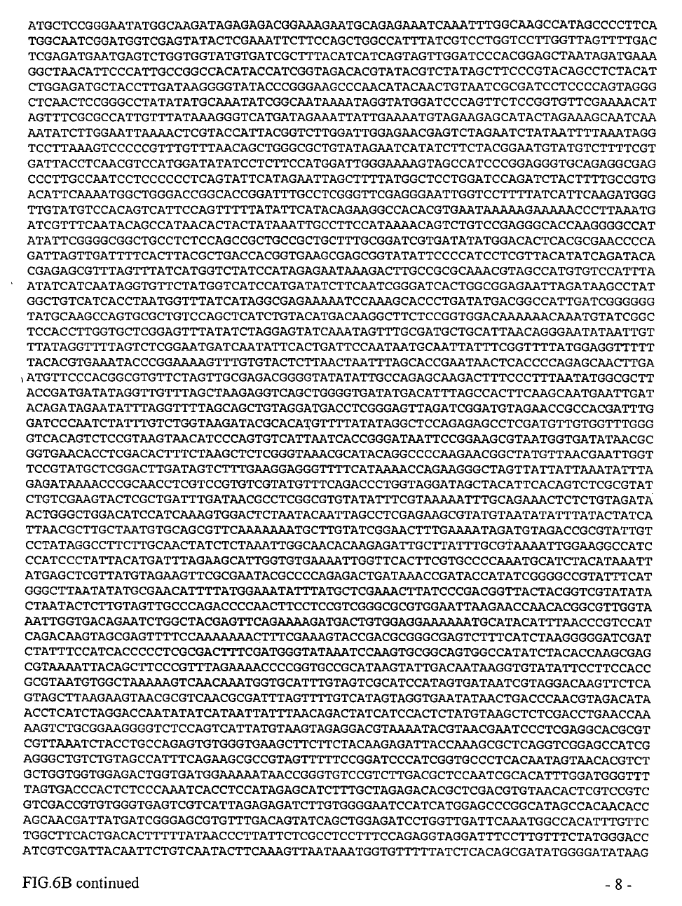

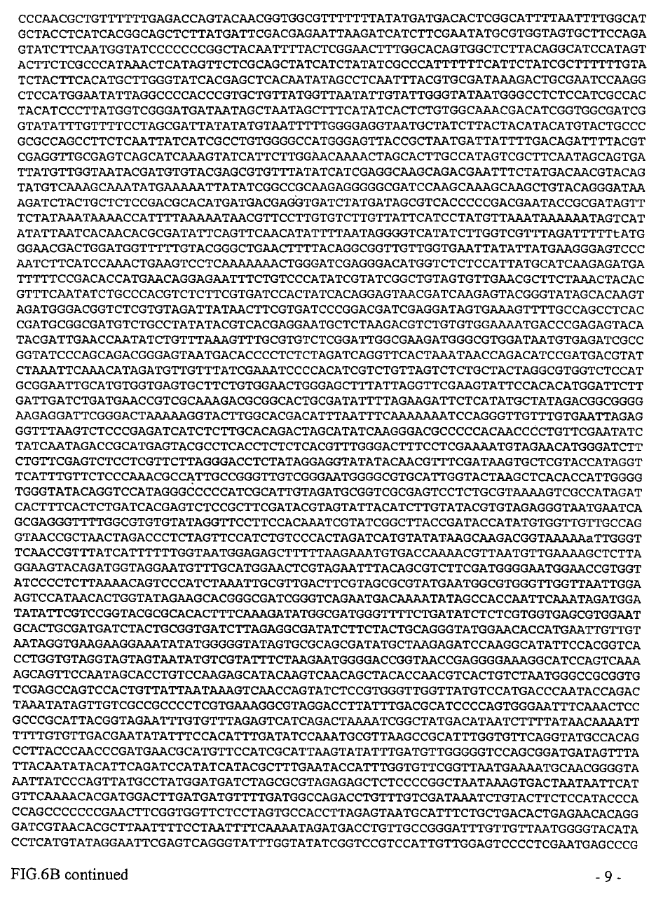

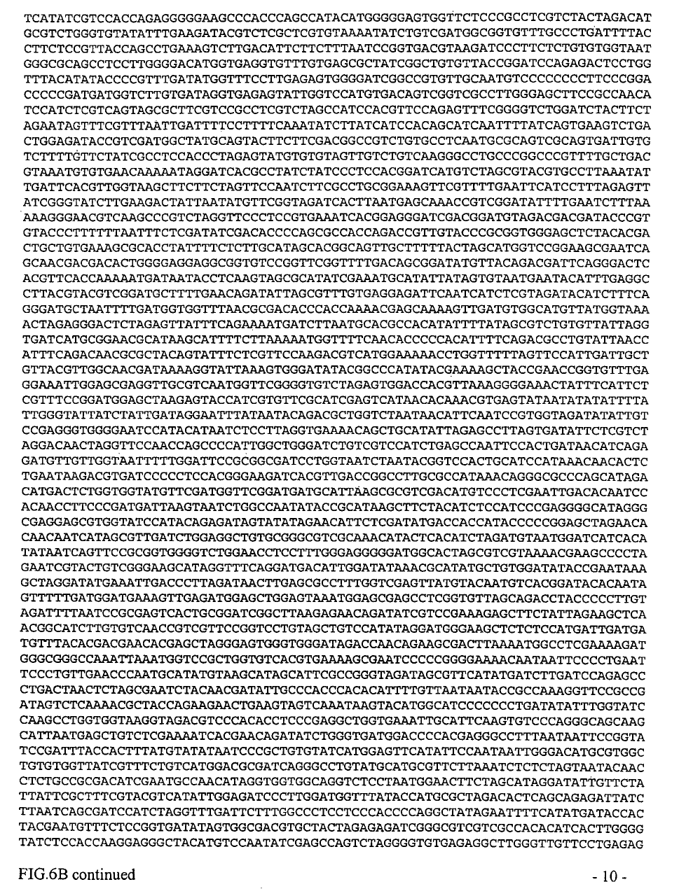

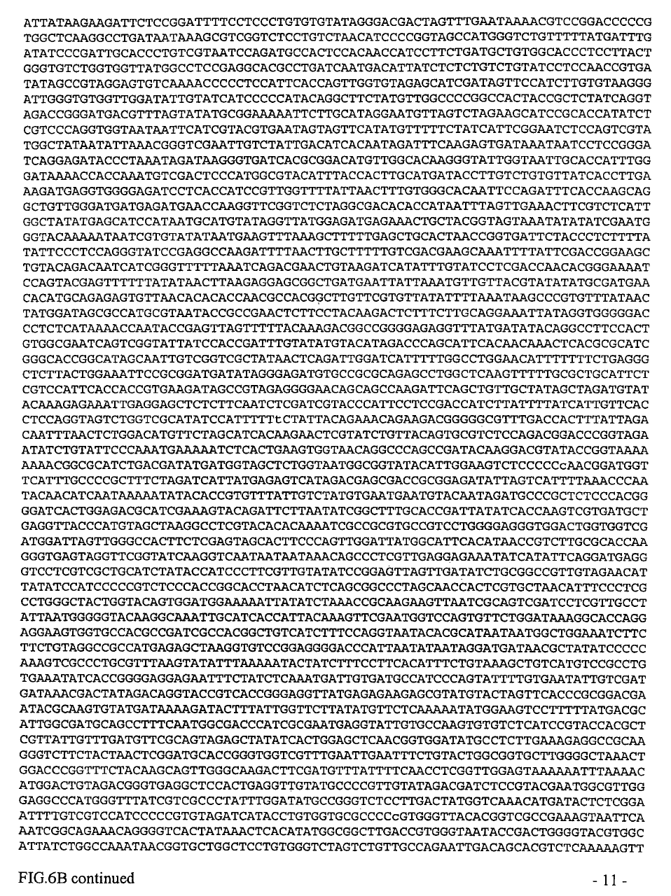

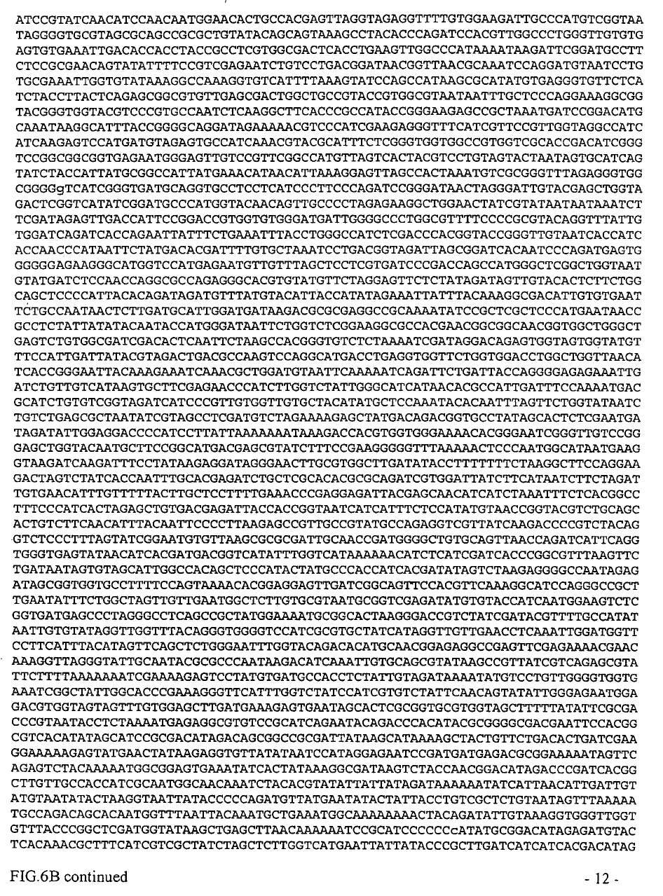

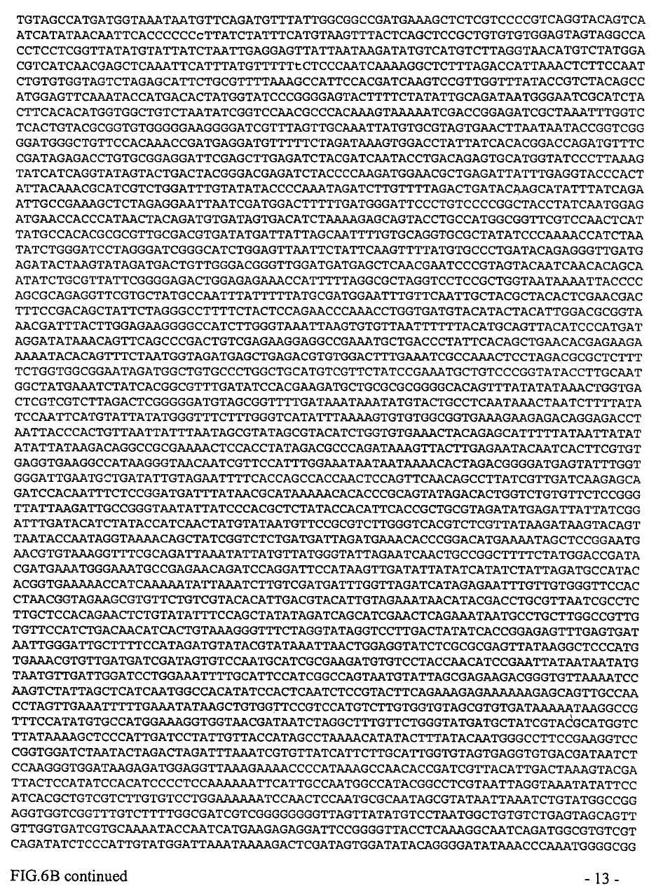

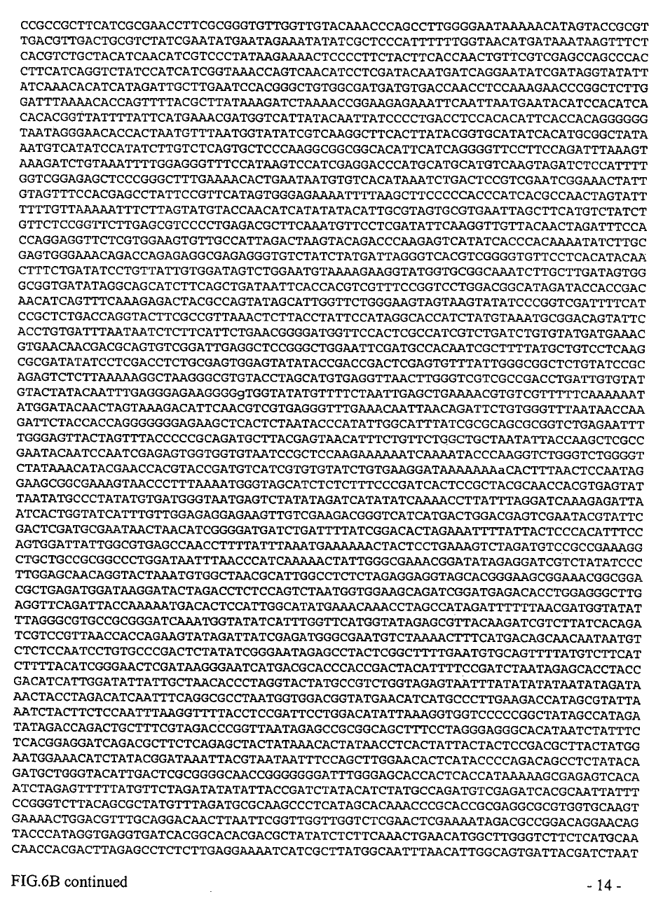

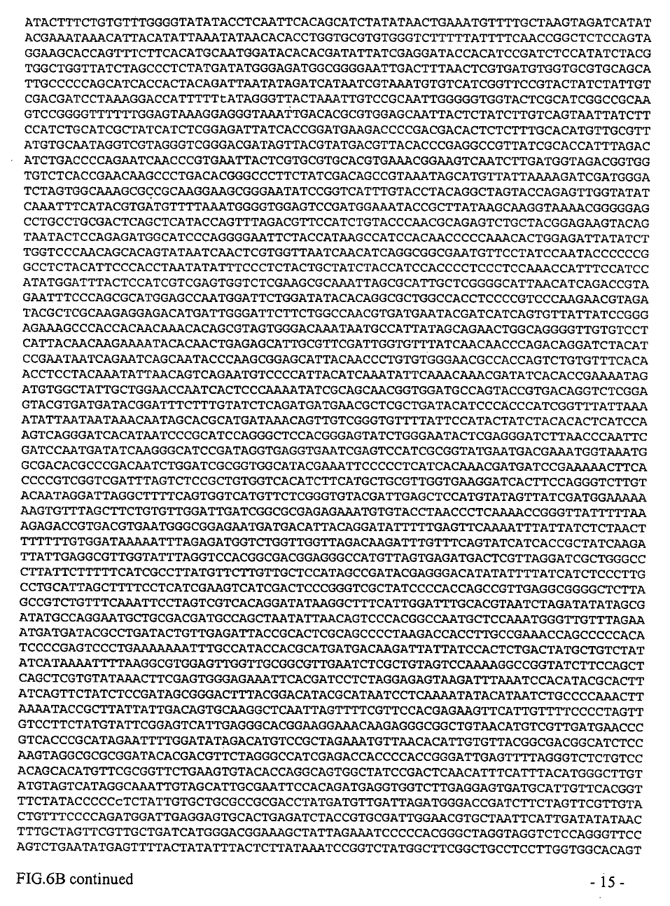

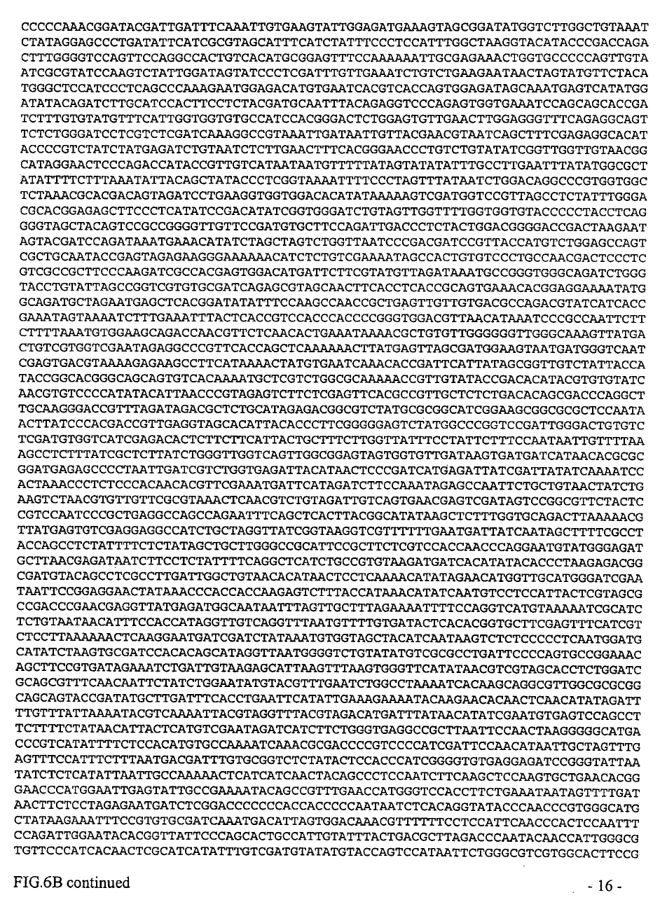

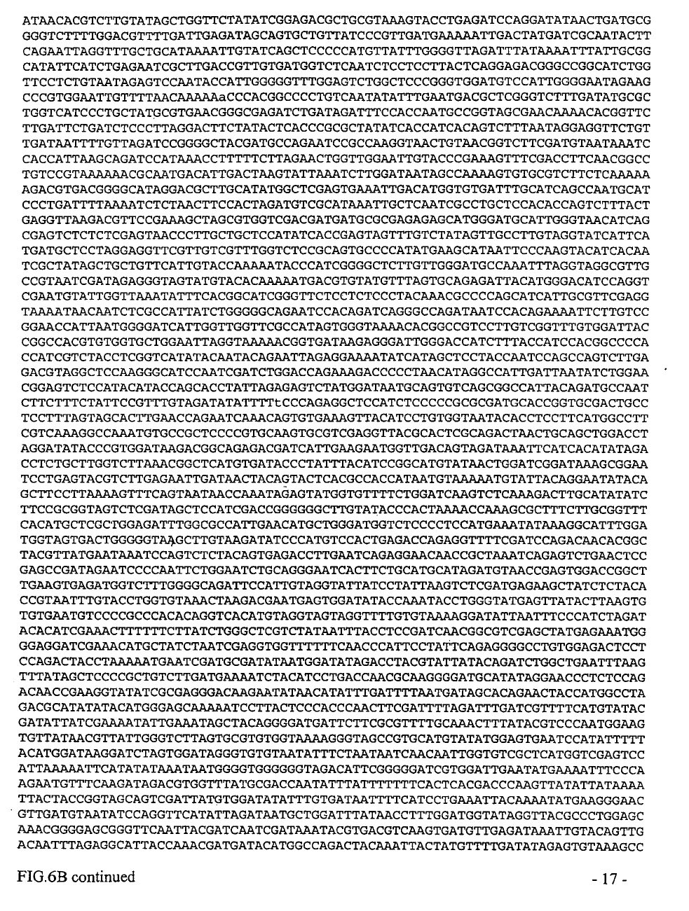

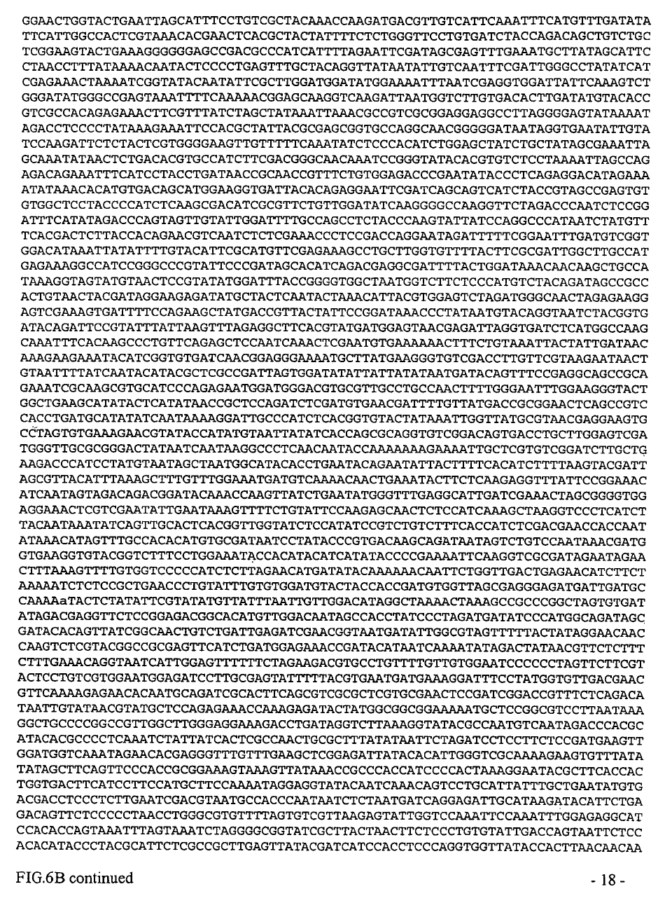

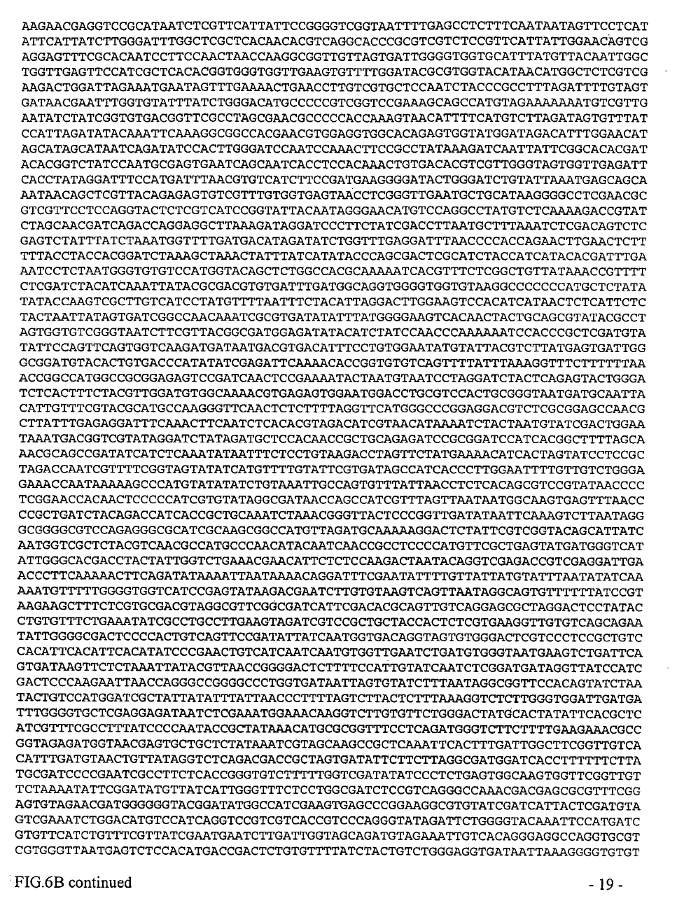

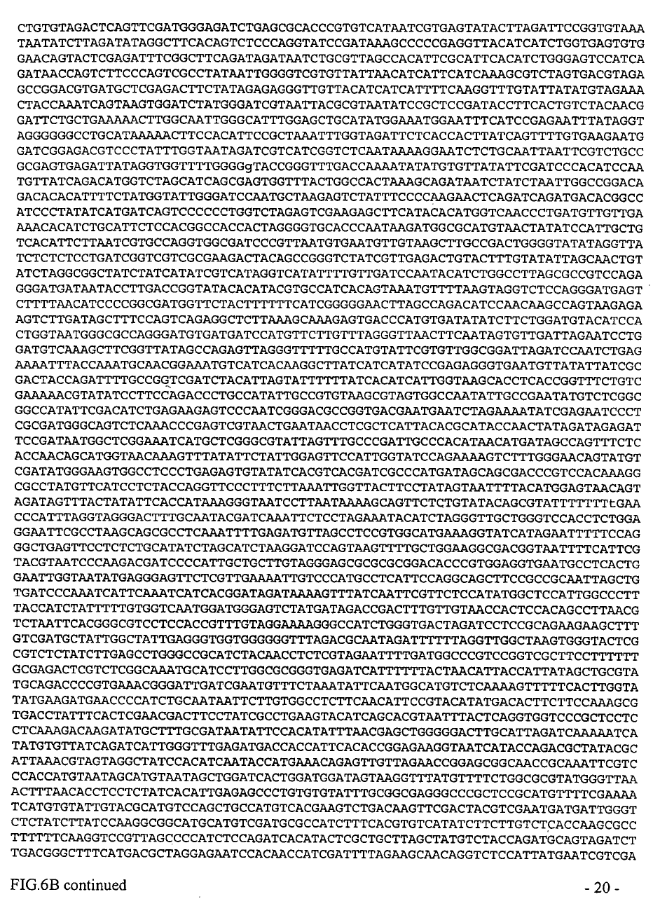

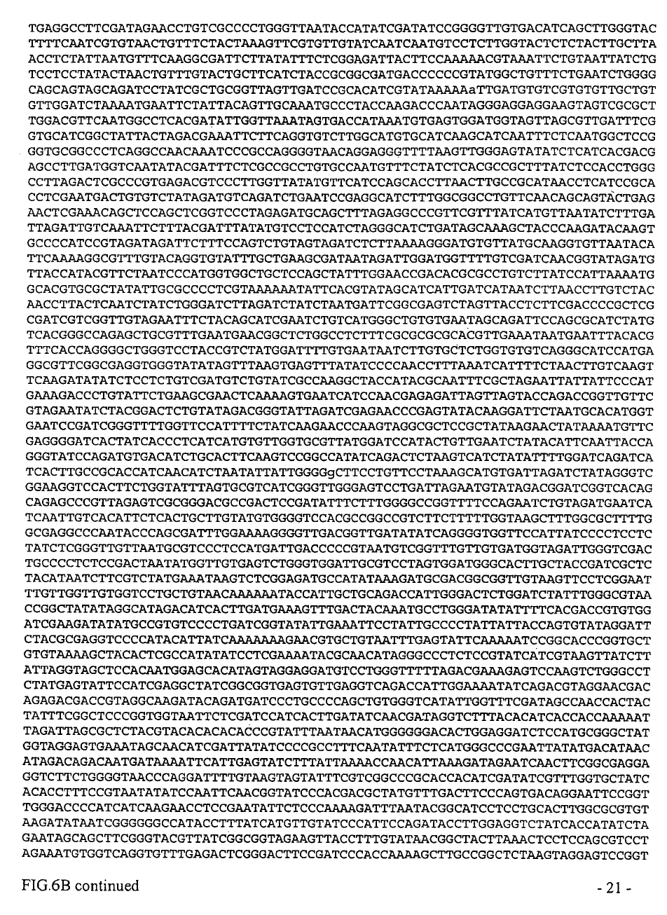

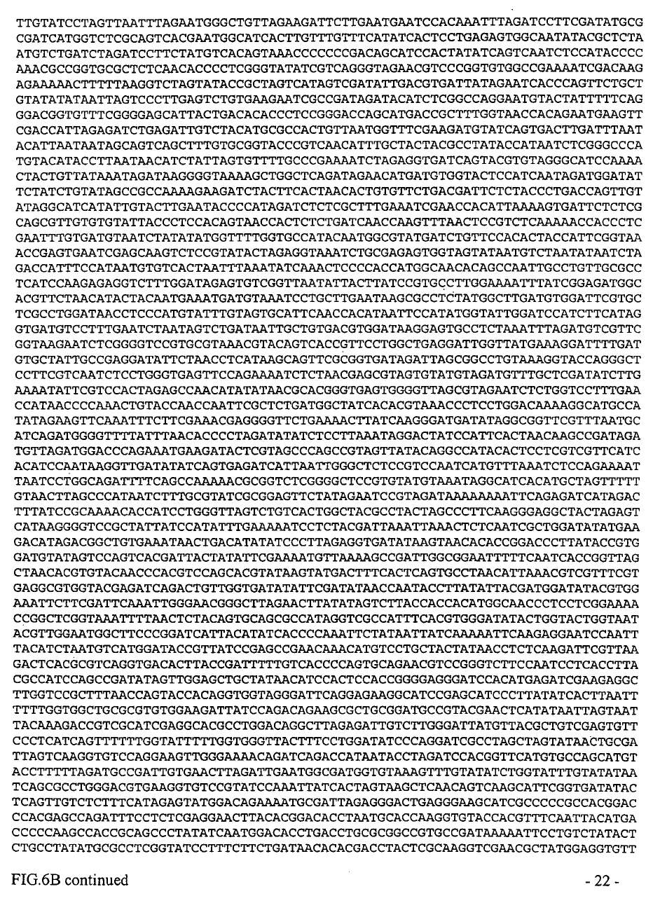

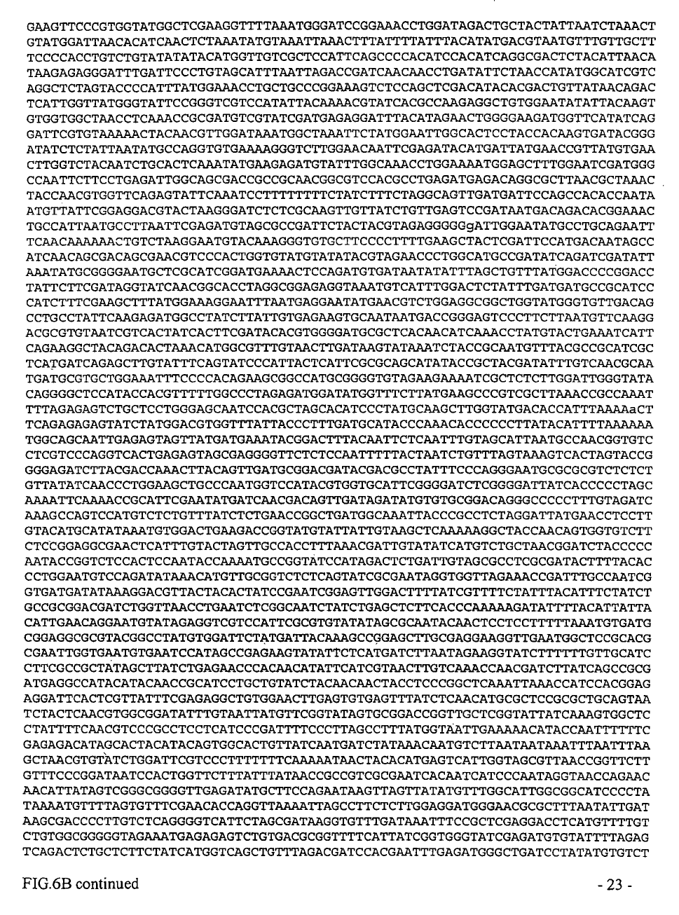

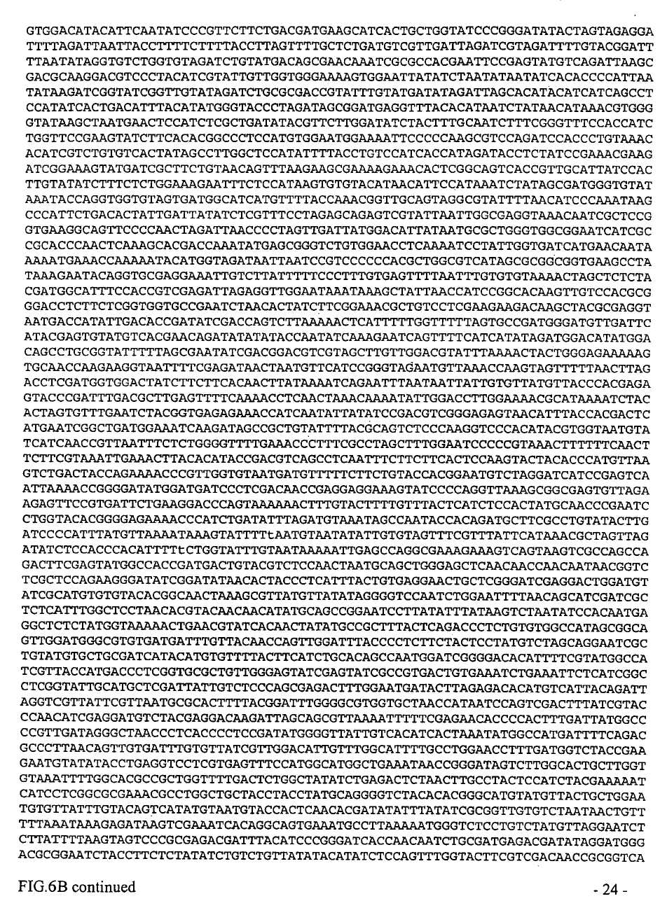

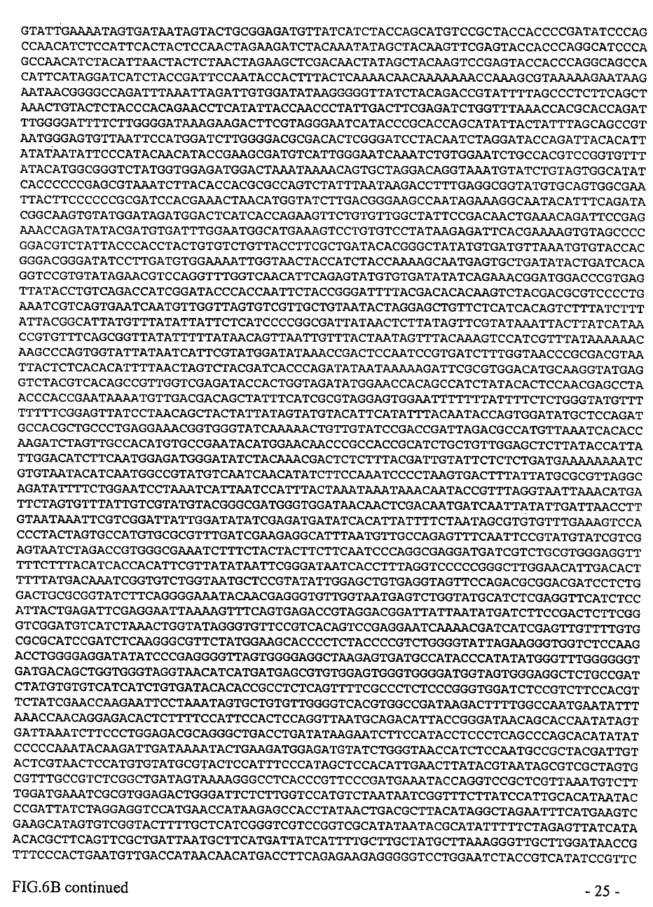

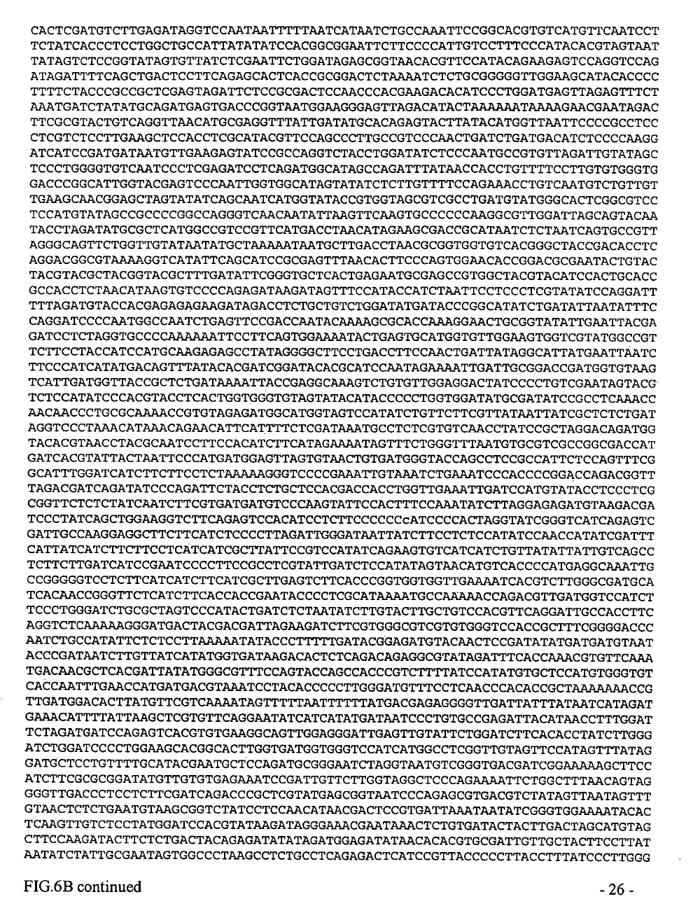

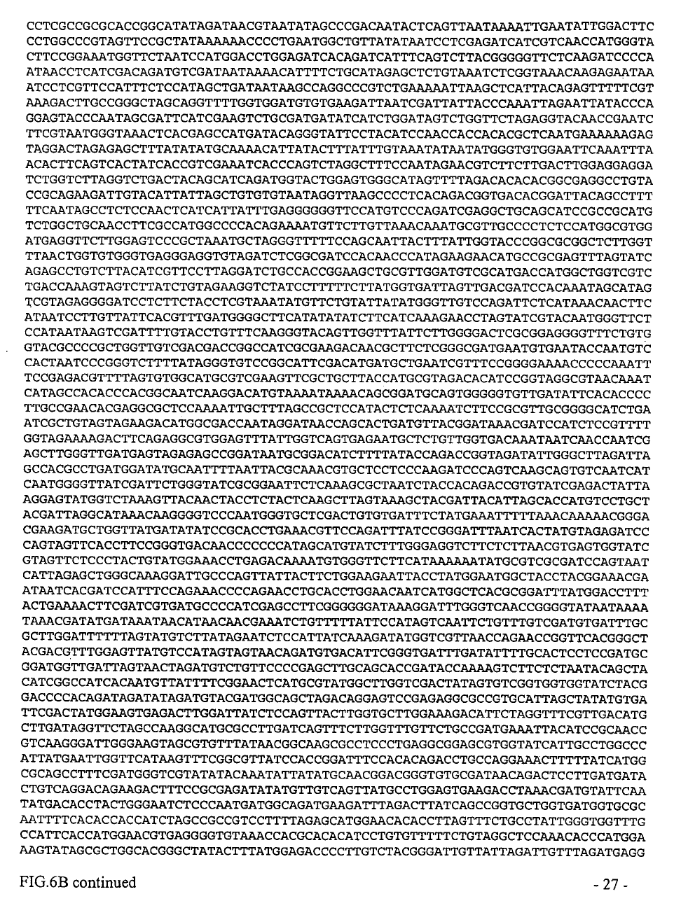

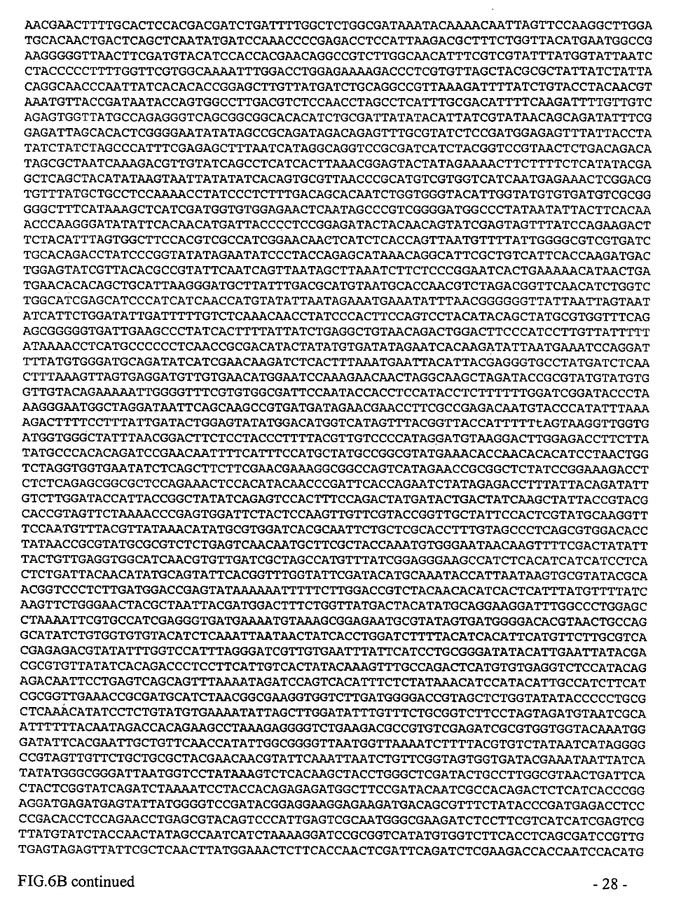

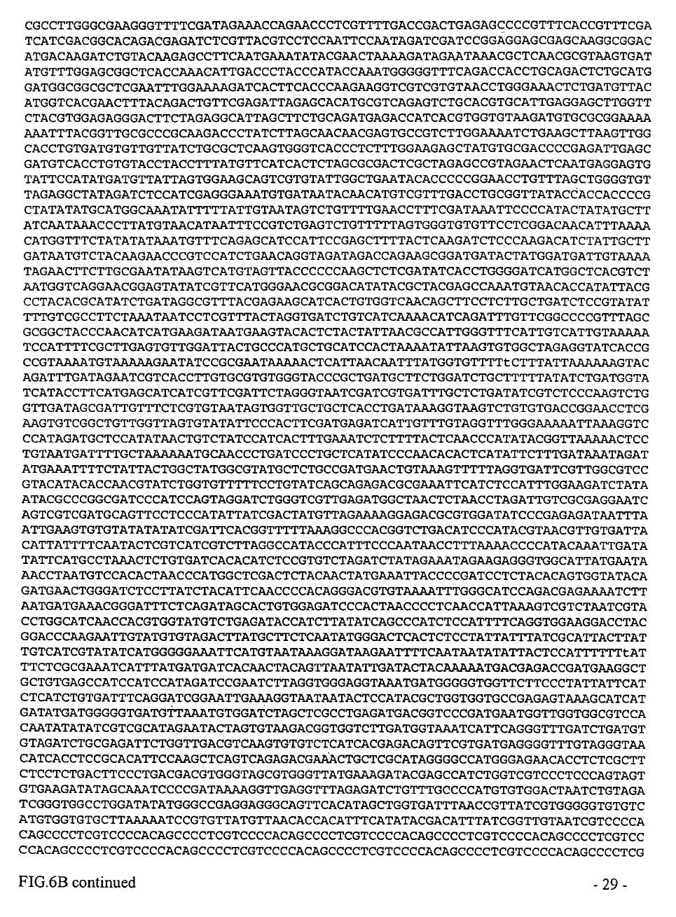

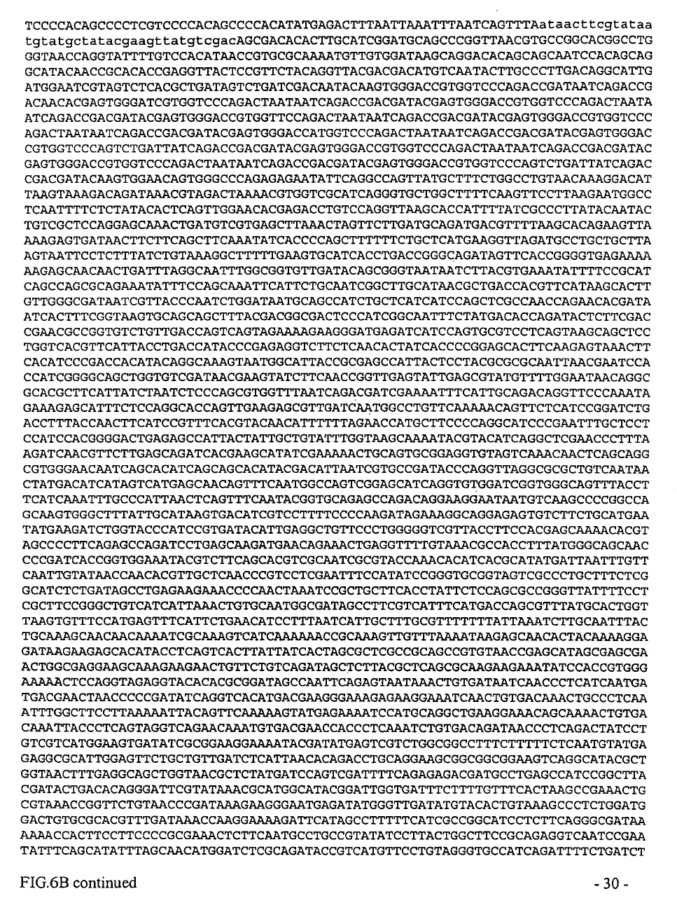

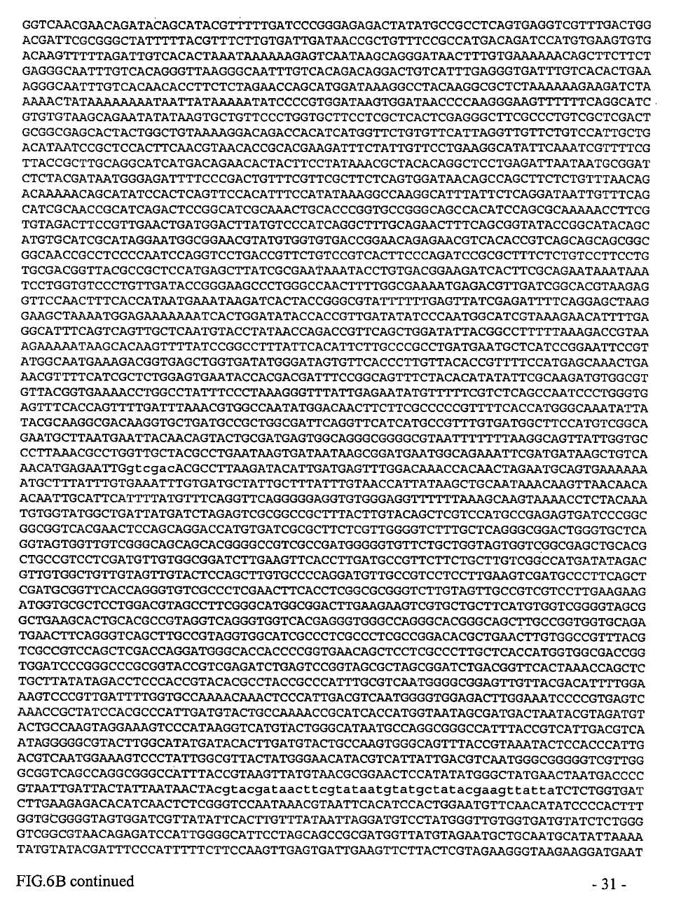

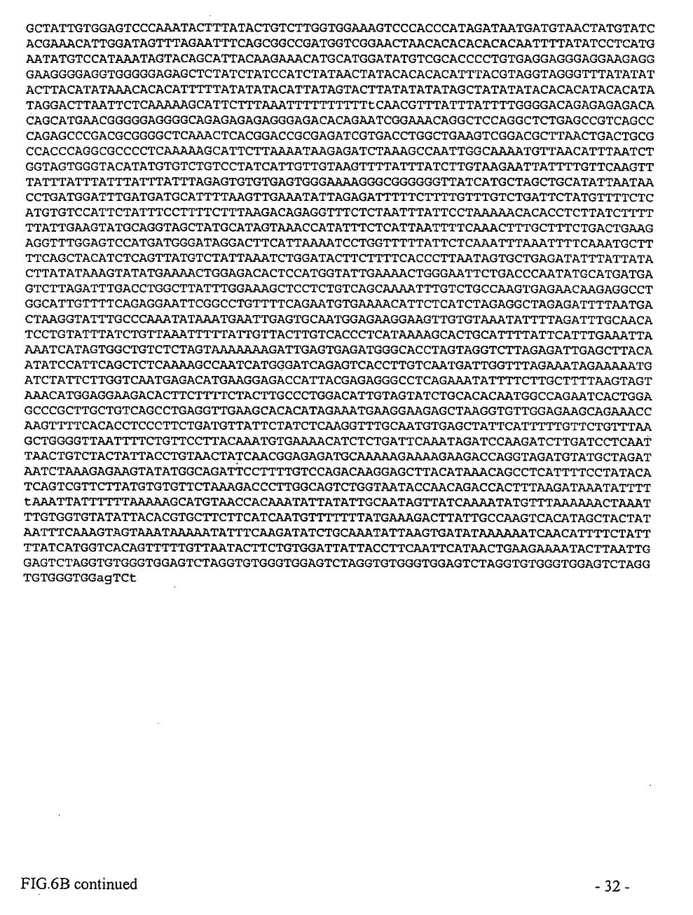

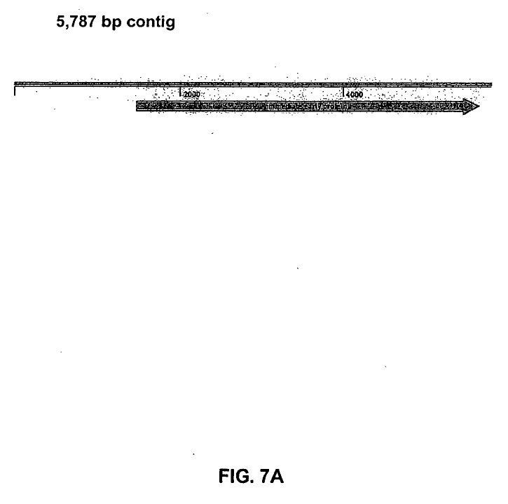

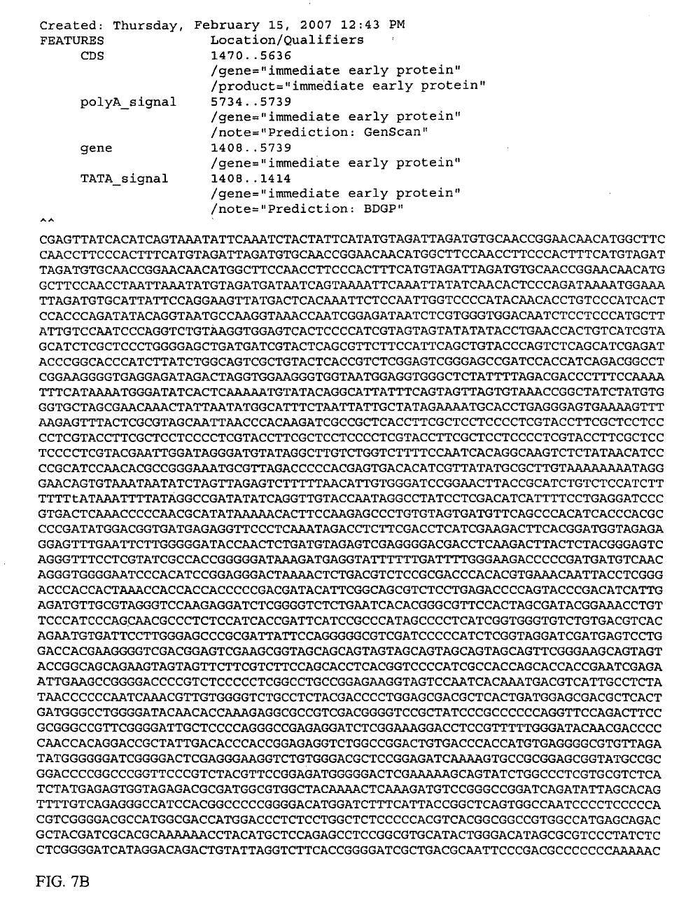

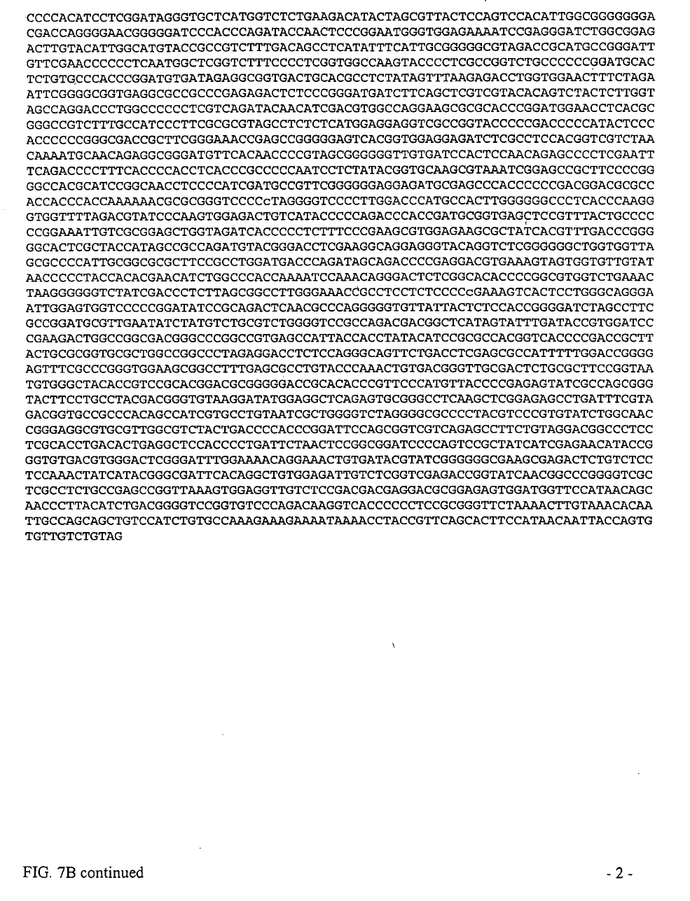

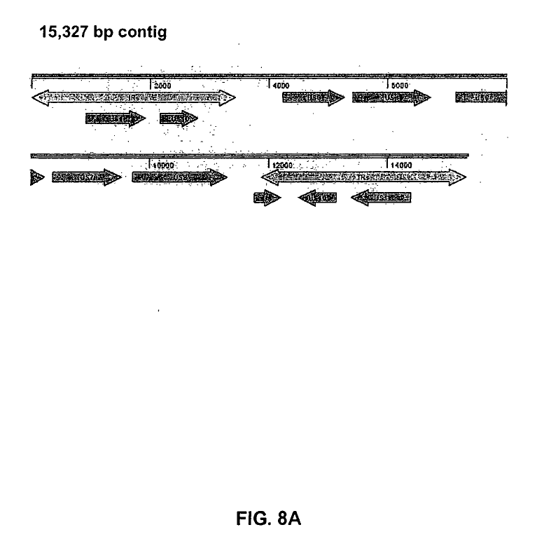

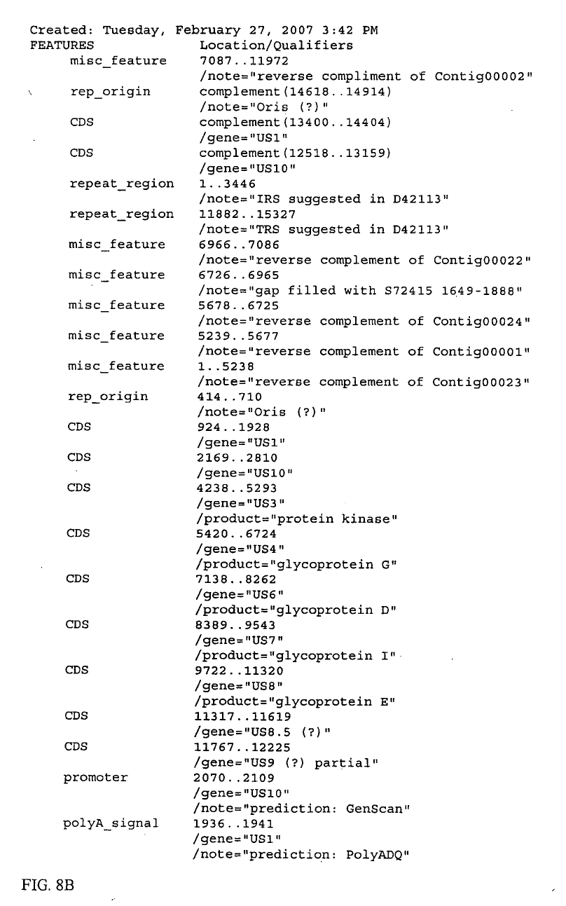

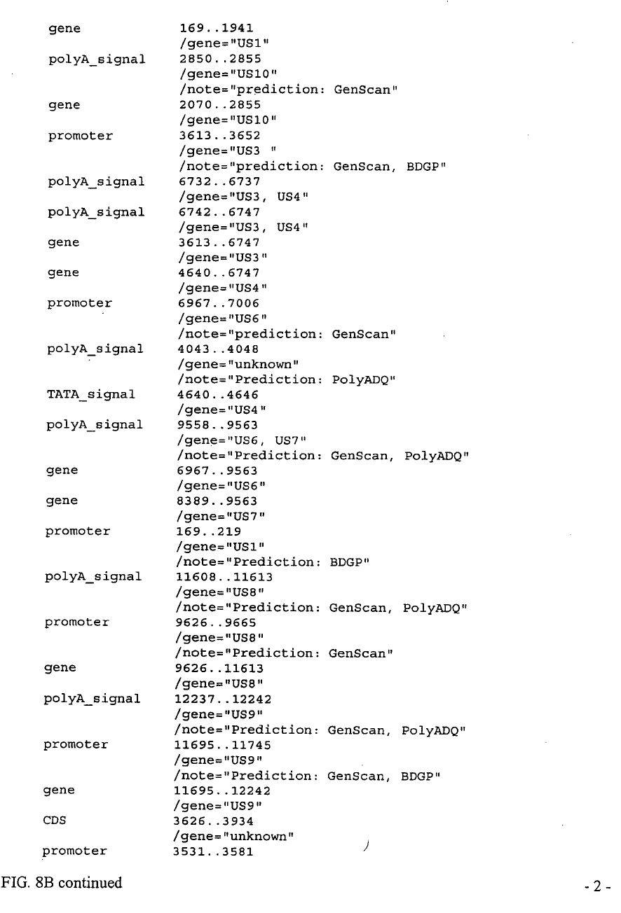

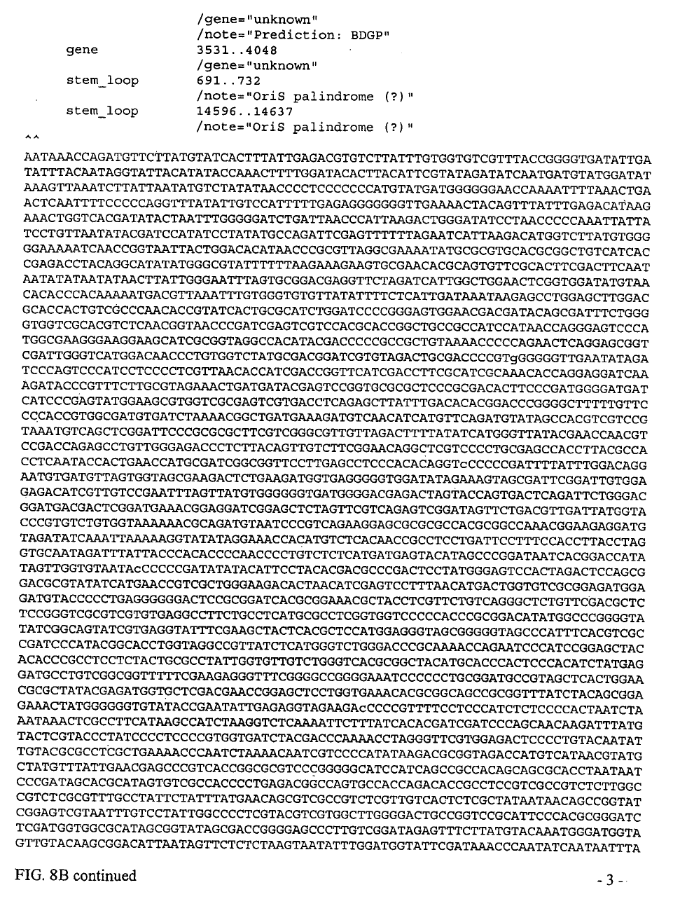

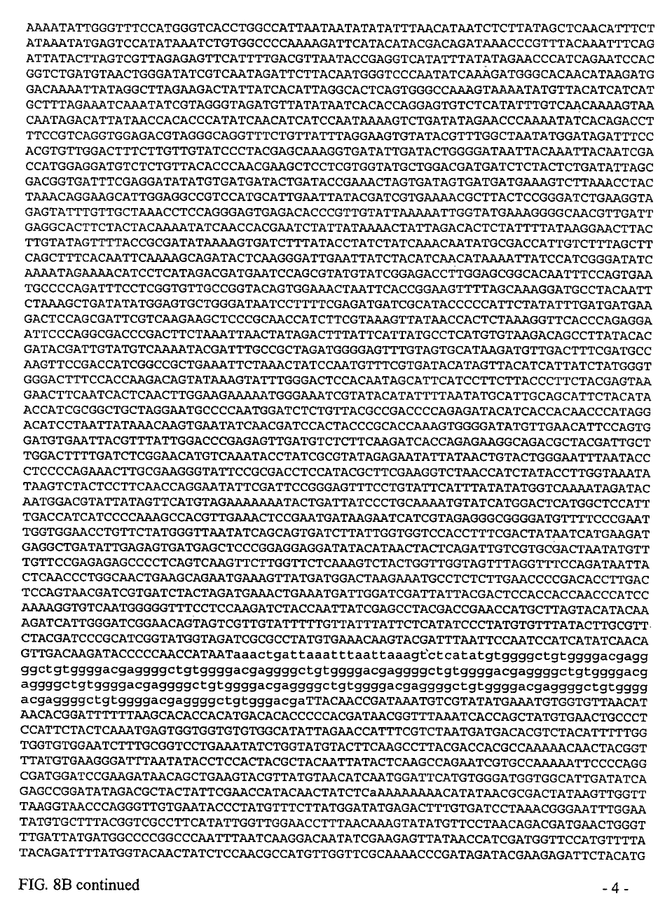

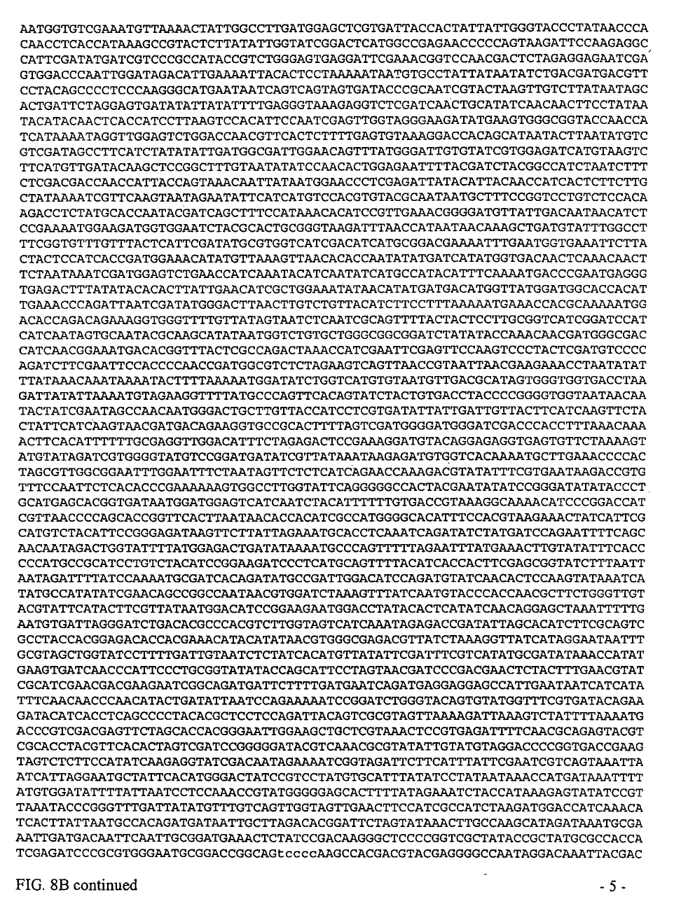

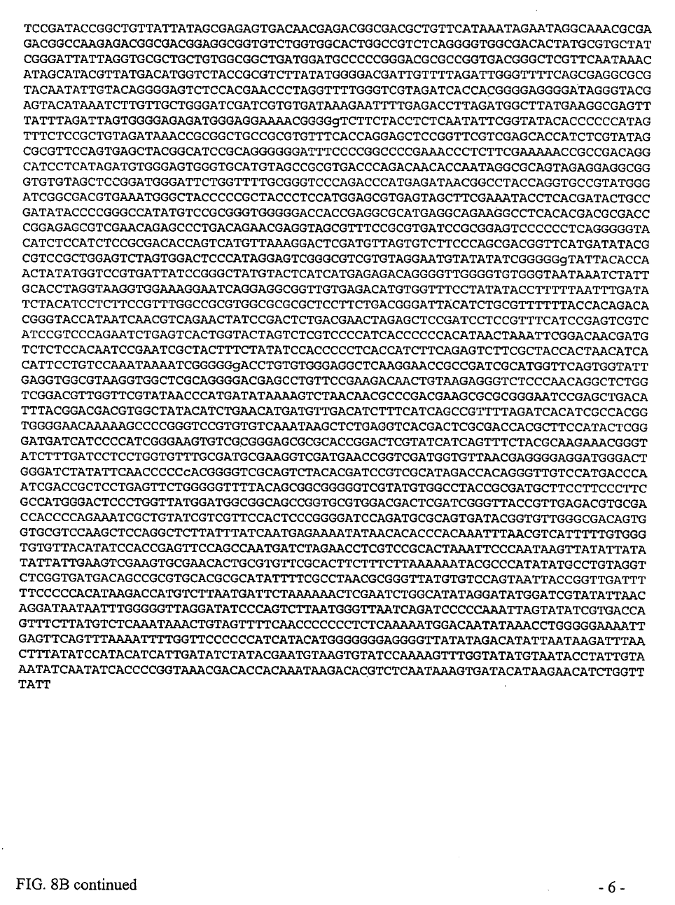

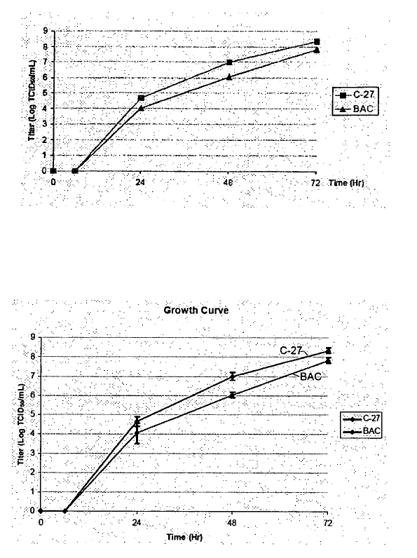

Sequencing of the FHV-1 BAC clone produced as described in the experimental examples has resulted in the first ever nearly complete (now totally complete) molecular description of an FHV-1 genome. As shown in FIGS. 6-8 and 13, the whole genome sequencing effort of the present invention allowed identification of FHV-1 genes, in part through comparison to sequences of related herpesviruses such as equine herpesvirus-1 (EHV-1), and pseudorabies virus (PRV). Possession of the FHV-1 sequence and an infectious FHV-1 BAC clone makes site-specific mutagenesis studies possible, and permits the characterization of FHV-1 genes that play a role in viral virulence, protective immunity and the induction, maintenance and reactivation phases of FHV-1 latency.

### IV. Utilities

In alphaherpesviruses, glycoprotein E (gE) and glycoprotein I (gI) form a heterodimer that functions in cell-to-cell spread of the virus and spread of infection throughout the host nervous system, causing neurovirulence. Generally, alphaherpesvirus mutants that lack these glycoproteins are replication-competent in cell culture but produce smaller plaques, due to reduced capacity for cell-to-cell spread. gI/gE deletion mutants of type 1 herpes simplex virus (HSV-1) and other alphaherpesviruses such as pseudorabies virus (PRV or Suid herpesvirus 1) and bovine herpesvirus 1 (BHV-1) show impaired ability to spread from cell-to-cell (Balan et al., J Gen Virol, 75:1245-1258, 1994; Dingwell et al., J Virol, 68:834-845, 1994; Dingwell et al., J Virol, 72:8933-8942, 1998; Otsuka and Xuan, Arch Virol, 141:57-71, 1996; and Zuckermann et al., J Virol, 62:4622-4626, 1988). The gI/gE heterodimer appears to play an even greater role in the spread of varicella-zoster virus (Cohen and Nguyen, J Virol, 71:6913-6920, 1997; Mallory et al., J Infect Dis, 178suppl1:S22-S26, 1998; and Mallory et al., J Virol, 71:8279-8288, 1997) and in Marek's disease virus serotype 1 (MDV-1 or Gallid herpesvirus 2), in which the gI/gE heterodimer has been found to be essential for growth in cultured cells (Schumacher et al., J Virol, 75:11037-11318, 2001). In the case of FHV-1, previous studies have shown that gI/gE is not essential for virus growth, although virulence is significantly reduced and the virus produces smaller plaques when gI/gE is deleted (Sussman et al., Virology, 214:12-20, 1995). The FHV-1 gI/gE mutant, when administered via oronasal route, can protect cats from developing clinical signs of infection, and significantly reduces viral loads in subsequent challenge with a high dose of a field virus (Kruger et al., Virology, 220:299-308, 1996; and Sussman et al., Virology, 228:379-382, 1997). However, this mutant at higher dose levels still retained partial virulence, producing clinical signs in inoculated cats.

To produce an FHV-1 vaccine with greater safety and efficacy, other viral genes of the FHV-1 BAC clone are modified. Glycoprotein G (gG) homologues have been described in several alphaherpesviruses as a minor non-essential glycoprotein (Baranowski et al., Vet Microbiol, 53:91-101, 1996). Based on the viral species, gG has been reported either as a structural or a non-structural protein. The protein encoded by FHV-1 gG gene exists under two different forms, a membrane-anchored form and a secreted form. The latter is generated by proteolytic cleavage of the former (Drummer et al., J Gen Virol, 79:1205-1213, 1998). Recently FHV-1 gG was found to belong to a newly discovered viral chemokine-binding protein (vCKBP) family, binding with high affinity to a broad spectrum of chemokines. Both the secreted form and the membrane-anchored form of gG expressed at the surface of virus-infected cells can bind chemokines. Furthermore, other vaccine preparations have made recombinant FHV by inserting foreign DNA into genomic fragments of the FHV gene (Cochran et al., U.S. Pat. No. 6,410,311, 2002, Sondermeijer et al., U.S. Pat. No. 6,521,236, 2003. See also, the following patents regarding insertion of foreign DNA into sequenced fragments of FHV-1 genome for generation of vaccines and other immunological compositions: U.S. Pat. Nos. 6,387,376; 6,074,649; 5,833,993; 5,783,192; and 5,652,119.) In addition, the expression of a secreted vCKBP activity is a general property of field FHV-1 strains (Costes et al., Microbes Infect, 8:2657-2667, 2006). Although gG is not essential for virus growth, several gG mutants of alphaherpesviruses have been shown to attenuate virulence of porcine PRV (Demmin et al., J Virol, 75:10856-10869, 2001), avian infectious laryngotracheitis virus (Devlin et al., Vaccine, 25:3561-3566, 2007), and equine herpesvirus-1 and -4 (Huang et al., Arch Virol, 150:2583-2592, 2005).

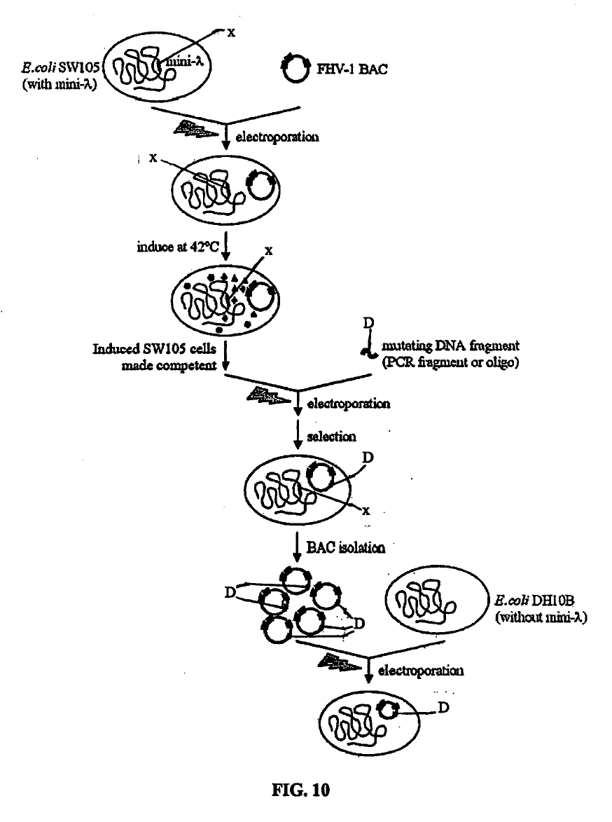

The FHV-1 BAC clone produced during development of the present invention, is used to produce recombinant FHV-1 bearing both gE and gG deletions. The gE/gG double mutant is contemplated to be a highly attenuated virus, thus providing recombinant viruses with greater safety for use in veterinary medicine. Moreover, the present invention is also used to produce recombinant FHV-1 bearing both gC and gE deletions. Furthermore, the gC/gE double mutant is contemplated to be a highly attenuated virus, thus also providing recombinant viruses with greater safety for use in veterinary medicine. Bacterial artificial chromosome (BAC) cloning and recombination-mediated genetic engineering (recombineering) are two state-of-the-art techniques to facilitate site-directed mutagenesis, as shown in FIG. 10. Recombineering is a powerful method for fast and efficient manipulation of BACs. It allows DNA cloned in E. coli to be modified via lambda (λ) Red-mediated homologous recombination, obviating the need for restriction enzymes and DNA ligases. Specific bacterial strains have been constructed for this purpose (Lee et al., Genomics, 73:56-65, 2001; Warming et al., Nucleic Acids Res, 33:e36, 2005; and Yu et al., Proc Natl Acad Sci USA, 97:5978-5983, 2000). A defective λ prophage (mini-λ), which encodes three genes that make recombineering possible: exo, bet and gam, is inserted into their bacterial genome. Exo is a 5′-3′ exonuclease that creates single-stranded overhangs on linear DNA introduced into the bacteria. Bet protects these overhangs and assists in the subsequent recombination process. Gam prevents degradation of linear DNA by inhibiting E. coli RecBCD protein. Exo, bet, and gam are transcribed from the λPL promoter. This promoter is repressed by the temperature-sensitive repressor c1857 at 32° C. and derepressed (the repressor is inactive) at 42° C. When bacteria containing mini-λ prophage (designated x in FIG. 10) are kept at 32° C., no recombination proteins are produced. However, after 15 minutes of heat shock at 42° C., sufficient amounts of recombination proteins are produced. Linear DNA (PCR product, oligonucleotide, etc.; designated D in FIG. 10) with sufficient homology in the 5′ and 3′ ends to a target DNA molecule already present in the bacteria (plasmid, BAC, or the bacterial genome itself) can be introduced into heat-shocked and electrocompetent bacteria using electroporation. The introduced DNA is modified by exo and bet and undergoes homologous recombination with the target molecule.

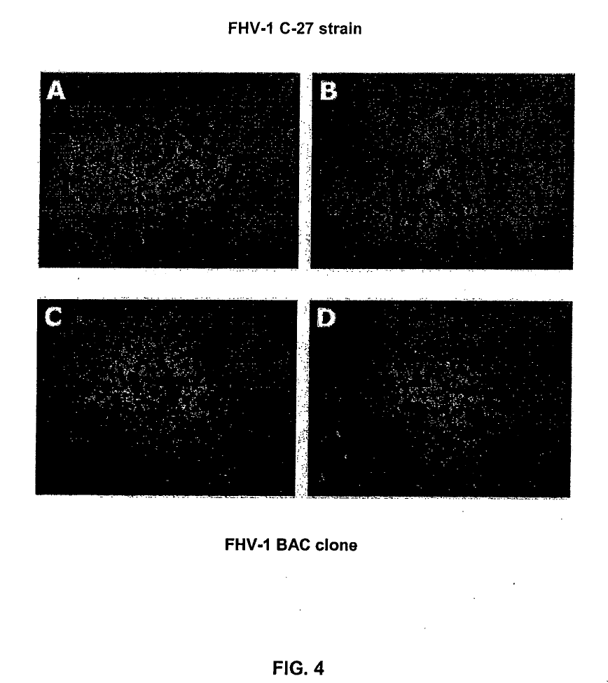

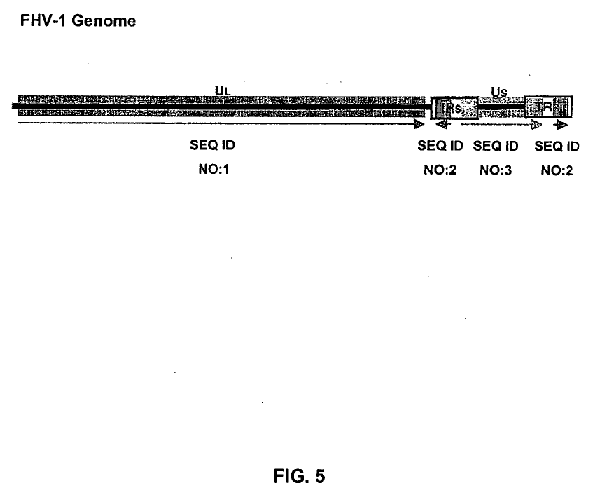

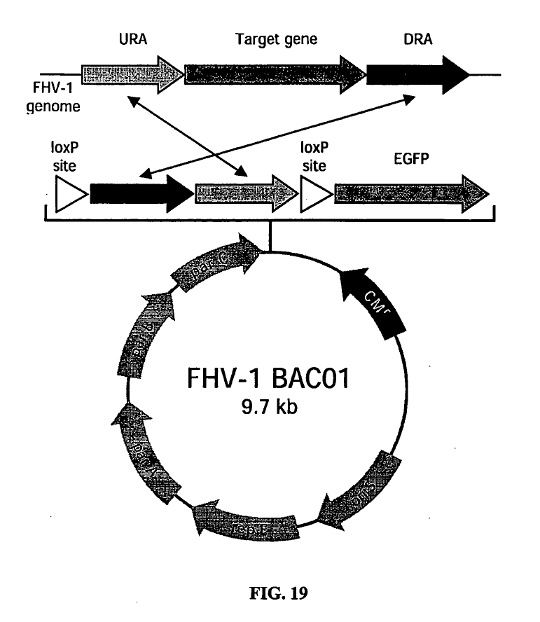

As described herein for FHV-1, once a viral genome is cloned into a BAC, it can be easily and efficiently manipulated within E. coli. Utilizing recombineering techniques, site-specific mutations can be introduced anywhere in the viral genome, provided that the genomic sequence is known. All mutagenesis steps can be strictly controlled and analyzed in E. coli, and the manipulated viral genome can be stably maintained in the E. coli. This is in contrast to other methods, where the recombination takes place in eukaryotic cells and the analyses can only start after the virus has been reconstituted and isolated. Unwanted additional changes that may have occurred in the viral genome, such as deletions, rearrangements or illegitimate recombinations frequently can only be observed after considerable time and effort. Using previous CAF funding, we have successfully cloned the whole FHV-1 genome into a BAC. Moreover, from this BAC we have derived almost the complete sequence so far, covering ˜95% of the estimated 134 kb, including the entire U_{L} and Us, as well as parts of the IRs and TRs (FIG. 1). Sixty-four genes were identified to be homologs of other closely related alphaherpesvirus genes. In further studies we have now derived the complete sequence of the FHV-1 genome (FIG. 19), and seventy-four distinct genes were identified to be homologs of other closely related alphaherpesvirus genes. The BAC vector was found to be inserted between U_{L} and IRs instead of replacing gG as initially planned. Nonetheless, after removal of the BAC vector through loxP recombination, the cloned virus produces morphologically similar plaques and can grow to a titer as high as wild type virus (10₇ TCID₅₀/mL), and is fully virulent in vivo, suggesting that this clone is very similar to the wild type in vitro, and in vivo and thus is suitable as a basis of mutagenesis, as shown in FIG. 4. With the knowledge of the genomic sequence and the FHV-1 BAC construct, we can now introduce deletions or point mutations anywhere in the FHV-1 genome in a fairly straightforward manner.

Further, as described herein PCR, Southern hybridization, and Northern hybridization techniques involves selection of “stringency” in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). Thus, it is believed that a polynucleotide at least 100 kb in length that hybridizes under highly stringent conditions to the nucleotide sequence set forth in SEQ ID NO:1. or a polynucleotide at least 5 kb in length that hybridizes under highly stringent conditions to the nucleotide sequence set forth in SEQ ID NO:2. or a polynucleotide at least 5 kb in length that hybridizes under highly stringent conditions to the nucleotide sequence set forth in SEQ ID NO:3, or a polynucleotide at least 135 kb in length that hybridizes under highly stringent conditions to the nucleotide sequence set forth in SEQ ID NO:4 or fragments thereof under highly stringent conditions or a polynucleotide that hybridizes under highly stringent conditions to the nucleotide sequence preferably with at least 90% similarity, more preferably at least 95% similarity, and most preferred 100% similarity to SEQ ID NO:1, 2, 3, or 4 might be used for generation of FHV-1 compositions and agents as herein described.

### V. Discussion of Results for Sequencing the Complete FHV-1 Genome

A. BAC Cloning

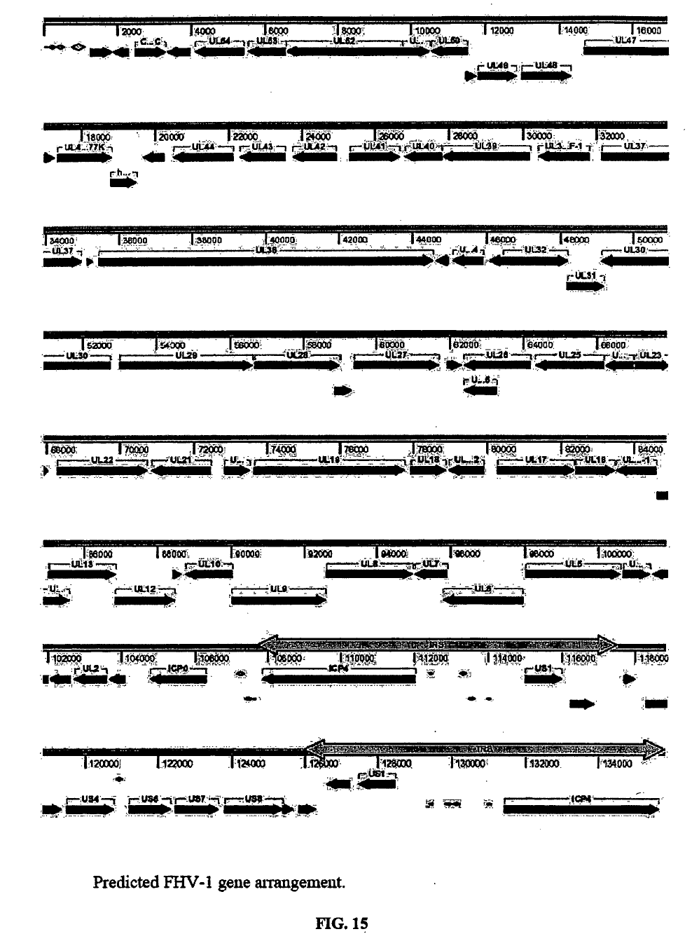

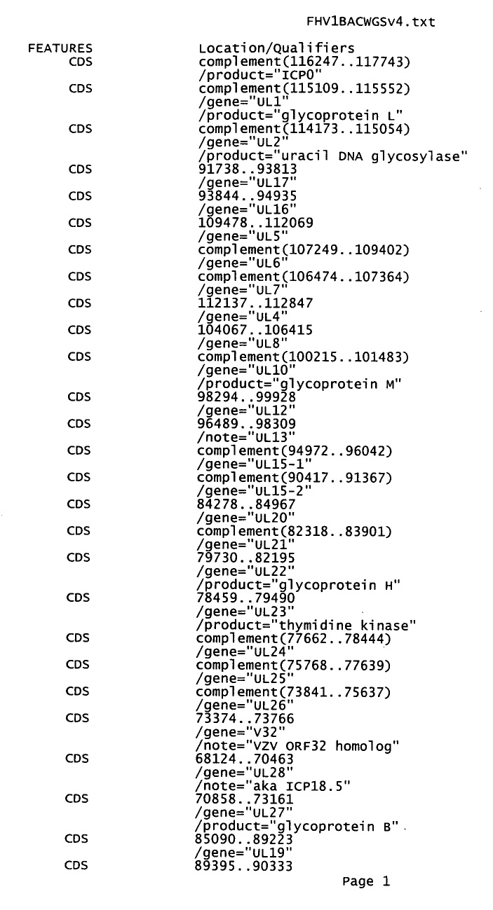

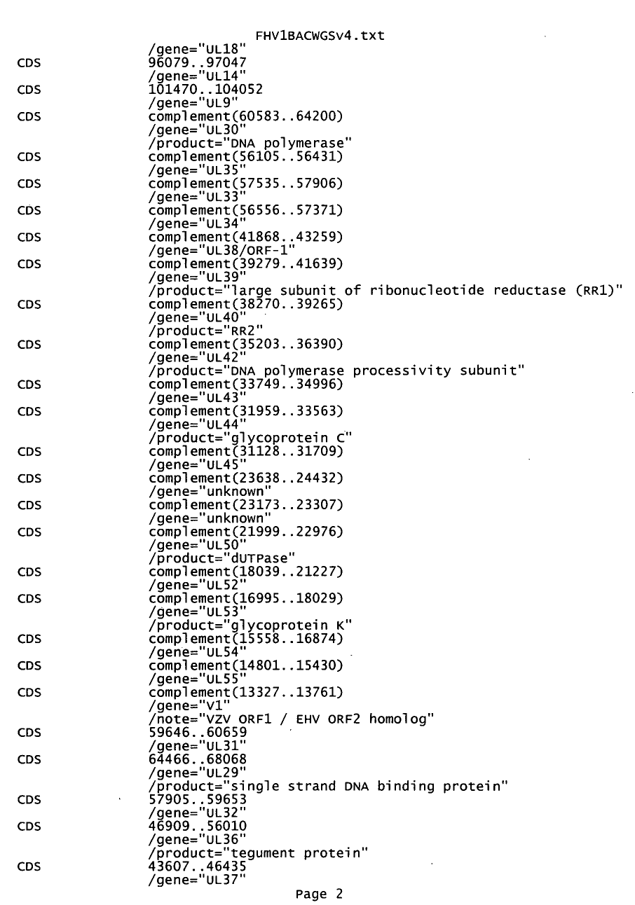

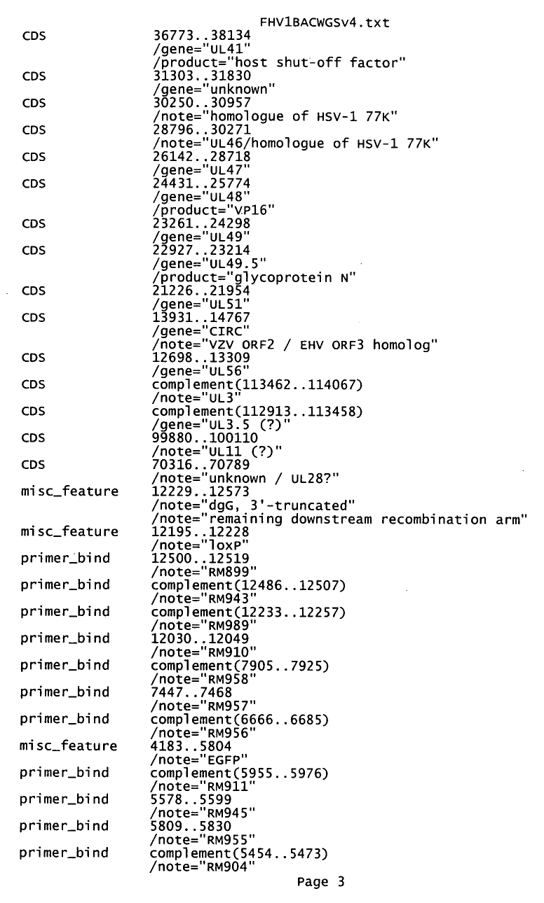

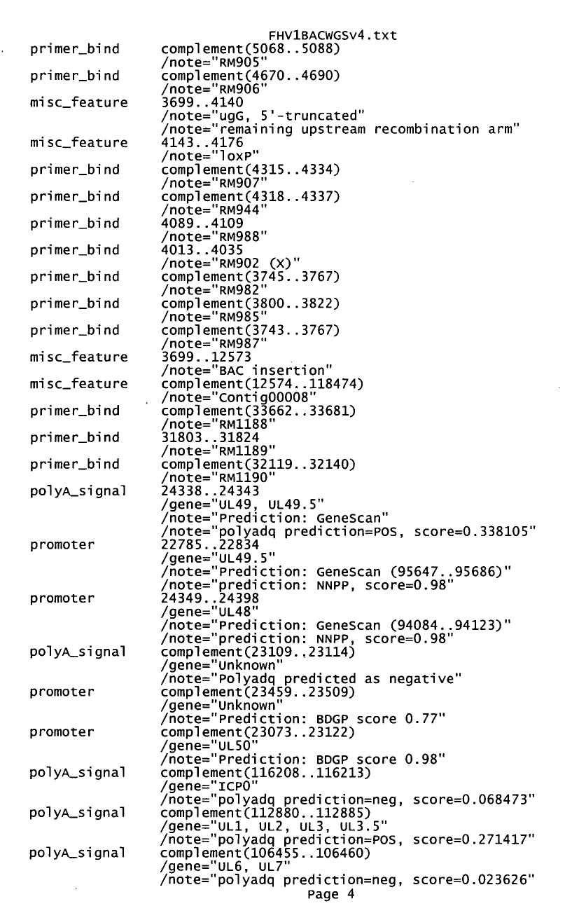

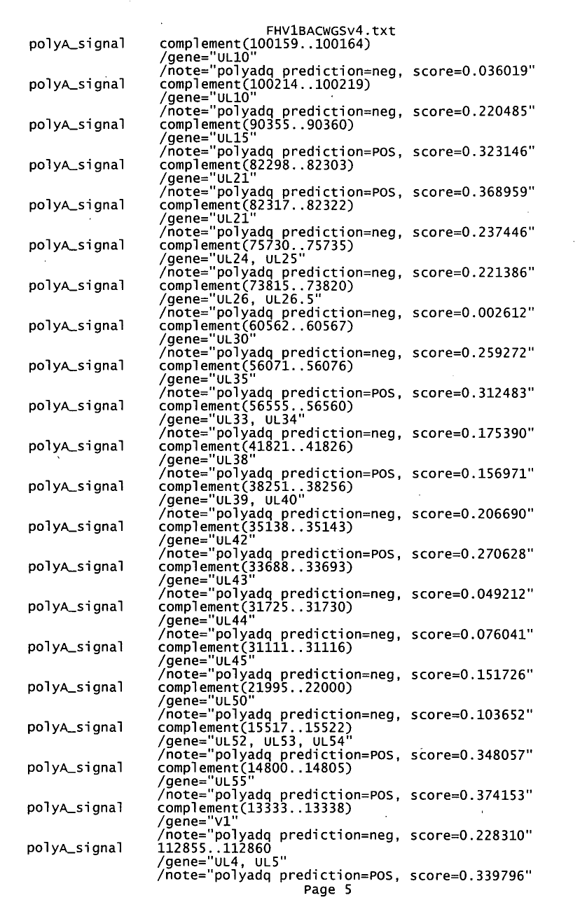

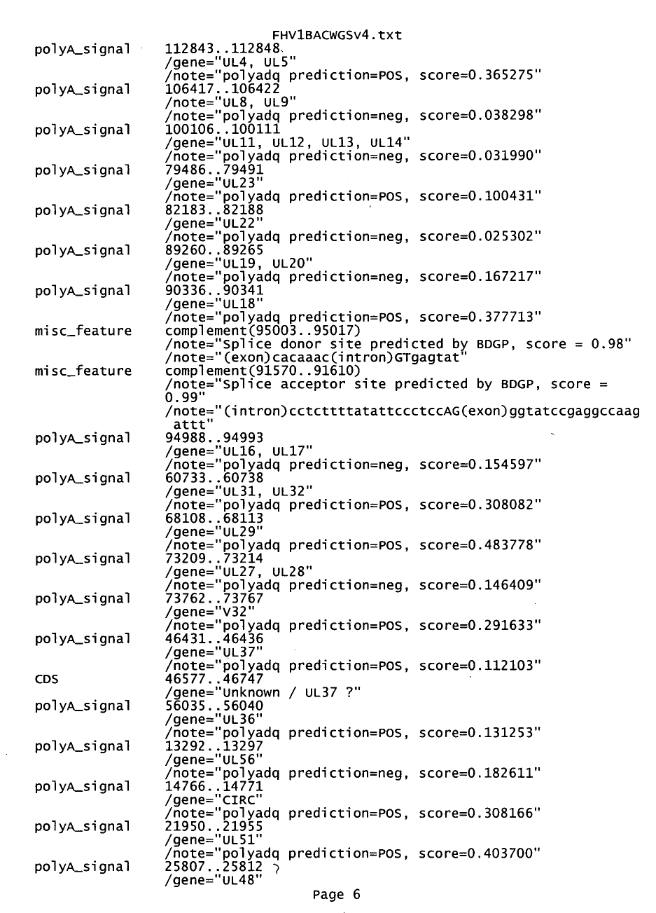

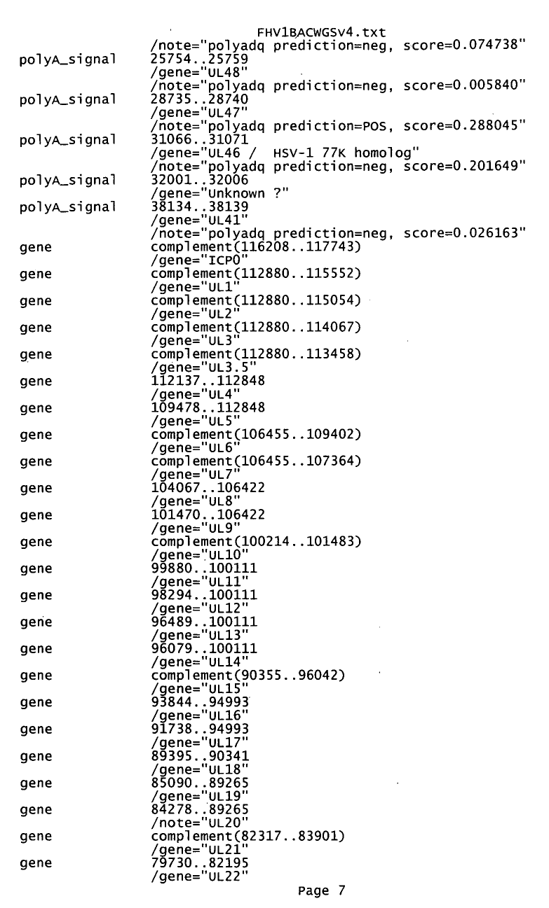

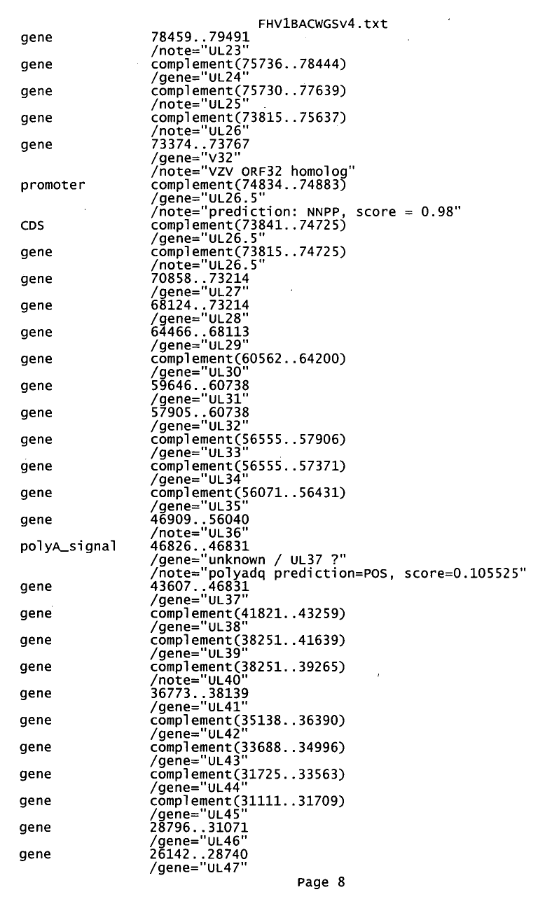

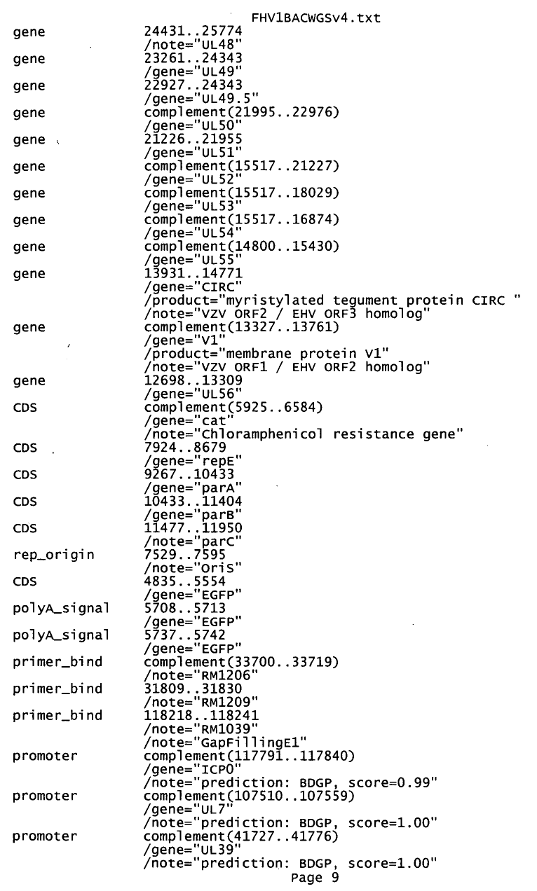

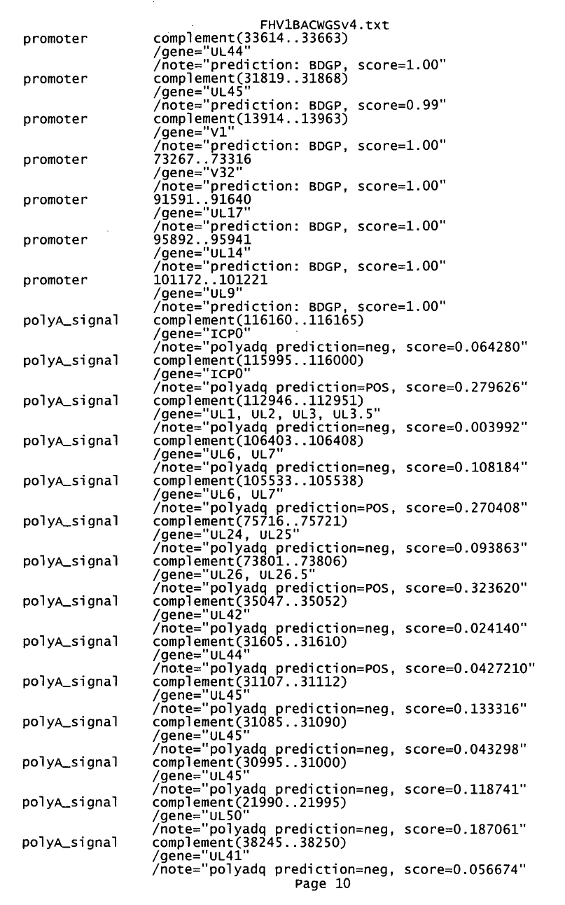

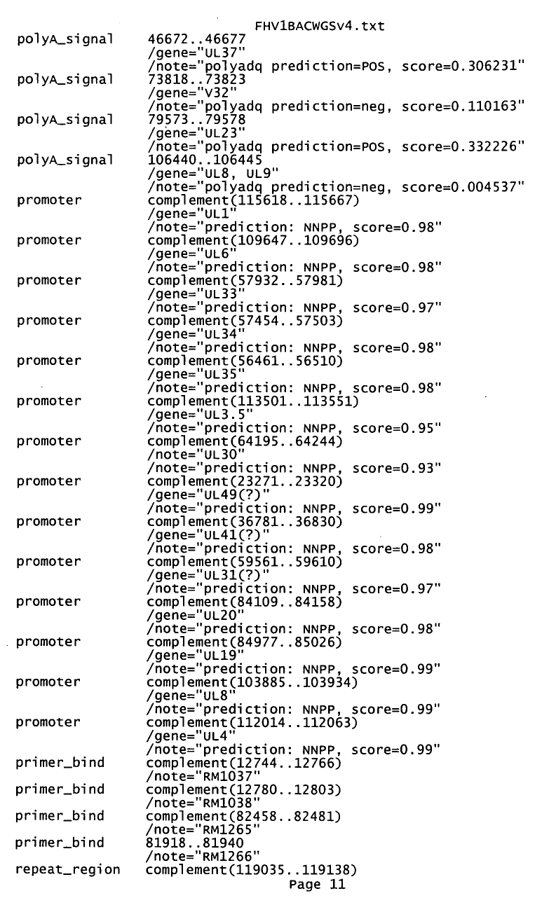

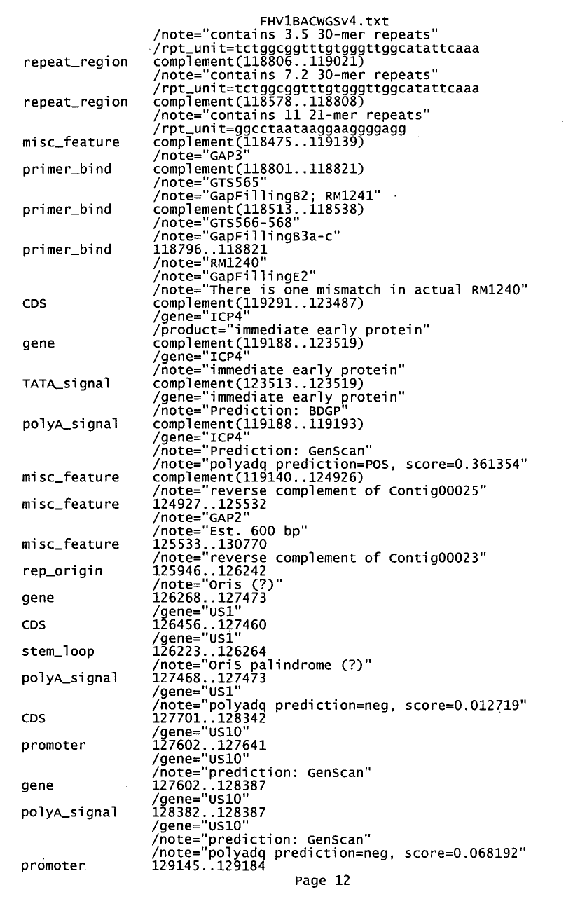

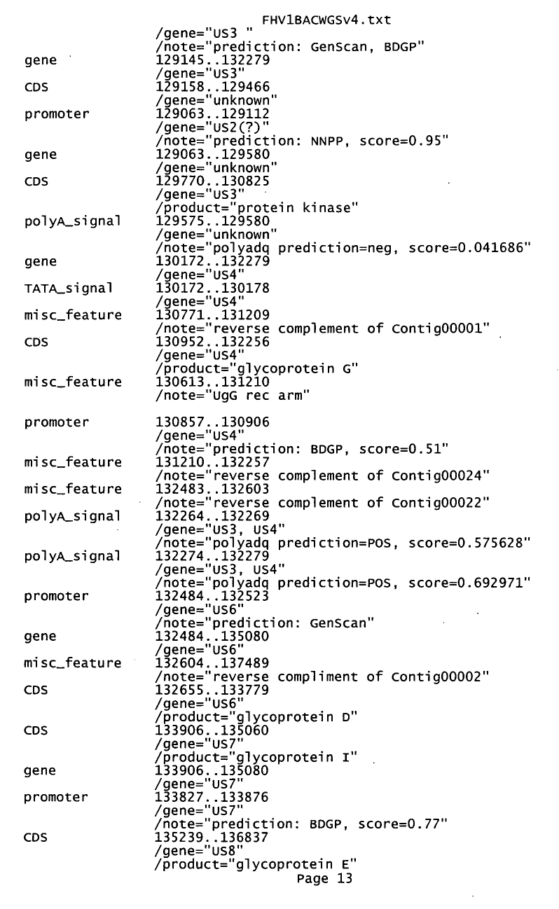

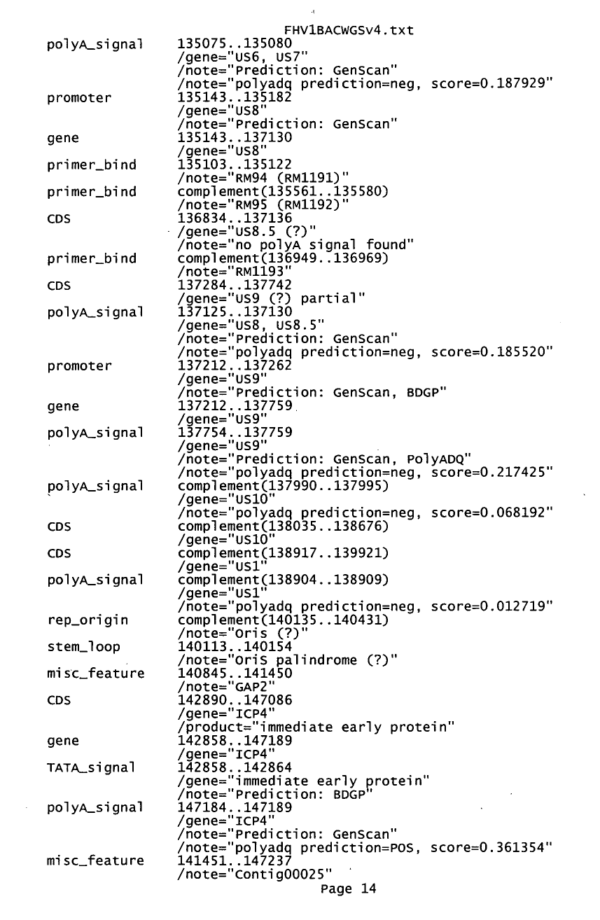

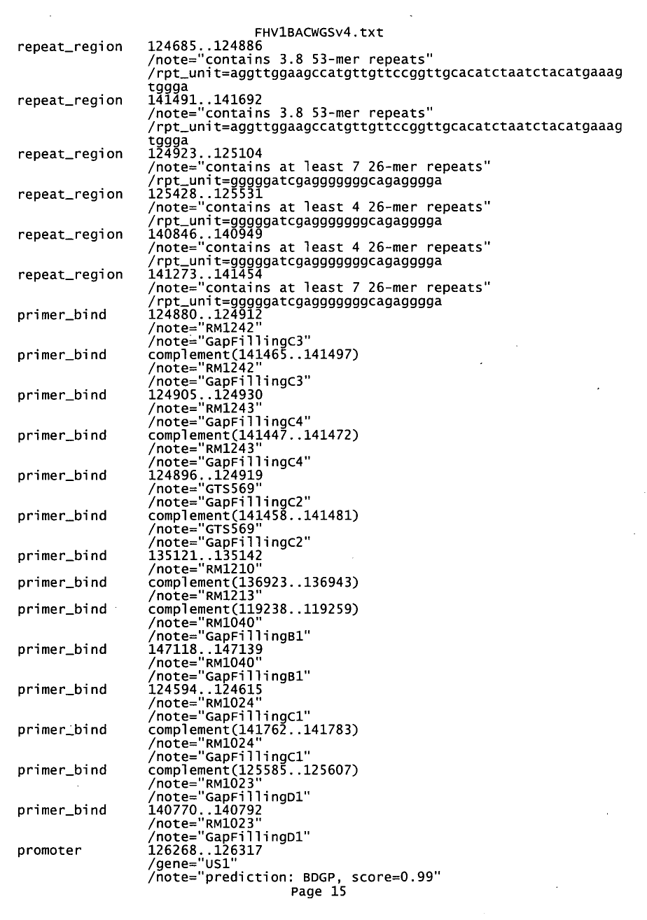

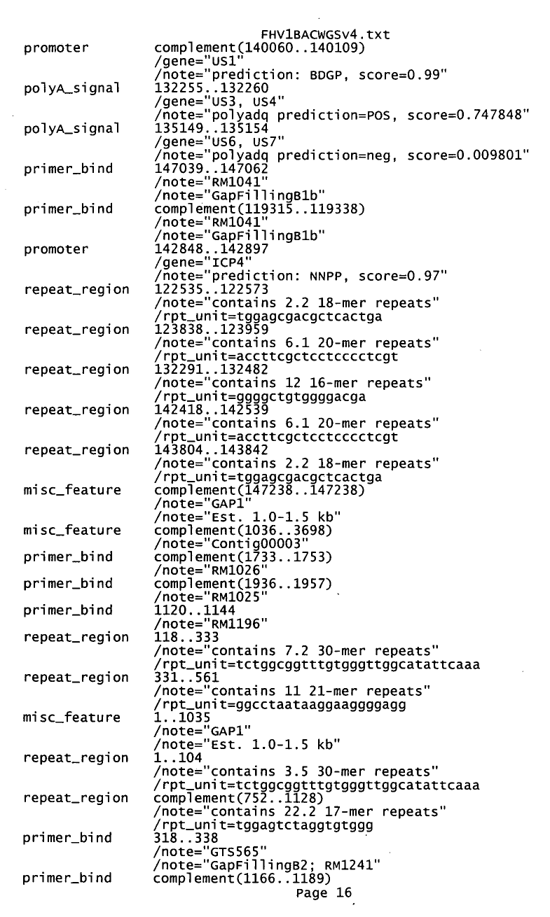

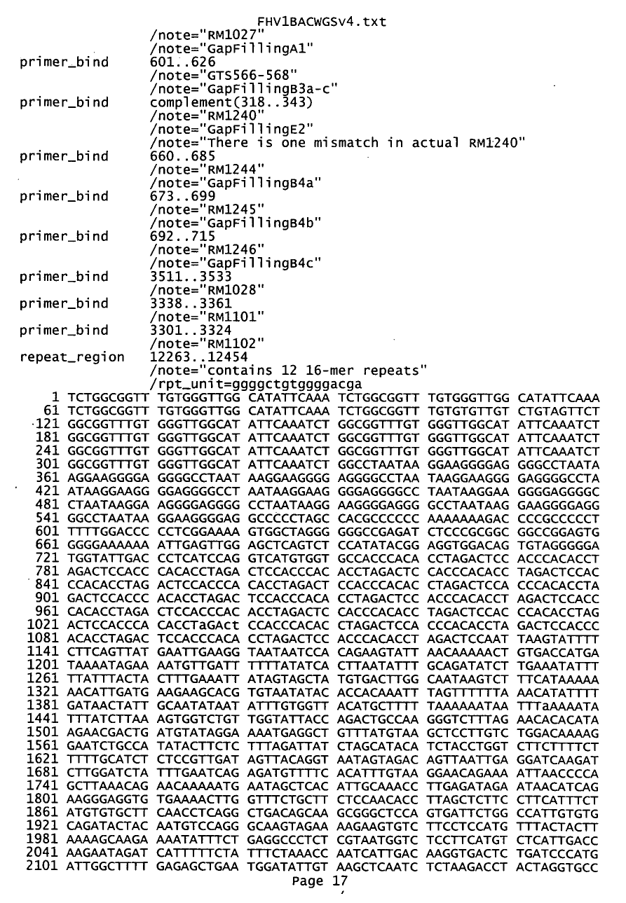

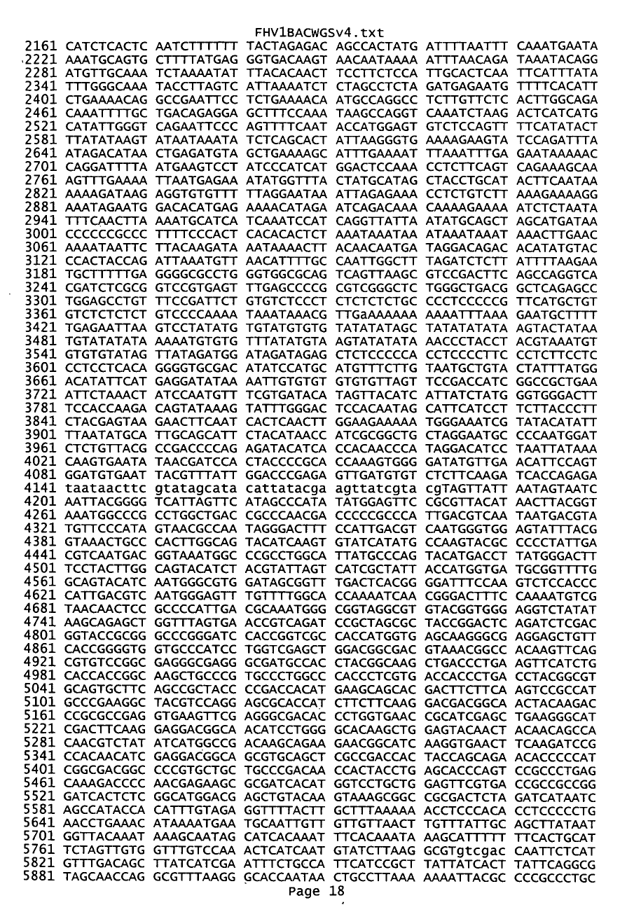

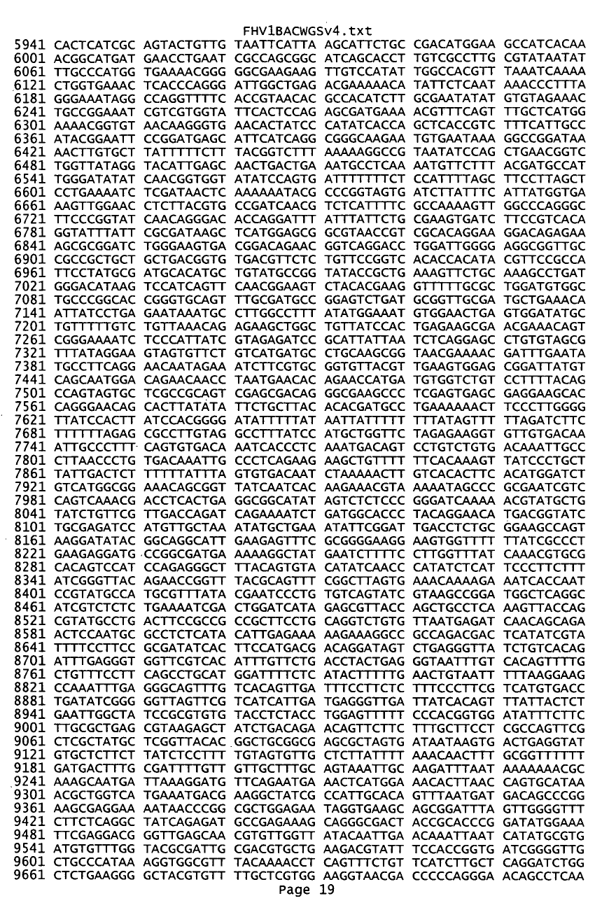

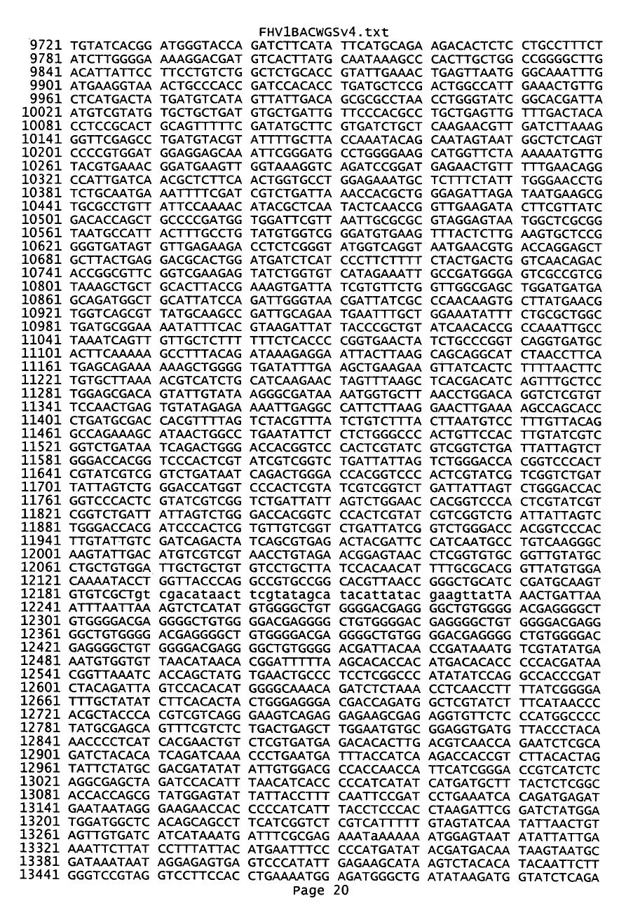

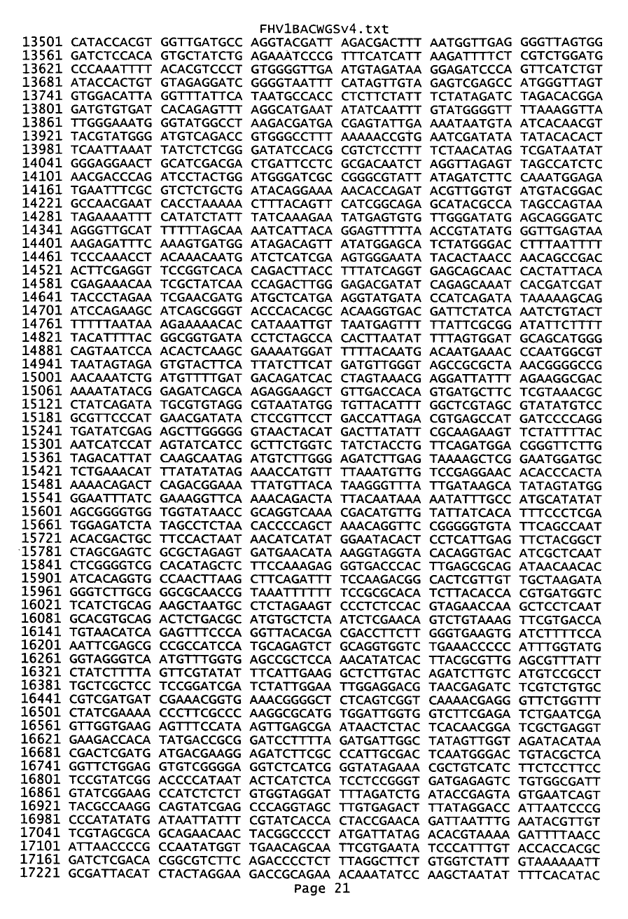

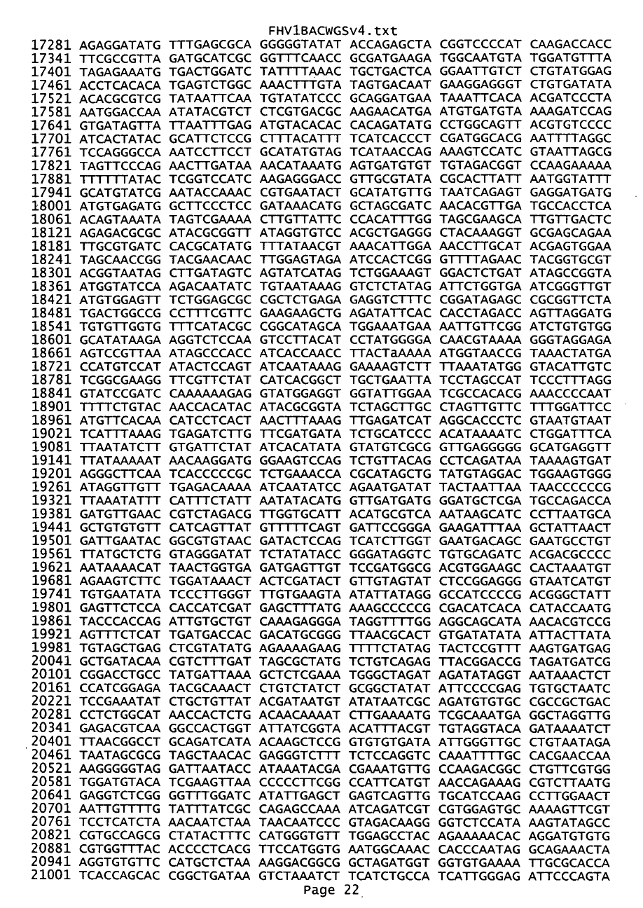

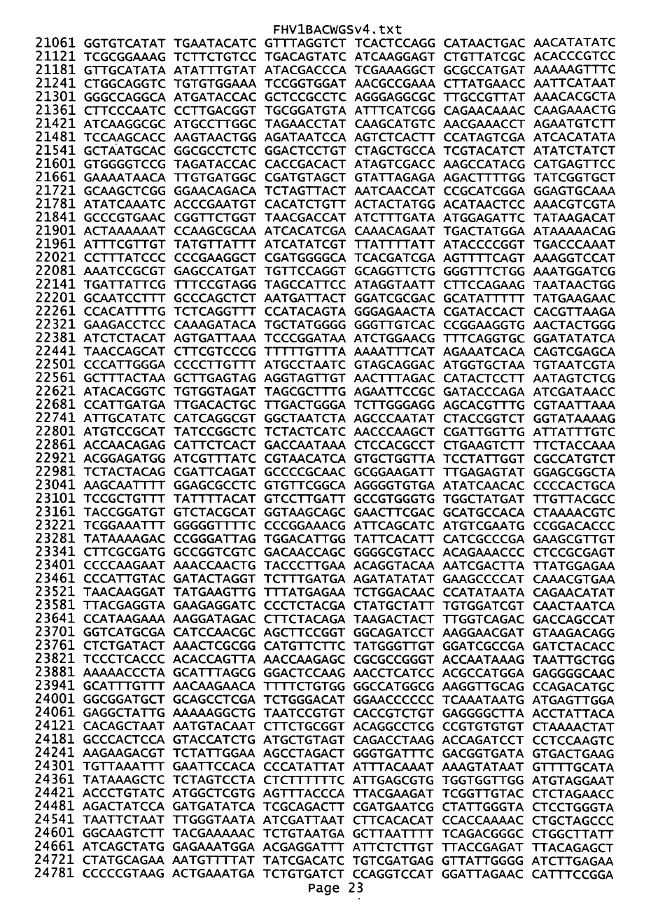

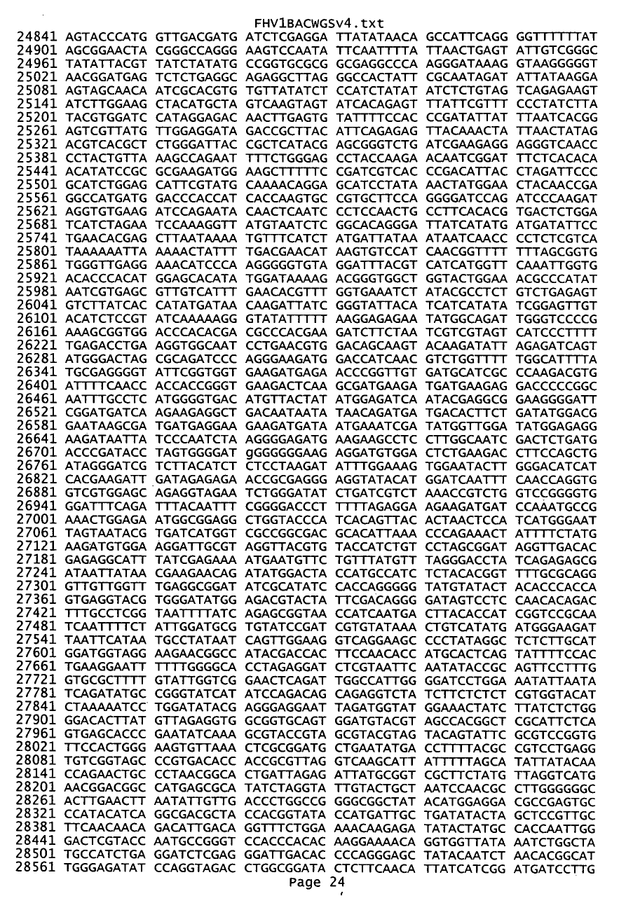

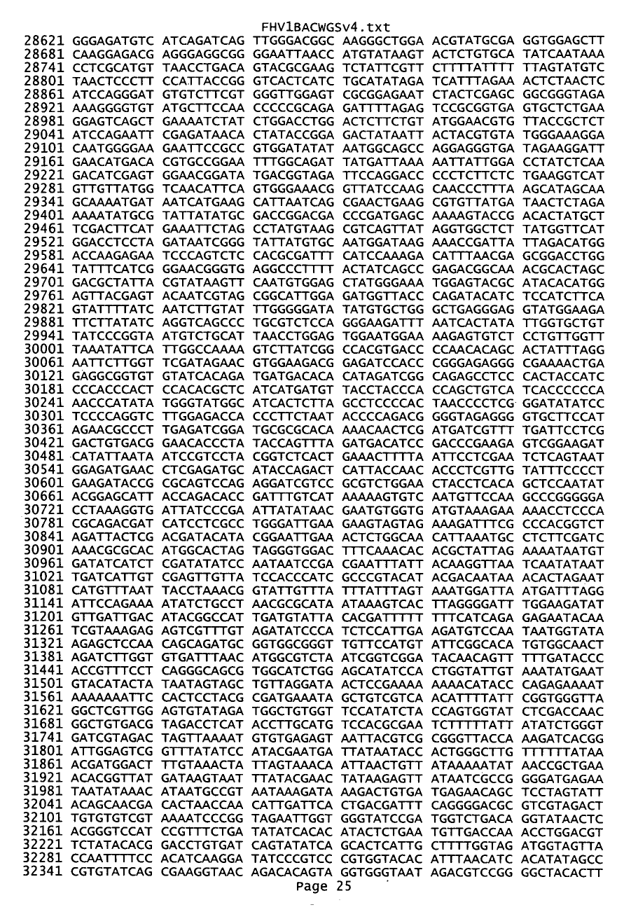

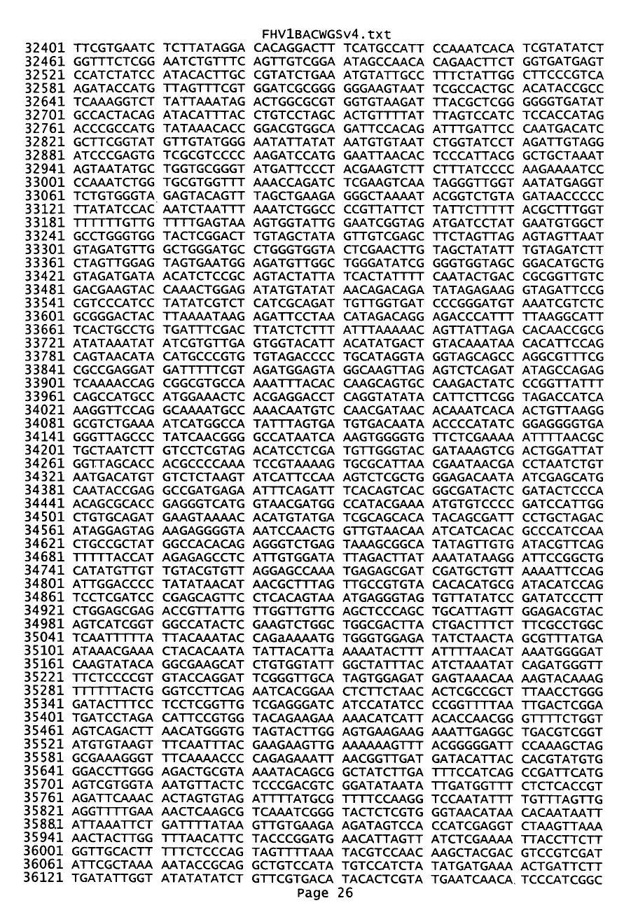

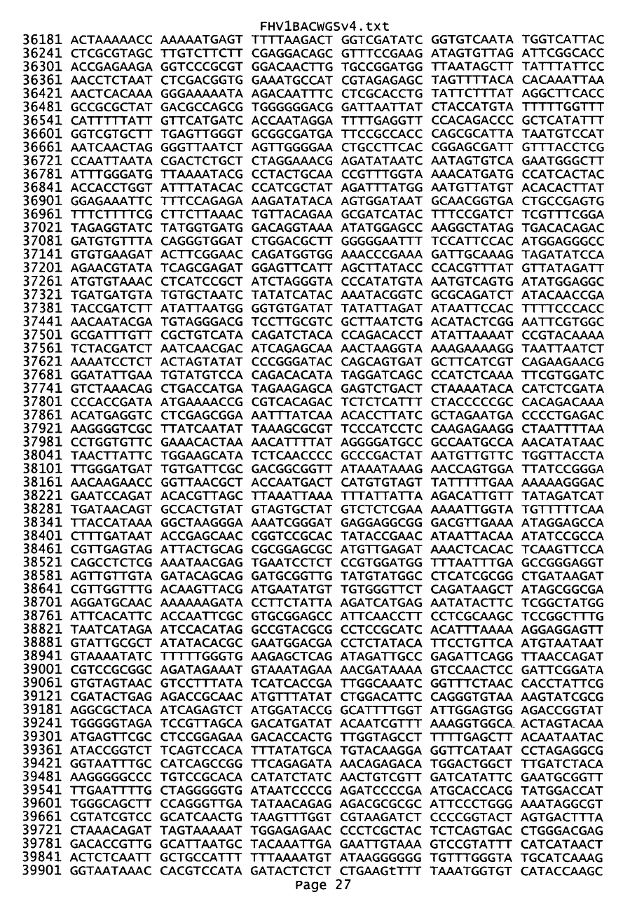

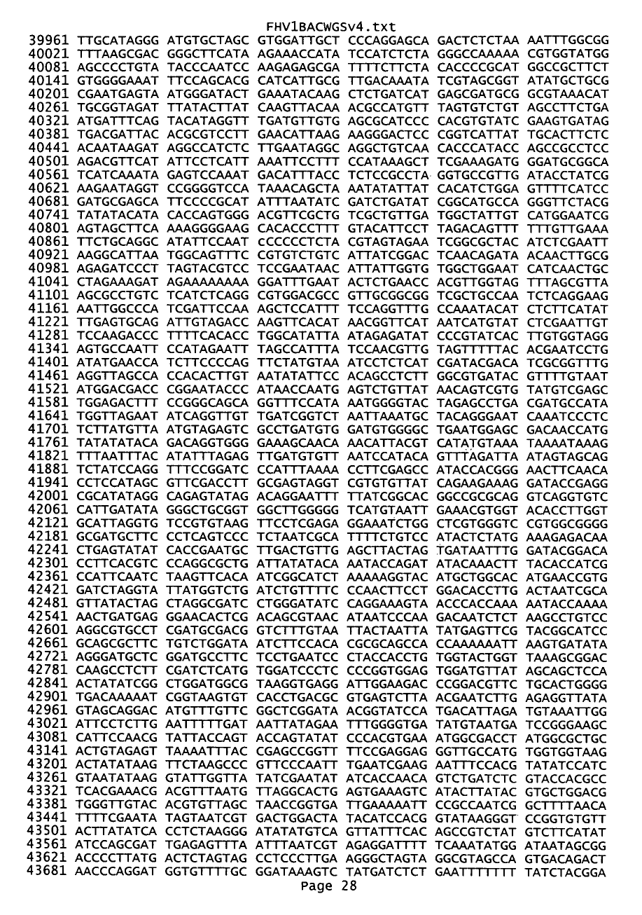

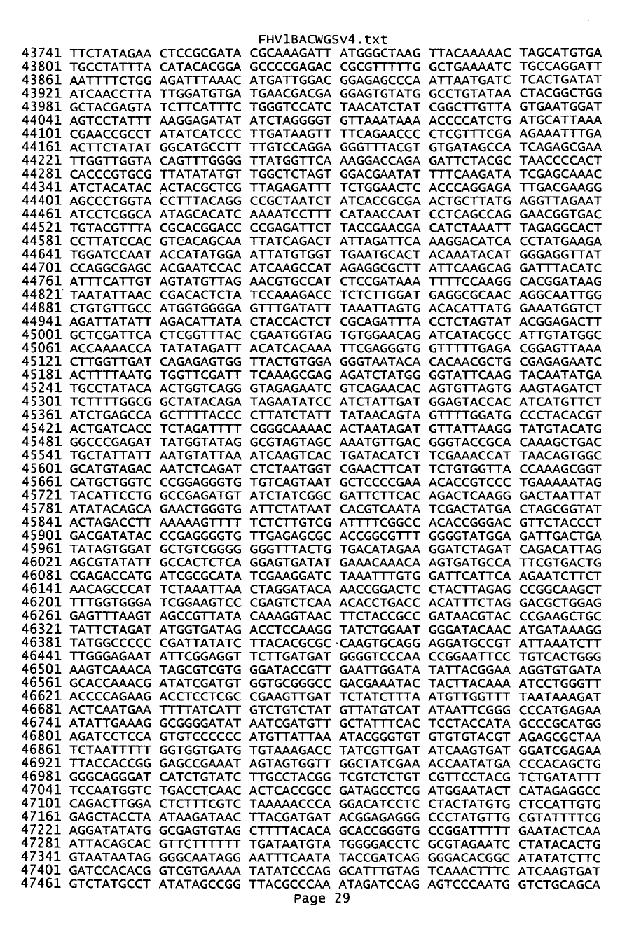

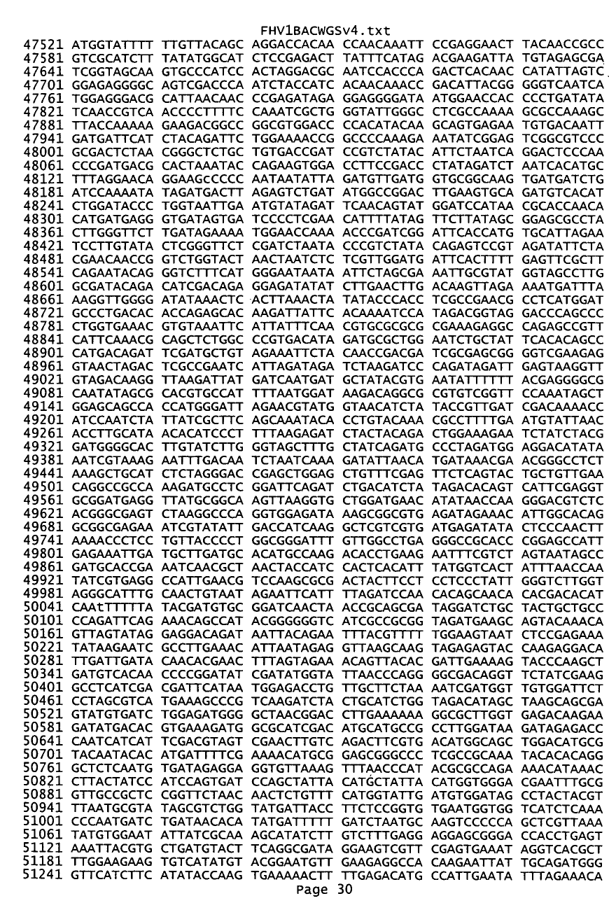

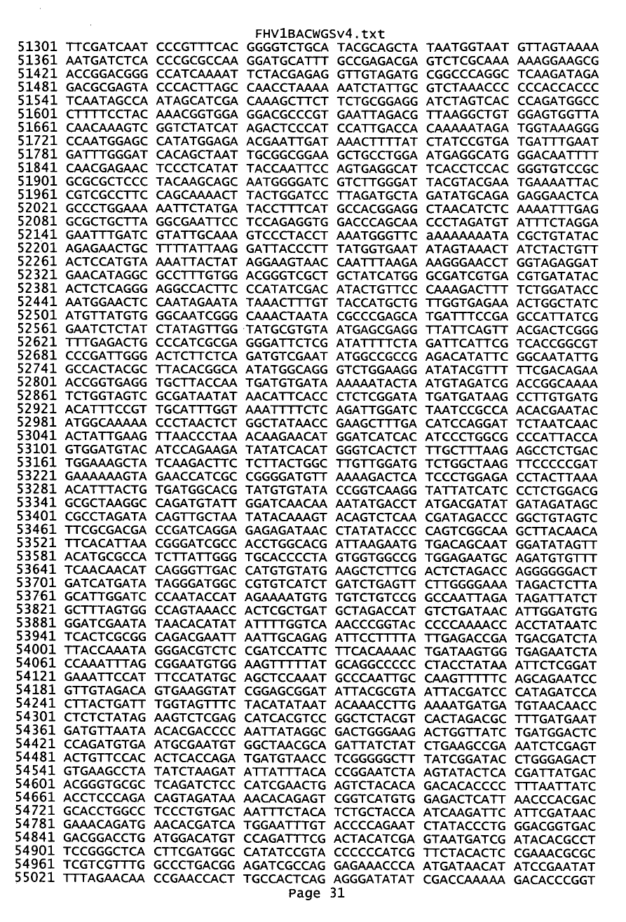

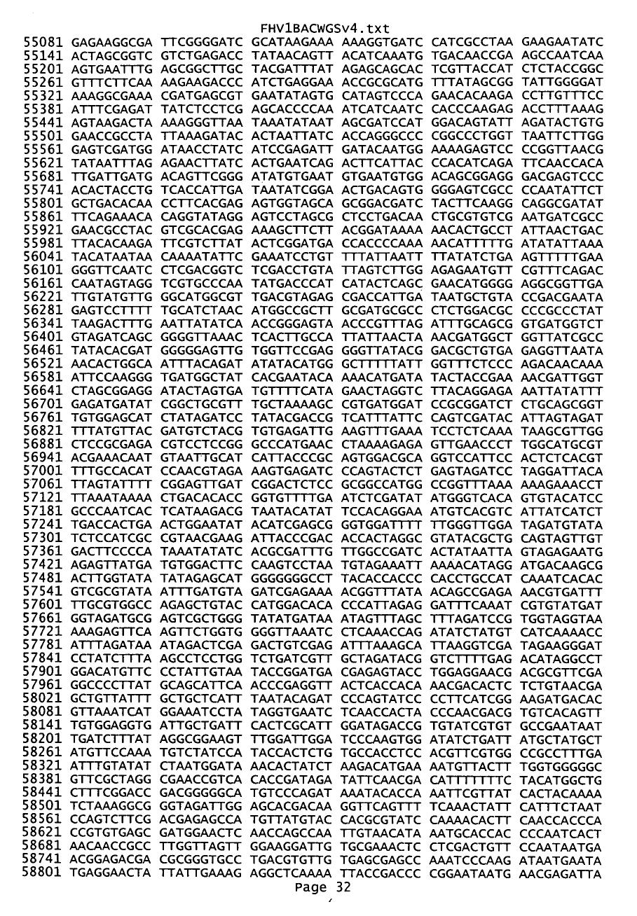

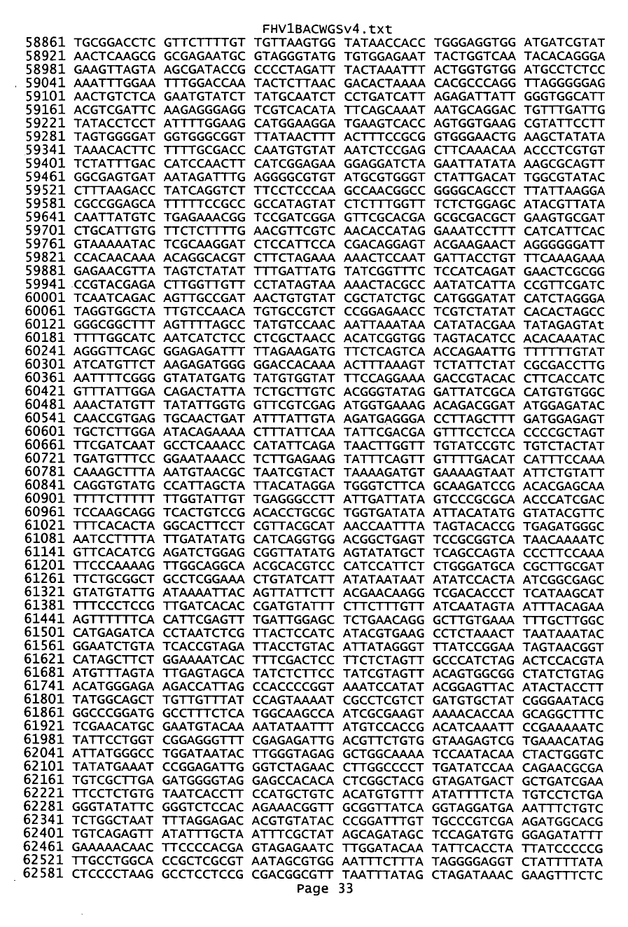

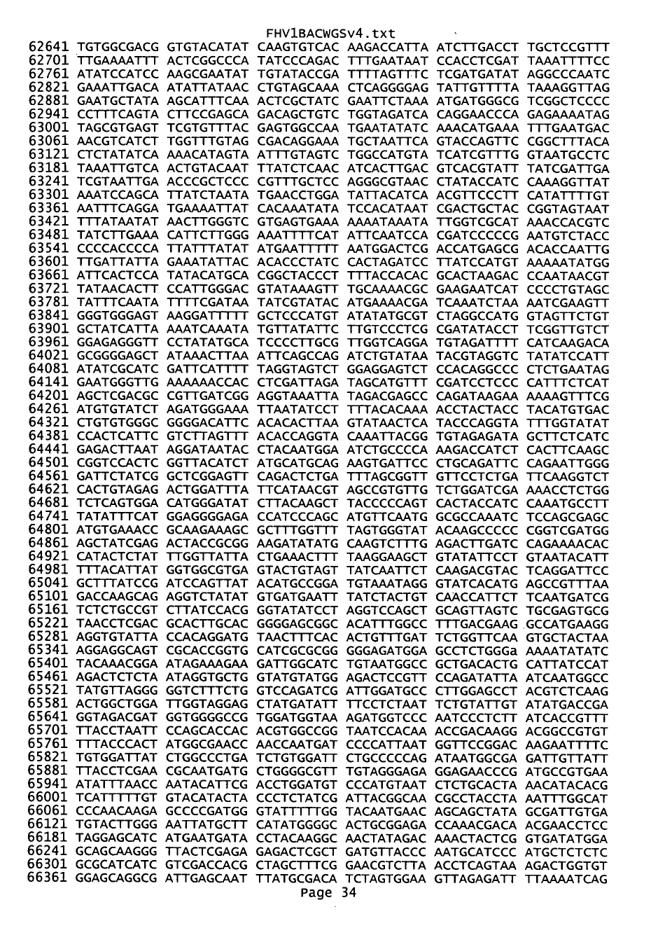

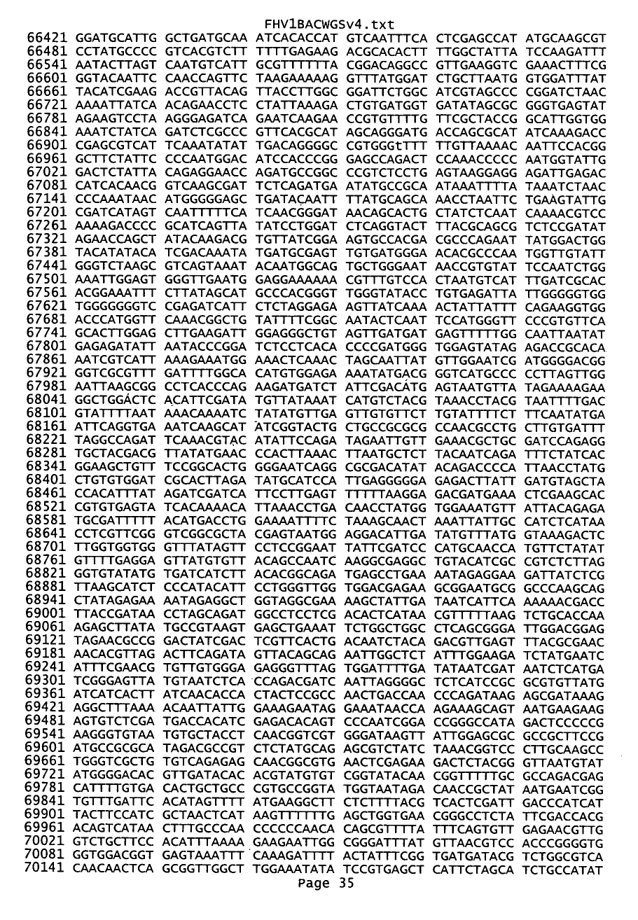

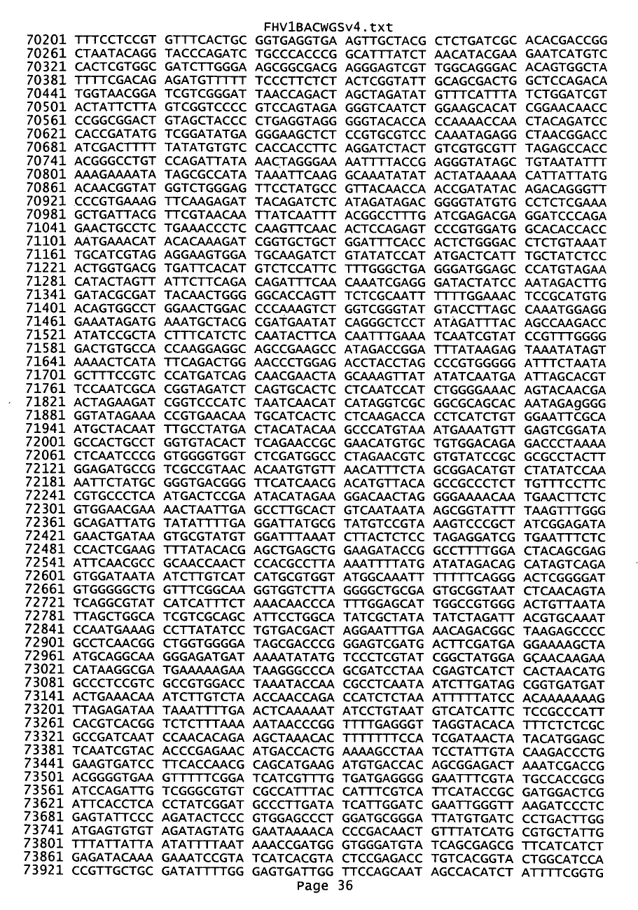

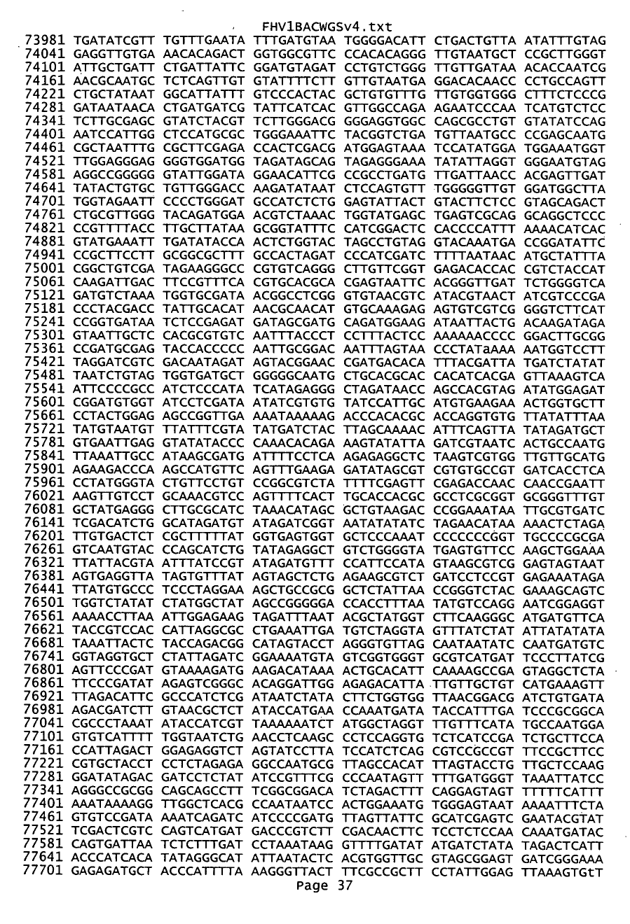

In an exemplary embodiment a BAC clone that contains the whole FHV-1 genome and is infectious in vitro and in vivo has been generated. FIG. 15A is a genomic map of the predicted FHV-1 gene arrangement while FIG. 15B is an annotated sequence listing for the complete FHV-1 genome. To reduce possible sequence alterations during in vitro passage in cell culture, low-passage C-27 virus (P4) was used for BAC cloning. The PCR, sequencing and primer walking results have shown that the BAC vector was not inserted in the gG gene as was expected but instead at the junction between the U_{L} and the TRs. Moreover, a 2.6-kb cellular DNA was inserted along with the BAC at the genomic termini. Herpesvirus genomes are linear and they carry at both ends directly repeated “A sequences” that contain the cis-acting signals for cleavage and packaging of the concatemeric genomes. It is known that many herpesviruses can acquire parts of host genomes, through an unknown mechanism, presumably during replication. In order to delete the 3′-end of gG but leave the surrounding sequences intact, the downstream recombination arm was designed to match the 1 kb region right next to the stop codon of gG, which inevitably contains a ˜200 by region of repetitive sequence. This repetitive sequence may have also contributed to the unexpected insertion of the BAC vector, since repetitive sequences, including the A sequences, are more prone to spontaneous recombinations.

B. Sequencing, Sequence Assembly

The first complete genomic DNA sequence of the FHV-1 genome has been completed. Prior to this study, sequencing efforts were focused on smaller fragments of the genome or individual genes. The sequences available were scattered throughout the genome. Most of these studies were carried out in the 1990s, using various strains. In addition to the previously identified 8 glycoprotein genes, 3 more were found in the genome. The sequence was obtained by sequencing the FHV-1 BAC clone using a newly developed automated high-throughput pyrosequencing system. This system is rapid, provides high read depth, and the price is comparable to that of the Sanger sequencing. The disadvantages of this method include short read length and difficulties in direct single nucleotide repeats. Compared to 500-1000 by read length of Sanger sequencing, the read length of pyrosequencing was averaging 100 bp, but was recently increased to 200-400 bp. Another inherent problem in pyrosequencing is the difficulty in determining the number of incorporated nucleotides in homopolymeric regions, due to the non-linear light response following incorporation of more than 5-6 identical nucleotides (Ronaghi et al., Comp. Funct. Genomics 3(1):51-6, 2002). It is possible that the lengths of such repeats in this genomic sequence are not entirely accurate. This issue could be resolved by sequencing these regions using the Sanger method. The short read length poses difficulties for assembly of repetitive sequences, including the repeat regions (IRS and TRS), as well as tandem repeats, such as the A sequences. Initial sequence assembly by the Newbler program assembled 9 contigs that are related to the BAC clone. All the ends of these contigs were bordering repetitive sequences. Contigs 1 and 22 were parts of the recombination arms, which appeared twice, once in the U_{S} and once in the BAC vector. Contig 2 was bordered by the downstream recombination arm and TRS. Contig 3, the cellular sequence, was bordered by the upstream recombination arm and the genomic terminus. Contig 6, the BAC vector, was bordered by the two recombination arms. Contig 8 was bordered by the downstream recombination arm and the internal A sequence at the junction of the U_{L} and the IRs. Contig 23 was bordered by Gap 2 and the upstream recombination arm. Contig 24 was the part of gG intended for BAC replacement, bordered by the two recombination arms. Contig 25 was bordered by the A sequence and Gap 2. Had the BAC vector inserted as planned, the U_{S} region could be automatically assembled into one or two contigs. The A sequences at the genomic termini, which consists of complex tandem repeats, could not be assembled.

It is believed that, this is the first report of applying pyrosequencing technology in sequencing a herpesvirus genome. Although there are shortcomings in resolving the tandem repeats, this method has proven to be very time- and cost-efficient in obtaining the vast majority of the genomic sequence of FHV-1. With its increasing power, it is expected that more herpesvirus genomes will be sequenced using this system. Moreover, different strains, including vaccine strains can be rapidly sequenced, making it easy for genome-wide analysis of strain variability, which can shed light on the causes for attenuation and phenotypic differences.

C. Genome Structure, Gene Arrangement, Codon Usage

The gene arrangement in the FHV-1 genome is collinear with that of many varicelloviruses, including BHV-1, BHV-5, EHV-1, EHV-4, and VZV. The shuffling of gene blocks found in the PRV genome did not appear in the FHV-1 genome. A few alphaherpesviruses, including HSV-1, HSV-2, BHV-1, and PRV, have evolved genomes with a relatively high G-1-C content. In these genomes, there is a pronounced periodicity in triplet base composition in the protein coding sequences. The third codon position is particularly biased towards G or C, since it is the most flexible concerning the amino acid encoded. The third position nucleotides have evolved to contribute the most to high G+C content of these genomes. Klupp et al. were able to easily identify all known functional PRV ORFs by screening for ORFs with a high G+C content on the third nucleotide position of codons (Klupp et al., J. Virol. 78(1):424-440, 2004). The FHV-1 genome doesn't seem to have evolved this characteristic of high G+C content. The average G+C percentage of FHV-1 genome was 45%. Therefore, this method was not applicable for FHV-1.

D. Restriction Map

The physical structure of the FHV-1 genome was first mapped in 1986 when Rota et al. reported a SalI restriction map of the C-27 strain (Rota et al., Virology 154(1):168-179, 1986). In 1991, Grail et al. mapped another strain of FHV-1, B927 (Grail et al., Arch Virol 116(1-4):209-220, 1991). A search for SalI sites in our genome sequence revealed that the SalI map of C-27 is actually very similar to the B927 strain, with only a few differences. The region in the B927 strain where there is a 16.5-kb SalI fragment contains a 13.5- and a 13.6-kb fragment in the C-27 strain. This could explain why the predicted length of B927 genome was 9 kb shorter to our sequence. The second and fourth fragments in the C-27 strain seem to have exchanged their locations in the B927 strain, implying a possible rearrangement of the genes, as seen in the PRV genome.

E. ORF Finding, Gene Composition, PolyA Signals, Promoters, Splicing Sites

For UL28, UL37, and UL46, two ORFs were found to be part of the same gene in homology searches. Possible explanations for this phenomenon includes: 1) The second ORF resulted from recombination/rearrangement, and is no longer used. However, in all three cases the gene products were shorter than their counterpart in other varicelloviruses without the second ORF. 2). Both ORFs are part of the gene, but a sequencing error, e.g. incorrect length of single nucleotide repeats which would cause frame-shift, resulted in early termination.

F. Origins of Replication, Tandem Repeats

The number of reiterated elements found in the FHV-1 genome is far less than in other varicelloviruses. Due to the short read length of the pyrosequencing, it is possible that multiple copies of the same repetitive unit are assembled into much fewer copies. However, most of the reiterated elements are shorter than 35 bp, which means in the sequence assembly there should still be at least 2 copies, if they are present, hence detectable by the Tandem Repeats Finder program. Therefore, it is also possible that the FHV-1 genome does not have as many tandem repeat elements.

### VI. In Vitro and In Vivo Characterization

In vitro Characterization: Plaque Morphology, Growth Curve. To reconstitute virus particles form the BAC, the BAC DNA was transfected into CRFK cells. The reconstituted virus, with the BAC vector in its genome, produced fluorescent plaques in CRFK monolayers (data not shown). To eliminate the effects the BAC vector might have on growth characteristics and virulence, the BAC vector was removed by co-transfecting the CRFK cells with the BAC DNA and pcDNA-Cre. pcDNA-Cre expresses Cre protein, which specifically recognizes the loxP sites flanking the BAC vector, excises it, and re-ligates the DNA leaving a single loxP site. Very few fluorescent plaques were found in the supernatant obtained from the co-transfected cells (data not shown), when the supernatant was plaque purified, suggesting that this is an efficient way to remove the BAC vector. The excision of BAC was also verified by PCR and sequence analysis. The BAC-excised BAC clone of FHV-1 (FHV1ΔBAC) was used for subsequent in vitro and in vivo characterizations. The FHV 1 iBAC virus can grow to a high titer similar to the parent strain at 72 hours p.i. (FIGS. 9A and 9B). Analysis of the growth curves by ANOVA suggested that there are significant differences at 48 (p=0.003) and 72 hours p.i. (p=0.013). After the removal of the BAC vector, the virus can produce plaques that are morphologically undistinguishable from those of the parent strain (FIG. 4).

In vivo Study. In order to investigate possible attenuation resulting from BAC cloning, a preliminary challenge experiment was carried out with four specific-pathogen-free cats. The positive control cat and the FHV1ΔBAC virus-infected cats all tested VI positive on days 3, 6, and 9, and became negative on day 14. The positive control cat and the FHV1ΔBAC virus-infected cats all showed nice seroconversions on day 14, and the neutralizing antibody titers of the FHV 1 ΔBAC virus-infected cats are similar to that of the parent C-27 strain-infected cat. The clinical scores of the BAC clone-infected cats were comparable to those of the wild type virus. Virus neutralization tests showed that both the parent strain and the BAC clone can induce similar neutralizing antibody titers (Table 3).

In the pilot study, only two SPF cats were inoculated with the BAC-cloned virus. Although the number was very small, the fact that both of the cats developed symptoms similar to the wild-type strain suggested that the BAC-cloned virus is infectious, and appears to be as virulent as the parent strain. In previous studies, we have learned that the symptoms induced by FHV-1 in experimentally infected cats are quite consistent (Kruger et al., Virology 220(2):299-308, 1996; Sussman et al., Virology 214(1):12-20, 1995). Therefore, it is predicted that the results shown here will be reproducible in a larger-scale experiment. The FHV-1 BAC clone that was constructed in this study, although the vector was inserted in an unexpected manner, most likely as a result of repetitive sequences in the downstream recombination arm, has been proven virulent in vitro and in vivo. In addition, it is easy to introduce both random and site-specific mutations in a BAC clone. These characteristics will make the BAC clone an excellent platform for further mutagenesis-based gene function studies as well as vaccine developments.

VII. Materials and Methods for Sequencing of the Complete FHV-1 Genome from an Infectious BAC Clone.

The following describes the materials and methods used to derive the complete FHV-1 genome from an infectious BAC clone but is meant as an example only and not as a limitation as to one with skill in the art of biotechnology and more particularly molecular biology and genomics.

Cells and Viruses. Crandell-Reese feline kidney (CRFK) cells (ATCC CCL-94, Manassas, Va.) were cultured in Eagle's minimum essential medium (EMEM) containing 10% fetal bovine serum (FBS) and 10 μg/mL ciprofloxacin. The FHV-1 prototype strain C-27 (ATCC VR-636, Manassas, Va.) was propagated in the CRFK cells and used as wild-type virus throughout this study.

Plasmids and Vectors. The 0.6-kb upstream homologous region, which includes the 3′-end of US3 gene, the intergenic region of US3 and US4, and the 5′-end of US4 (gG) gene, was amplified from purified FHV-1 genomic DNA by polymerase chain reaction (PCR) using the Extended High Fidelity PCR System (Roche Applied Science, Indianapolis, Ind.) with the following primers: RM0874, which contains a MluI site at the 5′-end; and RM0875, which contains a SwaI site, a BsiWI site, and a loxP site at the 5′-end (Table 6). The PCR product was cloned into a pCRII vector (Invitrogen, Carlsbad, Calif.), resulting in pCRII-UgG. The 1.0-kb downstream homologous region, which includes the intergenic region of US4 (gG) and US6 (gD), and the 5′-end of US6 (gD) gene, was amplified from purified FHV-1 genomic DNA by polymerase chain reaction (PCR) using the Extended High Fidelity PCR System with the following primers: RM0876, which contains a HindIII site, a SalI site, and a loxP site at the 5′-end; and RM0877, which contains a MluI site at the 5′-end (FIG. 14A). The PCR product was also cloned into a pCRII vector, resulting in pCRII-DgG. pCRII-EGFPm2 was constructed as follows. The vector pEGFP-N1 (Clontech Laboratories, Mountain View, Calif.) was digested by XhoI and SalI and re-ligated, eliminating Sad, HindIII, and SalI sites. The 1.626-kb region containing modified EGFP expression cassette, which includes the CMV immediate early promoter, the EGFP open reading frame (ORF), and SV40 early mRNA polyadenylation (poly(A)) signal, was amplified from the modified pEGFP-N1 by polymerase chain reaction (PCR) using Extended High Fidelity PCR System with the following primers: RM0872, which contains a BsiWI site at the 5′-end; and RM0873, which contains a Sad and SalI site at the 5′-end (FIG. 14A). The PCR product was cloned into a pCRII vector, resulting in pCRII-EGFPm2. pGEM3Zf-DUgG2, which contains two homologous regions and two loxP sites, was constructed by ligation of the following fragments: the MluI-SwaI fragment of pCRIIUgG; the HindIII-MluI fragment of pCRII-DgG; and the pGEM-3Zf vector (Promega, Madison, Wis.), digested with HindIII and SmaI. pGEM3Zf-DUgGEGFP2, which contains two homologous regions, two loxP sites, and a EGFP expression cassette, was constructed as follows. Both the pGEM3Zf-DUgG2 and pCRII-EGFPm2 were digested with BsiWI and Sad and ligated together. The transfer vector/donor plasmid pFHV1BACNo1, which contains the two (upstream and downstream) homologous regions between two loxP sites and an EGFP expression cassette, was constructed as follows. The BAC vector pBeloBAC11 (Invitrogen, Carlsbad, Calif.) was digested with SalI and dephosphorylated; the 6.4-kb fragment was purified and ligated with the SalI fragment of pGEM3Zf-DUgGEGFP2. pcDNA-Cre was constructed by cloning Cre gene into the pcDNA3.1 vector (Invitrogen, Carlsbad, Calif.). All the plasmid constructs were verified by both restriction digestion and sequencing. pFHV1BACNo1 was transformed into the E. coli strain DH10B (Invitrogen, Carlsbad, Calif.). All the other constructs were transformed into the E. coli strains TOP10 or DH5a (both from Invitrogen, Carlsbad, Calif.). The transformants were plated on selective agar that contained 75 μg/ml ampicillin and/or 34 μg/ml chloramphenicol.

DNA Extraction. Pure viral genomic DNA was prepared as follows. The FHV-1 C-27 strain was propagated in CRFK cells and virions were collected by pelleting the supernatant through a 20% potassium tartrate cushion at 24,000×g for 2 hours at 4° C. Purified virions were incubated in lysis buffer (0.1 M Tris-Cl, pH8.0, 1 mM EDTA, 1% SDS) with 50 μg/ml proteinase K at 37° C. DNA were subsequently extracted with phenol-chloroformisoamylalcohol (25:24:1), and precipitated with 100% ethanol. Care was being extended not to shear the DNA. High copy number plasmids were extracted using the Plasmid Mini Kit (Qiagen, Valencia, Calif.). Small-scale BAC DNA purifications were carried out using the alkaline lysis method (Sambrook et al, Molecular Cloning: A Laboratory Manual, Cold Spring Harbor Press, 2001). Large-scale and high-purity BAC DNA purifications were carried out using the Large Construct Kit (Qiagen, Valencia, Calif.) following the manufacturer's instructions.

BAC Cloning/Virus Construction. Purified FHV-1 genomic DNA and BAC04 were co-transfected into CRFK cells using Lipofectamine 2000 (Invitrogen, Carlsbad, Calif.) according to the manufacturer's instructions. The supernatant was collected for plaque purification. Briefly, the monolayer was overlaid with growth medium containing 1% low-melt agarose, fluorescent plaques produced by the recombinant virus were picked and the virus was eluted in medium at 4 C overnight. After two times of plaque purification, the recombinant virus was inoculated on a monolayer of CRFK cells. The cells were collected, lysed, and total DNA was extracted, purified and transformed into E. coli DH10B cells. Colonies grew on the chloramphenicol plate were examined for the presence of BAC by restriction digestion and PCR.

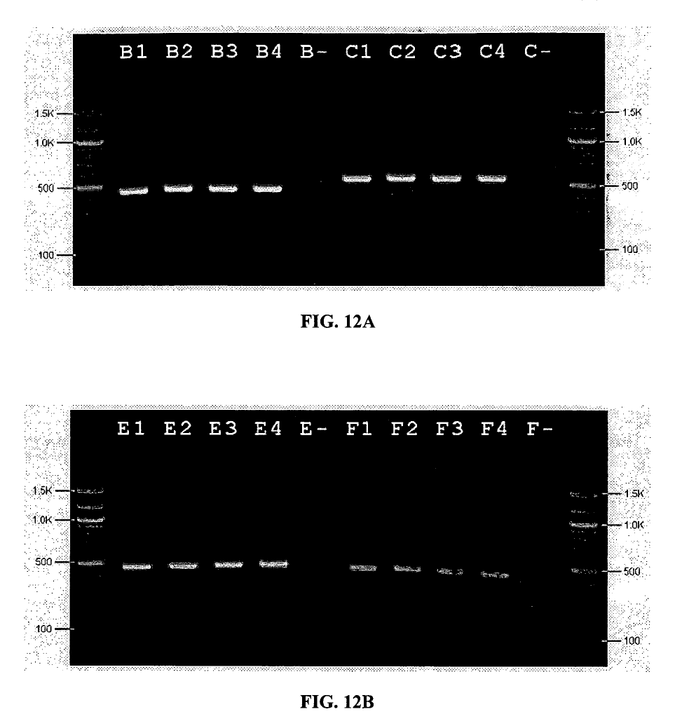

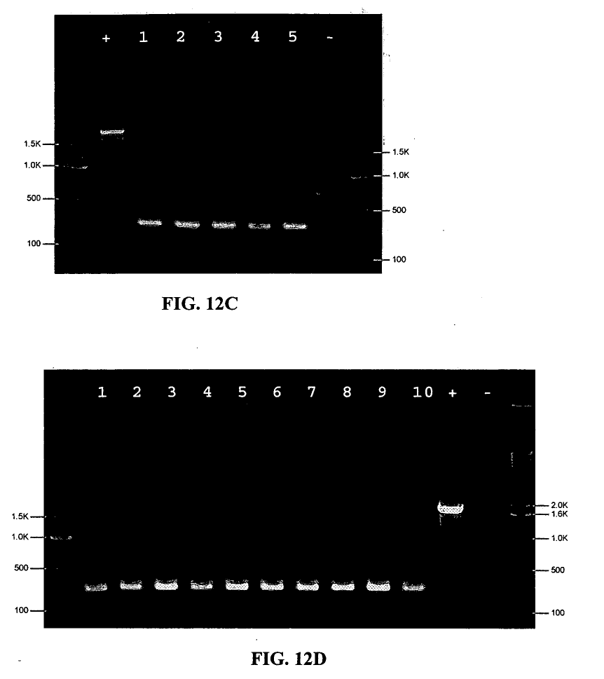

Restriction Enzyme Analysis. Genomic viral DNAs were treated overnight with restriction endonuclease Sail at 37° C. The digested DNAs were subjected to electrophoresis at 120 V for 2 h on a 0.7% agarose gel. After electrophoresis, the gels were stained with ethidium bromide (0.5 μg/ml) and photographed on a UV-light transilluminator (See FIG. 12). Reconstitution of infectious virus from BAC. The CRFK cells were co-transfected with 1 ug of the BAC DNA and 1 ug of pcDNA-Cre using Lipofectamine 2000 (Invitrogen, Carlsbad, Calif.) following the manufacturer's instructions.

Sequencing, Sequence Assembly and Gap Closure. The majority of the sequence was determined by shotgun sequencing at 30× redundancy, using the high-throughput pyrosequencing instrument Genome Sequencer 20 (Roche Applied Science, Indianapolis, Ind.) at the Genomics Core of Michigan State University (MSU) Research Technology Support Facility (RTSF). The reads were assembled using the Newbler assembly program (Roche Applied Science, Indianapolis, Ind.) at the Bioinformatics Core of RTSF. Gaps were closed by primer walking. Gap closure was carried out using BigDye Terminator 3.0 and dGTP BigDye Terminator chemistries and Gene Analyzer 3100 (Applied Biosystems, Foster City, Calif.).

ORF Finding and Sequence Analysis. To identify the genes encoded in the FHV-1 genome, all potential ORFs with a minimum length of 60 codons and a methionine as start codon were analyzed for homology to known proteins using BLASTX against non-redundant protein database. The FHV-1 genome sequence was submitted to PolyADQ, a eukaryotic polyadenylation [poly(A)] signal search engine (69). All cutoff parameters were initially set at zero to return the location of all AATAAA and ATTAAA consensus signals, along with an associated score between 0 and 1. For each potential poly(A) signal, all upstream genes were noted.

### VIII. Materials and Methods for In Vitro and In Vivo Characterization of the FHV-1 BAC Clone.

Plaque Morphology. The viruses to be tested were serially diluted and inoculated on CRFK monolayers in 6-well plates. After an one-hour adsorption, the diluted virus was removed, and the cells were overlaid with growth medium containing 1% SeaPlaque low-melt agarose (Cambrex, Rockland, Me.). The plates were be incubated at room temperature for 30 minutes and then at 37° C. in 5% CO2. After 5 days of incubation, 100 plaques were randomly selected and the diameters were measured. The means of the plaque diameter of each mutant and parent strain were compared by ANOVA, followed by Tukey's HSD post-hoc test.

Growth Curve. The wild type virus and the BAC clone were inoculated on CRFK monolayers at an m.o.i. of 0.01 and supernatants were collected at 0, 6, 24, 48, and 72 hours post inoculation (p.i.) and stored at −80° C. until titration. The mean titers of each time point were compared by ANOVA, followed by Tukey's HSD post-hoc test.

Cats. Four 12-week old, female SPF cats were used (Liberty Research, Waverly, N.Y.). Cats were housed in individual cages in rooms with controlled temperature, humidity, and lighting. They were fed a combination of dry and moist diets. Each group of cats was housed in a separate Biocontainment Level-2 room. All cats were acclimated for 13 days before virus exposure. Two cats were inoculated oronasally with 2×10₅ TCID₅₀ of the FHV1ΔBAC. The other two cats were inoculated oronasally with 2×10₅ TCID₅₀ of the C-27 strain wild type virus (positive control) or Eagle's Minimum Essential Medium (negative control). Clinical signs induced by inoculation of the viruses were scored as described in the USDA Supplemental Assay Method 311 (U.S. Department of Agriculture, Animal and Plant Health Inspection Service, National Animal Veterinary Services Laboratory, 1985; Table 1). Oral swabs were collected from each cat at days 0, 3, 6, 9, 14, and 21 post inoculation (p.i.) for virus isolation (VI). Serum samples were collected from each cat at days 0, 14, and 21 p.i. for virus neutralization (VN) testing. The cat study was reviewed and approved by the Animal Use Committee at Michigan State University.

IX. Differentiating Infected from Vaccinated Individuals (DIVA) Assay Concept

The use of vaccination to control infectious diseases has been practiced for a long time in human and veterinary medicine. In some areas it is beneficial to be able to distinguish between immunity generated from exposure to natural infection (e.g. field virus) and that obtained from vaccination. In some instances even though an individual has been vaccinated and would yield a positive test (i.e. presence of antibodies) result they are still subject to becoming infected with the disease because the individual does not possess active immunity (activation of memory T and B cells). Some countries also have regulations against importation of livestock that test positive for a particular disease because they are concerned about an outbreak. Therefore it would be desirable to be able to distinguish between individuals that exhibit immunity based upon actual infection or vaccination. The term DIVA was first reported by Dr. Van Oirschot in 1999, in a review discussing the effect of DIVA vaccines and related diagnostic tests for herpesviruses and pestiviruses in swine and cattle. Further, it was reported that pseudorabies and bovine herpesvirus 1 DNA vaccines had been demonstrated to reduce transmission of wild-type virus in pigs and cattle in the lab and in the field (Van Oirschot, J. Biotechnol. 73(2-3):195-205, 1999). Further, the DNA approach has been reported for avian influenza, pseudorabies (i.e. Aujeszky's disease), and proposed for foot and mouth disease (Capua et al., Dev Biol 119:229-33, 2004; Gomez-Sebastan et al., J. Virol. Methods 153(1):29-35, 2008; Pasik, Anim. Health Res. Rev. 5(2):257-262, 2004; Suarez, Biologicals 33(4):221-226, 2005).

Therefore it is contemplated that it would be of benefit to be able to distinguish between FHV-1 immunity due to infection or due to vaccination. Furthermore, this concept was recently shown to be possible with pseudorabies virus, where a recombinant gE gene was expressed in a baculovirus vector system in insect larvae. An indirect PRV gE-ELISA was developed which showed good correlation with two commercial assays and better sensitivity for certain swine sera samples (Gomez-Sebastan et al., J. Virol. Methods 153(1):29-35, 2008). Thus, a prothetic DIVA assay for detection of FHV-1 is proposed. The FHV-1 proposed assay includes detection of the complete FHV-1; FHV-1 mutants lacking gG gene, gI gene, gC gene, gE gene or at least one non-essential; FHV-1 genome as set forth in SEQ ID NO:1; FHV-1 genome as set forth in SEQ ID NO:2; FHV-1 genome as set forth in SEQ ID NO:3; FHV-1 genome as set forth in SEQ ID NO:4 and combinations thereof.

A possible format is an ELISA assay in which microwells are coated with purified virions (assay1) or with a specific viral protein which is not produced by the gene deleted vaccine (assay2). Cats that were naturally infected would have antibodies to the full complement of viral structural proteins in their serum and would test positive, both with assay 1 and assay 2. In contrast, cats that had been vaccinated with, for example, a gC-mutant would react positive in assay1, because this gene deleted vaccine is still highly immunogenic and has induced antibodies to all structural proteins present in it. These cats would however react negative in assay 2 (plate coated with FHV-1 gC in this example), since the absence of gC in the vaccine precluded formation of antibodies against this glycoprotein.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); μg (micrograms); ng (nanograms); l or L (liters); ml (milliliters); pi (microliters); cm (centimeters); mm (millimeters); μm (micrometers); nm (nanometers); ° C. (degrees Centigrade); U (units), mU (milliunits); min. (minutes); sec. (seconds); % (percent); kb (kilobase); by (base pair); ATCC (American Type Culture Collection); BAC (bacterial artificial chromosome); CRFK (Crandell Reese feline kidney); FBS (fetal bovine serum); FHV (feline herpes virus); MOI (multiplicity of infection); ORF (open reading frame); PCR (polymerase chain reaction).

### Example 1

### Construction of Infectious FHV-1 BAC Clones

Overview. BAC vectors containing the pBeloBAC11 (Invitrogen) backbone, chloramphenicol resistance gene, gfp gene, and targeting homologous regions were constructed. Purified FHV-1 viral genome and BAC vector DNA were co-transfected into Crandell Reese feline kidney (CRFK) cells by electroporation. Homologous recombination takes place in the CRFK cells resulting in replacement of the targeted FHV-1 gene with the BAC plasmid. Under selection, replication of nonrecombinant viral genomes was suppressed. Recombinant viruses, which produce fluorescent plaques, were isolated by plaque purification. Recombinant viral genomes were propagated in the CFRK cells, and as a natural process of herpesvirus replication, the viral genome becomes circularized. DNA was extracted from transfected cells when clear plaques were observed. Extracted DNA was transferred into E. coli DH10B electrocompetent cells, which are defective in the RecABCD recombination system. Only circularized DNA survived as an artificial chromosome/plasmid in the bacteria, whereas non-circularized DNA, including cellular genomic DNA and linear viral DNA did not. Only bacteria harboring a recombinant viral genome (FHV-1 BAC clone) survived antibiotic selection. FHV-1 BAC clone DNA was then isolated from selected colonies and the SalI restriction pattern analyzed (Rota et al., Virology, 154:168-179, 1986). Subsequently, the FHV-1 BAC DNA was transfected into CRFK cells to check its in vitro infectivity. CRFK cells were c-transfected with verified FHV-1 BAC clones and a Cre expression vector. FHV-1 BAC DNA was isolated and analyzed by restriction enzyme digestion and sequencing. In alternative embodiments, stable Cre-expressing CRFK cells are generated and transfected with verified FHV-1 BAC clones. FHV-1 BAC DNA is then isolated and analyzed by restriction enzyme digestion and sequencing.

Cells, Viruses, and E. coli strains. CRFK (ATCC No. CCL-94) were maintained in Dulbecco's modified Eagle medium (DMEM) supplemented with 10% heat-inactivated fetal bovine serum (FBS), 100 units/ml penicillin, and 100 μg/ml streptomycin. FHV-1 reference strain C-27 (ATCC No. VR-636) was propagated in CRFK cells by infection at a multiplicity of infection (MOI) of 0.1. E. coli DH10B electrocompetent cells (Invitrogen) were used to harbor the FHV-1 BAC clones.

Viral DNA isolation. After clear viral cytopathic effect (CPE) was observed, virions were isolated from cells and centrifuged in a sucrose gradient at 40,000 g for 2 hr at 4° C. (Rota et al., Virology, 154:168-179, 1986). Viral genomic DNA was isolated from a proteinase K (50 μg/ml)-digested virion pellet by formamide denaturation, and purified by dialysis against 50 mM

Tris-HCl/0.1 M NaCl for 72 hr (Kupiec et al., Anal Biochem, 164:53-59, 1987). Construction of BAC vectors (recombination plasmids). PCR was used for amplification of homologous fragments for insertion into BAC vectors. Oligonucleotide primers were synthesized according to the sequence of the target region, while sequences for loxP and restriction enzyme sites were included when desirable. Roche Expand High Fidelity PLUS PCR System was employed for the PCR reactions. Three BAC vectors with homologous regions designed to replace FHV-1 gE, gI, and thymidine kinase genes, respectively, were constructed. The BAC vectors consist of the pBeloBAC11 (Invitrogen) backbone, chloramphenicol resistance gene and gfp gene as selection markers in E. coli and CRFK cells, respectively. Sites for loxP were also included for future excision of the BAC plasmid backbone.

Construction of the recombinant FHVs. FHV-1 BAC clones were constructed by cotransfection of CRFK cells with intact FHV-1 viral DNA and BAC plasmids. The transfected cells were maintained in DMEM supplemented with 25 μg/ml mycophenolic acid, 250 μg/ml xanthine and 1×HAT supplement. Recombinant FHV-1 DNA was isolated and transfected into DH10B electrocompetent cells. Chloramphenicol resistant colonies were selected, and the presence of FHV-1 genome was confirmed by SalI digestion of purified FHV-1 BAC DNA.

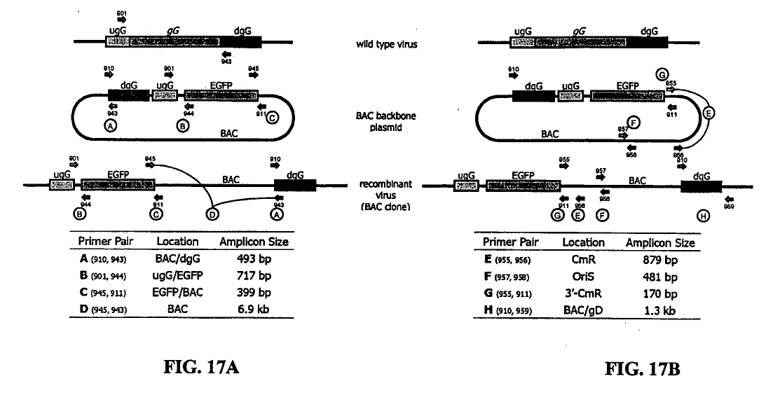

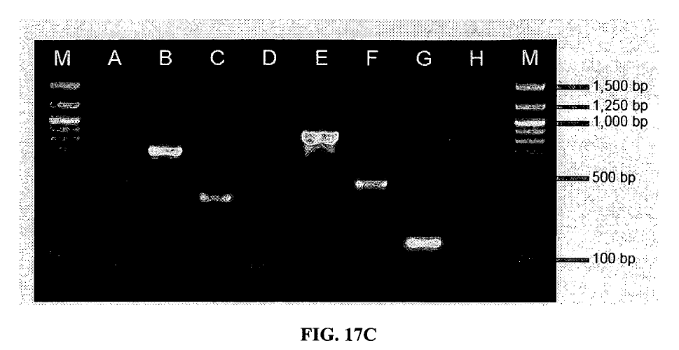

PCR Analyses. PCR was also employed to characterize the FHV-1 BAC clones. The primer pairs employed for PCR analysis are listed in Table 1, while the sequences of the primers are provided in Table 2. Referring to FIGS. 17A-C, FIGS. 17A and B show the location of PCR primer pairs A-D and E-H respectively on the DNA of wild type virus, the BAC backbone plasmid, and the recombinant virus. FIG. 17C shows the detection of different components in the recombinant virus Clone #11A1-01. A-H represents the PCR amplification product generated with primer pairs A-H while M is a 100 bp DNA ladder marker. All primer pairs generated expected amplicon sizes except for D, which did not amplify. BAC sequencing modified these conclusions. In Table 1, what was believed to be dgG was actually the downstream recombination arm (DRA); ugG, was actually the upstream recombination arm (URA); and CmR corresponds to the chloramphenicol resistance gene.

TABLE 1
Primer Pairs For FHV-1 BAC Characterization

| Primer Pair | Template Location | Amplicon Size (bp) |
|---|---|---|
| A (910, 943) | BAC/dgG | 493 |
| B (901, 944) | ugG/EGFP | 717 |
| C (945, 911) | EGFP/BAC | 399 |
| D (945, 943) | BAC | ~6,900 |
| E (955, 956) | CmR | 879 |
| F (957, 958) | OriS | 481 |
| G (955, 911) | 3′-CmR | 170 |
| H (910, 959) | BAC/gD | ~1,300 |

TABLE 2
Primer Sequences

| | SEQ | |
|---|---|---|
| Primer | ID NO | Sequence |
| 901 | 15 | ATACGATTGTATGTCAAAATAC |
| 910 | 16 | AACGGAGTAACCTCGGTGTG |
| 911 | 17 | AATGCTTAATGAATTACAACAG |
| 943 | 18 | AAATCCGTGTTATGTTAACACC |
| 944 | 19 | GCGTTACTATGGGAACATAC |
| 945 | 20 | ATCAGCCATACCACATTTGTAG |
| 955 | 21 | ACCAATTCTCATGTTTGACAGC |
| 956 | 22 | ATCGGCACGTAAGAGGTTCC |
| 957 | 23 | TGGACAGAACAACCTAATGAAC |
| 958 | 24 | ATGACAGATCCATGTGAAGTG |
| 959 | 25 | TAATCACGAGCTCCATCAAGGC |

Generation of Cre-expressing CRFK cells. Briefly, the DNA fragment encoding the cre gene is excised from the pGIKS Cre plasmid (ATCC) by KpnI and SmaI double digestion, followed by ligation to KpnI/SmaI digested pcDNA3.1 expression vector DNA (Invitrogen). This plasmid construct is transfected into CRFK cells by Lipofectin (Invitrogen) and selected in the presence of 100 μg/ml of Zeocin (Invitrogen). Colonies are trypsinized individually using cloning cylinders and further expanded under Zeocin selection. The expression of Cre recombinase is confirmed by immunoblot analysis with anti-Cre antibody (Novagen).

### Example 2

### FHV-1 Genome Sequencing and Gene Annotation

Overview. The recombinant virus clone #11A1-01 isolated as described in Example 1 was propagated in CRFK cells, and then used to infect CRFK monolayers. The circular form of the genomic DNA was extracted from infected cells and used to transform E. coli. Bacterial colonies containing the FHV-1 BAC DNA were extracted from the bacterial cells. The FHV-1 BAC DNA was digested with different restriction enzymes to analyze the integrity of the clone. The digested FHV-1 BAC DNA was separated in agarose gels, and transferred onto nylon membranes. The Southern blots were probed with labeled DNAs targeting the BAC backbone plasmid components. FHV-1 BAC DNA extracted from bacterial cells was also transfected into CRFK cells to demonstrate its ability to producing viral particles in vitro. After the genomic integrity and in vitro replication capacity of the FHV-1 BAC DNA were confirmed, the FHV-1 BAC clone was subjected to whole genome sequencing, followed by gene identification and annotation.

Virus Propagation and DNA Preparation. Recombinant virus Clone #11A1-01 was propagated in CRFK cells. After clear CPE was observed, the supernatant was collected, aliquoted and stored at −80° C. The infectivity of the virus stock was determined by plaque assays. To harvest the circular replication intermediate of the recombinant viral genome for transformation of E. coli, CRFK cells were infected with Clone #11A1-01 at 3 pfu/cell. After a 6-hour incubation period at 37° C. in a CO₂ incubator, infected cells were lysed and subjected to proteinase K digestion. The lysate was extracted twice with phenol-chloroform, and the DNA in the aqueous phase was ethanol precipitated. The precipitated DNA was then washed twice with 70% ethanol. Extraction of FHV-1 BAC DNA from E. coli was carried out using the Large Construct Kit (Qiagen, Valencia, Calif.).

Transformation. DNA samples used for transformation were dialyzed against deionized distilled water to remove residual salts. Electrocompetent E. coli DH10B cells (Invitrogen, Carlsbad, Calif.) were mixed with BAC DNA, and transferred to an electroporation chamber having a 1 mm gap. Electroporation procedures were carried out using a MicroPulser Electroporator (Bio-Rad, Hercules, Calif.), following the manufacturer's instructions. The electroporated cells were incubated in SOC medium for 1 hour and then spread on LB agar plates containing chloramphenicol. FHV-1 BAC DNAs were extracted from the bacterial colonies growing on the selection plate and analyzed.

Restriction Pattern and Southern Blot Analyses. FHV-1 BAC DNA extracted from E. coli DH10B cells, as well as genomic DNA of the FHV-1 C-27 strain, was subjected to single digestion with the restriction endonucleases BamHI, HindIII, and SalI (New England BioLabs, Ipswich, Mass.). The fragments were separated in 0.7% agarose gels. Restriction patterns of the FHV-1 BAC clone were compared to those of FHV-1 C-27 strain for confirmation of BAC insertion and genomic integrity. The fragments separated on the 0.7% agarose gel were transferred to Hybond N+nylon membranes (GE Healthcare, Piscataway, N.J.). DNA probes were synthesized by random priming with digoxigenin-dUTP (Roche Applied Science), using the BAC backbone plasmid without the homologous recombination arms as a template. The pre-hybridization, hybridization, post-hybridization washing and color detection procedures were carried out following the manufacturer's instructions.

DNA Sequencing. When used to sequence a genome, redundancy is required to improve the base-calling accuracy and contiguity of assembled sequence. Pyrosequencing is a relatively new method for real-time nucleotide sequencing. It has rapidly found applications in DNA sequencing, genotyping, single nucleotide polymorphism analysis, allele quantification and whole-genome sequencing. The pyrosequencing method involves four main stages: 1) amplification of target DNA using PCR; 2) conversion of double-stranded DNA into single-stranded DNA templates; 3) hybridization of oligonucleotide primers to a complementary sequence of interest; and 4) the pyrosequencing reaction itself, in which a reaction mixture of enzymes and substrates catalyses the synthesis of complementary nucleotides. Data are shown as a collection of signal peaks in a pyrogram. Recently, a commercial system, developed by 454 Life Sciences (Branford, Conn.) based on the pyrosequencing methodology, became available for automated, high-throughput sequencing (Margulies et al., Nature, 437:376-380, 2005). Once the construction of a BAC clone containing the full genome of FHV-1 was fully confirmed, it was subjected to pyrosequencing using the Genome Sequencer 20 (454 Life Sciences). DNA sequences were assembled into contigs by the software of the Genome Sequencer 20. Gaps between contigs were identified by aligning the contigs to genomic sequences of related alphaherpesviruses, including EHV-1, EHV-4, and PRV, using the LaserGene software package (DNAStar Inc., Madison, Wis.), BLAST, and other web-based tools. Gap closure was achieved by the following steps. First, identified gap regions were amplified using the Expand High Fidelity PLUS PCR System (Roche Applied Sciences, Indianapolis, Ind.), and cloned into a TA cloning vector (Invitrogen). Plasmid clones containing gap-spanning fragments were then sequenced in both directions, using dideoxy chain terminator sequencing chemistries (Sanger et al., Proc Natl Acad Sci USA, 74:5463-5467, 1977) and the Applied Biosystems (Foster City, Calif.) Prism 3100 automated DNA sequencer.

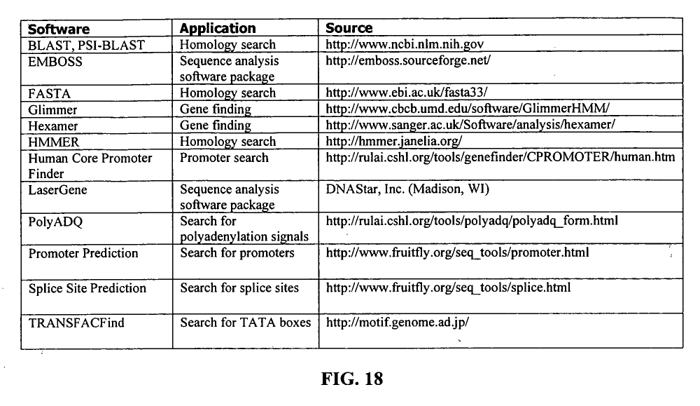

Sequence Analysis. In order to identify the complete set of genes of the FHV-1 genome, the genomic sequence was searched for open reading frames (ORFs), polyadenylation signals, and promoters. ORF searches were performed with coding region prediction software, including ORF Finder (Gish and States, Nat Genet, 3:266-272, 1993) and EMBOSS (Rice et al., Trends Genet, 16:276-277, 2000). ORFs encoding proteins of greater than or equal to 60 amino acids with a methionine start codon (Staden, Nucleic Acids Res, 10:2951-2961, 1982; and Staden and McLachlan, Nucleic Acids Res, 10:141-156, 1982) were evaluated for coding potential using the Hexamer and GlimmerHMM (Majoros et al., Bioinformatics, 20:2878-2879, 2004) programs. Other criteria for ORF identification include similarity to other herpesvirus proteins or to cellular proteins. Homology searches were conducted using BLAST (Altschul et al., J Mol Biol, 215:403-410, 1990), PSI-BLAST (Altschul et al., Nucleic Acid Res, 25:3389-3402, 1997), FASTA (Pearson, Methods Enzymol, 183:63-98, 1990), and HMMER (Burks, Nucleic Acids Res, 27:1-9, 1999) programs with the following databases: PROSITE, Pfam, Prodom, Sbase, Blocks, Domo, and GENBANK. Published mRNA and cDNA data was compared to the FHV-1 genomic sequence using the alignment programs of the LaserGene (DNAStar Inc.) and EMBOSS software packages. To search for polyadenylation signals, the FHV-1 genomic sequence was submitted to PolyADQ, a eukaryotic (human) polyadenylation signal search engine developed by Cold Spring Harbor Laboratory (Tabaska and Zhang, Gene, 231:77-86, 1999). For promoter search, the FHV-1 genomic sequence was submitted to the Berkeley Drosophila Genome Project's neural network-based Promoter Prediction program, an eukaryotic core promoter search engine and the Human Core-Promoter Finder developed by Cold Spring Harbor Laboratory. The core promoters found in this search were examined for the presence of a TATA box consensus using the TRANSFACFind search engine (Heinemeyer et al., Nucleic Acids Res, 27:318-322, 1999). Splice site searches were conducted using the Berkeley Drosophila Genome Project's neural network-based Splice Site Prediction program, and an eukaryotic search engine for donor and acceptor splice sites. All software programs listed here, except for LaserGene, were available over the internet (See FIG. 18 for a listing of software).

### Example 3

### Generation of gG−, gE− and gG−/gE− FHV-1 Mutants

Overview. The E. coli strain SW105, which is capable of recombineering, is made electrocompetent and transformed with FHV-1 BAC DNA. A site-specific mutagenesis procedure with a two step galK selection (Warming et al., Nucleic Acids Res, 33:e36, 2005) is employed to produce gG−, gE− and gG−/gE− mutants. In the first round of homologous recombination, the targeted sequence is replaced with a galK expression cassette, producing bacteria that are phenotypically Gal₊. Positive selection for galK expression is applied. In the second round, the galK cassette is completely removed, producing bacteria that are phenotypically Gal-. Negative selection for galK expression is then applied. Immediately after each step of selection, mini-preps of BAC DNA are prepared and analysed by restriction endonuclease digestion and Southern blot hybridization. Successfully engineered BAC clones, verified by the presence of the expected restriction pattern and Southern blot changes are extracted from SW105 cells and transformed into E. coli DH10B cells for long-term storage. The mutants are then characterized in vitro, by observation of plaque morphology and growth curves.

Cell, Virus and DNA. Crandell Reese feline kidney cells (CRFK) are cultured in Eagle's Minimum Essential Medium (EMEM) containing 10% fetal bovine serum (FBS) and 10 μg/mL ciprofloxacin. The FHV-1 reference strain C-27 is used as the wild type virus throughout this study. Mini-preps of BAC DNA are prepared using an alkaline lysis method. Large scale extraction of BAC DNA from E. coli is carried out using the Large Construct Kit (Qiagen).

Preparation of Electrocompetent Cells and Transformation by Electroporation An E. coli SW 105 colony is inoculated in a 3 mL overnight culture. Subsequently, 500 μl of the culture is inoculated in a flask containing 20 mL of LB medium. When the OD₆₀₀ reaches 0.6, heat shock induction is performed at 42° C. for 15 min. After immediate cooling on ice, the induced E. coli SW105 cells are washed twice with 10 mL ice-cold ddH₂O, and resuspended in 50 μL ice-cold ddH₂O. Electroporation procedures are carried out using a MicroPulser Electroporator (Bio-Rad), following the manufacturer's instructions. The electroporated cells are incubated in SOC medium for 1 hour and then spread on selection plates.

Construction of DNA Fragments to Introduce Mutations. A pair of composite primers, consisting of 50 by regions acting as recombination arms and 20 by regions specific for galK, are synthesized at the Michigan State University Research Technology Support Facility. These primers are used to produce a linear mutating DNA fragment by PCR, in which a galK expression cassette is flanked by 50 by of sequence homologous to the glycoprotein target on both sides. To reduce background, the pgalK plasmid template is digested by DpnI, and the product is gel purified.

Site-specific Mutagenesis. The purified mutating fragment carrying the galK expression cassette is concentrated by ethanol precipitation, and transformed into heat shock induced, electrocompetent SW105/FHV-1 BAC cells. The electroporated cells are incubated in 1 mL SOC medium at 32° C. for recovery, and washed twice in 1×M9 salts (6 g Na₂HPO₄, 3 g KH₂PO₄, 1 g NH₄Cl, and 0.5 g NaCl in 1 L ddH₂O). After the second wash, the cells are plated onto M63 minimal media plates (10 g (NH₄)₂SO₄, 68 g KH₂PO₄, 2.5 mg FeSO₄.7H₂O, and 15 g agar in 1 L ddH₂O) with 0.2% galactose (carbon source), 45 ug/mL L-leucine, 1 ug/mL d-biotin, and 12.5 μg/mL chloramphenicol. Washing in M9 salts is necessary to remove any rich media from the bacteria prior to selection on minimal media. After 3 days of incubation at 32° C., several colonies are streaked on MacConkey agar plates containing galactose and chloramphenicol to obtain single colonies. After 3 days of incubation at 32° C., single, bright red (Gal₊) colonies are picked, grown into 20 mL of LB, induced and made electrocompetent. These cells are subsequently transformed with mutating fragments designed to remove the galK cassette. Before being plated on M63 minimal media plates with 0.2% glycerol, 45 μg/mL L-leucine, 1 ug/mL d-biotin, 0.2% 2-deoxy-galactose (DOG), and 12.5 μg/mL chloramphenicol, the cells are washed twice in 1×M9 salts. After 3 days of incubation at 32° C., single colonies are selected for examination by endonuclease digestion and Southern blotting.

Restriction Pattern and Southern Blot Analyses. Mini-preps of the mutant BAC DNAs are prepared from E. coli SW105 cells and digested with appropriate restriction endonucleases, (e.g., SalI). The fragments are separated in 0.7% agarose gels. Restriction patterns of the mutant clones are compared to those of the parent BAC clone for confirmation of mutagenesis and genomic integrity. Subsequently, the fragments separated on the 0.7% agarose gel are transferred to Hybond N+nylon membranes (GE Healthcare). DNA probes specific for the target gene are synthesized by PCR using the PCR DIG Probe Synthesis Kit (Roche Applied Science). The pre-hybridization, hybridization, post-hybridization washing and color detection procedures are carried out following the manufacturer's instructions.

Plaque Morphology. The viruses to be tested are serially diluted and inoculated on CRFK monolayers in 6-well plates. After a one-hour adsorption period, the diluted virus is removed, and the cells are overlaid with growth medium containing 1% low-melt agarose. The plates are incubated at room temperature for 30 minutes and then at 37° C. in 5% CO₂. After 5 days of incubation, 100 plaques are randomly selected and the diameter is measured.

Multi-step Growth Curve. Triplicate monolayers of CRFK cells are infected at a multiplicity of infection (M01) of 0.01. After an incubation period of 2 hours, cells are washed with PBS, overlaid with EMEM containing 10% FBS, and incubated at 37° C. in 5% CO₂. Supernatants of infected cultures are harvested at successive intervals post infection and the amount of infectious virus is quantitated by titration assay on CRFK cells as described (Costes et al., Microbes Infect, 8:2657-2667, 2006). Further studies were performed to investigate additional embodiments as presented in further examples.

### Example 4

### In Vivo Characterization of A FHV-1 BAC Clone

In order to investigate possible attenuation resulting from BAC cloning, a preliminary experiment was carried out with four specific-pathogen-free (SPF) cats. Two cats were inoculated oronasally with 2×10⁵ TCID₅₀ of the FHV-1 BAC clone. The other two cats were inoculated oronasally with 2×10⁵ TCID₅₀ of the C-27 strain wild type virus (positive control) or Eagle's Minimum Essential Medium (negative control). Clinical signs induced by inoculation of the viruses were recorded daily and a score was calculated using a previously described system (USDA Supplemental Assay Method 311, U.S. Department of Agriculture, Animal and Plant Health Inspection Service, National Animal Veterinary Services Laboratory, 1985; Table 1). Oral swabs were collected from each cat at days 0, 3, 6, 9, 14, and 21 post inoculation for virus isolation (VI). Serum samples were collected from each cat at days 0, 14, and 21 post inoculation for virus neutralization (VN) testing using methods known in the art (Spatz et al., J Gen Virol, 75:1235-1244, 1994). All experiments were reviewed and approved by the Animal Use Committee at Michigan State University.

TABLE 3
Results Of In Vivo Analysis In SPF Cats

| | AJJ5 | | AJC2 | | AJC3 | | AJD3 | |
|---|---|---|---|---|---|---|---|---|
| days | C-27 | | BAC | | BAC | | (MEM) | |
| pi | VI | VN | VI | VN | VI | VN | VI | VN |
| 0 | − | <4 | − | <4 | − | <4 | − | <4 |
| 3 | + | ND | + | ND | + | ND | − | ND |
| 6 | + | ND | + | ND | + | ND | − | ND |
| 9 | + | ND | + | ND | + | ND | − | ND |
| 14 | − | 64 | − | 32 | − | 32 | − | <4 |
| 21 | − | 64 | − | 32 | − | 64 | − | <4 |

| | | | | |
|---|---|---|---|---|
| score | 22 | 22 | 26 | 0 |

ND: not determined.

### Example 5

### Generation of gC⁻, gE⁻ and gC⁻/gE⁻ FHV-1 Mutants

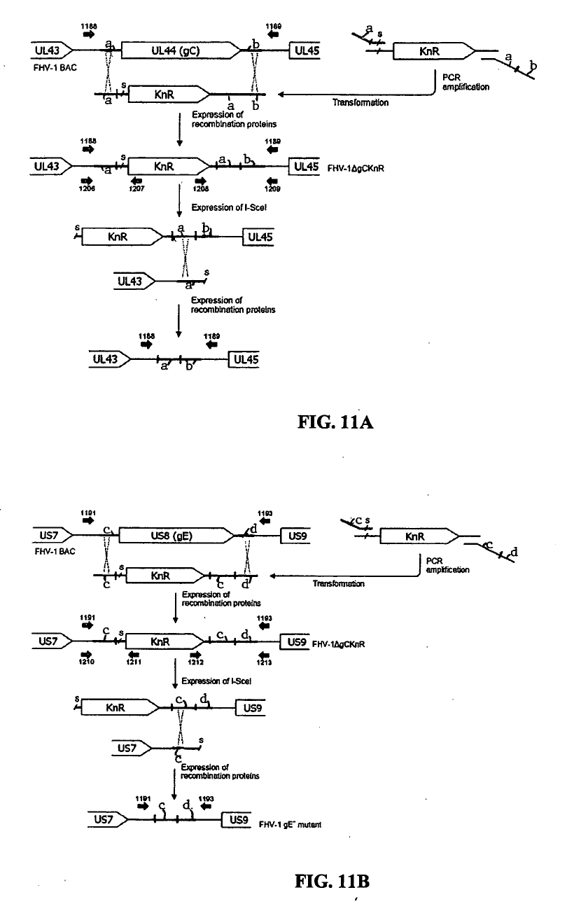

Experimental Design. The E. coli cells of strain SW105, which is capable of recombineering, will be made electrocompetent and transformed with FHV-1 BAC DNA. This will allow further engineering of the BAC. A two-step Red-mediated recombination procedure (Tischer et al., Biotechniques, 40:191-197, 2006) is employed to produce gC−, gE− and gC−/gE− mutants. This modified recombineering strategy is capable of introducing various types of site-specific mutations, including scarless deletion, for our purpose (FIG. 10). Briefly, in the first recombination, the target sequence is replaced by a kanamycin resistance gene expression cassette (KnR) and a recognition site of homing endonuclease I-SceI. Positive selection for KnR expression will be applied during this step. Single colonies will be picked and checked for correct recombination with PCR assays and restriction pattern analysis. Verified clones will be transformed with pBAD-I-sceI, a plasmid carrying homing endonuclease I-SceI and the ampicillin resistance gene. To remove the KnR, these bacteria will be induced for expression of I-SceI and recombination enzymes. To make a gC-/gE-mutant, the gC-mutant will be engineered to delete gE using the same technique. Referring to FIG. 11A, a diagram shows the process of gC engineering and approximate location of primers (arrows) used for PCR assays. The homologous recombination arms, designated as a and b, are shown along with S, I-SceI recognition site (Tischer et al., Biotechniques 40:191-197, 2006). FIG. 11B, shows a diagram of gE engineering and approximate location of primers (arrows) used for PCR assays. The homologous recombination arms, designated as c and d, are shown along with S, I-SceI recognition site (Tischer et al., Biotechniques 40:191-197, 2006). Successfully engineered BAC clones, verified by PCR, restriction pattern analysis and sequencing, will be extracted from SW105 cells and transformed into E. coli DH10B cells for long-term storage. The mutants are then characterized in vitro, by observation of plaque morphology and growth curves. Cell, Virus and DNA. Both Crandell Reese feline kidney cells (CRFK, ATCC CCL-94) and feline corneal epithelial (FCE) cells (Dr. Bienzle, Univ. of Guelph, Ontario, Canada) will be used for in vitro characterization. CRFK cells will be cultured in Eagle's Minimum Essential Medium (EMEM) containing 10% fetal bovine serum (FBS) and 10 ug/mL ciprofloxacin. The FCE cells will be cultured in supplemented hormonal epithelial medium (SHEM) that consists of DMEM-F12 containing 10% fetal bovine serum, 100 U/mL penicillin, 100 μg/mL streptomycin, 2 mM L-glutamine, 0.1 μg/mL cholera toxin, 10 ng/mL epithelial growth factor, 1 μg/mL hydrocortisone, and 5 μg/mL insulin (Sandmeyer et al., Am J Vet Res, 66:205-9, 2005). The FHV-1 reference strain C-27 (ATCC VR-636) will be used as wild type virus throughout this study. Mini-preps of BAC DNA will be carried out using the alkaline lysis method. Large-scale preparation of BAC DNA will be carried out using the Large Construct Kit (Qiagen).

Preparation of Electrocompetent Cells and Transformation by Electroporation. An E. coli SW105 colony will be inoculated in a 3 mL overnight culture. Subsequently, 500 μl of the culture will be inoculated with 20 mL of LB medium. When the OD₆₀₀ of the culture reaches 0.6, heat shock induction will be performed at 42° C. for 15 min. After immediate cooling on ice, the induced E. coli SW105 cells will be washed twice with 10 mL of ice-cold ddH₂O, and resuspended in 50 μL ice-cold ddH₂O. Electroporation procedures will be carried out using a MicroPulser Electroporator (Bio-Rad), following the manufacturer's instructions. The electroporated cells will be incubated in SOC medium for 1 hour and then spread on selection plates.

Construction of DNA Fragments to Introduce Mutation A pair of composite primers consisting of 50 by regions acting as recombination arms and 22-25 by regions specific for KnR will be commercially synthesized and polyacrylamide gel electrophoresis purified (Integrated DNA Technology). These primers will be used in a PCR to produce a linear mutating DNA fragment, in which the KnR is flanked by 50 by of sequence homologous to—20—the glycoprotein target on both sides. To reduce background, the pEPkan-S plasmid template will be digested by DpnI, and the PCR product will be gel purified.

Site-specific Mutagenesis. The purified mutating fragment carrying KnR will be transferred into heat shock induced, electrocompetent SW105/FHV-1 BAC cells. The electroporated cells will be incubated in 1 mL LB medium at 32° C. for recovery before being plated on LB agar containing chloramphenicol (Cm) and kanamycin (Kn). Single colonies obtained after 24-36 hours of incubation at 32° C. will be examined for correct recombination by PCR and restriction pattern analysis. Confirmed clones will be transformed with pBAD-ISceI and plated on LB agar containing Cm, Kn, and ampicillin (Amp). Colonies containing recombined BAC and pBAD-I-SceI will be grown at 32° C. until early logarithmic phase, followed by arabinose induction of ISceI expression and heat shock induction of Red recombination. After recovery at 32° C., the bacteria will be plated on LB agar containing Cm and Amp. Single colonies grow after 24-36 hours will be picked for PCR assays, restriction pattern and sequence analyses.

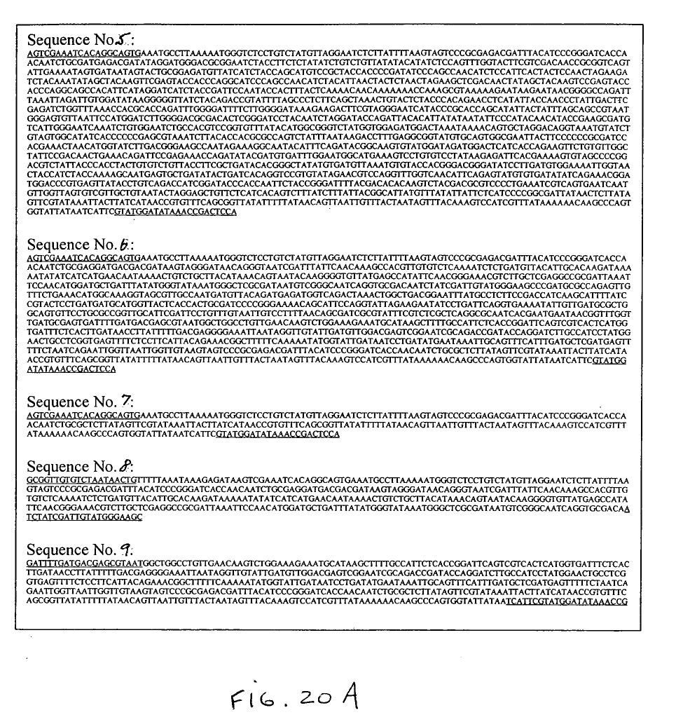

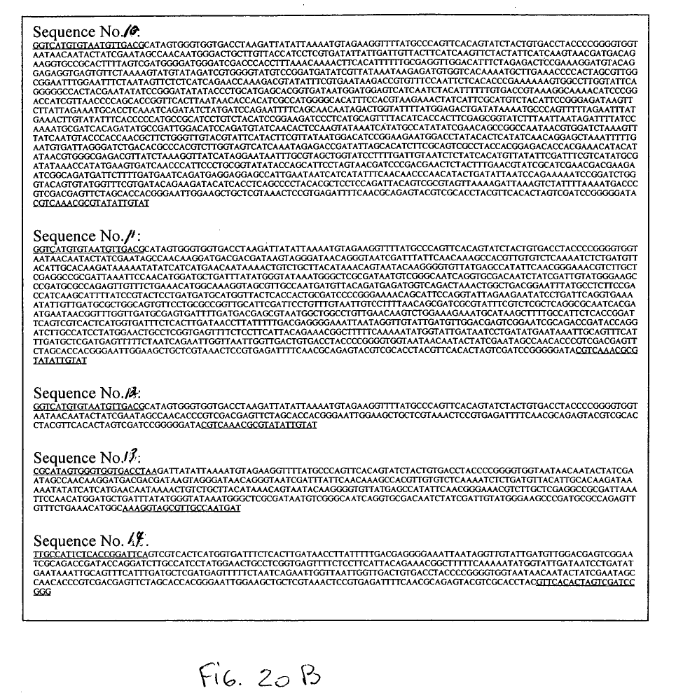

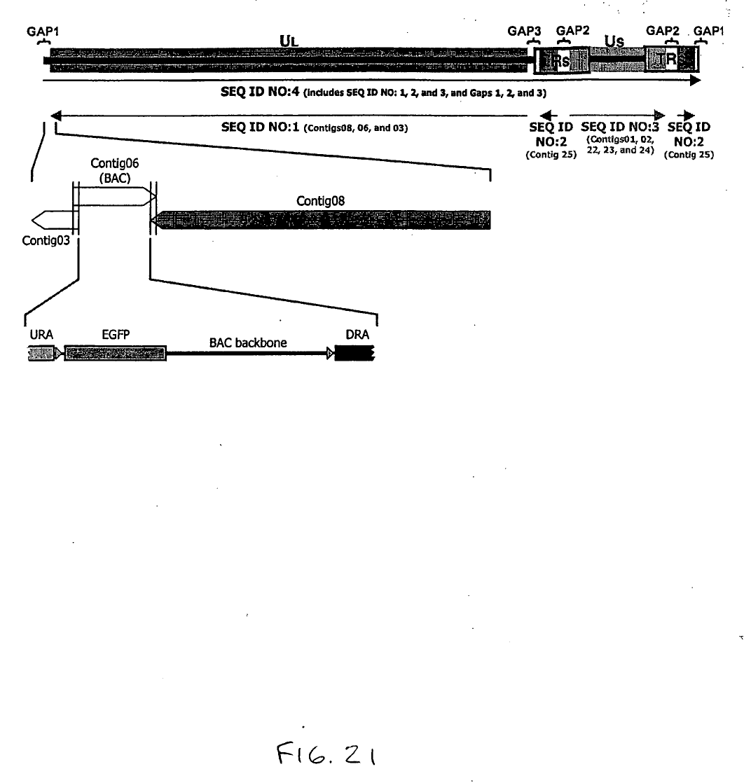

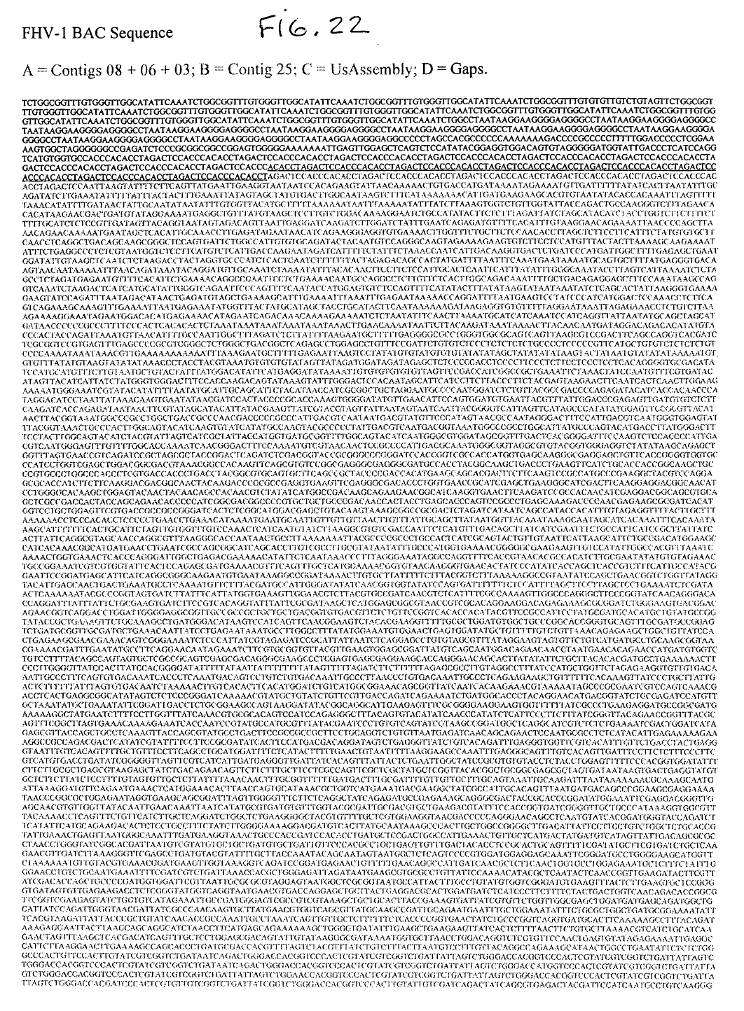

PCR Assays and Sequence Analysis. PCR was also employed to characterize the FHV-1 BAC clones (see FIGS. 11A and B for a schematic). The primer pairs employed for PCR analysis are listed in Table 4, the amplicon sequences (with the primers underlined) are shown in FIGS. 20A and 20B, while electrophoresis results are provided in FIG. 12. Turning to FIG. 12A, the PCR reactions were carried out using primer pairs B (lanes B1-B4) and C (lanes C1-C4), with four FHV-1ΔgCKnR clones as template DNA and parent FHV-1 BAC as negative control (lanes B− and C−). The PCR products were separated in a 1% agarose gel and stained with ethidium bromide. The fact that all the amplicons have the expected size (Table 4) suggests that the KnR has properly replaced gC in these FHV-1ΔgCKnR clones. Additionally, in FIG. 12B, the PCR reactions were carried out using primer pairs E (lanes E1-E4) and F (lanes F1-F4), with four FHV-1ΔgEKnR clones as template DNA and parent FHV-1 BAC as negative control (lanes E− and F−). The PCR products were separated in a 1% agarose gel and stained with ethidium bromide. The fact that all the amplicons have the expected size (Table 4) suggests that the KnR has properly replaced gE in these FHV-1ΔgEKnR clones. Further, in FIG. 12C, the PCR reactions were carried out using primers RM1188 and RM1189, with five FHV-1ΔgC clones as template (lanes 1-5), parent FHV-1 BAC as positive control (lane +), and water as negative control (lane −). The PCR products were separated in a 1% agarose gel and stained with ethidium bromide. While, in FIG. 12D, PCR reactions were carried out using primers RM1191 and RM1193, with ten FHV-1ΔgE clones as template (lanes 1-10), parent FHV-1 BAC as positive control (lane +), and water as negative control (lane −). The PCR products were separated in a 1% agarose gel and stained with ethidium bromide.

TABLE 4
PCR Assays Designed for Identification of BAC Clones

| Primer | | | Size of | |
|---|---|---|---|---|
| Pair | Primers | Template DNA | Amplicon | Seq. No. |
| A | 1188/1189 | FHV-1 BAC | 1,879 bp | 5 |
| A | 1188/1189 | FHV-1ΔgCKnR | 1,319 bp | 6 |
| A | 1188/1189 | FHV-1 gC⁻ mutant | 274 bp | 7 |
| B | 1206/1207 | FHV-1ΔgCKnR | 453 bp | 8 |
| C | 1208/1209 | FHV-1ΔgCKnR | 545 bp | 9 |
| D | 1191/1193 | FHV-1 BAC | 1,867 bp | 10 |
| D | 1191/1193 | FHV-1ΔgEKnR | 1,313 bp | 11 |
| D | 1191/1193 | FHV-1 gE⁻ mutant | 268 bp | 12 |
| E | 1210/1211 | FHV-1ΔgEKnR | 468 bp | 13 |
| F | 1212/1213 | FHV-1ΔgEKnR | 438 bp | 14 |

Restriction Pattern Analysis. Mini-preps of the mutant BAC DNAs will be made from E. coli SW105 cells and digested with appropriate restriction endonucleases, for example, SalI (New England BioLabs). The fragments will be separated in 0.7% agarose gels. Restriction patterns of the mutant clones will be compared to those of the parent BAC clone for confirmation of mutagenesis and genomic integrity.

### Example 6

### In Vitro Characterization of FHV-1 BAC Clone

Plaque Morphology. The viruses to be tested will be serially diluted and inoculated on CRFK and FCE monolayers in 6-well plates. After a one-hour adsorption, the diluted virus will be removed, and the cells will be overlaid with growth medium containing 1% low-melt agarose. The plates will be incubated at room temperature for 30 minutes and then at 37° C. in 5% CO₂. After 5 days of incubation, 100 plaques will be randomly selected and the diameter will be measured. As shown in FIG. 4, fluorescent antibody staining specific for FHV-1 of plaques produced by C-27 strain (A) and plaques produced by the BAC clone (B), two days after inoculating on CRFK monolayers.

Multi-step Growth Curve. Monolayers of CRFK and FCE cells were infected with deletion mutants in triplicate at a multiplicity of infection (MOI) of 0.01. After an incubation period of 2 hours, cells will be washed with PBS, overlaid with EMEM containing 10% FBS, and incubated at 37° C. in 5% CO₂. Supernatants of infected cultures will be harvested at successive intervals post infection and the amount of infectious virus will be titrated on CRFK cells as described previously (Costes et al., Microbes Infect, 8:2657-2667, 2006). As shown in FIGS. 9A and B (where 9B presents the same curve with error bars), the growth curve of the parent wild type strain C-27 and the BAC clone. The wild type virus and the BAC clone were inoculated on CRFK monolayers at an m.o.i. of 0.01 and supernatants were collected and titrated at 0, 6, 24, 48, and 72 hours post inoculation (p.i.). As is obvious from the graph (FIGS. 9A and 9B), both viruses grow to high titers at 72 hours p.i. Error bars on each data point represent ±1 standard deviation.

### Example 7

### Complete FHV-1 Genome Derived from an Infectious BAC Clone

Cloning of the FHV-1 Genome As A BAC. The FHV-1 genome has been cloned in a BAC. The procedure used relies on the use of spontaneous homologous recombinations in the transfected CRFK cells, similar to those used in previous reports (Niikura et al., Arch. Virol. 151(3):537-549, 2006), to insert the BAC vector into the FHV-1 genome. gG (US4) gene was selected as the target because it is not essential for virus growth. To target the BAC vector insertion site to the gG gene, a BAC plasmid, BAC04, was constructed. The BAC04 and, FHV-1 genomic DNA were co-transfected into CRFK cells and the viruses were harvested for plaque purification. By recovery of the virus from fluorescent plaques in CRFK monolayers, one virus clone that consistently produces fluorescent plaques was obtained. CRFK cells were inoculated with this virus and the circular form replication intermediate of the FHV-1 genome was harvested using the method of Hirt (Hirt et al., J. Mol. Biol., 26(2):365-369, 1967), and transformed into E. coli. BAC DNA was purified from E. coli. The SalI pattern of the BAC clone was very similar to that of the parent strain, except for an additional band of the BAC vector, and the end fragments, which were joined together because the genome was circularized (data not shown). In addition to the restriction pattern analysis, PCR assays were used to examine proper insertion of the BAC vector. PCR assays targeting different components, including the EGFP gene, the chloramphenicol resistance gene, the bacterial origin of replication, confirmed that the BAC was inserted into the FHV-1 genome (data not shown); however, primers targeted at the expected junction between the BAC vector and the viral sequence failed to produce expected results. In addition, a pair of primers amplified the full length of an intact gG gene. These results suggested that the BAC was actually not inserted into the gG gene.

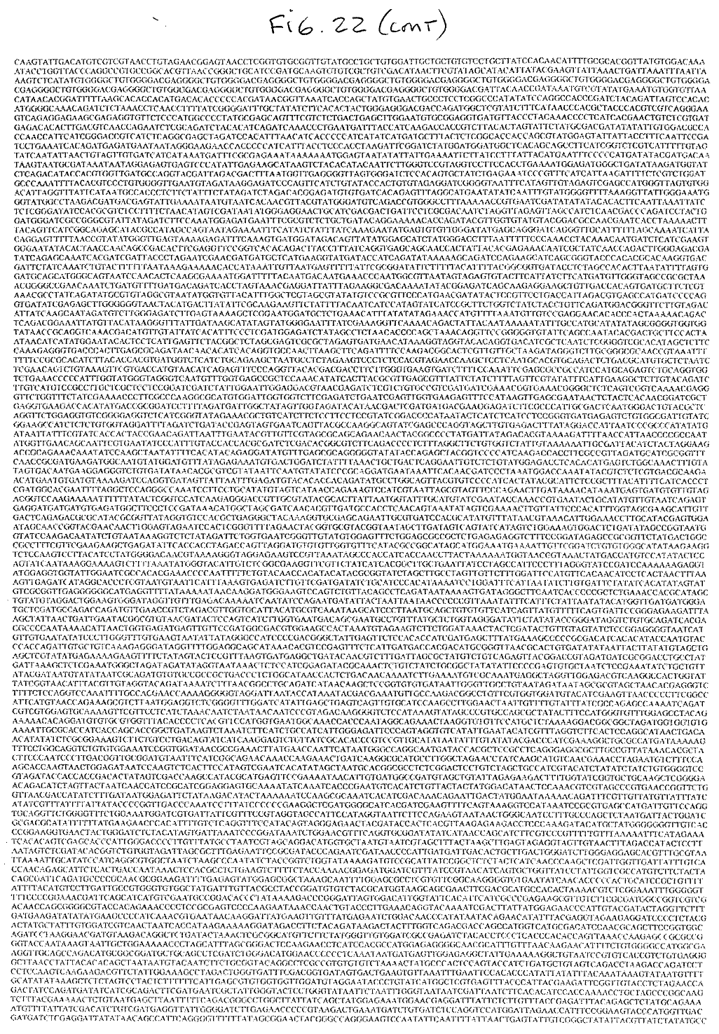

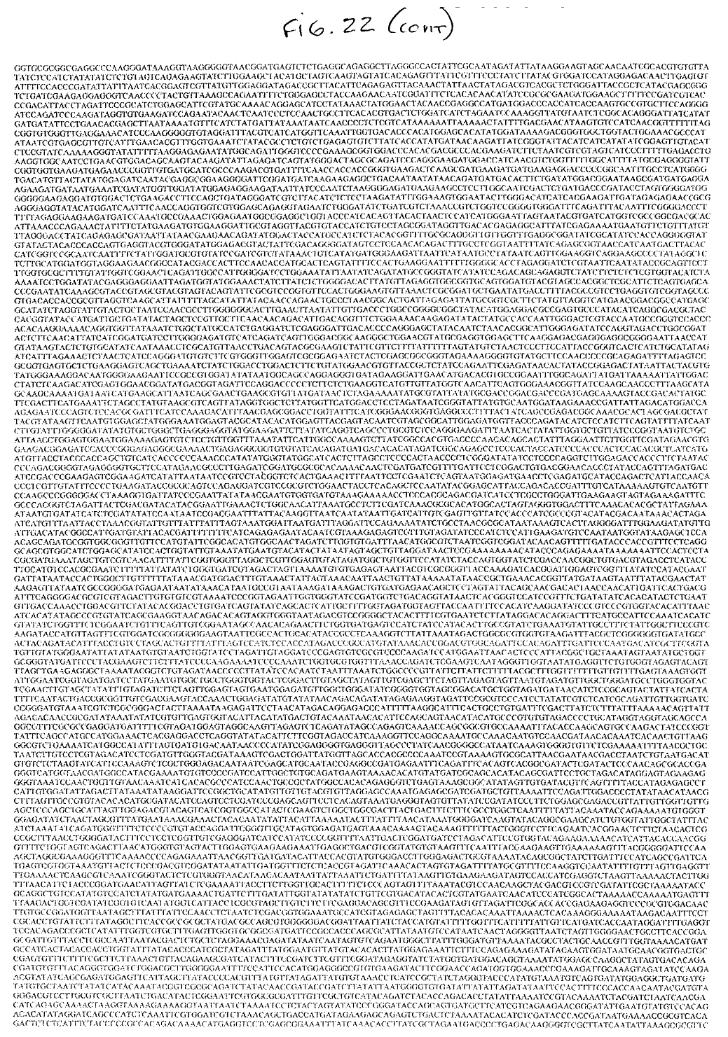

Sequencing, Sequence Assembly, GenBank Accession Number. The sequence reads were initially assembled into contigs using the Newbler assembly program from Roche/454. The assembly generated 25 contigs; 8 major contigs represented FHV-1 sequence, one represents the BAC vector (Contig 6), one represented nonherpesvirus sequence (Contig 3), and the remaining 14 short contigs (length between 94-178 bp; number of reads between 2 and 6) were determined to be bacterial or other contaminating sequences. More than 95% of the reads were utilized in the assembly. Statistics for these contigs are shown in Table 5 although some lengths have expanded. Contig 8, the largest, appears to cover the entirety (or nearly so) of the UL region. By aligning with previous sequence results generated in our laboratory and from GenBank, Contigs 1, 2, 22, 23, 24 were further assembled to form a large contig U_{S}-Assembly, spanning the entire Us and parts of the Repeat Short regions (IRs and TRs). Contig 25 covered most of the remaining Repeat Short regions. Referring to FIG. 21, a diagram shows the physical structure of the FHV-1 genome along with the locations of SEQ ID NO:1, 2, 3, and 4; previous Gap 1, 2, and 3 that are now filled; and a brief description of contigs. SEQ ID NO:4 is the complete nucleic acid listing for the FHV-1 genome. A BLASTN (Altschul et al., Nucleic Acids Res., 25(17):3389-3402, 1997) search against the nucleotide collection database (nr/nt, all GenBank+EMBL+DDBJ+PDB sequences but no EST, STS, GSS, environmental samples or phase 0, 1 or 2 HTGS sequences) found that Contig 3 did, not resemble any known herpesvirus sequence. Primer walking from the ends of Contigs 3, 6, and 8 demonstrated that these three contigs are in fact linked together in the BAC clone. Three gaps were remaining at this point. The gap between Contigs 3+6+8 and 25, which represents the junction of UL and IRS, was designated as Gap 1. The gap between Contigs 25 and Us-Assembly, located inside both Repeat Short regions, was designated as Gap 2. Gap 3 consisted of the beginning and the end of the genome, or the junction of TRS and UL when the genome is circularized. Based on the SalI restriction pattern, Gap 1 was estimated to be 1.0 kb, Gap 2 0.6 kb, and Gap 3 1.0 kb. Attempts to close these gaps by primer walking resulted in partial success. Gaps 1 and 3 were completely closed; the sequence length was as predicted. Gap 2 could not be fully sequenced. Primer walking results revealed that this region contains repetitive units of very high G+C content (77%). Both BigDye Terminator 3.0 and dGTP BigDye Terminator chemistries failed to sequence through this gap. The remaining 314 by of the unsequenced part was filled with the same repetitive unit found at both ends of the gap.

TABLE 5
Contigs Initially Assembled by Newbler Program

| | Contig Length | Number of | Average Read |
|---|---|---|---|
| Contig ID | (bp)* | Reads | Depth |
| Contig00001 | 439 | 317 | 75 |
| Contig00002 | 4,886 | 1,565 | 33 |
| Contig00003 | 2,663 | 842 | 33 |
| Contig00006 | 8,044 | 2,380 | 31 |
| Contig00008 | 105,901 | 31,939 | 31 |
| Contig00022 | 121 | 80 | 69 |
| Contig00023 | 5,238 | 2,489 | 49 |
| Contig00024 | 1,048 | 374 | 37 |
| Contig00025 | 5,787 | 3,476 | 62 |

*Note: Some contigs have been expanded.

The sequence is deposited in GenBank under the accession number xxxxxxx. These sequencing results demonstrated that the BAC vector was inserted between the UL and the IRS, with an addition of a 2.663-kb non-herpesvirus sequence. It is known that many herpesviruses can acquire parts of host genomes, presumably during replication. It has also been shown that this can occur during BAC cloning (Niikura et al., Arch. Virol. 151(3):537-49, 2006). A BLAST search in the cat genome project's WGS Contigs database revealed that a 220 by portion near the 3′-end of Contig 3 is highly similar to the cat DNA. PCR primers RM1025 and RM1026 (See FIGS. 14A and 14B) targeting a part at the 3′-end of Contig 3 successfully amplified the same sequence from the DNA of un-infected CRFK cells. Therefore, it is very likely that Contig 3 is a part of the cat DNA, acquired by the virus during homologous recombination in CRFK cells.

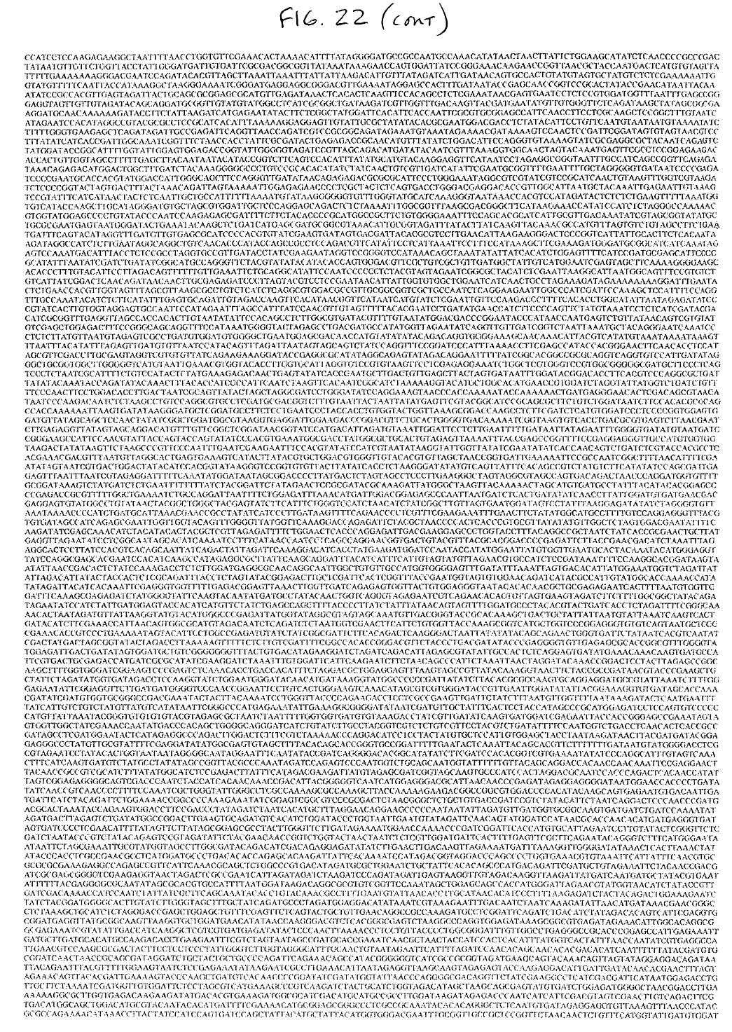

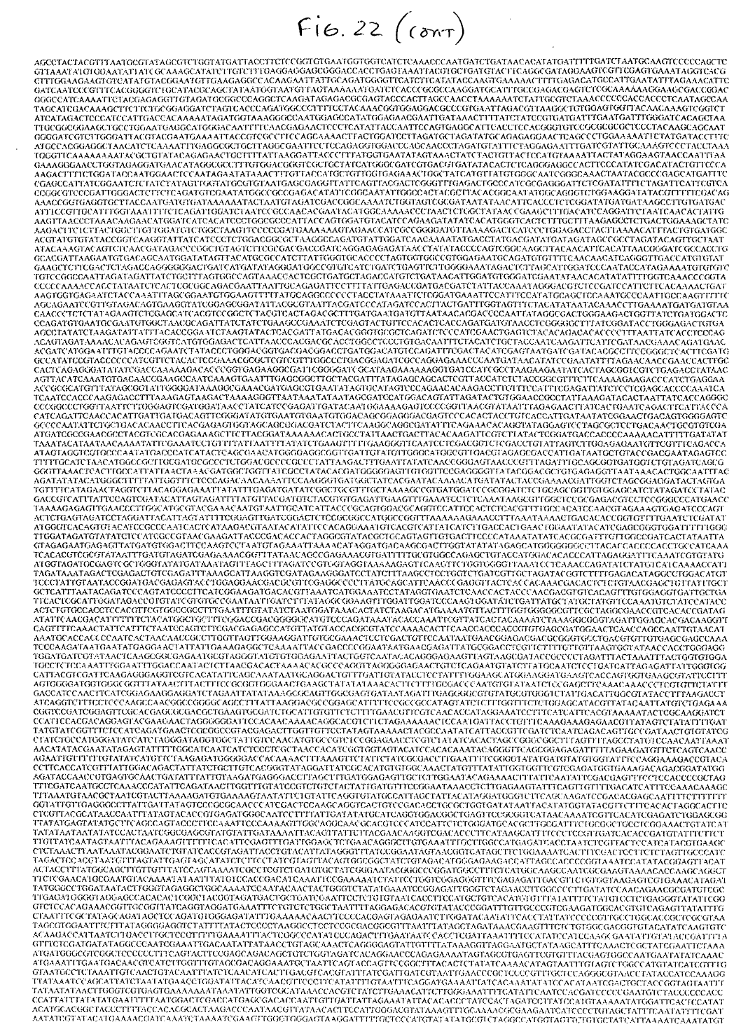

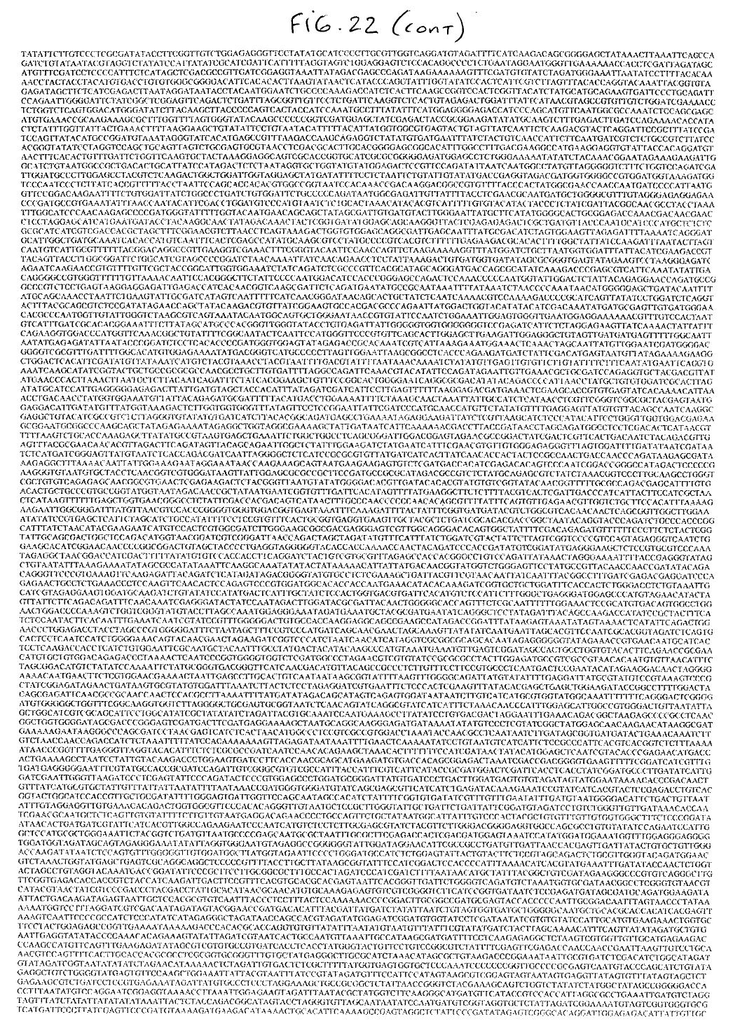

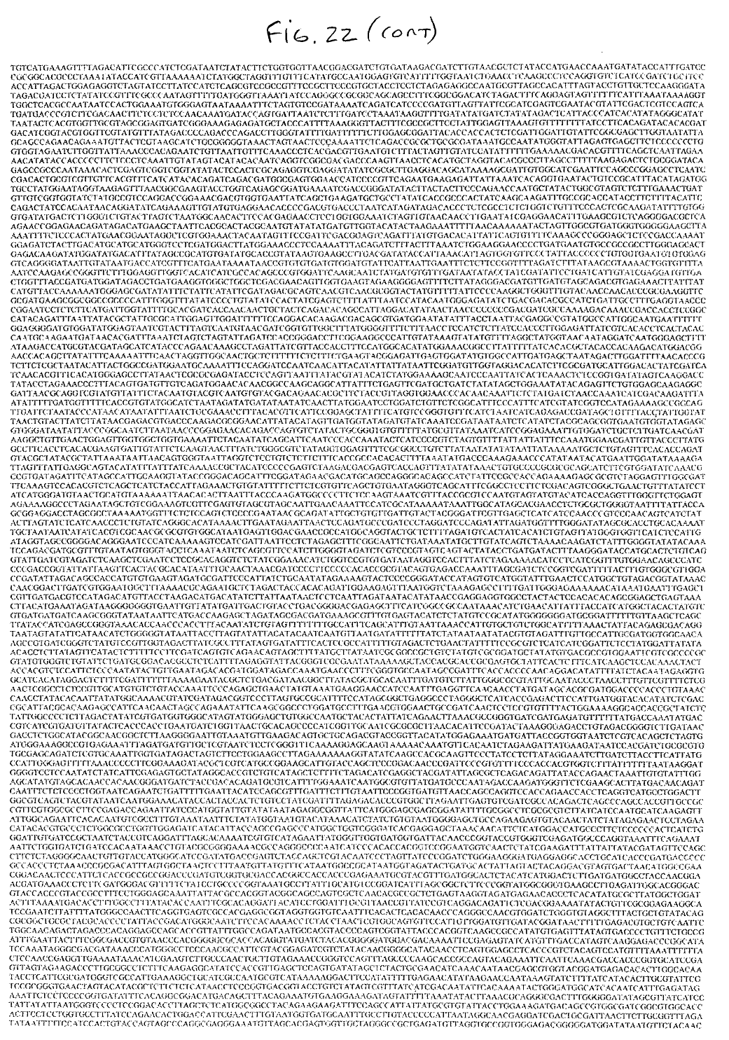

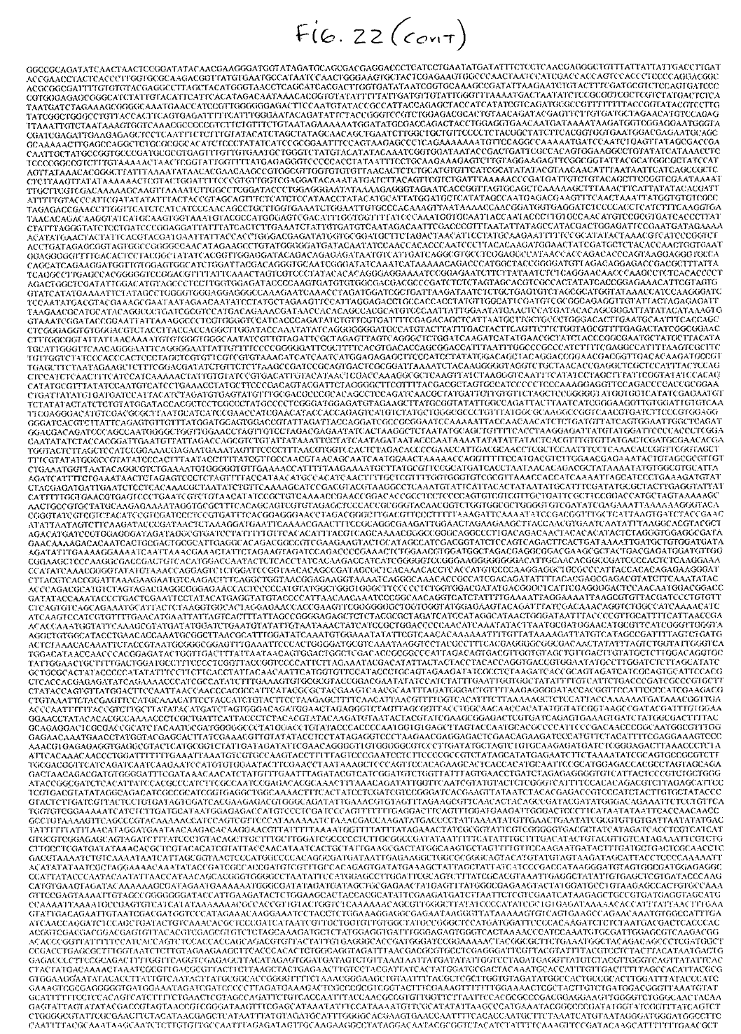

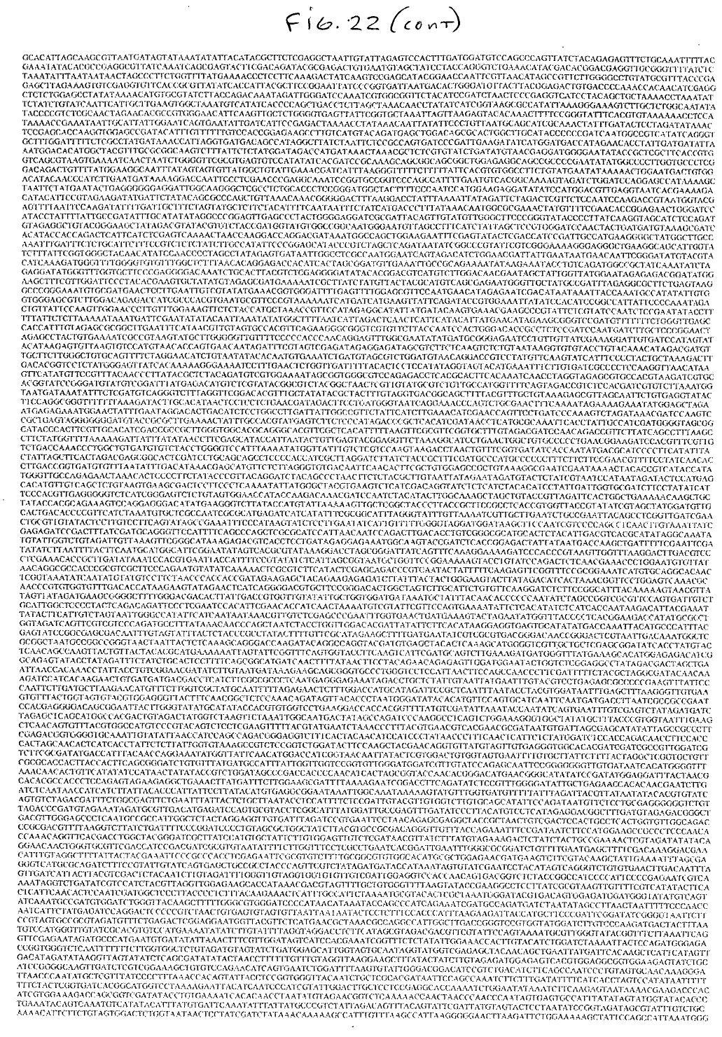

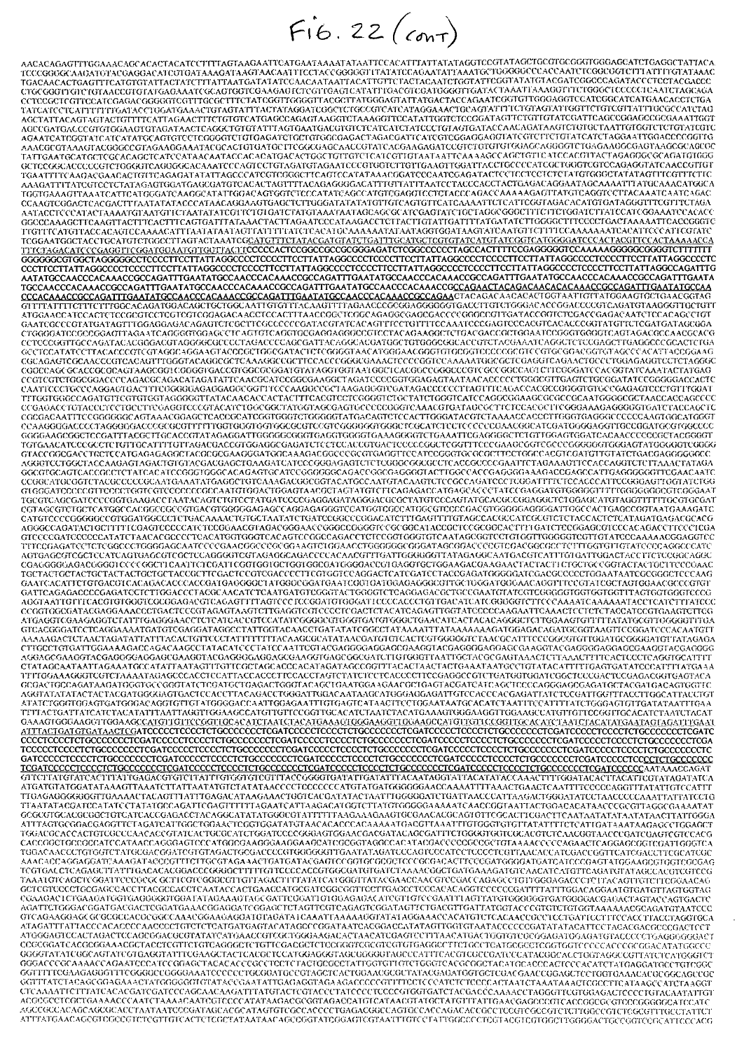

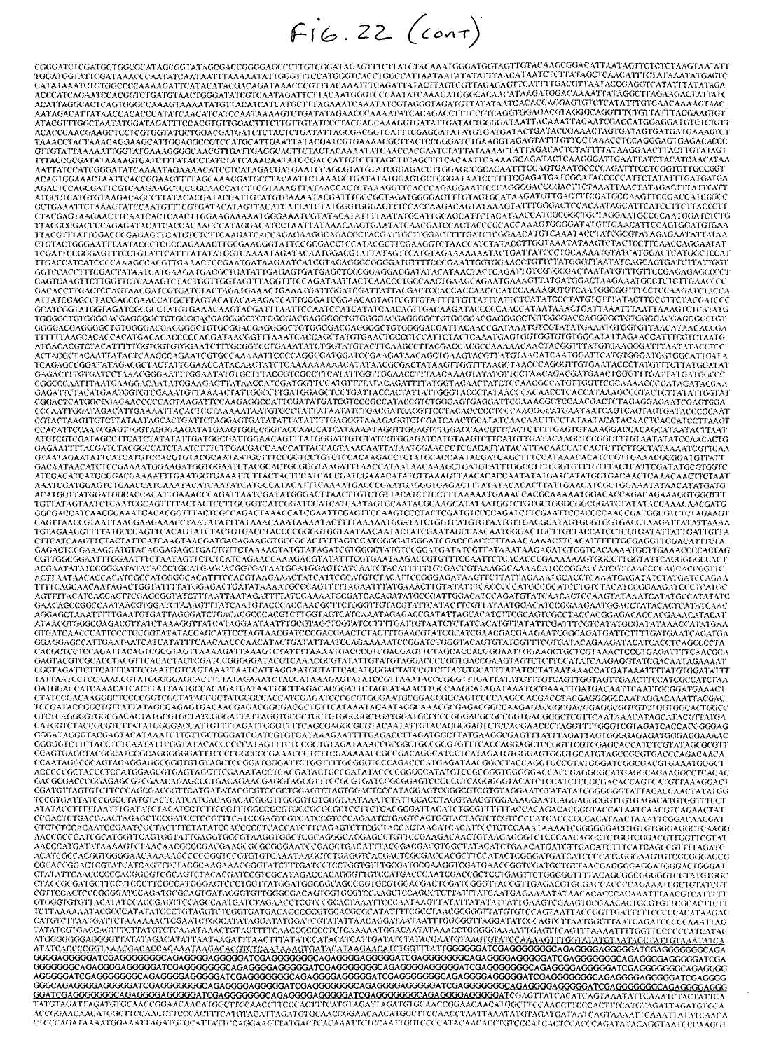

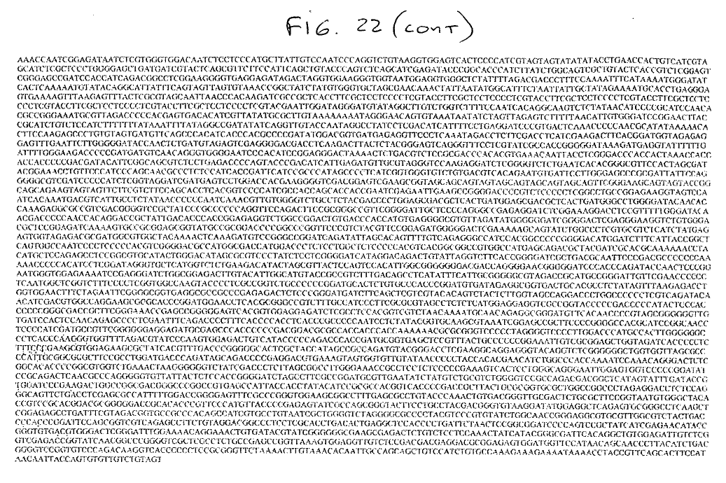

Genome Structure. The FHV-1 genome is approximately 135,796 by in size. The overall G+C content of FHV-1 genome is 45%. The genome consists of a 105,901 by long U_{L} and an 8,440 by long U_{S} region, with the latter being flanked by inverted IRs and TRs elements of 10,496 by each. ORF finding, gene content, gene arrangement. The complete FHV-1 gene arrangement is shown in FIG. 15A while a complete annotated sequence listing of the exemplary embodiment of 147,238 by FHV-1 BAC clone (SEQ. ID. NO:4) is provided in FIG. 15B along with a grouped nucleic acid sequence listing in FIG. 22. Further, FIG. 21 provides a diagram showing the physical structure of the FHV-1 genome. The locations of SEQ ID:1, 2, 3, and 4; Gap 1, 2, and 3; and a listing of corresponding contigs. The figure is not to scale. Two methods were used to identify all proteins encoded in the genome. First, ORFs encoding proteins of >60 amino acids with a methionine start codon were evaluated for coding potential by searching for homologs in other alphaherpesviruses. Homology searches were conducted using BLASTX with the non-redundant protein sequence database nr (Altschul et al., Nucleic Acids Res., 25(17):3389-3402, 1997). Other criteria used included compact gene arrangements on both strands with little gene overlap. As a second approach to verify the annotation/identify new genes in the FHV-1 genome, the sequence was submitted to GeneMarkS (Besemer et al., Nucleic Acids Res. 29(12):2607-2618, 2001). GeneMarkS predicted 10 fewer genes, namely UL3, UL11, UL24, UL26.5, UL43, UL53, US2, US6, US8.5, and US9. However, these genes, except for US2 and US8.5, are consistently present among varicelloviruses (Table 6) and were easily identified using BLASTX.

TABLE 6
Difference in Gene Composition Among Varicelloviruses

| Gene | FHV-1 | EHV-1 | EHV-4 | BHV-1 | BHV-5 | PRV | VZV |
|---|---|---|---|---|---|---|---|
| ICP0 | + | + | + | + | + | + | + |
| UL1 | + | + | + | + | + | + | + |
| UL2 | + | + | + | + | + | + | + |
| UL3 | + | + | + | + | + | + | + |
| UL3.5 | + | + | + | + | + | + | + |
| UL4 | + | + | + | + | + | + | + |
| UL5 | + | + | + | + | + | + | + |
| UL6 | + | + | + | + | + | + | + |
| UL7 | + | + | + | + | + | + | + |
| UL8 | + | + | + | + | + | + | + |
| UL9 | + | + | + | + | + | + | + |
| UL10 | + | + | + | + | + | + | + |
| UL11 | + | + | + | + | + | + | + |
| UL12 | + | + | + | + | + | + | + |
| UL13 | + | + | + | + | + | + | + |
| UL14 | + | + | + | + | + | + | + |
| UL15 | + | + | + | + | + | + | + |
| UL16 | + | + | + | + | + | + | + |
| UL17 | + | + | + | + | + | + | + |
| UL18 | + | + | + | + | + | + | + |
| UL19 | + | + | + | + | + | + | + |
| UL20 | + | + | + | + | + | + | + |
| UL21 | + | + | + | + | + | + | + |
| UL22 | + | + | + | + | + | + | + |
| UL23 | + | + | + | + | + | + | + |
| UL24 | + | + | + | + | + | + | + |
| UL25 | + | + | + | + | + | + | + |
| UL26 | + | + | + | + | + | + | + |
| UL26.5 | + | + | + | + | + | + | + |
| V32 | + | + | + | − | − | − | + |
| UL27 | + | + | + | + | + | + | + |
| UL28 | + | + | + | + | + | + | + |
| UL29 | + | + | + | + | + | + | + |
| UL30 | + | + | + | + | + | + | + |
| UL31 | + | + | + | + | + | + | + |
| UL32 | + | + | + | + | + | + | + |
| UL33 | + | + | + | + | + | + | + |
| UL34 | + | + | + | + | + | + | + |
| UL35 | + | + | + | + | + | + | + |
| UL36 | + | + | + | + | + | + | + |
| UL37 | + | + | + | + | + | + | + |
| UL38 | + | + | + | + | + | + | + |
| UL39 | + | + | + | + | + | + | + |
| UL40 | + | + | + | + | + | + | + |
| UL41 | + | + | + | + | + | + | + |
| UL42 | + | + | + | + | + | + | + |
| UL43 | + | + | + | + | + | + | + |
| UL44 | + | + | + | + | + | + | + |
| UL45 | + | + | + | − | − | − | − |
| VZV | − | − | − | − | − | − | + |
| ORF13 | | | | | | | |
| UL46 | + | + | + | + | + | + | + |
| UL47 | + | + | + | + | + | + | + |
| UL48 | + | + | + | + | + | + | + |
| UL49 | + | + | + | + | + | + | + |
| UL49.5 | + | + | + | + | + | + | + |
| UL50 | + | + | + | + | + | + | + |
| UL51 | + | + | + | + | + | + | + |
| UL52 | + | + | + | + | + | + | + |
| UL53 | + | + | + | + | + | + | + |
| UL54 | + | + | + | + | + | + | + |
| UL55 | + | + | + | − | − | − | + |
| CIRC | + | + | + | + | + | − | + |
| V1 | + | + | + | − | − | − | + |
| UL56 | + | + | + | − | − | + | + |
| V67 | − | + | + | + | + | − | + |
| ICP4 | + | + | + | + | + | + | + |
| US1 | + | + | + | + | + | + | + |
| US10 | + | + | + | − | − | − | + |
| US2 | + | + | + | + | + | + | − |
| US3 | + | + | + | + | + | + | + |
| US4 | + | + | + | + | + | + | − |
| US5 | − | + | + | − | − | − | − |
| US6 | + | + | + | + | + | + | − |
| US7 | + | + | + | + | + | + | + |
| US8 | + | + | + | + | + | + | + |
| US8.5 | − | + | + | − | − | − | + |
| US9 | + | + | + | + | + | + | + |

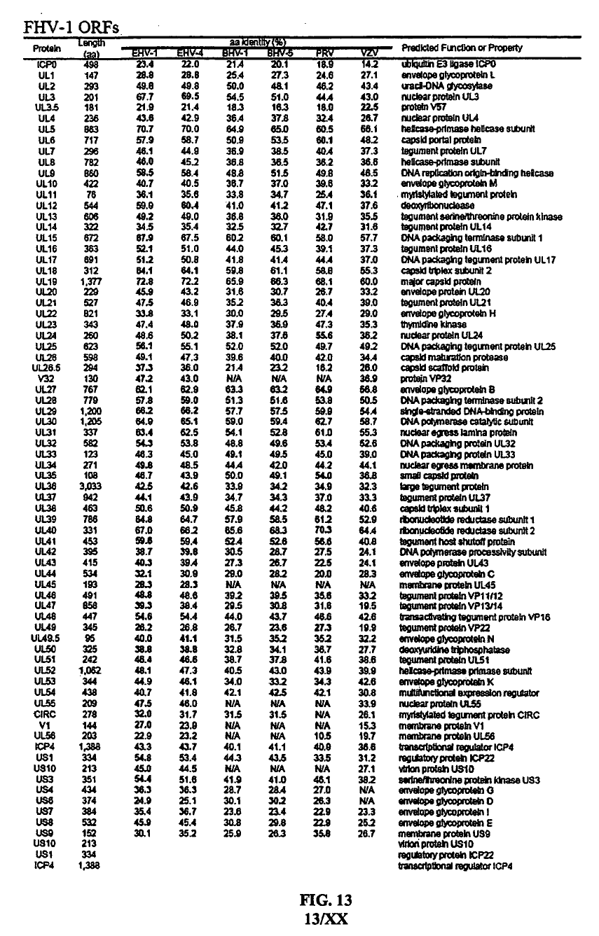

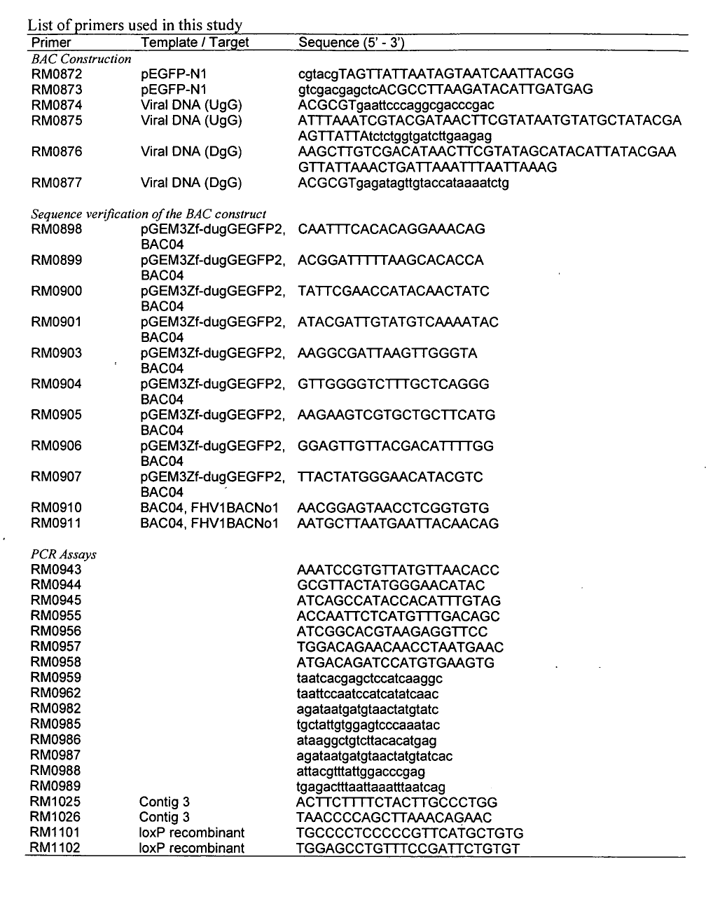

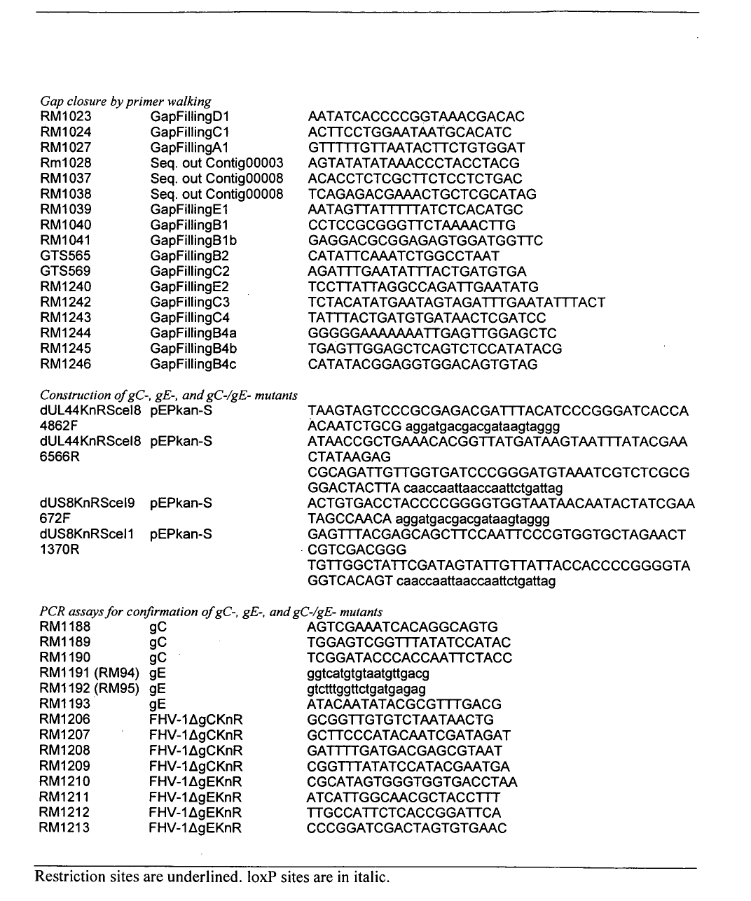

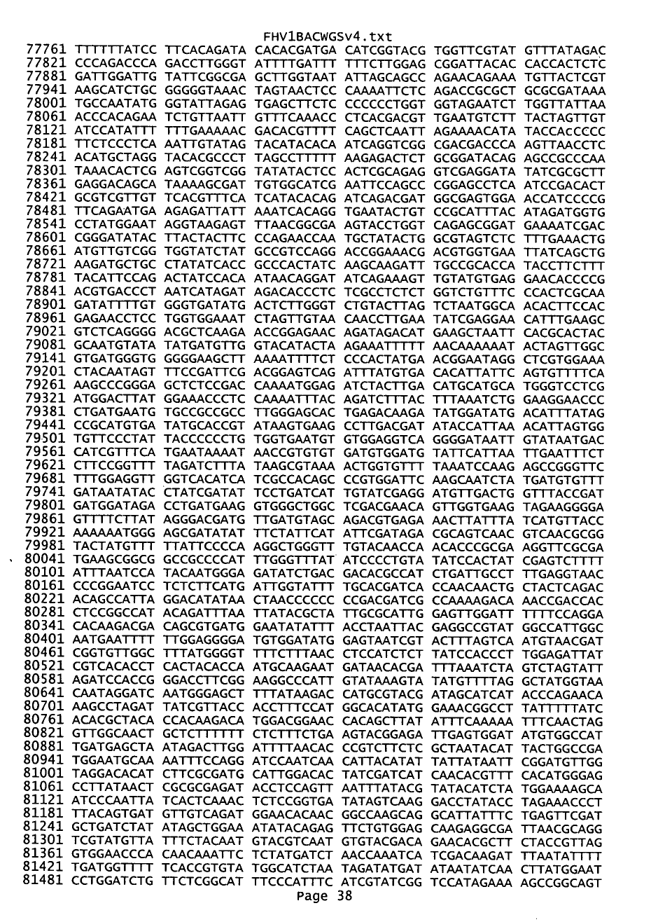

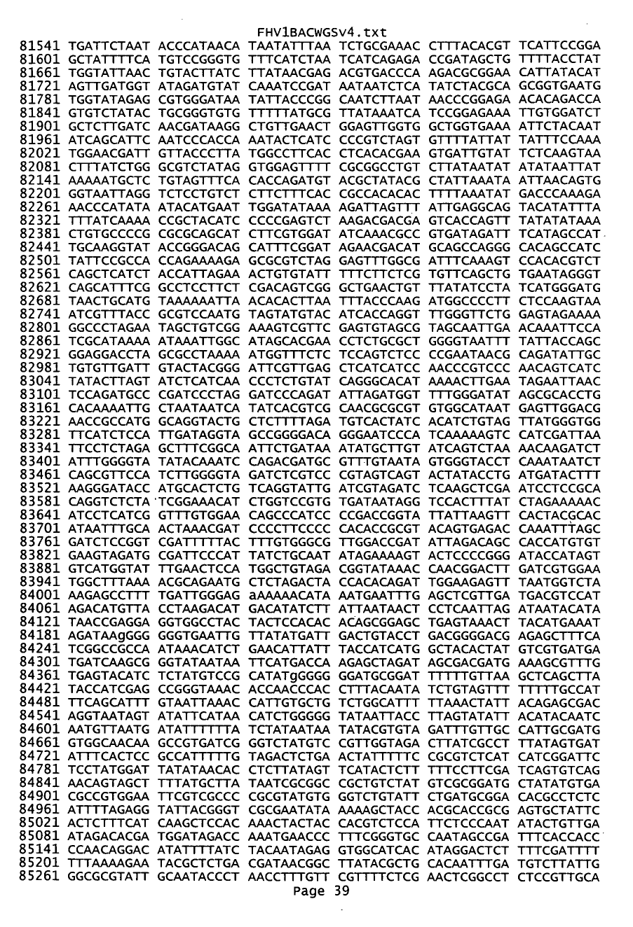

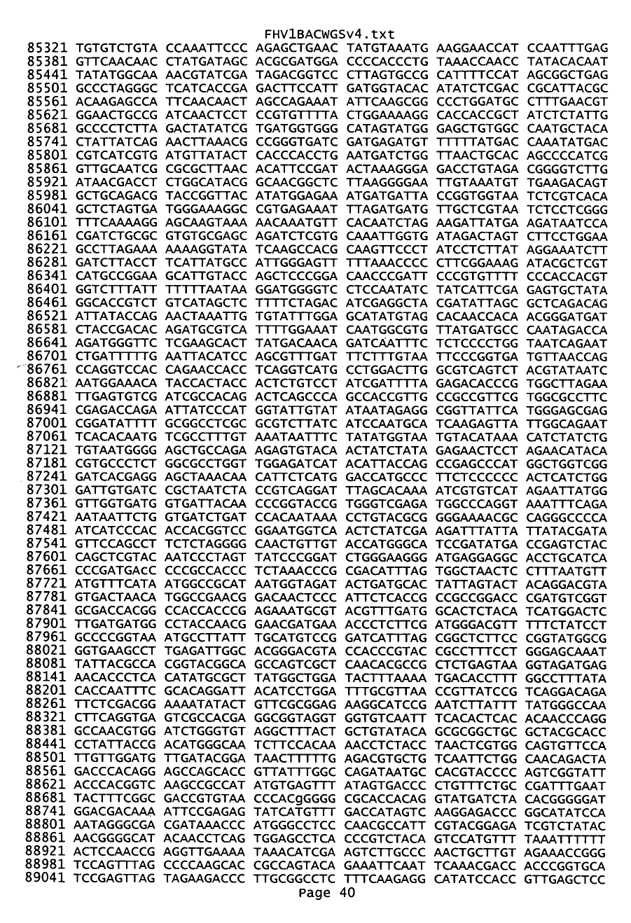

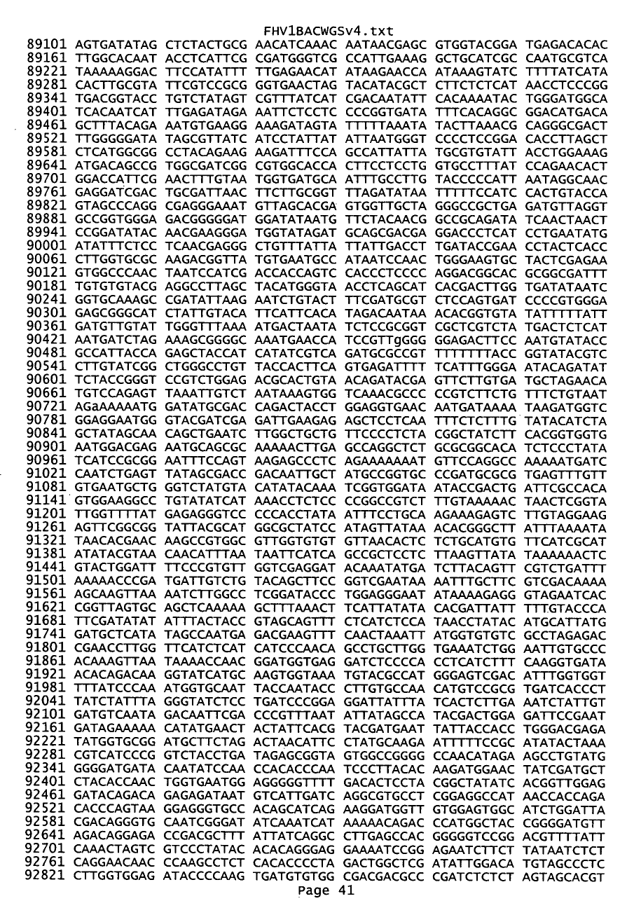

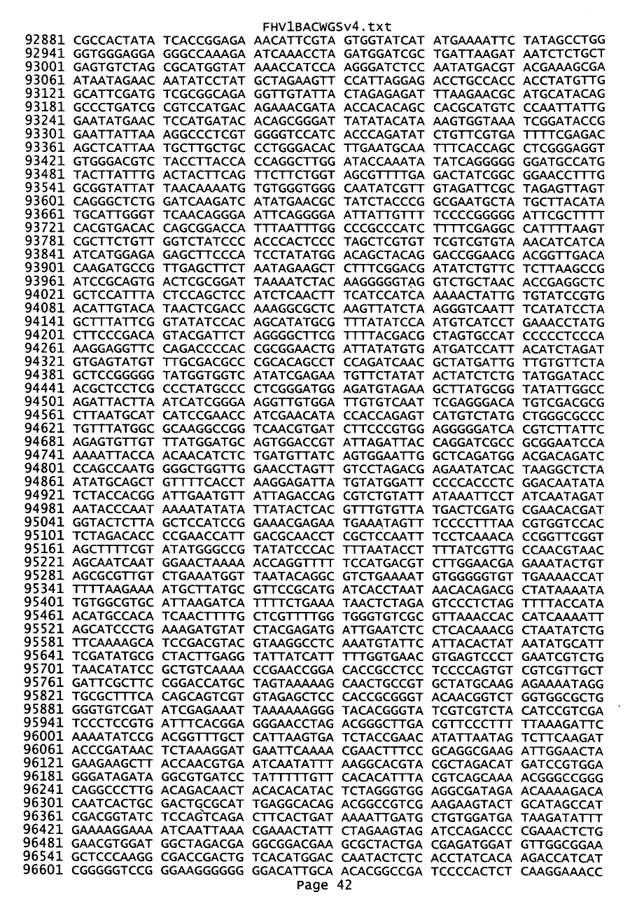

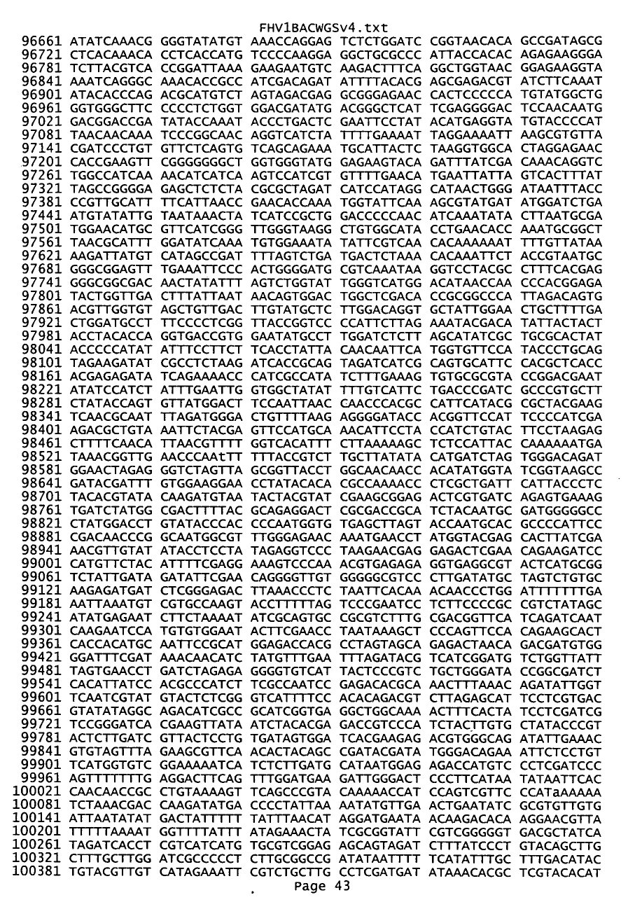

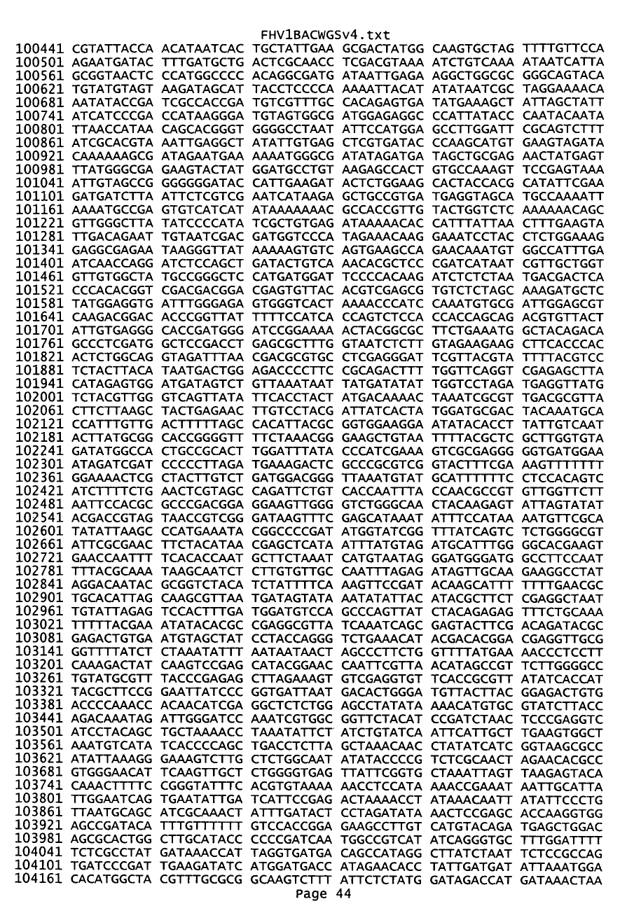

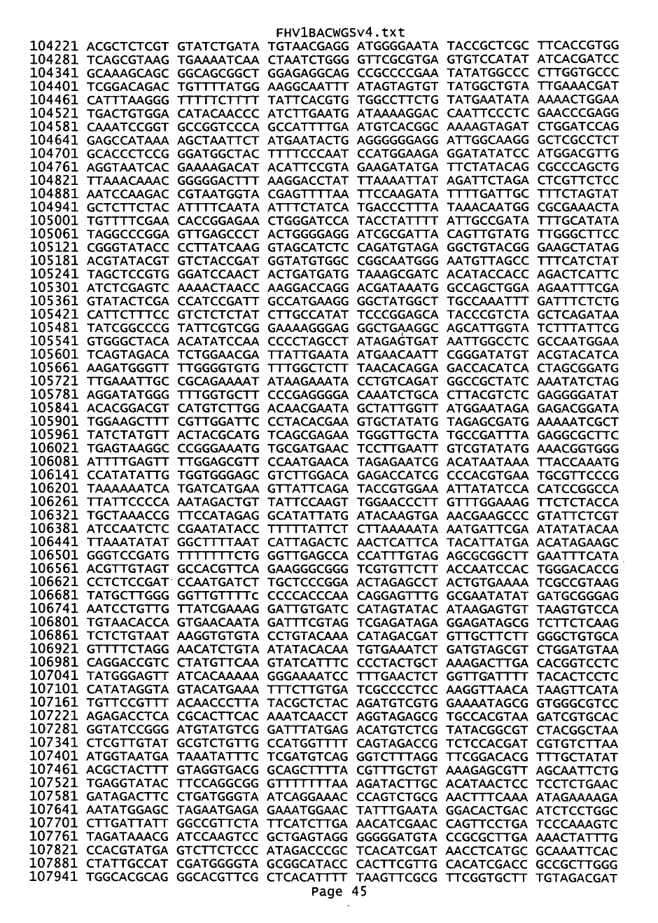

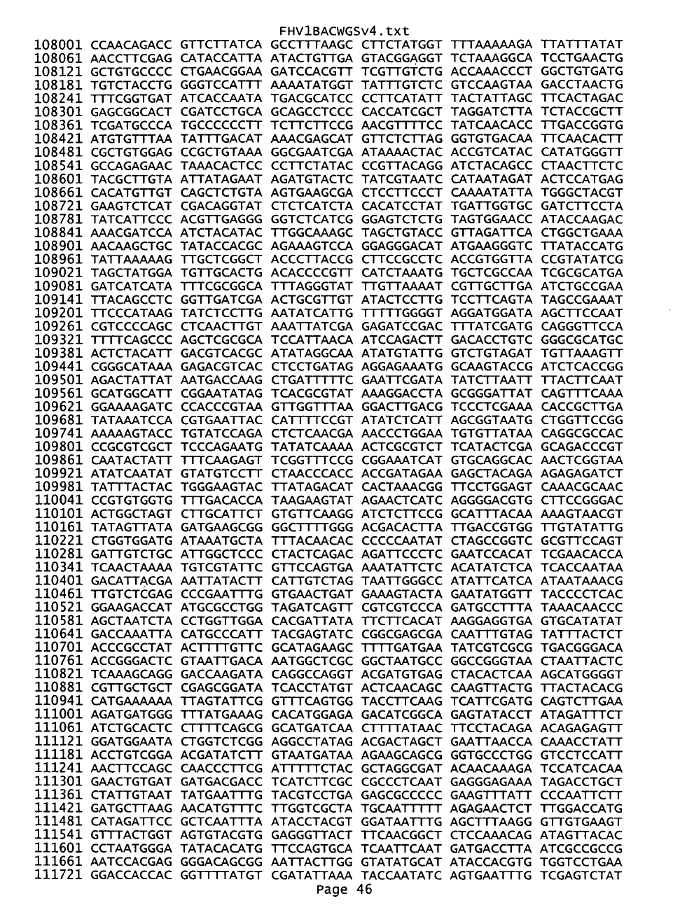

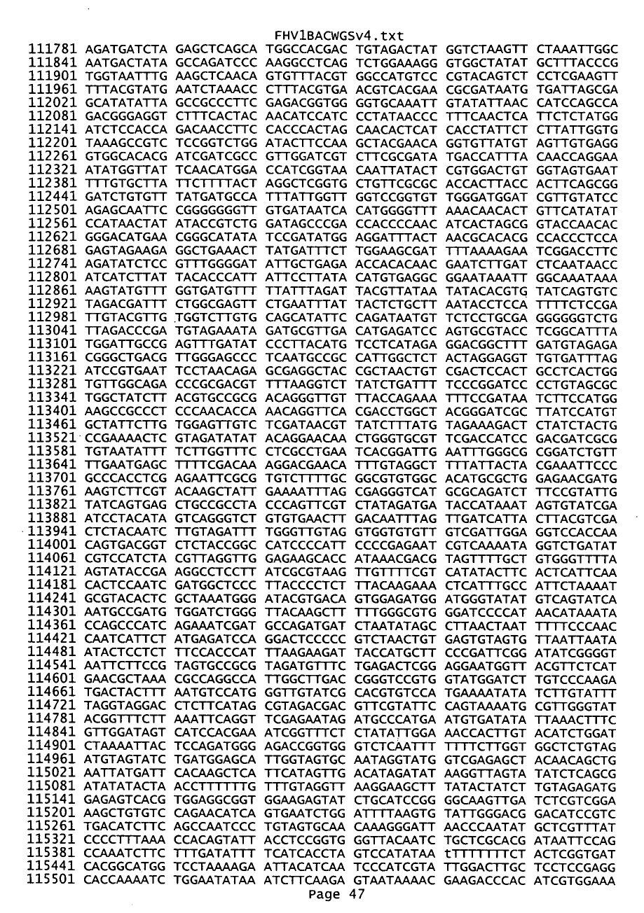

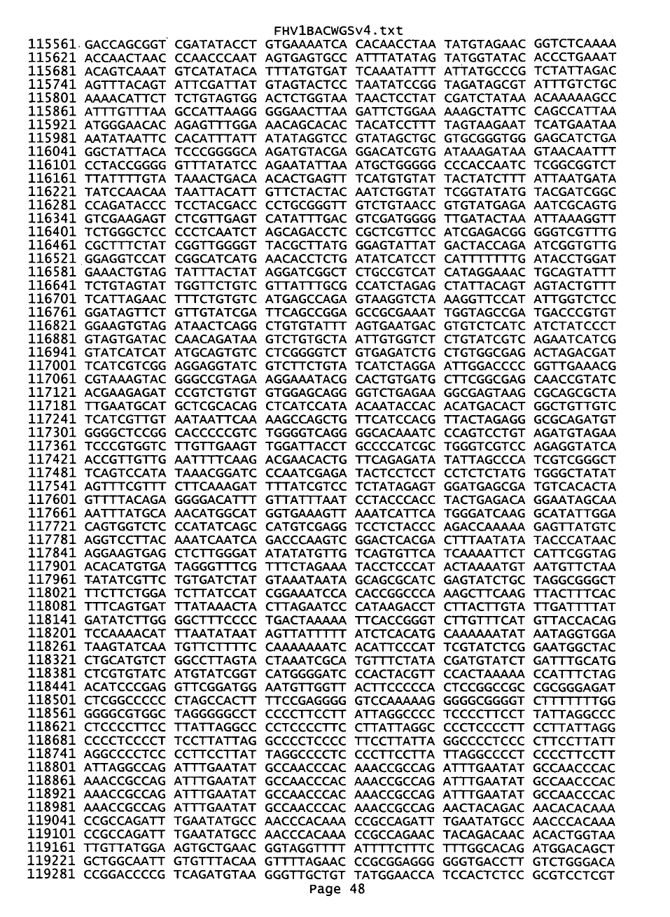

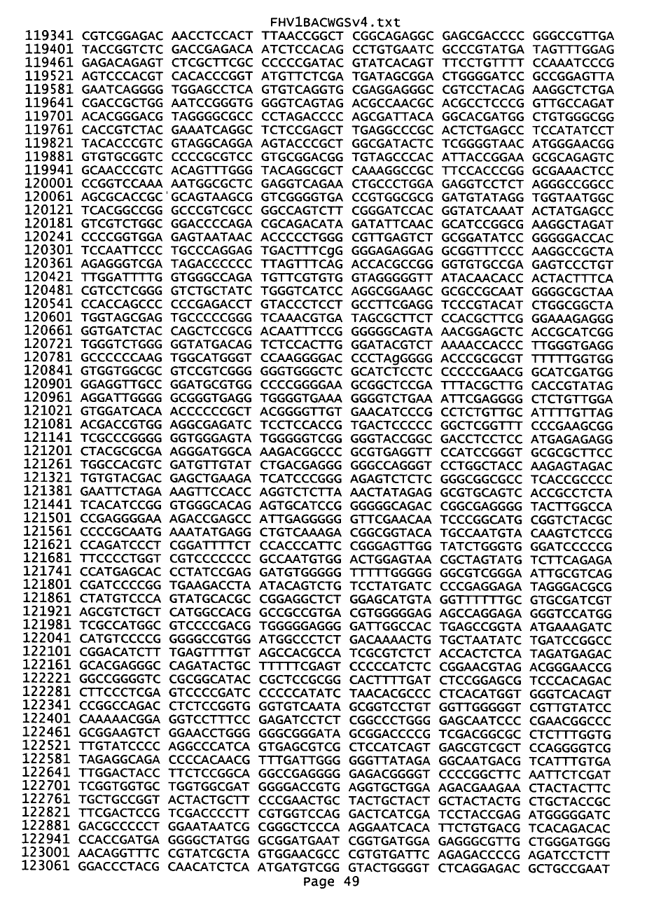

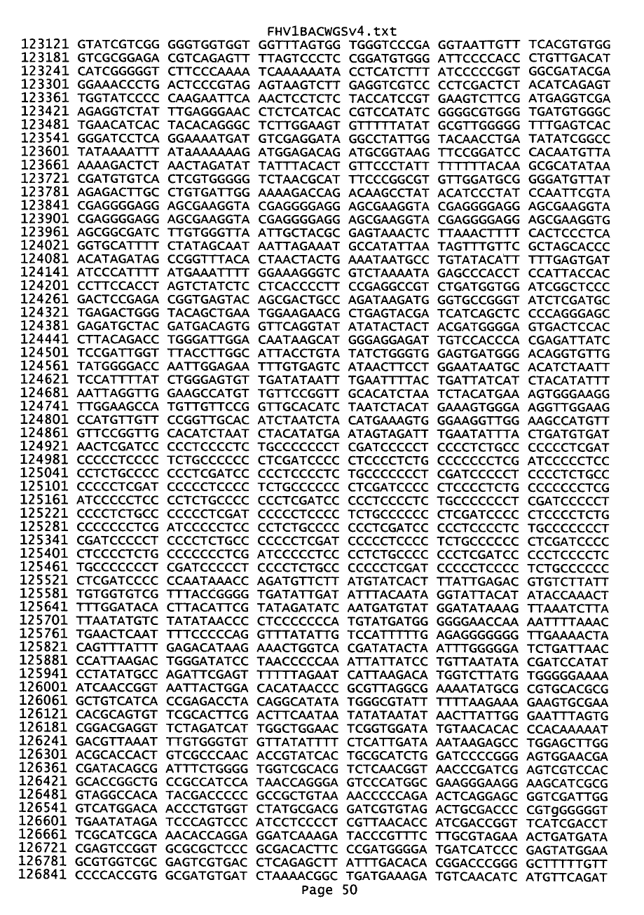

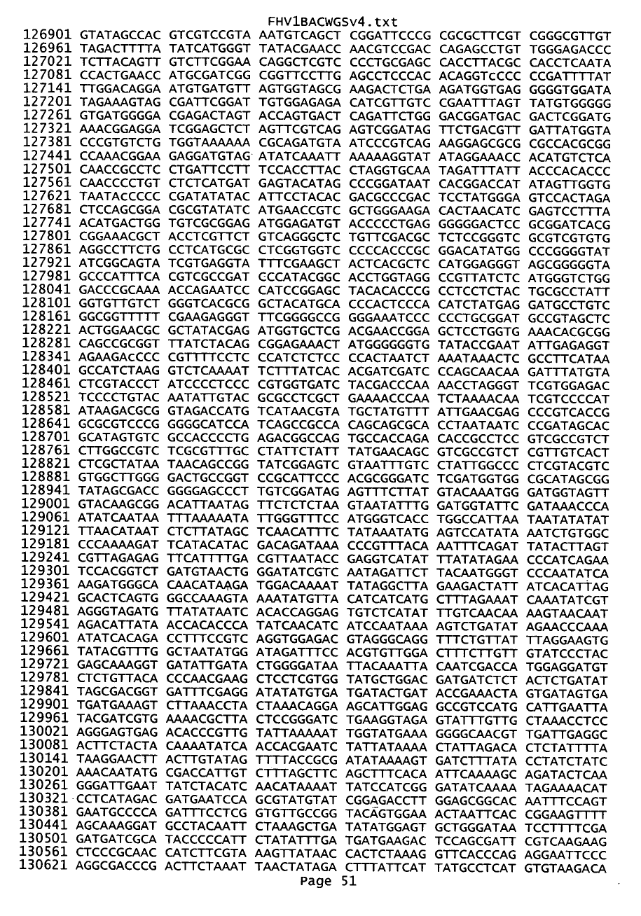

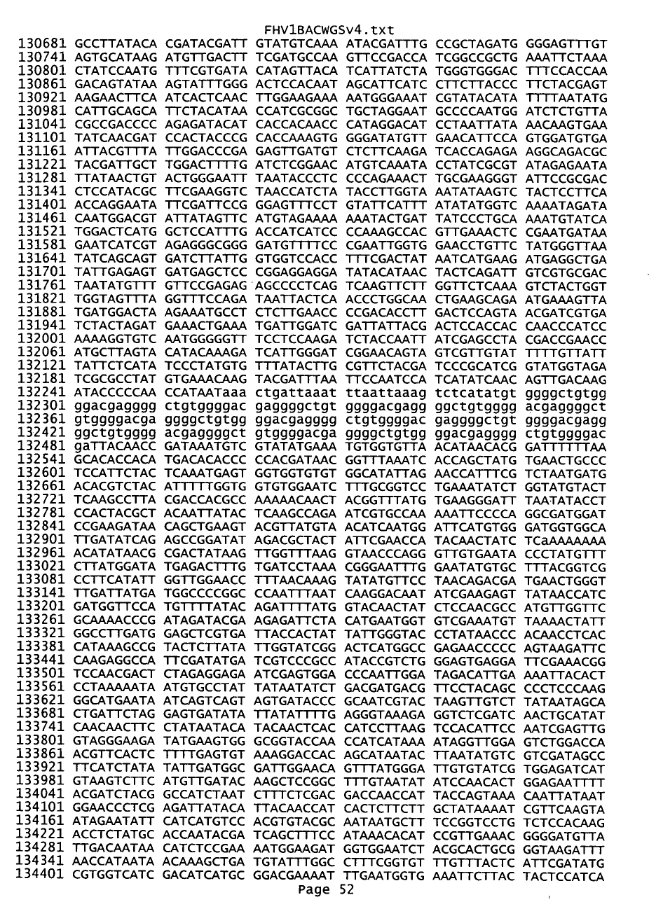

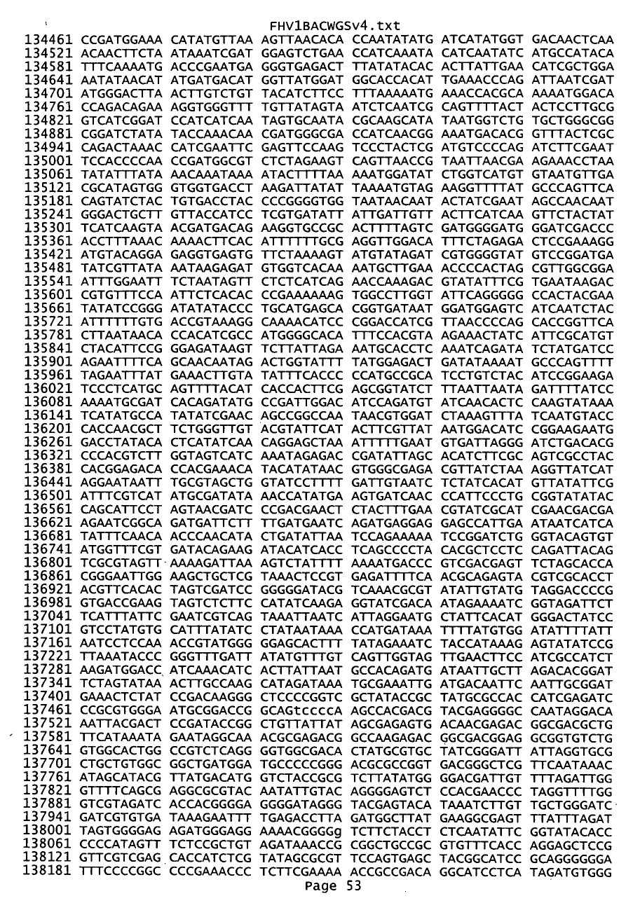

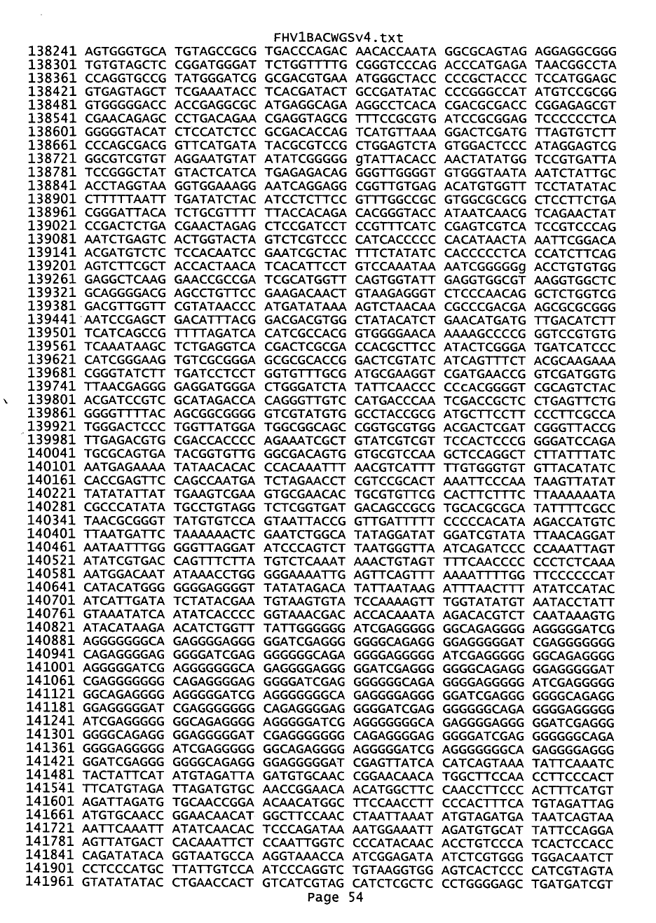

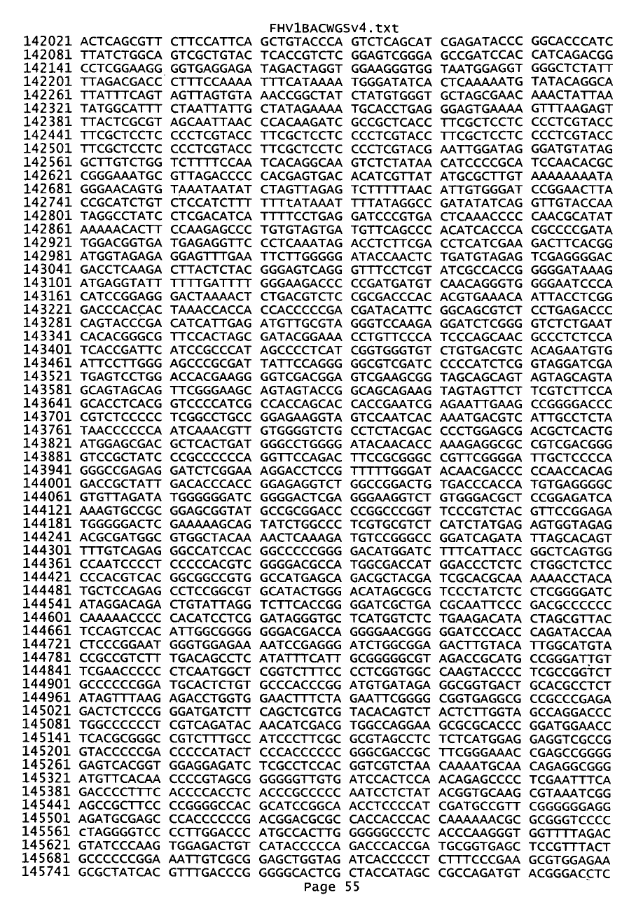

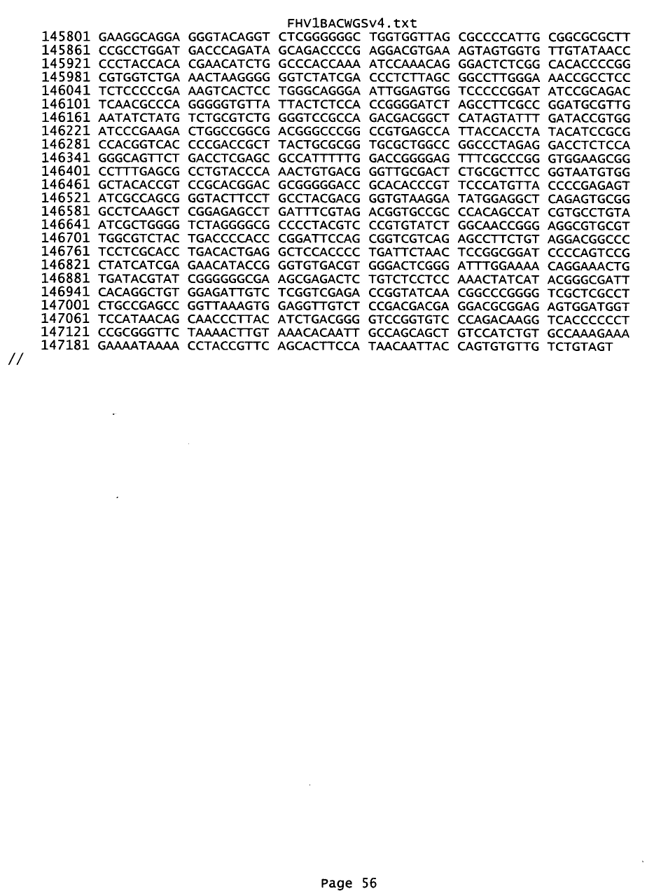

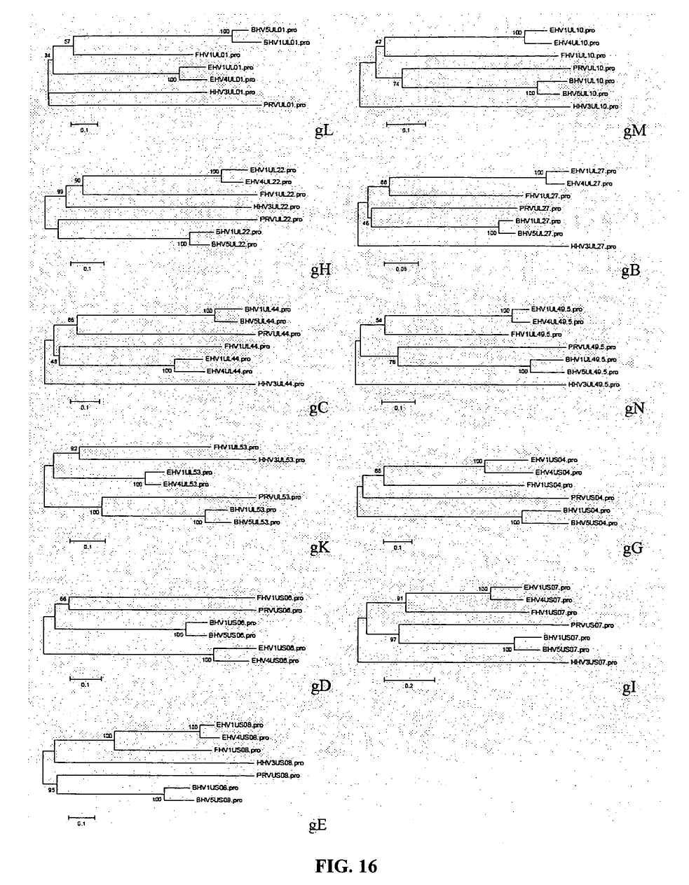

US8.5 was first identified based on the relative position similar to its HSV-1 homolog (Willemse et al., Virology 208(2):704-711, 1995). Despite the absence of sequence homology, US2 was also identified based on relative position similar to other varicelloviruses. FIG. 13 lists all FHV-1 genes identified in the genomic sequence and summarizes the characteristics of the gene products. Seventy-eight ORFs were predicted in the FHV-1 genome, encoding 74 different proteins, as the genes encoding the ICP4, US1, and US10 proteins are found twice, once in the IRS and once in the TRS, and the UL15 consists of 2 ORFs. Seventy-two ORFs present as single copies, among them 64 locate in the UL, 7 locate entirely in the US, and 1 initiates in the US and ends in the TRS. Three ORFs locate entirely in the repeat region, each present once in IRS and once in TRS. All ORF start locations were assumed to be the first possible ATG, unless demonstrated otherwise. The name of each protein was given based on its homology to HSV-1 and VZV genes. The properties and functions assigned to each predicted gene were based on those assigned to other varicelloviruses in the GenBank entries of reference genome sequence. In addition to the ORFs listed in FIG. 13, 53 ORFs with a coding capacity of more than 60 amino acids were identified: 25 were found on the top strand and 28 were on the bottom strand. However, searches for cellular or viral homologs of these ORFs failed to find any significant match, and none of these ORFs was considered a strong candidate for a new gene. FIG. 13 also lists the amino acid sequence similarity of each protein to their counterparts in the other varicelloviruses. All FHV-1 gene products showed some degree of homology to the gene products of the other varicelloviruses. Genes involved in nucleotide metabolism, DNA replication and packaging are among the most conserved. Glycoprotein genes are less conserved, although gB did show a high degree of similarity to the gB of the other varicelloviruses. FHV-1 proteins are most similar to homologues of EHV-1. Phylogenetic tree analysis of FHV-1's glycoproteins is shown in FIG. 16, the trees were generated using the neighbor-joining method. Boostrap values (1000 replicates) are given for each branch. The scale bars represent the number of amino acid substitutions per site. The tree analysis suggests that the FHV-1 is closest to EHV-1. The arrangement of FHV-1 genes is collinear with VZV, BHV-1, BHV-5, EHV-1 and EHV-4. The gene content is highly similar to those of varicelloviruses, especially EHV-1 and -4, with a few exceptions (Table 6). FHV-1 lacks the homologs of V67, US2, and US5, while all three are present in EHV-1 and -4. V67 was found in EHV-1, EHV-4, BHV-1, BHV-5, and VZV, but is absent in PRV. The US5 gene, encoding gJ, was found only in EHV-1 and -4. None of the genes identified were unique to FHV-1. Because the BAC vector was unexpectedly inserted into the genome after UL56, it was unknown if the sequence assembly FHV-1 encodes more genes after UL56. However, no genes were found after UL56 in the other varicelloviruses. The FHV-1 gene arrangement is collinear with the other varicelloviruses, it is believed that UL56 is followed by the A-type sequence, the junction between long and short segments. In addition, the highly repetitive A-type sequence and the presence of the repetitive sequence in downstream recombination arm is probably the cause of the BAC's unexpected insertion here during homologous recombination.

PolyA, Promoter and Splice Sites. It is widely assumed that the genes in herpesviruses are transcribed as capped and polyadenylated mRNAs by host RNA polymerase II, as shown in HSV-1 (Tabaska et al., Gene 231(1-2):77-86, 1999). Computer prediction programs were used to identify RNA polymerase II transcriptional control elements, including core promoters, splice sites, and polyadenylation sites. The PolyADQ program was used to search for all potential polyadenylation signals in the FHV-1 genome. This program was designed to detect and evaluate potential poly(A) signals, AAUAAA or AUUAAA, in human DNA sequences using weight matrices for base composition and position in the downstream element, a U- or GU-rich sequence located 20 to 40 bases after the cleavage site. The search found 323 sites; each was given a score between 0 and 1, and each was predicted as positive or negative by comparing to the default cutoff score. In the PRV study, experimental results were used to determine a cut off score, as low as 0.05. Since there are very few, if any, data for the mapping of FHV-1 transcripts, all signals found were marked on the map initially. Many of the signals did not associate with any upstream gene, but very often there are multiple polyA signals for a given ORF or end of co-terminal transcripts. The polyA signals for each gene were annotated based on the assumption that each transcript either ends near the termination codon or is a member of a 3′-coterminal family. The ones closest to the stop codon and have the highest scores were annotated. The Neural Network Promoter Prediction program, an eukaryotic core promoter prediction program, was used to search for promoters (Reese et al., Comput. Chem. 26(1):51-56, 2001). An initial high stringency search (cutoff score=0.99) of the entire FHV-1 genome found core promoter for only 11 of the 74 genes. These genes are: ICP0, UL7, UL9, UL14, UL17, V32, UL39, UL44, UL45, V1, and US1. To find promoters for the remaining genes, the search parameters were relaxed. However, the searches often identified more than one putative promoter for a given ORF, in close proximity to each other. UL15 is made up of two exons and is well conserved among herpesviruses. The region between and including the two ORFs of UL15 were submitted to the Neural Network Splice Site Prediction program conditioned for human splice site recognition. The predicted donor site with the highest score (0.98) located 39 by upstream from the end of the first ORF of UL15. The predicted acceptor site with the highest score (0.99) located between the ORF of UL17 and the second ORF of UL15, partially overlapping with the predicted promoter of UL17. The spliced mRNA would encode a protein of 734 amino acids. Whether this predicted splice site is really functional remains to be determined with experimental evidence.

Origin of Replication and Tandem Repeats. OriS was found in the IRs and the TRs. OriL has been found between UL21 and UL22 in other varicelloviruses, including EHV-1, EHV-4, PRV, and VZV. However, no palindromic sequences existed in this region. Using the Tandem Repeat Finder program, we found 8 different types of tandem repeat elements at 16 locations in the FHV-1 genome (Table 7). One tandem repeat element was found in the US, 4 in the IRS/TRS, and 3 in the genomic termini. None of these repeats located within or overlapped with predicted ORFs. Unexpectedly, no repeats were found in the UL region. In the UL region the Tandem Repeat Finder only found an imperfect 78-mer repeat in the ORF of UL44 (gC). The 2 repeat units were 91% match with each other.

TABLE 7
Tandem Repeats

| | Rpt Unit | Copy | | Total | |
|---|---|---|---|---|---|
| Location | Size (bp) | No. | Sequence | Length | Note |
| 117347-117723 | 17 | 22.2 | tggagtctaggtgtggg | 377 | Gap1 |
| 117914-118144 | 21 | 11 | ggcctaataaggaaggggagg | 231 | Gap1 |
| (146908-147138) | | | | | (Gap3) |
| 118142-118357 | 30 | 7.2 | tctggcggtttgtgggttggc | 216 | Gap1 |
| (146695-146910 | | | atattcaaa | | (Gap3) |
| 118371-118474 | 30 | 3.5 | | 104 | Gap1 |
| (146578-146681) | | | | | (Gap3) |
| 121871-121909 | 18 | 2.2 | tggagcgacgctcactga | 39 | ICP4 (IRS) |
| (143143-143181) | | | | | (TRS) |
| 123174-123295 | 20 | 6.1 | accttcgctcctcccctcgt | 122 | Between ICP4 |
| (141757-141878) | | | | | and US1 (IRS) |
| | | | | | (TRS) |
| 124021-124222 | 53 | 3.8 | aggttggaagccatgttgtt | 202 | Between ICP4 |
| (140830-141031) | | | ccggttgcacatctaatc | | and US1 (IRS) |
| | | | tacatgaaagtggga | | (TRS) |
| 124259-124861 | 26 | Est. 23 | gggggatcgagggggggcag | ~600 | Gap2 (IRS) |
| (140191-140793) | | (11 | agggga | | (TRS) |
| | | seq'd) | | | |
| 131621-131827 | 16(?) | 13(?) | ggggctgtggggacga | ~200 | US4-US6 |
| | | | | | intergenic |
| | | | | | region |
| | | | | | (DgG)(US) |

In vitro characterization: plaque morphology, growth curve. To reconstitute virus particles from the BAC, the BAC DNA was transfected into CRFK cells. The reconstituted virus, with the BAC vector in its genome, produced fluorescent plaques in CRFK monolayers (FIG. 4). To eliminate the effects the BAC vector might have on growth characteristics and virulence, the BAC vector was removed by co-transfecting the CRFK cells with the BAC DNA and pcDNA-Cre. pcDNA-Cre expresses Cre protein, which specifically recognizes the loxP sites flanking the BAC vector, excises it and re-ligate the DNA, leaving a scar of one loxP site. Very few fluorescent plaques were found in the supernatant obtained from the co-transfected cells (data not shown). Since the supernatant was plaque purified, this was an efficient way to remove the BAC vector. The excision of BAC was also verified by PCR and sequence analysis. The BAC-excised BAC clone of FHV-1 (FHV1 BAC) was used for subsequent in vitro and in vivo characterizations. The FHV 1 BAC virus can grow to a high titer similar to the parent strain at 72 hours p.i. (FIGS. 9A and 9B). ANOVA tests suggested that there are significant differences at 48 (p=0.003154) and 72 hours p.i. (p=0.013236). After the removal of the BAC vector, the virus can produce plaques that are morphologically undistinguishable from those of the parent strain.

In vivo Study. In order to investigate possible attenuation resulting from BAC cloning, a preliminary experiment was carried out with four specific-pathogen-free cats. The positive control cat and the FHV1ΔBAC virus-infected cats all tested VI positive on days 3, 6, and 9, and became negative on day 14. The positive control cat and the FHV1ΔBAC virus-infected cats all showed nice seroconversions on Day 14, and the neutralizing antibody titers of the FHV1ΔBAC virus-infected cats are similar to that of the parent C-27 strain-infected cat. The clinical scores of the BAC clone-infected cats were comparable to those of the wild type virus. Virus neutralization tests showed that both the parent strain and the BAC clone can induce similar neutralizing antibody titers. Also see discussion for Example 4.

### Example 8

### Differentiating Infected from Vaccinated Individuals (DIVA) Assay Concept

In this example a DIVA assay will be developed to distinguish between individuals infected with FHV-1 and those who were vaccinated. A possible format is an ELISA assay in which microwells are coated with a ligand, such as purified virions (assay1) or with a specific viral protein, which is not produced by the gene deleted vaccine (assay2). A serum sample from the cat will be collected and analyzed for the presence of antibodies. Cats that were naturally infected would have antibodies to the full complement of ligands, viral structural proteins, in their serum and would test positive, both with assay 1 and assay 2. In contrast, cats that had been vaccinated with, for example, a gC-mutant would react positive in assay1, because this gene deleted vaccine is still highly immunogenic and has induced antibodies to all structural proteins present in it. These cats would however react negative in assay 2 (plate coated with FHV-1 gC in this example), since the absence of gC in the vaccine precluded formation of antibodies against this glycoprotein. Thus, it is contemplated that various assay formats could be available depending on which gene deleted vaccine the cat was inoculated with (Harlow et al., Antibodies: A Laboratory Manual, 1988; Harlow et al., Using Antibodies: A Laboratory Manual, 1999; discussing use of antibodies, monoclonal antibodies, antigens, and assay formats). Furthermore, other assay formats include use of qRT-PCR (J Virol Methods, 152:85-90, 2008) along with rapid immuno-migrations (RIM) antigen detecting assay and detection of the presence of virus. The proposed DIVA assay includes detection of the complete FHV-1, FHV-1 mutants lacking gG gene, gI gene, gC gene, gE gene or any non-essential gene and combinations thereof, FHV-1 genome as set forth in SEQ ID NO:1, FHV-1 genome as set forth in SEQ ID NO:2, FHV-1 genome as set forth in SEQ ID NO:3, FHV-1 genome as set forth in SEQ ID NO:4, fragments thereof, and combinations thereof.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in molecular biology, genetics, or related fields are intended to be within the scope of the following claims.

What is claimed is:

1. A bacterial artificial chromosome (BAC) comprising a feline herpes virus type I (FHV-1) genome.

2. The BAC of claim 1, further comprising loxP sites flanking said BAC.

3. The BAC of claim 2, wherein said FHV-1 genome comprises a unique long (UL) region.

4. The BAC of claim 3, wherein said FHV-1 genome further comprises a unique short (Us) region.

5. The BAC of claim 4, wherein said FHV-1 genome further comprises one or both of an inverted repeat short (IRs) region and a terminal repeat short (TRs) region.

6. The BAC of claim 1, wherein said FHV-1 genome comprises a polynucleotide with a sequence selected from the group consisting of SEQ ID NO:1, SEQ ID NO:2, SEQ ID NO:3, and SEQ ID NO:4.

7. The BAC of claim 1, further comprising a marker for selection in eukaryotic cells.

8. A host cell transformed with the BAC of claim 1.

9. A host cell co-transformed with the BAC of claim 1 and a polynucleotide encoding Cre recombinase in operable combination with a promoter.

10. A method for producing feline herpes virus type 1 (FHV-1), comprising
a) providing:
i) a cell line permissive for FHV-1 infection,
ii) a bacterial artificial chromosome (BAC) flanked by loxP sites comprising a FHV-1 genome, and
iii) an expression vector comprising a polynucleotide encoding Cre recombinase, in operable combination with a promoter,
b) contacting said cell line with said BAC and said expression vector to produced a transfected cell line; and
c) culturing said transfected cell line under conditions so that FHV-1 is produced.

11. The method of claim 10, further comprising step d) purifying said FHV-1.

12. A composition comprising FHV-1 produced by the method of claim 10.

13. The composition of claim 12, further comprising a pharmaceutically acceptable carrier.

14. A method for immunizing a cat against feline herpesvirus 1 (FHV-1), comprising administering to a cat the composition of claim 13.

15. The method of claim 14, wherein said administering comprises intramuscular, subcutaneous, intradermal, or intranasal inoculation.

16. A kit for producing feline herpes virus type 1 (FHV-1), comprising:
a) a cell line permissive for FHV-1 infection;
b) a bacterial artificial chromosome (BAC) flanked by loxP sites comprising a FHV-1 genome;
c) an expression vector comprising a polynucleotide encoding Cre recombinase, in operable combination with a promoter; and
d) instructions for contacting the cell line with the BAC and the expression vector to produced a transfected cell line, and culturing the transfected cell line under conditions suitable for production of FHV-1.

17. The kit of claim 16, further comprising:
i) a first marker for selection, in operable combination with an endonuclease recognition site;
ii) a plasmid comprising a homing endonuclease I-SceI, in operable combination with a second marker for selection;
iii) an expression vector comprising a polynucleotide encoding Cre recombinase, in operable combination with a promoter; and
iv) instructions for contacting the host cell with the BAC, expression vector, and said first marker for selection to produce a first transformed host cell, and growing the first transformed host cell under conditions suitable for selection of a first transformed host cell, and contacting said first transformed host cell with the plasmid to produce a second transformed host cell, and growing said second transformed host cell under conditions suitable for selection of said second transformed host cell.

18. A method for producing a feline herpes virus type 1 (FHV-1) mutant, comprising:
a) providing:
i) a host cell permissive for FHV-1 infection;
ii) a bacterial artificial chromosome (BAC) flanked by loxP sites comprising a FHV-1 genome;
iii) a first marker for selection, in operable combination with an endonuclease recognition site; and
b) contacting said host cell with said BAC, and said first marker for selection, and said plasmid to produce a first transformed host cell;
c) growing said first transformed host cell under conditions suitable for selection of said first marker for selection wherein said selection includes expression of a protein.

19. The method of claim 18, further comprising:
d) providing:
i) a plasmid comprising a homing endonuclease I-SceI, in operable combination with a second marker for selection;
e) contacting said first transformed host cell with said plasmid to produce a second transformed host cell whereby said first marker for selection is deleted from said second transformed host cell; and
f) growing said second transformed host cell under conditions suitable for selection of said second marker for selection.

20. The method of claim 18, wherein said first marker for selection recombines with a target gene or portion thereof whereby said target gene or portion thereof is replaced by said first marker for selection.

21. The method of claim 20, wherein said target gene is selected from the group consisting of: gG gene, gI gene, gC gene, and gE gene.

22. The method of claim 18, further comprising step d) purifying said first transformed host cell.

23. The method of claim 19, further comprising step g) purifying said second transformed host cell.

24. The method of claim 23 further comprising step h) repeating steps b) through g) to produce a feline herpes virus type 1 (FHV-1) double mutant.

25. A composition comprising FHV-1 produced by the method of claim 24.

26. The composition of claim 25, further comprising a pharmaceutically acceptable carrier.

27. A method for immunizing a cat against feline herpesvirus 1 (FHV-1), comprising administering to a cat the composition of claim 26.

28. The method of claim 27, wherein said administering comprises intramuscular, subcutaneous, intradermal, or intranasal inoculation.

29. A method for differentiating between immunity resulting from vaccinations with BAC-derived gene-deleted FHV-1 or field virus, comprising:
a) providing;
i) a patient suspected of having FHV-1;
ii) a biological sample derived from said patient, wherein said sample is serum wherein said serum contains an FHV-1 antibody capable of interacting with a ligand wherein said ligand is an FHV-1 antigen;
b) incubating said sample with said ligand under conditions such that said sample binds to said ligand thereby forming a sample-ligand complex; and
c) detecting said sample-ligand complex, thereby differentiating between said FHV-1 antibodies generated from vaccination and infection.

30. A composition, comprising the BAC of claim 1, wherein said FHV-1 genome comprises a polypeptide selected from the group consisting of a nucleotide sequence of at least 135 kb in length that hybridizes under high stringency conditions to the nucleotide sequence set forth in SEQ. ID No:4, a nucleotide sequence of at least 100 kb in length that hybridizes under high stringency conditions to the nucleotide sequence set forth in SEQ. ID No:1, a nucleotide sequence of at least 5 kb in length that hybridizes under high stringency conditions to the nucleotide sequence set forth in SEQ. ID No:2, a nucleotide sequence of at least 5 kb in length that hybridizes under high stringency conditions to the nucleotide sequence set forth in SEQ. ID No:3.

31. The composition of claim 30, further comprising a pharmaceutically acceptable carrier.

32. A method for immunizing a cat against feline herpesvirus 1 (FHV-1), comprising administering to a cat the composition of claim 31.

33. The method of claim 32, wherein said administering comprises intramuscular, subcutaneous, intradermal, or intranasal inoculation.


SEQUENCE LISTING (105 sequences)

SEQ ID NO: 1 - DNA, 117439 bp, Feline herpesvirus 1
aagtaaccaacattccatccgaacctcgggatgtctagaaatggtttttagtggaacgtagtgggatccccatgaccgatacatgatacacgagcatgcaaatcagatacatcgtatagaaacatgcgatttagtactaaggccagacatgcaggtagccattccgagatacgaatgggaatgtgatttttttggaaaagaacattgatacttatccacctattatattttttgcatgtgagataaaaataactattatattaaatgttttggactgtggtaacatgaaacaagacccggtgaatttttagtcaggggaaagccccaagatatcataaaatcaatacaagtaagaggtcttatgggattctaagtagtttataaatcactgaaagtgaaagtaacttgaagctttgggccggtgtggatttccgatggataagatccagaagaaagcccgcctagcagatactcgatgcgctgctattatttacatagatcacagaacgatatattagaacattacattttagtatgggaggtatttctagaaacgaaaccctatcacatgtgtctaccgaatgagaattttgatgaacactgacaacatatatatcccaagagctcacttcctgttatgggtatatattaaagtcgtgagtccgacttgggtctgattgatttgtaaggacctgacataactctttttggtctgggtagaggacctcgacatggctgatatgggagaccactgtccaatatgccttgatcccatgaatgatttaactttcaccatgccatgtttgcataaattttgctattcctgtctcagtaggtgggtaggattaaataacaaatgtcccctctgtaaaactagtgtgacatcgctcatccactctatagaggacgataaaatctttgaagaaacgaaactatatagcccacatagagaggaggaggagtatctcgattgggatccgtttatatggactgaagcccgacgatgggctaatatatctctgaacagtgttcgtcttgaaaattcaacaacggttgatacctctggacgacccagcgatggggcaggtaatccaacttcaacaagaccacgggattctacatctacaggactgggatttgtgcccctgaccccagacgggggtgccggagccccacatctgcgccctctagtaacgtggatgaacagctggcttttgaattattacaacgatgagacaacagccagtgtcatgtgtggtattgttatggatgagctgtgcgagcatgcattcaatagcgctgcgcttactcgccttctcagacccctgctccacacacagacggatctcttcgtgatacggttgctcgccgaagcatcacagtgcgtatttccttctacggcccgtactttacgcgtttcaaccggggtccaattcctagatgatacagaagacgatacctcctccgacgatgaatcgtctagtctcgccacagcagatctcacagaccccgaggacactgcatatgatgataccgatgattctgacgatacagagaccacaattagcacagacttatctgttggtatcactacagggatagatgatgagacacgtcattcactaaatacacagcctgagttatctacacttccacacgggtcatcggctaccaatttcgcggctccggctgaatcgatacaacagaactatccggagaccaatatggaacctttagaccttactctggctcatgacacagaaagttctaatgaaaacagtactactgtaatagctctagatggcgcaaataacgacagaaccaatactacagaaaatactgcagtttcctatgatgacggcagagccgatcctatagtaaatactacagtttcatccaggtatcaaaaaaatgaggatgatatcagaggtgttcatgatgccgatggacctcccaacaccgattctggtagtcataatactcccataagcgtaaccccaaccgatagaaagcgcaaacgacccccgtctcgatggaacgagcggaggtctgctagattgagggggagcccagaaacctttaatttagtatcaaccccatcgacgtcaaatatgactcaacgagactcttcgaccactgcgatttctcatacacggttacagacaacccgcaggggtcgtaggagggtatctgggccgatcgtacatataccgaataccagattgtagtagaacaatgtaattattgttggatatatcattaataaagatagtaatacacatgaaactcagtgttgtcagtttatacaaaataaagaccgccgagattggtgggcccccagcatttaatattctggatataaacccccggtaggaaattgttacttatctttatcacgatgtcctcgtacatcttgccccgggatgtaatagcctcagatgctcccacccgcacgcagctatacggacctatataataaatgtggaattatattttattcatgaattcttactaaaaggatgtagtgtgctgtttccaaactctgtgttcccatttaatggctggaatagctttttccagaatcttaagttcccccttaatggcttaaacaaatggctttttgtttatagatcgataggagttattaccagagtccactacagaagaatgttttgcagacaaatacgctatctaccggatattaggagtactacataatcgaatactgtaaactgtctaatagacgggcataataaatatttgaatcacataaatgtatatgacatttgactgtatttcagggtgtataccatactatataaatggcactcactattgggttgggttagttggtttttgagaccgttctacatattaggttgtgtgattttcacaggtatatcgaccgctggtctttccacgatgtgggtcttcgttttattactcttgaagatttatattccagattttggtgcctcggaggagcaagtccaatacgatgggattgatgtaattcttttaggaccatgccgtgatcaccgagtagaaaaaaaattatatggactaggtgatgaaaatatcaaagaagatttggctggaattatcgtgcgagcagattgtaacccaccggaggtaatactgtggtttaaaggggataaacgagcatattgggttaatccctttgttgcactacagggattggctgaagatgtcagacggatgtccgtcccaatacacttaaaatccagattcactgatgttctggacacagctttccgacgagatcaacttgccccggatgcagatactcttccaccgcctccacgtgactctccatctctacaagatagtataaagcttccttaacctacaaacaaaaaaggttagtatatatcgctgagatatactaaccttatatctatgtcaactatgaatgagcttgtgaatcataattcagctgttgtagctctcgaccatacctattgcactaccaatgctccatcagatactacatctacagagccaccaagaaaaaaattgagacccaccggtctcccatctggagtaattttagatccagatgtacaagtggtttccaatatagagaaaccgatttcgtggatgactatccaacgaaagtttaatatatcacattcatgggcatctattctcgaacctgaatttaagaaaccgtatacccaacgcattttactggaatacgaacgtcgtctacgctatgaagaggtcctacctaaaatacaagatatattttcatggacacgtgcgatacaacccatggacattaaagtagtcatcttgggacaagatccataccacggacccggtcaagccaatggcctggcgtttagcgttcatgagaacgtaaccattcctccgagtctcagaaacatctacgcggcactacggaagaattaccccgatatccgaatcgggaagcatggtaatcttcttaaatgggtggaaagaggagtattattaattaacactacactcacagttagacgggggagtcctggatctcatagaatgattggttgggaaaaattagttaaggctatattagatcatctggcatcgatttctgatgggctggtatttatgttatggggatcccacgcccaaaaagcttgtaacccagatccacatcggcatttgatactgacatatacccatccatctccactgtcacgtatcccatttagcgagtgtacgcattttagaatggcaaatgagtttcttgtaaagaggggtaagggagccatcgattggagtgttgaatgagtgaagtatatgacgaaaacaacttacgcgataaggaggccttcggtatacttaaaacccacagcaaaactacgtcgtttatggtgcttctccaacctaacgtagatggacgatatcagacctattttgacgattctcggggaatggggatggccggtagagaccgtcactgttggtggacctccaatcgacaacacaccacctacaacccaaaatctacaagattgtagagtcgacgtaagtaatgatcaactaaattgtcaagttcacacagaccctgactatgtaggattcgatacactatttatggtatcatctatagacgaactgggtaggcggcagctcactgatacaatacggaaagatctgcgcatgaccctcgctaaattttcaatagcttgtacgaagacttcatcgttctccagcgcatgtgccacacgccgcaaaagacacgcgaattctcgaggtgggcgggaatttcgtagtaataaaagcctacaaatgttcgtcctttgtcgaaaagctcattcaaaacagatccgcgcccaaattcaatccgtgattcaggcgaggaaaccaagaaaatattacacgcgatcgtcggatggtcgaacgcacccagttgttcctgtatatatctacgagttttcggcagtagatagagtctttctacataaagataacgttatcgagacaactccacaagaatagcacatggataagcgatcccgtagccaggtcgtgaacctgtttggtgttgggagggcggcttccatggaagattatcggaaatttctggtaaacaaccctgtcgcggcacgtaagatagccagcgctacaggggatccgggaaaatcagataagaccttaaaacgtcgcgggtctgccaacaccagtgaggcagtggagtcgacagttagcggtagcctcgctctgttaggaattcacggatctaaatcacaacctcctagtagagccaatggcggcattgagggctcccaacgtcagcccgtctctacatcaaagccgtcctctatgaggacatgtaagggatatcaaactcggcaatccataaatgccgaggtacgcactggatctcatgtcaacgcatctatttctacatcgggtctaacagacccccctcgcaggagaacattatctggaatatgctgcacaagaccacaacgtacaatcggagaaaatggaggtattaagcagagtaataaattcagaactcgccagaaatcgtctagacactgatacacgtgtatattataacgtaatctaaataaaaacatcaccaaacatactttttatttgccaatttattccgcctcacatgtataaggaataatgggtgtaataagatgatggttattgagatcaagattcgttgtgtggttctcagcaatatccccaaacggagatatctgaaggtccgattcttttaaaatcgcttccaagaaatcataagtttcagcctcttctactctggagggtggcgtgtgcgttagtaaatcctccatatcggatatatgcccgttcatgtcccgtgttggtaccgctagtgatgttggggtggtcgggctatccagacggtatatagttatggatatatgaacagtgttgtttaaaccccatgtgattatcacaacccccccggaattgctctggatacaacgatccatcccaacaccggaccaaccaataaatggcatcataaacacagatcccgctgaagtggtaagtggtgcgcgaacagcaccgagcctagtaaaagaataagcacaaaattcactaccacagtccacgagtataattgttaccgatggtccatgttgaataaccatatttcctggttgtaaatggtcatatcgcgaagacgatccaacggcgatcgatcgtgtgccaccctcacaactacataacacctgttcgtagcttggaagtatccagaccggagacggctttacaccaataagagaataggtgatgagtgttgctagtgggtggaaggttgtctggtggagatccatagagaatgagttgaaagggttatagggatggatgttgtagtgaaagacctcccgtctggctggatggttaatatacaatttgcaccccaccgtctcgaagggcggctaatatatgctcgctaatcacattatcgcgttcgtgacgttcacgtaaagggtttagattcatacgtaaaaacttcgaggagactgtacgggacatggccacgtaaacactgttgagcttcaaattaccacgggtaaagcatatagccaccctttccagactgaggccttgggatctggctatagtcattgccaatttagaacttagaccatagtctacagtcgtggccatgctgagctctagatcatctatagactcgacaaattcactgatattggtatttaatatcgacataaaaccgtggtggtccttcaggaccacacgtggtatatgcatatacccaagtaattccgctgtcccctcgtggattcggcggcgattaaggtcatcattgaattgatgcactggaacatgtgtatatcccattagggtgtaactatctgtttggagagccgttgaaagtaaccctccacgtacactaccagtaaacacttcacaacccttaaagctcaaattatccacgtaggtattaaattgagcggaatctatgcatggtccaaagagttctctaaaaattgcatagcgaccaagaaacatgttcttaagcatcaagaattgggaataaacttcgggggcgctctcaggacgtacaaattcataattacaatagagcaggtctatttctccctcattgagggcggcgaagatgaggtcgtcatcatcacagttcttgtgatggatctttgttgtatcgcctagcgtagaaaaatcgaagggttggctggaagttaatggaggacccagggcacccgctgcttctttatcattacaagatatcgttccgacaggtaataggtttgtggttaattcagctagtcgtctataggcctccgagaccagtattccatccaactctctgttctgtaggaagttataaaagttgatcatgccgctgaaaaggagtgcagatagaaatctataggtatactctgccgatgtctctccatgtgctttcataaacccatcatctttcaagactgcatcgaatgacttgaaggtaccactgaaaccgaatactaattttttcatgcgtgtagtaacagtaacttggctgttgagtacataggtgatatccgctcgagcagcaacgaccccatgctttgagtgtagctcacatcgtacctggcctgtatcttggtccctgctttgagagtaattagttacccggccggcattagccgcgagccatttgtcaattacgagtcccggttgtcccgtcacgcgacgatattcatcaaaagcttctatgcgaacaaaagtataggcgggtagagtaaatactacaaattgtcgctcgccggatactcgtaaatgggcatgtaatttggtcatatatgcactcacctccttatgtgaagaatataatcgtgtccaaccaggtagattagctgggttgtttataaaggcatctgggacgacgaactgatctaccaggcgcatatggtcttccgtgaggggtaaaccatattctagtactttcatcagttcaccaaattcgggctcgagacaacgtttattattgatgaatatggcccaattactagacaatgaagtataatttcgtaatgtcttattggtgatgagatatgtgagaatattttcactggaacgaatacgacattttagttgatggtgttcgaatgtggattcgagggaatctgtctgagtaggggagccaatgcagacaatcactggaacgcgaccggctagatattggggggtgttgtaaatagcatttatcatccaccagcaatatacaaccacggtcaataagtgtcgtcccaaaagccccgcttcatctataactataacgttactttttgtaaatgccggaagagatccttgaacacagaatgcaagactagccagtgtcccggaagcacgtcccctgatgagttctatacttcttatggtgtcaaacaccacacgggttgcgtttgactccaggaaccgtttagtgatgtctataagtacttcccagtagtaaataagatctctcttctgtagctcttctatcggtggtgggttagaaggacatacatattgatatttaccgagttgtgcctgcacatgatttccgcggaaaccgaactcttgaaaaatagtattgacgggtctgctcgagtatgaagacgcgagtttttgatatacattctgggaagcgacgcgggtggcgcctgttataacacattccagggtttcgttgagagtctggatacaggtactttttccggaaccagcattaccgctaatgagatatacggaaaatggtaattcacgtggatttatatcaagcggtgtttcgagggacgtcaagtccttaaaccaacttacgggtgggatcttttcctttgaaactgataatcccgctaggtcctttatacgcgtgactatattccgaatgccatgcattgaagtaaaattaagatatatcgaattcgaaaaatcagcttggtcattataatagtctccggtgagatcggtacttgccatttctcctctatcaggaggtgacgtctctttatgcccgaactttaacaatctacagaccaatacatatttgcctatatgcgtgacgtcaatgtagagtgcatgcgcccgacaggtgtcaagtctggattgttaatggatgcgcgagctgggctgaaaatggaaccctgcatcgataaagtcggatctctcgataatttacaagttgaggctggggacgattggaagcttatccatcctaccccaaaaacaatgatattcaaggagatacttatgggaaatttcggctatactgaaggacaaggagtatacaacgcagttcgatcaaccgaggctgtaattcggcagattcaagcaacgattttaacaaataccctaaatgccgcgaaatatgatgatctcatgcgcgattggcgagcacatttagatgaacggggtgtcagtgcaacatccatagctacgatatacggtaaccacggtgaggcggaagcggtaagggtagccgagcaactttttaatacatggtataagacccttcatatgtccctcctggactttctgcgtggtatagcagcttgtttttcagccagtgaatctaacggtacagctagctttgccaagtatgtagattggatcgtttgtcttggtatggttccactacagagactcccgatgagaccccctcaacgtgggaatgatataggaagatcgcaccaatcaataggatgtgtagatgagagatacctgtcgatgagacttcacgtagcccataatattttgagggaaggagtcgcttcacttacagagctgacaacatgtgctcatggagtatctattatggattacgatagagtacatctattctataattacaagcgtagagaagttagggctgtagatcctgtaacgggtatagaaggggagtgtttagttctctggcaacccatatggtatgacggtgtagttttattcgattcgcctttacagcggctccacagcgaagtgttgaattgtcacaccctaagagaacatgctcgtttatgtcaaatattaaacacatcaccggtcaaggtgttgataggaaaacgttcggaagaagaaaggggggcatgggcatcgaaagcggtagataagatcctaagcgatggtggggaggctgctgcaggatcgagtgccgctcgtctagtgaagctaatagtaaatatgaaggggatgcgtcatattggtgatatcaccgaaacagttaggtcttacttggacgagacaaataaccatattttaaatggaccccaggtagacacatcacagccagggtttggtcagacaacgaaacgtggatcttccgttcagggggcacagccagttcaggatgcctttagaacctccgtactcaacagtattaatggtatgctcgaaggttatataaataatctttttaaaaccatagaaggcttaaaggctgataagaacggtctgttggatcgtctacaaagcaccgaacgcgaacttaaaaatgtgagcgaacgtgccctgcgtgccacccaagcggcggtcgatgtgcaacgaagtgggtatgccgctaccccatcgatggcaataggtgaatttgcgcatgaggttatcgatgtgagcgggtctatgggagaagactcatacgtggcaaatagttttcaagcgcggtacatccccccctactcagcggacttggatcgtttatctagactttgggatcaggaactggttcgatgtttcaagatgaatagaacggccaataatcaaggccaggagatgtcagtgtcctattcaaatagttccatttctctcattctagctccatatttcttttctattttgaaagttcgcagactgggtttcctgattacccatcaggaagtctatcgttcagaggaggagttatgtgcaagtatctttaaaaaaacccgcctggaagtatacctcacagaattgctaacgctctttacagcaaacgtaaaagctgccgtcacctacaaagtagcgtatatagcaaacgtgtccgaacctaaagaccctgacatcgagaaatatttatcattaccatttaagacacgatcgtggagacggtctactgaaaaccatggcaacagacgcatacaacgagttagccgtagacgccgtatacgagacatgtctcataaatccgacatacatcccggataccgtgcacgatcttacgtggcacgctctacctaggttgatttgtgaagtgcgtgaggtctctggacgcccaccgctattttccacgacatctgtagagcgtataagggttgtaaacggaacatatgaacttatgttaaccttggaggggcgatcacaagaaatttcatgtactacctatatggaggagtgtaaaaatcaaccagagttcaaaggattttccctttttgtgataactcccatagaggaccgtgtcaagtctttagcagtaggggaaatgatacttgaacataggacggtcctgttacatccagacgctacatcagatttcacattgtgtatattacagatgttcctagaaaactgcacagcccaagaagcaacatcgtctatgtttgtacaggtacacaccttattacagagacttgagaagacgctatctcctctatctcgactacgaaatctattgttcactggtgttacatggacacttaacactcttatgtatactatggatcacaatcctttcgataacaacaggattctcccgcatcatatattcgccaaactcctgttgggtgggggaaaacaacccccaagcatacttacggcgattttcacagtaggctctagttccgggagcaagatcattggatcggagaggcggtgtcccagtggattggtaagaacacgacccgcccttctgaacgtggcactacaacgttatgaaattcaagccgcgctctacaaatggtggctcaacccagaaaaaaacatcggacccgcttctatgttcataatgtatgaatgagttgagtctaatgattaaaagccatatatttaattgtatatattcgaatcatttatttttaagagaataaaaaggtatattcggagattggatacgagaatacgggcttcgtttcacttgtatcataatatgcctctatggaacggtttagcatggtagagaactttccaaacaagggttccaacttggaataacagtctatttggggaataatggccggatgtggatataatttccacggtatctgaataacttcatgatcatgattttttacgggaacgcattcacgtgggcgatggtctctgtccaagacgctcccaccacaatatatggcatttggtaatttattatgtcgattctctatgttcattggaacgctccaaaactcaaaatcccaccgtttcatatacgacaattcaaggagttcatcgcacatttcccgggccttactcagaagcgcctctaaatcggcatagcaacccattctcgctgacatgcgtagtaacatagataagcgatttttcatcgctctacatatagcacttcgtgtagggaatccaacgaaagcttccatatccgtctctctattccataaccaatagctattcgttgtccaagacatgacgtccgtgtatatcccctcgagacgtaagtgcagatttgtcccctcgggaagcaccaaacccatatcctctagatatttgatagcggccatctgacaggtatttcttatattttctgcggcaatttcaacatccgctagtgatgtggtctcctgtgttaaagagccaaacacaccccaaaacccatctttgatgtacgtacatatcccgaattgttcattattcaataatcgttccagatgtctactgattccattggcgaggccaattatcactctataggctaggggttggatatgttgtagcccaccgaataaagataccaatgctgccttcagccctcccttttcccgacgaatacgggccgatattatctgagctagacgggtatgctccgggaatatggcaagatagagagacggaaagaatgcagagaaatcaaatttggcaagccatagccccttcatggcaatcggatggtcgagtatactcgaaattcttccagctggccatttatcgtcctggtccttggttagttttgactcgagatgaatgagtctggtggtatgtgatcgctttacatcatcagtagttggatcccacggagctaatagatgaaaggctaacattcccattgccggccacataccatcggtagacacgtatacgtctatagcttcccgtacagcctctacatctggagatgctaccttgataaggggtatacccgggaagcccaacatacaactgtaatcgcgatcctccccagtagggctcaactccgggcctatatatgcaaatatcggcaataaaataggtatggatcccagttctccggtgttcgaaaacatagtttcgcgccattgtttataaagggtcatgatagaaattattgaaaatgtagaagagcatactagaaagcaatcaaaatatcttggaattaaaactcgtaccattacggtcttggattggagaacgagtctagaatctataattttaaataggtccttaaagtcccccgtttgtttaacagctgggcgctgtatagaatcatatcttctacggaatgtatgtcttttcgtgattacctcaacgtccatggatatatcctcttccatggattgggaaaagtagccatcccggagggtgcagaggcgagcccttgccaatcctcccccctcagtattcatagaattagcttttatggctcctggatccagatctacttttgccgtgacattcaaaatggctgggaccggcaccggatttgcctcgggttcgagggaattggtccttttatcattcaagatgggttgtatgtccacagtcattccagtttttatattcatacagaaggccacacgtgaataaaaagaaaaacccttaaatgatcgtttcaatacagccataacactactataaattgccttccataaaacagtctgtccgagggcaccaaggggccatatattcggggcggctgcctctccagccgctgccgctgctttgcggatcgtgatatatggacactcacgcgaaccccagattagttgattttcacttacgctgaccacggtgaagcgagcggtatattccccatcctcgttacatatcagatacacgagagcgtttagtttatcatggtctatccatagagaataaagacttgccgcgcaaacgtagccatgtgtccatttaatatcatcaataggtgttctatggtcatccatgatatcttcaatcgggatcactggcggagaattagataagcctatggctgtcatcacctaatggtttatcataggcgagaaaaatccaaagcaccctgatatgacggccattgatcggggggtatgcaagccagtgcgctgtccagctcatctgtacatgacaaggcttctccggtggacaaaaaacaaatgtatcggctccaccttggtgctcggagtttatatctaggagtatcaaatagtttgcgatgctgcattaacagggaatataattgtttataggttttagtctcggaatgatcaatattcactgattccaataatgcaattatttcggttttatggaggttttttacacgtgaaatacccggaaaagtttgtgtactcttaactaatttagcaccgaataactcaccccagagcaacttgaatgttcccacggcgtgttctagttgcgagacggggtatatattgccagagcaagactttccctttaatatggcgcttaccgatgatataggttgtttagctaagaggtcagctggggtgatatgacatttagccacttcaagcaatgaattgatacagatagaatatttaggttttagcagctgtaggatgacctcgggagttagatcggatgtagaaccgccacgatttggatcccaatctatttgtctggtaagatacgcacatgttttatataggctccagagagcctcgatgttgtggtttggggtcacagtctccgtaagtaacatcccagtgtcattaatcaccgggataattccggaagcgtaatggtgatataacgcggtgaacacctcgacactttctaagctctcgggtaaacgcatacaggccccaagaacggctatgttaacgaattggttccgtatgctcggacttgatagtctttgaaggagggttttcataaaaccagaagggctagttattattaaatatttagagataaaacccgcaacctcgtccgtgtcgtatgtttcagaccctggtaggatagctacattcacagtctcgcgtatctgtcgaagtactcgctgatttgataacgcctcggcgtgtatatttcgtaaaaatttgcagaaactctctgtagataactgggctggacatccatcaaagtggactctaatacaattagcctcgagaagcgtatgtaatatatttatactatcattaacgcttgctaatgtgcagcgttcaaaaaaatgcttgtatcggaactttgaaaatagatgtagaccgcgtattgtcctataggccttcttgcaactatctctaaattggcaacacaagagattgcttatttgcgtaaaattggaaggccatcccatccctattacatgatttagaagcattggtgtgaaaattggttcacttcgtgccccaaatgcatctacataaattatgagctcgttatgtagaagttcgcgaatacgccccagagactgataaaccgataccatatcggggccgtatttcatgggcttaatatatgcgaacattttatggaaatatttatgctcgaaacttatcccgacggttactacggtcgtatatactaatactcttgtagttgcccagaccccaacttcctccgtcgggcgcgtggaattaagaaccaacacggcgttggtaaattggtgacagaatctggctacgagttcagaaaagatgactgtggaggaaaaaatgcatacatttaacccgtccatcagacaagtagcgagttttccaaaaaaactttcgaaagtaccgacgcgggcgagtctttcatctaagggggatcgatctatttccatcaccccctcgcgactttcgatgggtataaatccaagtgcggcagtggccatatctacaccaagcgagcgtaaaattacagcttcccgtttagaaaaccccggtgccgcataagtattgacaataaggtgtatattccttccaccgcgtaatgtggctaaaaagtcaacaaatggtgcatttgtagtcgcatccatagtgataatcgtaggacaagttctcagtagcttaagaagtaacgcgtcaacgcgatttagttttgtcatagtaggtgaatataactgacccaacgtagacataacctcatctaggaccaatatatcataattatttaacagactatcatccactctatgtaagctctcgacctgaaccaaaagtctgcggaaggggtctccagtcattatgtaagtagaggacgtaaaatacgtaacgaatccctcgaggcacgcgtcgttaaatctacctgccagagtgtgggtgaagcttcttctacaagagattaccaaagcgctcaggtcggagccatcgagggctgtctgtagccatttcagaagcgccgtagtttttccggatcccatcggtgccctcacaatagtaacacgtctgctggtggtggagactggtgatggaaaaataaccgggtgtccgtcttgacgctccaatcgcacatttggatgggttttagtgacccactctcccaaatcacctccatagagcatctttgctagagacacgctcgacgtgtaacactcgtccgtcgtcgaccgtgtgggtgagtcgtcattagagagatcttgtggggaatccatcatggagcccggcatagccacaacaccagcaacgattatgatcgggagcgtgtttgacagtatcagctggagatcctggttgattcaaatggccacatttgttctggcttcactgacactttttataacccttattctcgcctcctttccagaggtaggatttccttgtttctatgggaccatcgtcgattacaattctgtcaatacttcaaagttaataaatggtgtttttatctcacagcgatatggggatataagcccaacgctgttttttgagaccagtacaacggtggcgtttttttatatgatgacactcggcattttaattttggcatgctacctcatcacggcagctcttatgattcgacgagaattaagatcatcttcgaatatgcgtggtagtgcttccagagtatcttcaatggtatcccccccggctacaattttactcggaactttggcacagtggctcttacaggcatccatagtacttctcgcccataaactcatagttctcgcagctatcatctatatcgcccattttttcattctatcgcttttttgtatctacttcacatgcttgggtatcacgagctcacaatatagcctcaatttacgtgcgataaagactgcgaatccaaggctccatggaatattaggccccacccgtgctgttatggttaatattgtattgggtataatgggcctctccatcgccactacatcccttatggtcgggatgataatagctaatagctttcatatcactctgtggcaaacgacatcggtggcgatcggtatatttgttttcctagcgattatatatgtaatttttggggaggtaatgctatcttactacatacatgtactgcccgcgccagccttctcaattatcatcgcctgtggggccatgggagttaccgctaatgattattttgacagattttacgtcgaggttgcgagtcagcatcaaagtatcattcttggaacaaaactagcacttgccatagtcgcttcaatagcagtgattatgttggtaatacgatgtgtacgagcgtgtttatatcatcgaggcaagcagacgaatttctatgacaacgtacagtatgtcaaagcaaatatgaaaaattatatcggccgcaagagggggcgatccaagcaaagcaagctgtacagggataaagatctactgctctccgacgcacatgatgacgaggtgatctatgatagcgtcacccccgacgaataccgcgatagtttctataaataaaaccattttaaaaataacgttccttgtgtcttgttattcatcctatgttaaataaaaaatagtcatatattaatcacaacacgcgatattcagttcaacatattttaataggggtcatatcttggtcgtttagattttttatgggaacgactggatggtttttgtacgggctgaacttttacaggcggttgttggtgaattatattatgaagggagtcccaatcttcatccaaactgaagtcctcaaaaaaactgggatcgagggacatggtctctccattatgcatcaagagatgatttttccgacaccatgaacaggagaatttctgtcccatatcgtatcggctgtagtgttgaacgcttctaaactacacgtttcaatatctgcccacgtctcttcgtgatccactatcacaggagtaacgatcaagagtacgggtatagcacaagtagatgggacggtctcgtgtagattataacttcgtgatcccggacgatcgaggatagtgaaagttttgccagcctcaccgatgcggcgatgtctgcctatatacgtcacgaggaatgctctaagacgtctgtgtggaaaatgacccgagagtacatacgattgaaccaatatctgtttaaagtttgcgtgtctcggattggcgaagatgggcgtggataatgtgagatcgccggtatcccagcagacgggagtaatgacacccctctctagatcaggttcactaaataaccagacatccgatgacgtatctaaattcaaacatagatgttgtttatcgaaatccccacatcgtctgttagtctctgctactaggcgtggtctccatgcggaattgcatgtggtgagtgcttctgtggaactgggagctttattaggttcgaagtattccacacatggattcttgattgatctgatgaaccgtcgcaaagacgcggcactgcgatattttagaagattctcatatgctatagacggcggggaagaggattcgggactaaaaaggtacttggcacgacatttaatttcaaaaaaatccagggttgtttgtgaattagagggtttaagtctcccgagatcatctcttgcacagactagcatatcaagggacgcccccacaacccctgttcgaatatctatcaatagaccgcatgagtacgcctcacctctctcacgtttgggactttcctcgaaaatgtagaacatgggatcttctgttcgagtctcctcgttcttagggacctctataggaggtatatacaacgtttcgataagtgctcgtaccataggttcatttgttctcccaaacgccattgccgggttgtcgggaatggggcgtgcattggtactaagctcacaccattggggtgggtatacaggtccatagggcccccatcgcattgtagatgcggtcgcgagtcctctgcgtaaaagtcgccatagatcactttcactctgatcacgagtctccgcttcgatacgtagtattacatcttgtatacgtgtagagggtaatgaatcagcgagggttttggcgtgtgtataggttccttccacaaatcgtatcggcttaccgataccatatgtggttgttgccaggtaaccgctaactagaccctctagttccatctgtcccactagatcatgtatataagcaagacggtaaaaaattgggttcaaccgtttatcattttttggtaatggagagctttttaagaaatgtgaccaaaacgttaatgttgaaaagctcttaggaagtacagatggtaggaatgtttgcatggaactcgtagaatttacagcgtcttcgatggggaatggaaccgtggtatcccctcttaaaacagtcccatctaaattgcgttgacttcgtagcgcgtatgaatggcgtgggttggttaattggaagtccataacactggtatagaagcacgggcgatcgggtcagaatgacaaaatatagccaccaattcaaatagatggatatattcgtccggtacgcgcacactttcaaagatatggcgatgggttttctgatatctctcgtggtgagcgtggaatgcactgcgatgatctactgcggtgatcttagaggcgatatcttctactgcagggtatggaacaccatgaattgttgtaataggtgaagaaggaaatatatgggggtatagtgcgcagcgatatgctaagagatccaaggcatattccacggtcacctggtgtaggtagtagtaatatgtcgtatttctaagaatggggaccggtaaccgaggggaaaggcatccagtcaaaagcagttccaatagcacctgtccaagagcatacaagtcaacagctacaccaacgtcactgtctaatgggccgcggtgtcgagccagtccactgttattaataaagtcaaccagtatctccgtgggttggttatgtccatgacccaataccagactaaatatagttgtcgccgcccctcgtgaaaggcgtaggaccttatttgacgcatccccagtgggaatttcaaactccgcccgcattacggtagaatttgtgtttagagtcatcagactaaaatcggctatgacataatcttttataacaaaattttttgtgttgacgaatatatttccacatttgatatccaaatgcgttaagccgcatttggtgttcaggtatgccacagccttacccaacccgatgaacgcatgttccatcgcattaagtatatttgatgttgggggtccagcggatgatagtttattacaatatacattcagatccatatcatacgctttgaataccatttggtgttcggttaatgaaaatgcaacggggtaaattatcccagttatgcctatggatgatctagcgcgtagagagctctccccggctaataaagtgactaataattcatgttcaaaacacgatggacttgatgatgttttgatggccagacctgtttgtcgataaatctgtacttctccatacccaccagcccccccgaacttcggtggttctcctagtgccaccttagagtaatgcatttctgctgacactgagaacacagggatcgtaacacgcttaattttcctaattttcaaaatagatgacctgttgccgggatttgttgttaatggggtacatacctcatgtataggaattcgagtcagggtatttggtatatcggtccgtccattgttggagtcccctcgaatgagcccgtcatatcgtccaccagagggggaagcccacccagccatacatgggggagtggttctcccgcctcgtctactagacatgcgtctgggtgtatatttgaagatacgtctcgctcgtgtaaaatatctgtcgatggcggtgtttgccctgattttaccttctccgttaccagcctgaaagtcttgacattcttctttaatccggtgacgtaagatcccttctctgtgtggtaatgggcgcagcctccttggggacatggtgaggtgtttgtgagcgctatcggctgtgttaccggatccagagactcctggtttacatataccccgtttgatatggtttccttgagagtggggatcggccgtgttgcaatgtccccccccttcccggacccccgatgatggtcttgtgataggtgagagtattggtccatgtgacagtcggtcgccttgggagcttccgccaacatccatctcgtcagtagcgcttcgtccgcctcgtctagccatccacgttccagagtttcggggtctggatctacttctagaatagtttcgtttaattgattttccttttcaaatatcttatcatccacagcatcaattttatcagtgaagtctgactggagataccgtcgatggctatgcagtacttcttcgacggccgtctgtgcctcaatgcgcagtcgcagtgattgtgtcttttgttctatcgcctccaccctagagtatgtgtgtagttgtctgtcaagggcctgcccggcccgttttgctgacgtaaatgtgtgaacaaaaataggatcacgcctatctatccctccacggatcatgtctagcgtacgtgccttaaatattgattcacgttggtaagcttcttctagttccaatcttcgcctgcggaaagttcgttttgaattcatcctttagagttatcgggtatcttgaagactattaatatgttcggtagatcacttaatgagcaaaccgtcggatattttgaatctttaaaaagggaacgtcaagcccgtctaggttccctccgtgaaatcacggagggatcgacggatgtagacgacgatacccgtgtacccttttttaatttctcgatatcgacaccccagcgccaccagaccgttgtacccgcggtgggagctctacacgactgctgtgaaagcgcacctattttctcttgcatagcacggcagttgctttttactagcatggtccggaagcgaatcagcaacgacgacactggggaggaggcggtgtccggttcggttttgacagcggatatgttacagacgattcagggactcacgttcaccaaaaatgataatacctcaagtagcgcatatcgaaatgcatattatagtgtaatgaatacatttgaggccttacgtacgtcggatgcttttgaacagatattagcgtttgtgaggagattcaatcatctcgtagatacatctttcagggatgctaattttgatggtggtttaacgcgacacccaccaaaacgagcaaaagttgatgtggcatgttatggtaaaactagagggactctagagttatttcagaaaatgatcttaatgcacgccacatattttatagcgtctgtgttattaggtgatcatgcggaacgcataagcattttcttaaaaatggttttcaacacccccacattttcagacgcctgtattaaccatttcagacaacgcgctacagtatttctcgttccaagacgtcatggaaaaacctggtttttagttccattgattgctgttacgttggcaacgataaaaggtattaaagtgggatatacggcccatatacgaaaagctaccgaaccggtgtttgaggaaattggagcgaggttgcgtcaatggttcggggtgtctagagtggaccacgttaaaggggaaactatttcattctcgtttccggatggagctaagagtaccatcgtgttcgcatcgagtcataacacaaacgtgagtataatatatattttattgggtattatctattgataggaatttataatacagacgctggtctaataacattcaatccgtggtagatatattgtccgagggtggggaatccatacataatctccttaggtgaaaacagctgcatattagagccttagtgatattctcgtctaggacaactaggttccaaccagccccattggctgggatctgtcgtccatctgagccaattccactgataacatcagagatgttgttggtaatttttggattccgcggcgatcctggtaatctaatacggtccactgcatccataaacaacactctgaataagacgtgatccccctccacgggaagatcacgttgaccggccttgcgccataaacagggcgcccagcatagacatgactctggtggtatgttcgatggttcggatgatgcattaagcgcgtcgacatgtccctcgaattgacacaatccacaaccttcccgatgattaagtaatctggccaatataccgcataagcttctacatctccatcccgaggggcatagggcgaggagcgtggtatccatacagagatagtatatagaacattctcgatatgaccaccatacccccggagctagaacacaacaatcatagcgttgatctggaggctgtgcgggcgtcgcaaacatactcacatctagatgtaatggatcatcacatataatcagttccgcggtggggtctggaacctcctttgggagggggatggcactagcgtcgtaaaacgaagcccctagaatcgtactgtcgggaagcataggtttcaggatgacattggatataaacgcatatgctgtggatataccgaataaagctaggatatgaaattgacccttagataacttgagcgcctttggtcgagttatgtacaatgtcacggatacacaatagtttttgatggatgaaagttgagatggagctggagtaaatggagcgagcctcggtgttagcagacctacccccttgtagattttaatccgcgagtcactgcggatcggcttaagagaacagatatcgtccgaaagagcttctattagaagctcaacggcatcttgtgtcaaccgtcgttccggtcctgtagctgtccatataggatgggaagctctctccatgattgatgatgtttacacgacgaacacgagctagggagtgggtgggatagaccaacagaagcgacttaaaatggcctcgaaaagatgggcgggccaaattaaatggtccgctggtgtcacgtgaaaagcgaatcccccggggaaaacaataattcccctgaattccctgttgaacccaatgcatatgtaagcatagcattcgccgggtagatagcgttcatatgatcttgatccagagccctgactaactctagcgaatctacaacgatattgcccacccacacattttgttaataataccgccaaaggttccgccgatagtctcaaaacgctaccagaagaactgaagtagtcaaataagtacatggcatccccccctgatatatttggtatccaagcctggtggtaaggtagacgtcccacacctcccgaggctggtgaaattgcattcaagtgtcccagggcagcaagcattaatgagctgtctcgaaaatcacgaacagatatctgggtgatggaccccacgagggcctttaataattccggtatccgatttaccactttatgtatataatcccgctgtgtatcatggagttcatattccaataattgggacatgcgtggctgtgtggttatcgtttctgtcatggacgcgatcagggcctgtatgcatgcgttcttaaatctctctagtaatacaacctctgccgcgacatcgaatgccaacataggtggtggcaggtctcctaatggaacttctagcataggatattgttctattattcgctttcgtacgtcatattggagatcccttggatggtttataccatgcgctagacactcagcagagattatcttaatcagcgatccatctaggtttgattctttggccctcctcccaccccaggctatagaattttcatatgataccactacgaatgtttctccggtgatatagtggcgacgtgctactagagagatcgggcgtcgtcgccacacatcacttggggtatctccaccaaggagggctacatgtccaatatcgagccagtctaggggtgtgagaggcttgggttgttcctgagagattataagaagattctccggattttcctccctgtgtgtatagggacgactagtttgaataaaacgtccggacccccgtggctcaaggcctgataataaagcgtcggtctcctgtctaacatccccggtagccatgggtctgtttttatgatttgatatcccgattgcaccctgtcgtaatccagatgccactccacaaccatccttctgatgctgtggcaccctccttactgggtgtctggtggttatggcctccgaggcacgcctgatcaatgacattatctctctgtctgtatcctccaaccgtgatatagccgtaggagtgtcaaaaccccctccattcaccagttggtgtagagcatcgatagttccatcttgtgtaagggattgggtgtggttggatattgtatcatcccccatacaggcttctatgttggccccggccactaccgctctatcaggtagaccgggatgacgtttagtatatgcggaaaaattcttgcataggaatgttagtctagaagcatccgcaccatatctcgtcccaggtggtaataattcatcgtacgtgaatagtagttcatatgtttttctatcattcggaatctccagtcgtatggctataatattaaacgggtcgaattgtctattgacatcacaatagatttcaagagtgataaataatcctccgggatcaggagataccctaaatagataagggtgatcacgcggacatgttggcacaagggtattggtaattgcaccatttgggataaaaccaccaaatgtcgactcccatggcgtacatttaccacttgcatgataccttgtctgtgttatcaccttgaaagatgaggtggggagatcctcaccatccgttggttttattaactttgtgggcacaattccagatttcaccaagcaggctgttgggatgatgagatgaaccaaggttcggtctctaggcgacacaccataatttagttgaaacttcgtctcattggctatatgagcatccataatgcatgtataggttatggagatgagaaactgctacggtagtaaatatatatcgaatgggtacaaaaataatcgtgtatataatgaagtttaaagctttttgagctgcactaaccggtgattctaccctcttttatattccctccagggtatccgaggccaagattttaacttgctttttgtcgacgaagcaaattttattcgaccggaagctgtacagacaatcatcgggtttttaaatcagacgaactgtaagatcatatttgtatcctcgaccaacacgggaaaatccagtacgagttttttatataacttaagaggagcggctgatgaattattaaatgttgttacgtatatatgcgatgaacacatgcagagagtgttaacacacaccaacgccacggcttgttcgtgttatattttaaataagcccgtgtttataactatggatagcgccatgcgtaataccgccgaactcttcctacaagactctttcttgcaggaaattataggtgggggaccctctcataaaaccaataccgagttagtttttacaaagacggccggggagaggtttatgatatacaggccttccactgtggcgaatcagtcggtattatccaccgatttgtatatgtacatagacccagcattcacaacaaactcacgcgcatcgggcaccggcatagcaattgtcggtcgctataactcagattggatcatttttggcctggaacatttttttctgagggctcttactggaaattccgcggatgatatagggagatgtgccgcgcagagcctggctcaagtttttgcgctgcattctcgtccattcaccaccgtgaagatagccgtagaggggaacagcagccaagattcagctgttgctatagctagatgtatacaaagagaaattgaggagctctcttcaatctcgatcgtacccattcctccgaccatcttattttatcattgttcacctccaggtagtctggtcgcatatccattttttctattacagaaacagaagacgggggcgtttgaccactttattagacaatttaactctggacatgttctagcatcacaagaactcgtatctgttacagtgcgtctccagacggacccggtagaatatctgtattcccaaatgaaaaatctcactgaagtggtaacaggcccagccgatacaaggacgtataccggtaaaaaaaacggcgcatctgacgatatgatggtagctctggtaatggcggtatacattggaagtctccccccaacggatggttcatttgccccgctttctagatcattatgagagtcatagacgagcgaccgcggagatattagtcattttaaacccaatacaacatcaataaaaatatacaccgtgtttattgtctatgtgaatgaatgtacaatagatgcccgctctcccacggggatcactggagacgcatcgaaagtacagattcttaatatcggctttgcaccgattatatcaccaagtcgtgatgctgaggttacccatgtagctaaggcctcgtacacacaaaatcgccgcgtgccgtcctggggagggtggactggtggtcgatggattagttgggccacttctcgagtagcacttcccagttggattatggcattcacataaccgtcttgcgcaccaagggtgagtaggttcggtatcaaggtcaataataataaacagccctcgttgaggagaaatatcatattcaggatgagggtcctcgtcgctgcatctataccatcccttcgttgtatatccggagttagttgatatctgcggccgttgtagaacattatatccatcccccgtctcccaccggcacctaacatctcagcggccctagcaaccactcgtgctaacatttccctcgcctgggctactggtacagtggatggaaaaattatatctaaaccgcaagaagttaatcgcagtcgatcctcgttgcctattaatgggggtacaaggcaaattgcatcaccattacaaagttcgaatggtccagtgttctggataaaggcaccaggaggaagtggtgccacgccgatcgccacggctgtcatctttccaggtaatacacgcataataatggctggaaatcttcttctgtaggccgccatgagagctaaggtgtccggaggggacccattaatataataggatgataacgctatatcccccaaagtcgccctgcgtttaagtatatttaaaaatactatctttccttcacatttctgtaaagctgtcatgtccgcctgtgaaatatcaccggggaggagaatttctatctcaaatgattgtgatgccatcccagtattttgtgaatattgtcgatgataaacgactatagacaggtaccgtcaccgggaggttatgagagaagagcgtatgtactagttcacccgcggacgaatacgcaagtgtatgataaaagatactttattggttcttatatgttctcaaaaatatggaagtcctttttatgacgcattggcgatgcagcctttcaatggcgacccatcgcgaatgaggtattgtgccaagtgtgtctcatccgtaccacgctcgttattgtttgatgttcgcagtagagctatatcactggagctcaacggtggatatgcctcttgaaagaggccgcaagggtcttctactaactcggatgcaccgggtggtcgtttgaattgaatttctgtactggcggtgcttggggctaaactggacccggtttctacaagcagttgggcaagacttcgatgtttattttcaacctcggttggagtaaaaaatttaaaacatggactgtagacgggtgaggctccactgaggttgtatgccccgttgtatagacgatctccgtacgaatggcgttgggaggcccatgggtttatcgtcgccctatttggatatgccgggtctccttgactatggtcaaacatgatactctcggaattttgtcgtccatcccccgtgtagatcatacctgtggtgcgcccccgtgggttacacggtcgccgaaagtaattcaaatcggcagaaacaggggtcactataaactcacatatggcggcttgaccgtgggtaataccgactggggtacgtggcattatctggccaaataacggtgctggctcctgtgggtctagtctgttgccagaattgacagcacgtctcaaaaagttatccgtatcaacatccaacaatggaacactgccacgagttaggtagaggttttgtggaagattgcccatgtcggtaataggggtgcgtagcgcagccgcgctgtatacagcagtaaagcctacacccagatccacgttggccctgggttgtgtgagtgtgaaattgacaccacctaccgcctcgtggcgactcacctgaagttggcccataaaataagattcggatgccttctccgcgaacagtatattttccgtcgagaatctgtcctgacggataacggttaacgcaaatccaggatgtaatcctgtgcgaaattggtgtataaaggccaaaggtgtcattttaaagtatccagccataagcgcatatgtgagggtgttctcatctaccttactcagagcggcgtgttgagcgactggctgccgtaccgtggcgtaataatttgctcccaggaaaggcggtacgggtggtacgtcccgtgccaatctcaaggcttcacccgccataccgggaagagccgctaaatgatccggacatgcaaataaggcatttaccggggcaggatagaaaaacgtcccatcgaagagggtttcatcgttccgttggtaggccatcatcaagagtccatgatgtagagtgccatcaaacgtacgcatttctcgggtggtggccgtggtcgcaccgacatcgggtccggcggcggtgagaatgggagttgtccgttcggccatgttagtcactacgtcctgtagtactaatagtgcatcagtatctaccattatgcggccattatgaaacataacattaaaggagttagccactaaatgtcgcgggtttagagggtggcgggggtcatcgggtgatgcaggtgcctcctcatcccttcccagatccgggataactagggattgtacgagctggtagactcggtcatatcggatgcccatggtacaacagttgcccctagagaaggctggaactatcgtataataataaatcttcgatagagttgaccattccggaccgtggtgtgggatgattggggccctggcgttttccccgcgtacaggtttattgtggatcagatcaccagaattatttctgaaatttacctgggccatctcgacccacggtaccgggttgtaatcaccatcaccaacccataattctatgacacgattttgtgctaaatcctgacggtagattagcggatcacaatcccagatgagtggggggagaagggcatggtccatgagaatgttgtttagctcctcgtgatcccgaccagccatgggctcggctggtaatgtatgatctccaaccaggcgccagagggcacgtgtatgttctaggagttctctatagatagttgtacactcttctggcagctccccattacacagatagatgtttatgtacattaccatatagaaattatttacaaaggcgacattgtgtgaattctgccaataactcttgatgcattggatgataagacgcgcgaggccgcaaaatatccgctcgctcccatgaataaccgcctctattatatacaataccatgggataattctggtctcggaaggcgccacgaacggcggcaacggtggctgggctgagtctgtggcgatcgacactcaattctaagccacgggtgtctctaaaatcgataggacagagtggtagtggtatgtttccattgattatacgtagactgacgccaagtccaggcatgacctgaggtggttctggtggacctggctggttaacatcaccgggaattacaaagaaatcaaacgctggatgtaattcaaaaatcagattctgattaccaggggagagaaattgatctgttgtcataagtgcttcgagaacccatcttggtctattgggcatcataacacgccattgatttccaaaatgacgcatctgtgtcggtagatcatcccgttgtggttgtgctacatatgctccaaatacacaatttagttctggtataatctgtctgagcgctaatatcgtagcctcgatgtctagaaaagagctatgacagacggtgcctatagcactctcgaatgatagatattggaggaccccatccttattaaaaaaataaagaccacgtggtgggaaaacacgggaatcgggttgtccgggagctggtacaatgcttccggcatgacgagcgtatctttccgaagggggtttaaaaactcccaatggcataatgaaggtaagatcaagatttcctataagaggatagggaacttgcgtggcttgatatacctttttttctaaggcttccaggaagactagtctatcaccaatttgcacgagatctgctcgcacacgcgcagatcgtggattatcttcataatcttctagattgtgaacatttgtttttacttgctccttttgaaacccgaggagattacgagcaacatcatctaaatttctcacggcctttcccatcactagagctgtgacgagattaccaccggtaatcatcatttctccatatgtaaccggtacgtctgcagcactgtcttcaacatttacaattccccttaagagccgttgccgtatgccagaggtcgttatcaagaccccgtctacaggtctccctttagtatcggaatgtgttaagcgcgcgattgcaaccgatggggctgtgcagttaaccagatcattcaggtgggtgagtataacatcacgatgacggtcatatttggtcataaaaaacatctcatcgatcacccggcgtttaagttctgataatagtgtagcattggccacagctcccatactatgcccaccatcacgatatagtctaagaggggccaatagagatagcggtggtgccttttccagtaaaacacggaggagttgatcggcagttccacgttcaaaggcatccagggccgcttgaatatttctggctagttgttgaatggctcttgtgcgtaatgcggtcgagatatgtgtaccatcaatggaagtctcggtgatgagccctagggcctcagccgctatggaaaatgcggcactaagggaccgtctatcgatacgttttgccatataattgtgtataggttggtttacagggtggggtccatcgcgtgctatcataggttgttgaacctcaaattggatggttccttcatttacatagttcagctctgggaatttggtacagacacatgcaacggagaggccgagttcgagaaaacgaacaaaggttagggtattgcaatacgcgcccaataagacatcaaattgtgcagcgtataagccgttatcgtcagagcgtattcttttaaaaaaatcgaaaagagtcctatgtgatgccacctctattgtagataaaatatgtcctgttggggtggtgaaatcggctattggcacccgaaagggttcatttggtctatccatcgtgtctattcaacagtatattgggagaatggagacgtggtagtagtttgtggagcttgatgaaagagtgaatagcactcgcggtgcgtggtagctttttatattcgcgacccgtaatacctctaaaatgagaggcgtgtccgcatcagaatacagacccacatacgcggggcgacgaattccacggcgtcacatatagcatccgcgacatagacagcggccgcgattataagcataaaagctactgttctgacactgatcgaaggaaaaagagtatgaactataagaggtgttatataatccataggagaatccgatgatgagacgcggaaaaatagttcagagtctacaaaaatggcggagtgaaatatcactataaaggcgataagtctaccaacggacatagacccgatcacggcttgttgccaccatcgcaatggcaacaaatctacacgtatattattatagataaaaaatatcattaacattgattgtatgtaatatactaaggtaattatacccccagatgttatgaatatactattacctgtcgctctgtaatagtttaaaaatgccagacagcacaatggtttaattacaaatgctgaaatggcaaaaaaaactacagatattgtaaaggtgggttggtgtttacccggctcgatggtataagctgagcttaacaaaaaatccgcatcccccccatatgcggacatagagatgtactcacaaacgctttcatcgtcgctatctagctcttggtcatgaattattatacccgcttgatcatcatcacgacatagtgtagccatgatggtaaataatgttcagatgtttattggcggccgatgaaagctctcgtccccgtcaggtacagtcaatcatataacaattcacccccccttatctatttcatgtaagtttactcagctccgctgtgtgtggagtagtaggccacctcctcggttatatgtattatctaattgaggagttattaataagatatgtcatgtcttaggtaacatgtctatggacgtcatcaacgagctcaaattcatttatgttttttctcccaatcaaaaggctctttagaccattaaactcttccaatctgtgtggtagtctagagcattctgcgttttaaagccattccacgatcaagtccgttggtttataccgtctacagccatggagttcaaataccatgacactatggtatcccggggagtacttttctatattgcagataatgggaatcgcatctacttcacacatggtggctgtctaatatcggtccaacgcccacaaagtaaaaatcgaccggagatcgctaaatttggtctcactgtacgcggtgtgggggaaggggatcgtttagttgcaaattatgtgcgtagtgaacttaataataccggtcggggatgggctgttccacaaaccgatgaggatgtttttctagataaagtggacctattatcacacggaccagatgtttccgatagagacctgtgcggaggattcgagcttgagatctacgatcaatacctgacagagtgcatggtatcccttaaagtatcatcaggtatagtactgactacgggacgagatctaccccaagatggaacgctgagattatttgaggtacccactattacaaacgcatcgtctggatttgtatataccccaaatagatcttgttttagactgatacaagcatatttatcagaattgccgaaagctctagaggaattaatcgatggactttttgatgggattccctgtccccggctacctatcaatggagatgaaccacccataactacagatgtgatagtgacatctaaaagagcagtacctgccatggcggttcgtccaactcattatgccacacgcgcgttgcgacgtgatatgattattagcaattttgtgcaggtgcgctatatcccaaaaccatctaatatctgggatcctagggatcgggcatctggagttaattctattcaagttttatgtgccctgatacagagggttgatgagatactaagtatagatgactgttgggacgggttggatgatgagctcaacgaatcccgtagtacaatcaacacagcaatatctgcgttattcggggagactggagagaaaccatttttaggcgctaggtcctccgctggtaataaaattaccccagcgcagaggttcgtgctatgccaatttatttttatgcgatggaatttgttcaattgctacgctacactcgaacgactttccgacagctattctagggccttttctactccagaacccaaacctggtgatgtacatactacattggacgcggtaaacgatttacttggagaaggggccatcttgggtaaattaagtgtgttaattttttacatgcagttacatcccatgataggatataaacagttcagcccgactgtcgagaaggaggccgaaatgctgaccctattcacagctgaacacgagaagaaaaatacacagtttctaatggtagatgagctgagacgtgtggactttgaaatcgccaaactcctagacgcgctcttttctggtggcggaatagatggctgtgccctggctgcatgtcgttctatccgaaatgctgtcccggtataccttgcaatggctatgaaatctatcacggcgtttgatatccacgaagatgctgcgcgcggggcacagtttatatataaactggtgactcgtcgtcttagactcgggggatgtagcggttttgataaataaatatgtactgcctcaataaactaatcttttatatccaattcatgtattatatgggtttctttgggtcatatttaaaagtgtgtggcggtgaaagaagagacaggagacctaattacccactgttaattatttaatagcgtatagcgtacatctggtgtgaaactacagagcatttttataattatatatattataagacaggccgcgaaaactccacctatagacgcccagataaagttacttgagaatacaatcacttcgtgtgaggtgaaggccataagggtaacaatcgttccatttggaaataataataaaacactagacggggatgagtatttggtgggattgaatgctgatattgtagaattttcaccagccaccaactccagttcaacagccttatcgttgatcaagagcagatccacaatttctccggatgatttataacgcataaaaacacacccgcagtatagacactggtctgtgttctccgggttattaagattgccgggtaatattatcccacgctctataccacattcaccgctgcgtagatatgagattattatcggatttgatacatctataccatcaactatgtataatgttccgcgtcttgggtcacgtctcgttataagataagtacagttaataccaataggtaaaacagctatcggtctctgatgattagatgaaacacccggacatgaaaatagctccggaatgaacgtgtaaaggtttcgcagattaaatattatgttatgggtattagaatcaactgccggcttttctatggaccgatacgatgaaatgggaaatgccgagaacagatccaggattccataagttgatattatatcatatctattagatgccatacacggtgaaaaaccatcaaaaatattaaatcttgtcgatgatttggttagatcatagagaatttgttgtgggttccacctaacggtagaagcgtgttctgtcgtacacattgacgtacattgtagaaataacatacgacctgcgttaatcgcctcttgctccacagaactctgtatatttccagctatatagatcagcatcgaactcagaaataatgcctgcttggccgttgtgttccatctgacaacatcactgtaaagggtttctaggtataggtccttgactatatcaccggagagtttgagtgataattgggattgcttttccatagatgtatacgtataaattaactggaggtatctcgcgcgagttataaggctcccatgtgaaacgtgttgatgatcgatagtgtccaatgcatcgcgaagatgtgtcctaccaacatccgaattataataatatgtaatgttgattggatcctggaaattttgcattccatcggccagtaatgtattagcgagaagacgggtgttaaaatccaagtctattagctcatcaatggccacatatccactcaatctccgtacttcagaaagagaaaaaagagcagttgccaacctagttgaaatttttgaaatataagctgtggttccgtccatgtcttgtggtgtagcgtgtgataaaaataaggccgtttccatatgtgccatggaaaggtggtaacgataatctaggctttgttctgggtatgatgctatcgtacgcatggtcttataaaagctcccattgatcctattgttaccatagcctaaaacatatactttatacaatgggccttccgaaggtcccggtggatctaatactagactagatttaaatcgtgttatcattcttgcattggtgtagtgaggtgtgacgataatctccaagggtggataagagatggaggttaaagaaaaccccataaagccaacaccgatcgttacattgactaaagtacgattactccatatccacatcccctccaaaaaattcattgccaatggccatacggcctcgtaattaggtaaatatattccatcacgctgtcgtcttgtgtcctggaaaaaatccaactccaatgcgcaatagcgtataattaaatctgtatggccggaggtggtcggtttgtcttttggcgatcgtcggggggggttagttatatgtcctaatggctgtgtctgagtagcagttgttggtgatcgtgcaaaataccaatcatgaagagaggattccggggttacctcaaaggcaatcagatggcgtgtcgtcagatatctcccattgtatggattaaataaaagactcgatagtggatatacaggggatataaacccaaatggggcggccgccgcttcatcgcgaaccttcgcgggtgttggttgtacaaacccagccttggggaataaaaacatagtaccgcgttgacgttgactgcgtctatcgaatatgaatagaaatatatcgctcccattttttggtaacatgataaataagtttctcacgtctgctacatcaacatcgtccctataagaaaactccccttctacttcaccaactgttcgtcgagccagcccaccttcatcaggtctatccatcatcggtaaaccagtcaacatcctcgatacaatgatcaggaatatcgataggtatattatcaaacacatcatagattgcttgaatccacgggctgtggcgatgatgtgaccaacctccaaagaacccggctcttggatttaaaacaccagttttacgcttataaagatctaaaaccggaagagaaattcaattaatgaatacatccacatcacacacggttattttattcatgaaacgatggtcattatacaattatcccctgacctccacacattcaccacaggggggtaatagggaacaccactaatgtttaatggtatatcgtcaaggcttcacttatacggtgcatatcacatgcggctataaatgtcatatccatatcttgtctcagtgctcccaaggcggcggcacattcatcaggggttccttccagatttaaagtaaagatctgtaaattttggagggtttccataagtccatcgaggacccatgcatgcatgtcaagtagatctccattttggtcggagagctcccgggctttgaaaacactgaataatgtgtcacataaatctgactccgtcgaatcggaaactattgtagtttccacgagcctattccgttcatagtgggagaaaattttaagcttcccccacccatcacgccaactagtattttttgttaaaaatttcttagtatgtaccaacatcatatatacattgcgtagtgcgtgaattagcttcatgtctatctgttctccggttcttgagcgtcccctgagacgcttcaaatgttcctcgatattcaaggttgttacaactagatttccaccaggaggttctcgtggaagtgttgccattagactaagtacagacccaagagtcatatcacccacaaaatatcttgcgagtgggaaacagaccagagaggcgagagggtgtctatctatgattagggtcacgtcggggtgttcctcacatacaactttctgatatcctgttattgtggatagtctggaatgtaaaagaaggtatggtgcggcaaatcttgcttgatagtgggcggtgatataggcagcatcttcagctgataattcaccacgtcgtttccggtcctggacggcatagataccaccgacaacatcagtttcaaagagactacgccagtatagcattggttctgggaagtagtaagtatatcccggtcgattttcatccgctctgaccaggtacttcgccgttaaactcttacctattccataggcaccatctatgtaaatgcggacagtattcacctgtgatttaataatctcttcattctgaacggggatggttccactcgccatcgtctgatctgtgtatgatgaaacgtgaacaacgacgcagtgtcggattgaggctccgggctggaattcgatgccacaatcgcttttatgctgtcctcaagcgcgatatatcctcgacctctgcgagtggagtatataccgaccgactcgagtgtttattgggcggctctgtatccgcagagtctcttaaaaaggctaagggcgtgtacctagcatgtgaggttaacttgggtcgtcgccgacctgattgtgtatgtactatacaatttgagggagaagggggtggtatatgttttctaattgagctgaaaacgtgtcgtttttcaaaaaatatggatacaactagtaaagacattcaacgtcgtgagggtttgaaacaattaacagattctgtgggtttaataaccaagattctaccaccagggggggagaagctcactctaatacccatattggcatttatcgcgcagcgcggtctgagaattttgggagttactagtttacccccgcagatgcttacgagtaacatttctgttctggctgctaatattaccaagctcgccgaatacaatccaatcgagagtggtggtgtaatccgctccaagaaaaaatcaaaatacccaaggtctgggtctggggtctataaacatacgaaccacgtaccgatgtcatcgtgtgtatctgtgaaggataaaaaaaacactttaactccaataggaagcggcgaaagtaaccctttaaaatgggtagcatctctctttcccgatcactccgctacgcaaccacgtgagtattaatatgccctatatgtgatgggtaatgagtctatatagatcatatatcaaaaccttatttaggatcaaagagattaatcactggtatcatttgttggagaggagaagttgtcgaagacgggtcatcatgactggacgagtcgaatacgtattcgactcgatgcgaataactaacatcggggatgatctgattttatcggacactagaaattttattactcccacatttccagtggattattggcgtgagccaaccttttatttaaatgaaaaaactactcctgaaagtctagatgtccgccgaaaggctgctgccgcggccctggataatttaacccatcaaaaactattgggcgaaacggatatagaggatcgtctatatcccttggagcaacaggtactaaatgtggctaacgcattggcctctctagaggaggtagcacgggaagcggaaacggcggacgctgagatggataaggatactagacctctccagtctaatggtggaagcagatcggatgagacacctggagggcttgaggttcagattaccaaaaatgacactccattggcatatgaaacaaacctagccatagattttttaacgatggtatatttagggcgtgccgcgggatcaaatggtatatcatttggttcatggtatagagcgttacaagatcgtcttatcacagatcgtccgttaaccaccagaagtatagattatcgagatgggcgaatgtctaaaactttcatgacagcaacaataatgtctctccaatcctgtgcccgactctatatcgggaatagagcctactcggcttttgaatgtgcagttttatgtcttcatcttttacatcgggaactcgataagggaatcatgacgcacccaccgactacattttccgatctaatagagcacctaccgacatcattggatattattgctaacaccctaggtactatgccgtctggtagagtaatttatatatataatatagataaactacctagacatcaatttcaggcgcctaatggtggacggtatgaacatcatgcccttgaagaccatagcgtattaaatctacttctccaatttaaggttttacctccgattcctggacatattaaaggtggtcccccggctatagccatagatatagaccagactgctttcgtagacccggttaatagagccgcggcagctttcctagggagggcacataatctatttctcacggaggatcagacgcttctcagagctactataaacactataacctcactattactactccgacgcttactatggaatggaaacatctatacggataaattacgtaataatttccagcttggaacactcataccccagacagcctctatacagatgctgggtacattgactcgcggggcaaccgggggggatttgggagcaccactcaccataaaaagcgagagtcacaatctagagtttttatgttctagatatatattaccgatctatacatctatgccagatgtcgagatcacgcaattatttccgggtcttacagcgctatgtttagatgcgcaagccctcatagcacaaacccgcaccgcgaggcgcgtggtgcaagtgaaaactggacgtttgcaggacaacttaattcggttggttggtctcgaactcgaaaatagacgccggacaggaacagtacccataggtgaggtgatcacggcacacgacgctatatctcttcaaactgaacatggcttgggtcttctcatgcaacaaccacgacttagagcctctcttgaggaaaatcatcgcttatggcaatttaacattggcagtgattacgatctaatatactttctgtgtttggggtatatacctcaattcacagcatctatataactgaaatgttttgctaagtagatcatatacgaaataaacattacatattaaatataacacacctggtgcgtgtgggtctttttattttcaaccggctctccagtaggaagcaccagtttcttcacatgcaatggatacacacgatattatcgaggataccacatccgatctccatatctacgtggctggttatctagccctctatgatatgggagatggcggggaattgactttaactcgtgatgtggtgcgtgcagcattgcccccagcatcaccactacagattaatatagatcataatcgtaaatgtgtcatcggttccgtactatctattgtcgacgatcctaaaggaccattttttatagggttactaaattgtccgcaattgggggtggtactcgcatcggccgcaagtccggggttttttggagtaaaggagggtaaattgacacgcgtggagcaattactctatcttgtcagtaattatcttccatctgcatcgctatcatctcggagattatcaccggatgaagaccccgacgacactctctttgcacatgttgcgttatgtgcaataggtcgtagggtcgggacgatagttacgtatgacgttacacccgaggccgttatcgcaccatttagacatctgaccccagaatcaacccgtgaattactcgtgcgtgcacgtgaaacggaagtcaatcttgatggtagacggtggtgtctcaccgaacaagccctgacacgggcccttctatcgacagccgtaaatagcatgttattaaaagatcgatgggatctagtggcaaagcgccgcaaggaagcgggaatatccggtcatttgtacctacaggctagtaccagagttggtatatcaaatttcatacgtgatgttttaaatggggtggagtccgatggaaataccgcttataagcaaggtaaaacgggggagcctgcctgcgactcagctcataccagtttagacgttccatctgtacccaacgcagagtctgctacggagaagtacagtaatactccagagatggcatcccaggggaattctaccataagccatccacaacccccaaacactggagattatatcttggtcccaacagcacagtataatcaactcgtggttaatcaacatcaggcggcgaatgttcctatccaataccccccggcctctacattcccacctaatatatttccctctactgctatctaccatccacccctccctccaaaccatttccatccatatggatttactccatcgtcgagtggtctcgaagcgcaaattagcgcattgctcggggcattaacatcagaccgtagaatttcccagcgcatggagccaatggattctggatatacacaggcgctggccacctccccgtcccaagaacgtagatacgctcgcaagaggagacatgattgggattcttctggccaacgtgatgaatacgatcatcagtgttattatccgggagaaagcccaccacaacaaacacagcgtagtgggacaaataatgccattatagcagaactggcaggggttgtgtcctcattacaacaagaaaatacacaactgagagcattgcgttcgattggtgtttatcaacaacccagacaggatctacatccgaataatcagaatcagcaatacccaagcggagcattacaaccctgtgtgggaacgccaccagtctgtgtttcacaacctcctacaaatattaacagtcagaatgtccccattacatcaaatattcaaacaaacgatatcacaccgaaaatagatgtggctattgctggaaccaatcactcccaaaatatcgcagcaacggtggatgccagtaccgtgacaggtctcggagtacgtgatgatacggatttctttgtatctcagatgatgaacgctcgctgatacatcccacccatcggtttattaaaatattaataataaacaatagcacgcatgataaacagttgtcgggtgttttattccatactatctacacactcatccaagtcagggatcacataatcccgcatccagggctccacgggagtatctgggaatactcgagggatcttaacccaattcgatccaatgatatcaagggcatccgataggtgaggtgaatcgagtccatcgcggtatgaatgacgaaatggtaaatggcgacacgcccgacaatctggatcgcggtggcatacgaaattccccctcatcacaaacgatgatccgaaaaacttcaccccgtcggtcgatttagtctccgctgtggtcacatcttcatgctgcgttggtgaaggatcacttccagggtcttgtacaataggattaggcttttcagtggtcatgttctcgggtgtacgattgagctccatgtatagttatcgatggaaaaaaagtgtttagcttctgtgttggattgatcggcgcgagagaaatgtgtacctaaccctcaaaaccgggttatttttaaagagaccgtgacgtgaatgggcggagaatgatgacattacaggatatttttgagttcaaaatttattatctctaactttttttgtggataaaaatttagagatggtctggttggttagacaagatttgtttcagtatcatcaccgctatcaagattattgaggcgttggtatttaggtccacggcgacggagggccatgttagtgagatgactcgttaggatcgctgggcccttattctttttcatcgccttatgttcttgttgctccatagccgatacgagggacatatattttatcatctcccttgcctgcattagcttttcctcatcgaagtcatcgactcccgggtcgctatccccaccagccgttgaggcggggctcttagccgtctgtttcaaattcctagtcgtcacaggatataaggctttcattggatttgcacgtaatctagatatatagcgatatgccaggaatgctgcgacgatgccagctaatattaacagtcccacggccaatgctccaaatgggttgtttagaaatgatgatacgcctgatactgttgagattaccgcactcgcagcccctaagaccaccttgccgaaaccagcccccacatccccgagtccctgaaaaaaatttgccataccacgcatgatgacaagattattatccactctgactatgctgtctatatcataaaattttaaggcgtggagttggttgcggcgttgaatctcgctgtagtccaaaaggccggtatcttccagctcagctcgtgtataaacttcgagtgggagaaattcacgatcctctaggagagtaagatttaaatccacatacgcacttatcagttctatctccgatagcgggactttacggacatacgcataatcctcaaaatatacataatctgccccaaacttaaaataccgcttattattgacagtgcaaggctcaattagttttcgttccacgagaagttcattgttttcccctagttgtccttctatgtattcggagtcattgagggcacggaaggaaacaagagggcggctgtaacatgtcgttgatgaacccgtcacccgcatagaattttggatatagacatgtccgctagaaatgttaacacattgtgttacggcgacggcatctccaagtaggcgcgcggatacacgacgttctagggccatcgagaccaccccaccgggattgagttttagggtctctgtccacagcacatgttcgcggttctgaagtgtacaccaggcagtggctatccgactcaacatttcatttacatgggcttgtatgtagtcataggcaaattgtagcattgcgaattccacagatgaggtggtcttgaggagtgatgcattgttcacggtttctatacccccctctattgtgctgcgccgcgacctatgatgttgattagatgggaccgatcttctagttcgttgtactgtttccccagatggattgaggagtgcactgagatctaccgtgcgattggaacgtgctaattcattgatatataactttgctagttcgttgctgatcatgggacggaaagctattagaaatcccccacgggctaggtaggtctccagggttccagtctgaatatgagttttactatatttactcttataaatccggtctatggcttcggctgcctccttggtggcacagtcccccaaacggatacgattgatttcaaattgtgaagtattggagatgaaagtagcggatatggtcttggctgtaaatctataggagccctgatattcatcgcgtagcatttcatctatttccctccatttggctaaggtacatacccgaccagactttggggtccagttccaggccactgtcacatgcggagtttccaaaaaattgcgagaaactggtgcccccagttgtaatcgcgtatccaagtctattggatagtatccctcgatttgttgaaatctgtctgaagaataactagtatgttctacatgggctccatccctcagcccaaagaatggagacatgtgaatcacgtcaccagtggagatagcaaatgagtcatatggatatacagatcttgcatccacttcctctacgatgcaatttacagaggtcccagagtggtgaaatccagcagcaccgatctttgtgtatgtttcattggtggtgtgccatccacgggactctggagtgttgaacttggagggtttcagaggcagttctctgggatcctcgtctcgatcaaaggccgtaaattgataattgttacgaacgtaatcagctttcgagaggcacataccccgtctatctatgagatctgtaatctcttgaactttcacgggaaccctgtctgtatatcggttggttgtaacggcataggaactcccagaccataccgttgtcataataatgtttttatagtatatatttgccttgaatttatatggcgctatattttctttaaatattacagctataccctcggtaaaattttccctagtttataatctggacaggcccgtggtggctctaaacgcacgacagtagatcctgaaggtggtggacacatataaaaagtcgatggtccgttagcctctatttgggacgcacggagagcttccctcatatccgacatatcggtgggatctgtagttggttttggtggtgtaccccctacctcaggggtagctacagtccgccggggttgttccgatgtgcttccagattgaccctctactggacggggaccgactaagaatagtacgatccagataaatgaaacatatctagctagtctggttaatcccgacgatccgttaccatgtctggagccagtcgctgcaataccgagtagagaagggaaaaaacatctctgtcgaaaatagccactgtgtccctgccaacgactccctcgtcgccgcttcccaagatcgccacgagtggacatgattcttcgtatgttagataaatgccgggtgggcagatctgggtacctgtattagccggtcgtgtgcgatcagagcgtagcaacttcacctcaccgcagtgaaacacggaggaaaatatggcagatgctagaatgagctcacggatatatttccaagccaaccgctgagttgttgtgacgccagacgtatcatcaccgaaatagtaaaatctttgaaatttactcaccgtccacccaccccgggtggacgttaacataaatcccgccaattcttcttttaaatgtggaagcagaccaacgttctcaacactgaaataaaacgctgtgttggggggttgggcaaagttatgactgtcgtggtcgaatagaggcccgttcaccagctcaaaaaacttatgagttagcgatggaagtaatgatgggtcaatcgagtgacgtaaaagagaagccttcataaaactatgtgaatcaaacaccgattcattatagcggttgtctattaccataccggcacgggcagcagtgtcacaaaatgctcgtctggcgcaaaaaccgttgtataccgacacatacgtgtgtatcaacgtgtccccatatacattaacccgtagagtcttctcgagttcacgccgttgctctctgacacagcgacccaggcttgcaagggaccgtttagatagacgctctgcatagagacggcgtctatgcgcggcatcggaagcggcgcgctccaataacttatcccacgaccgttgaggtagcacattacacccttcgggggagtctatggcccggtccgattgggactgtgtctcgatgtggtcatcgagacactcttcttcattactgctttcttggttatttcctattctttccaataattgttttaaagcctctttatcgctcttatctgggttggtcagttggcggagtagtggtgttgataagtgatgatcataacacgcgcggatgagagcccctaattgatcgtctggtgagattacataactcccgatcatgagattatcgattatatcaaaatccactaaaccctctcccacaacacgttcgaaatgattcatagatcttccaaatagagccaattctgctgtaactatctgaagtctaacgtgttgttcgcgtaaactcaacgtctgtagattgtcagtgaacgagtcgatagtccggcgttctactccgtccaatcccgctgaggccagccagaatttcagctcacttacggcatataagctctttggtgcagacttaaaaacgttatgagtgtcgaggaggccatctgctaggttatcggtaaggtcgttttttgaatgattatcaatagcttttcgcctaccagcctctattttctctatagctgcttgggccgcattccgcttctcgtccaccaacccaggaatgtatgggagatgcttaacgagataatcttcctctattttcaggctcatctgccgtgtaagatgatcacatatacaccctaagagacggcgatgtacagcctcgccttgattggctgtaacacataactcctcaaaacatatagaacatggttgcatgggatcgaataattccggaggaactataaacccaccaccaagagtctttaccataaacatatcaatgtcctccattactcgtagcgccgacccgaacgaggttatgagatggcaataatttagttgctttagaaaattttccaggtcatgtaaaaatcgcatctctgtaataacatttccaccataggttgtcaggtttaatgttttgtgatactcacacggtgcttcgagtttcatcgtctccttaaaaaactcaaggaatgatcgatctataaatgtggtagctacatcaataagtctctccccctcaatggatgcatatctaagtgcgatccacacagcataggttaatggggtctgtatatgtcgcgcctgattccccagtgccggaaacagcttccgtgatagaaatctgattgtaagagcattaagtttaagtgggttcatataacgtcgtagcacctctggatcgcagcgtttcaacaattctatctggaatatgtacgtttgaatctggcctaaaatcacaagcaggcgttggcgcgcggcagcagtaccgatatgcttgatttcacctgaattcatattgaaagaaaatacaagaacacaactcaacatatagattttgtttattaaaatacgtcaaaattacgtaggtttacgtagacatgatttataacatatcgaatgtgagtccagccttcttttctataacattactcatgtcgaatagatcatcttctgggtgaggccgcttaattccaactaagggggcatgacccgtcatattttctccacatgtgccaaaatcaaacgcgaccccgtccccatcgattccaacataattgctagtttgagtttccatttctttaatgacgatttgtgcggtctctatactccacccatcggggtgtgaggagatccgggtattaatatctctcatattaattgccaaaaactcatcatcaactacagccctccaatcttcaagctccaagtgctgaacacgggaacccatggaattgagtattgccgaaaatacagccgtttgaaccatgggtccaccttctgaaataatagttttgataacttctcctagagaatgatctcggacccccccaccacccccaataatctcacaggtatacccaacccgtgggcatgctataagaaatttccgtgtgcgatcaaatgacattagtggacaaacgttttttcctccattcaacccactccaatttccagattggaatacacggttattcccagcactgccattgtatttactgacgcttagacccaatacaaccattgggcgtgttcccatcacaactcgcatcatatttgtcgatgtatatgtaccagtccataattctgggcgtcgtggcacttccgataacacgtcttgtatagctggttctatatcggagacgctgcgtaaagtacctgagatccaggatataactgatgcggggtcttttggacgttttgattgagatagcagtgctgttatcccgttgatgaaaaattgactatgatcgcaatacttcagaattaggtttgctgcataaaattgtatcagctcccccatgttatttggggttagatttataaaatttattgcggcatattcatctgagaatcgcttgaccgttgtgatggtctcaatctcctccttactcaggagacgggccggcatctggttcctctgtaatagagtccaataccattgggggtttggagtctggctcccgggtggatgtccattggggaatagaagcccgtggaattgttttaacaaaaaacccacggcccctgtcaatatatttgaatgacgctcgggtctttgatatgcgctggtcatccctgctatgcgtgaacgggcgagatctgatagatttccaccaatgccggtagcgaacaaaacacggttcttgattctgatctcccttaggacttctatactcacccgcgctatatcaccatcacagtctttaataggaggttctgttgataattttgttagatccggggctacgatgccagaatccgccaaggtaactgtaacggtcttcgatgtaataaatccaccattaagcagatccataaacctttttcttagaactggttggaattgtacccgaaagtttcgaccttcaacggcctgtccgtaaaaaacgcaatgacattgactaagtattaaatcttggataatagccaaaagtgtgcgtcttctcaaaaaagacgtgacggggcataggacgcttgcatatggctcgagtgaaattgacatggtgtgatttgcatcagccaatgcatccctgattttaaaatctctaacttccactagatgtcgcataaattgctcaatcgcctgctccacaccagtctttactgaggttaagacgttccgaaagctagcgtggtcgacgatgatgcgcgagagagcatgggatgcattgggtaacatcagcgagtctctctcgagtaacccttgctgctccatatcaccgagtagtttgtctatagttgccttgtaggtatcattcatgatgctcctaggaggttcgttgtcgtttggtctccgcagtgccccatatgaagcataattcccaagtacatcacaatcgctatagctgctgttcattgtaccaaaaatacccatcggggctcttgttgggatgccaaatttaggtaggcgttgccgtaatcgatagagggtagtatgtacacaaaaatgacgtgtatgtttagtgcagagattacatgggacatccaggtcgaatgtattggttaaatatttcacggcatcgggttctcctctccctacaaacgccccagcatcattgcgttcgaggtaaaataacaatctcgccattatctgggggcagaatccacagatcagggccagataatccacagaaaattcttgtccggaaccattaatggggatcattggttggttcgccatagtgggtaaaacacggccgtccttgtcggtttgtggattaccggccacgtgtggtgctggaattaggtaaaaacggtgataagagggattgggaccatctttaccatccacggccccaccatcgtctacctcggtcatatacaatacagaattagaggaaaatatcatagctcctaccaatccagccagtcttgagacgtaggctccaagggcatccaatcgatctggaccagaaagaccccctaacataggccattgattaatatctggaacggagtctccatacataccagcacctattagagagtctatggataatgcagtgtcagcggccattacagatgccaatcttctttctattccgtttgtagatatatttttcccagaggctccatctcccccgcgcgatgcaccggtgcgactgcctcctttagtagcacttgaaccagaatcaaacagtgtgaaagttacatcctgtggtaatacacctccttcatggccttcgtcaaaggccaaatgtgccgctccccgtgcaagtgcgtcgaggttacgcactcgcagactaactgcagctggacctaggatatacccgtggataagacggcagagacgatcattgaagaatggttgacagtagataaattcatcacatatagacctctgcttggtcttaaacggctcatgtgataccctatttacatccggcatgtataactggatcggataaagcggaatcctgagtacgtcttgagaattgataactacagtactcacgccaccataatgtaaaaatgtattacaggaatatacagcttccttaaaagtttcagtaataaccaaatagagtatggtgttttctggatcaagtctcaaagacttgcatatatcttccgcggtagtctcgatagctccatcgaccggggggcttgtatacccactaaaaccaaagcgctttcttgcggtttcacatgctcgctggagatttggcgccattgaacatgctgggatggtctcccctccatgaaatataaaggcatttggatggtagtgactgggggtaagcttgtaagatatcccatgtccactgagaccagaggttttcgatccagacaacacggctacgttatgaataaatccagtctctacagtgagaccttgaatcagaggaacaaccgctaaatcagagtctgaactccgagccgatagaatccccaattctggaatctgcagggaatcacttctgcatgcatagatgtaaccgagtggaccggcttgaagtgagatggtctttggggcagattccattgtaggtattatcctattaagtctcgatgagaagctatctctacaccgtaatttgtacctggtgtaaactaagacgaatgagtggatataccaaatacctgggtatgagttatacttaagtgtgtgaatgtccccgcccacacaggtcacatgtaggtagtaggttttgtgtaaaaggatattaatttcccatctagatacacatcgaaacttttttcttatctgggctcgtctataatttacctccgatcaacggcgtcgagctatgagaaatggggaggatcgaaacatgctatctaatcgaggtggttttttcaacccattcctattcagaggggcctgtggagactcctccagactacctaaaaatgaatcgatgcgatataatggatatagacctacgtattatacagatctggctgaatttaagtttatagctccccgctgtcttgatgaaaatctacatcctgaccaacgcaaggggatgcatataggaaccctctccagacaaccgaaggtatatcgcgagggacaagaatataacatatttgattttaatgatagcacagaactaccatggcctagacgcatatatacatgggagcaaaaatccttactcccacccaacttcgattttagatttgatcgttttcatgtatacgatattatcgaaaatattgaaatagctacaggggatgattcttcgcgttttgcaaactttatacgtcccaatggaagtgttataacgttattgggtcttagtgcgtgtggtaaaagggtagccgtgcatgtatatggagtgaatccatatttttacatggataaggatctagtggatagggtgtgtaatatttctaataatcaacaattggtgtcgctcatggtcgagtccattaaaaattcatatataaataatggggtggggggtagacattcgggggatcgtggattgaatatgaaaatttcccaagaatgtttcaagatagacgtggtttatgcgaccaatatttatttttttcactcacgacccaagttatattataaaattactaccggtagcagtcgattatgtggatatatttgtgataattttcatcctgaaattacaaaatatgaagggaacgttgatgtaatatccaggttcatattagataatgctggatttataacctttggatggtataggttacgccctggagcaaacggggagcgggttcaattacgatcaatcgataaatacgtgacgtcaagtgatgttgagataaattgtacagttgacaatttagaggcattaccaaacgatgatacatggccagactacaaattactatgttttgatatagagtgtaaagccggaactggtactgaattagcatttcctgtcgctacaaaccaagatgacgttgtcattcaaatttcatgtttgatatattcattggccactcgtaaacacgaactcacgctactattttctctgggttcctgtgatctaccagacagctgtctgctcggaagtactgaaaggggggagccgacgcccatcattttagaattcgatagcgagtttgaaatgcttatagcattcctaacctttataaaacaatactcccctgagtttgctacaggttataatattgtcaatttcgattgggcctatatcatcgagaaactaaaatcggtatacaatattcgcttggatggatatggaaaatttaatcgaggtggattattcaaagtctgggatatgggccgagtaaattttcaaaaacggagcaaggtcaagattaatggtcttgtgacacttgatatgtacaccgtcgccacagagaaacttcgtttatctagctataaattaaacgccgtcgcggaggaggccttaggggagtataaaatagacctcccctataaagaaattccacgctattacgcgagcggtgccaggcaacgggggataataggtgaatattgtatccaagattctctactcgtggggaagttgtttttcaaatatctcccacatctggagctatctgctatagcgaaattagcaaatataactctgacacgtgccatcttcgacgggcaacaaatccgggtatacacgtgtctcctaaaattagccagagacagaaatttcatcctacctgataaccgcaaccgtttctgtggagacccgaatataccctcagaggacatagaaaatataaacacatgtgacagcatggaaggtgattacacagaggaattcgatcagcagtcatctaccgtagccgagtgtgtggctcctaccccatctcaagcgacatcgcgttctgttggatatcaaggggccaaggttctagacccaatctccggatttcatatagacccagtagttgtattggattttgccagcctctacccaagtattatccaggcccataatctatgtttcacgactcttaccacagaacgtcaatctctcgaaaccctccgaccaggaatagatttttcggaatttgatgtcggtggacataaattatattttgtacattcgcatgttcgagaaagcctgcttggtgttttacttcgcgattggcttgccatgagaaaggccatccgggcccgtattcccgatagcacatcagacgaggcgattttactggataaacaacaagctgccataaaggtagtatgtaactccgtatatggatttaccggggtggctaatggtcttctcccatgtctacagatagccgccactgtaactacgataggaagagatatgctactcaatactaaacattacgtggagtctagatgggcaactagagaaggagtcgaaagtgattttccagaagctatgaccgttactattccggataaaccctataatgtacaggtaatctacggtgatacagattccgtatttattaagtttagaggcttcacgtatgatggagtaacgagattaggtgatctcatggccaagcaaatttcacaagccctgttcagagctccaatcaaactcgaatgtgaaaaaactttctgtaaattactattgataacaaagaagaaatacatcggtgtgatcaacggagggaaaatgcttatgaagggtgtcgaccttgttcgtaagaataactgtaattttatcaatacatacgctcgccgattagtggatatattattatataatgatacagtttccgaggcagccgcagaaatcgcaagcgtgcatcccagagaatggatgggacgtgcgttgcctgccaacttttgggaatttggaagggtactggctgaagcatatactcatataaccgctccagatctcgatgtgaacgattttgttatgaccgcggaactcagccgtccacctgatgcatatatcaataaaaggattgcccatctcacggtgtactataaattggttatgcgtaacgaggaagtgcctagtgtgaaagaacgtataccatatgtaattatatcaccagcgcaggtgtcggacagtgacctgcttggagtcgatgggttgcgcgggactataatcaataaggccctcaacaataccaaaaaaagaaaattgctcgtgtcggatcttgctgaagacccatcctatgtaatagctaatggcatacacctgaatacagaatattacttttcacatcttttaagtacgattagcgttacatttaaagctttgtttggaaatgatgtcaaaacaactgaaatacttctcaagaggtttattccggaaacatcaatagtagacagacggatacaaaccaagttatctgaatatgggtttgaggcattgatcgaaactagcggggtggaggaaactcgtcgaatattgaataaagttttctgtattccaagagcaactctccatcaaagctaaggtccctcatcttacaataaatatcagttgcactcacggttggtatctccatatccgtctgtctttcaccatctcgacgaaccaccaatataaacatagtttgccacacatgtgcgataatcctatacccgtgacaagcagataatagtctgtccaataaacgatggtgaaggtgtacggtctttcctggaaataccacatacatcatataccccgaaaattcaaggtcgcgatagaatagaactttaaagttttgtggtcccccatctcttagaacatgatatacaaaaaacaattctggttgactgagaacatcttctaaaaatctctccgctgaaccctgtatttgtgtggatgtactaccaccgatgtggttagcgagggagatgattgatgccaaaaatactctatattcgtatatgttatttaattgttggacataggctaaaactaaagccgcccggctagtgtgatatagacgaggttctccggagacggcacatgttggacaatagccacctatccctagatgatatcccatggcagatagcgatacacagttatcggcaactgtctgattgagatcgaacggtaatgatattggcgtagtttttactataggaacaaccaagtctcgtacggccgcgagttcatctgatggagaaaccgatacataatcaaaatatagactataacgttctctttctttgaaacaggtaatcattggagttttttctagaagacgtgcctgttttgttgtggaatcccccctagttcttcgtactcctgtcgtggaatggagatccttgcgagtatttttacgtgaatgatgaaaggatttcctatggtgttgacgaacgttcaaaagagaacacaatgcagatcgcacttcagcgtcgcgctcgtgcgaactccgatcggaccgtttctcagacataattgtataacgtatgctccagagaaaccaaagagatactatggcggcggaaaaatgctccggcgtccttaataaaggctgccccggccgttggcttgggaggaaagacctgataggtcttaaaggtatacgccaatgtcaatagacccacgcatacacgcccctcaaatctattatcactcgccaactgcgctttatataattctagatcctccttctccgatgaagttggatggtcaaatagaacacgagggtttgtttgaagctcggagattatacacattgggtcgcaaaagaagtgtttatatatagcttcagttcccaccgcggaaagtaaagttataaaccgcccaccatccccactaaaggaatacgcttcaccactggtgacttcatccttccatgcttccaaaataggaggtatacaatcaaacagtcctgcattatttgctgaatatgtgacgacctccctcttgaatcgacgtaatgccacccaataatctctaatgatcaggagattgcataagatacattctgagacagttctccccctaacctgggcgtgttttagtgtcgttaagagtattggtccaaattccaaatttggagaggcatccacaccagtaaatttagtaaatctaggggcggtatcgcttactaacttctccctgtgtattgaccagtaattctccacacataccctacgcattctcgccgcttgagttatacgatcatccacctcccaggtggttataccacttaacaacaaaagaacgaggtccgcataatctcgttcattattccggggtcggtaattttgagcctctttcaataatagttcctcatattcattatcttgggatttggctcgctcacaacacgtcaggcacccgcgtcgtctccgttcattattggaacagtcgaggagtttcgcacaatccttccaactaaccaaggcggttgttagtgattggggtggtgcatttatgttacaattggctggttgagttccatcgctcacacggtgggtggttgaagtgttttggatacgcgtggtacataacatggctctcgtcgaagactggattagaaatgaatagtttgaaaactgaaccttgtcgtgctccaatctacccgcctttagattttgtagtgataacgaatttggtgtatttatctgggacatgcccccgtcggtccgaaagcagccatgtagaaaaaaatgtcgttgaatatctatcggtgtgacggttcgcctagcgaacgcccccaccaaagtaacattttcatgtcttagatagtgtttatccattagatatacaaattcaaaggcggccacgaacgtggaggtggcacagagtggtatggatagacatttggaacatagcatagcataatcagatatccacttgggatccaatccaaacttccgcctataaagatcaattattcggcacacgatacacggtctatccaatgcgagtgaatcagcaatcacctccacaaactgtgacacgtcgttgggtagtggttgagattcacctataggatttccatgatttaacgtgtcatcttccgatgaaggggatactgggatctgtattaaatgagcagcaaataacagctcgttacagagagtgtcgtttgtggtgagtaacctcgggttgaatgctgcataaggggcctcgaacgcgtcgttcctccaggtactctcgtcatccggtattacaatagggaacatgtccaggcctatgtctcaaaagaccgtatctagcaacgatcagaccaggaggcttaaagataggatcccttctatcgaccttaatgctttaaatctcgacagtctcgagtctatttatctaaatggttttgatgacatagatatctggtttgaggatttaaccccaccagaacttgaactctttttacctaccacggatctaaagctaaactatttatcatatacccagcgactcgcatctaccatcatacacgatttgaaatcctctaatgggtgtgtccatggtacagctctggccacgcaaaaatcacgtttctcggctgttataaaccgttttctcgatctacatcaaattatacgcgacgtgtgatttgatggcaggtggggtggtgtaaggcccccccatgctctatatataccaagtcgcttgtcatcctatgttttaatttctacattaggacttggaagtccacatcataactctcattctctactaattatagtgatcggccaacaaatcgcgtgatatatttatggggaagtcacaactactgcagcgtatacgcctagtggtgtcgggtaatcttcgttacggcgatggagatatacatctatccaacccaaaaaatccacccgctcgatgtatattccagttcagtggtcaagatgataatgacgtgacatttcctgtggaatatgtattacgtcttatgagtgattgggcggatgtacactgtgacccatatatcgagattcaaaacaccggtgtgtcagttttatttaaaggtttcttttttaaaccggccatggccgcggagagtccgatcaactccgaaaatactaatgtaatcctaggatctactcagagtactgggatctcactttctacgttggatgtggcaaaacgtgagagtggaatggacctgcgtccactgcgggtaatgatgcaattacattgtttcgtacgcatgccaagggttcaactctcttttaggttcatgggcccggaggacgtctcgcggagccaacgcttatttgagaggatttcaaacttcaatctcacacgtagacatcgtaacataaaatctactaatgtatcgactggaataaatgacggtcgtataggatctatagatgctccacaaccgctgcagagatccgcggatccatcacggcttttagcaaacgcagccgatatcatctcaaatataatttctcctgtaagacctagttctatgaaaacatcactagtatcctccgctagaccaatcgttttcggtagtatatcatgttttgtattcgtgatagccatcacccttggaattttgttgtctgggagaaaccaataaaaagcccatgtatatatctgtaaattgccagtgtttattaacctctcacagcgtccgtataacccctcggaaccacaactcccccatcgtgtataggcgataaccagccatcgtttagttaataatggcaagtgagtttaaccccgctgatctacagaccatcaccgctgcaaatctaaacgggttactcccggttgatataattcaaagtcttaatagggcggggcgtccagagggcgcatcgcaagcggccatgttagatgcaaaaaggactctattcgtcggtacagcattatcaatggtcgctctacgtcaacgccatgcccaacatacaatcaaccgcctccccatgttcgctgagtatgatgggtcatattgggcacgacctactattggtctgaaacgaacattctctccaagactaatacaggtcgagaccgtcgaggattgaacccttcaaaaacttcagatataaaattaataaaacaggatttcgaatattttgttattatgtatttaatatatcaaaaatgtttttggggtggtcatccgagtataagacgaatcttgtgtaagtcagttaataggcagtgttttttatccgtaagaagctttctcgtgcgacgtaggcgttcggcgatcattcgacacgcagttgtcaggagcgctaggactcctatacctgtgtttctgaaatatcgcctgccttgaagtagatcgtccgctgctaccactctcgtgaaggttgtgtcagcagaatattggggcgactccccactgtcagttccgatattatcaatggtgacaggtagtgtgggactcgtccctccgctgtccacattcacattcacatatcccgaactgtcatcaatcaatgtggttgaatctgatgtgggtaatgaagtctgattcagtgataagttctctaaattatacgttaaccggggactcttttccattgtatcaatctcggatgataggttatccatcgactcccaagaattaaccagggccggggccctggtgataattagtgtatctttaataggcggttccacagtatctaatactgtccatggatcgctattatatttattaacccttttagtcttactctttaaaggtctcttgggtggattgatgatttggggtgctcgaggagataatctcgaaatggaaacaaggtcttgtgttctgggactatgcactatattcacgctcatcgtttcgcctttatccccaataccgctataaacatgcgcggtttcctcagatgggtcttcttttgaagaaacgccggtagagatggtaacgagtgctgctctataaatcgtagcaagccgctcaaattcactttgattggcttcggttgtcacatttgatgtaactgttataggtctcagacgaccgctagtgatattcttcttaggcgatggatcaccttttttcttatgcgatccccgaatcgccttctcaccgggtgtctttttggtcgatatatccctctgagtggcaagtggttcggttgttctaaaatattcggatatgttatcattgggtttctcctggcgatctccgtcagggccaaacgacgagcgcgtttcggagtgtagaacgatggggggtacggatatggccatcgaagtgagcccggaaggcgtgtatcgatcattactcgatgtagtcgaaatctggacatgtccatcaggtccgtcgtcaccgtcccagggtatagattctggggtacaaattccatgatcgtgttcatctgtttcgttatcgaatgaatcttgattggtagcagatgtagaaattgtcacagggaggccaggtgcgtcgtgggttaatgagtctccacatgaccgactctgtgttttatctactgtctgggaggtgataattaaaggggtgtgtctgtgtagactcagttcgatgggagatctgagcgcacccgtgtcataatcgtgagtatacttagattccggtgtaaataatatcttagatataggcttcacagtctcccaggtatccgataaagcccccgaggttacatcatctggtgagtgtggaacagtactcgagatttcggcttcagatagataatctgcgttagccacattcgcattcacatctgggagtccatcagataaccagtcttcccagtcgcctataattggggtcgtgttattaacatcattcatcaaagcgtctagtgacgtagagccggacgtgatgctcgagacttctatagagagggttgttacatcatcattttcaaggtttgtattatatgtagaaactaccaaatcagtaagtggatctatgggatcgtaattacgcgtaatatccgctccgataccttcactgtctacaacggattctgctgaaaaacttggcaattgggcatttggagctgcatatggaaatggaatttcatccgagaatttataggtaggggggcctgcataaaaacttccacattccgctaaatttggtagattctcaccacttatcagttttgtgaagaatggatcggagacgtccctatttggtaatagatcgtcatcggtctcaataaaaggaatctctgcaattaattcgtctgccgcgagtgagattataggtggttttgggggtaccgggtttgaccaaaatatatgtgttatattcgatcccacatccaatgttatcagacatggtctagcatcagcgagtggtttactggccactaaagcagataatctatctaattggccggacagacacacattttctatggtattgggatccaatgctaagagtctatttccccaagaactcagatcagatgacacggccatccctatatcatgatcagtcccccctggtctagagtcgaagagcttcatacacatggtcaaccctgatgttgttgaaaacacatctgcattctccacggccaccactaggggtgcacccaataagatggcgcatgtaactatatccattgctgtcacattcttaatcgtgccaggtggcgatcccgttaatgtgaatgttgtaagcttgccgactggggtatataggttatctctctcctgatcggtcgtcgcgaagactacagccgggtctatcgttgagactgtactttgtatattagcaactgtatctaggcggctatctatcatatcgtcataggtcatattttgttgatccaatacatctggccttagcgccgtccagagggatgataataccttgaccggtatacacatacgtgccatcacagtaaatgttttaagtaggtctccagggatgagtcttttaacatccccggcgatggttctacttttttcatcgggggaacttagccagacatccaacaagccagtaagagaagtcttgatagctttccagtcagaggctcttaaagcaaagagtgacccatgtgatatatcttctggatgtacatccactggtaatgggcgccagggatgtgatgatccatgttcttgtttagggttaacttcaatagtgttgattagaatcctggatgtcaaagcttcggttatagccagagttagggtttttgccatgtattcgtgttggcggattagatccaatctgagaaaatttaccaaatgcaacggaaatgtcatcacaaggcttatcatcatatccgagagggtgaatgttatattatcgcgactaccagattttgccggtcgatctacattagtattttttatcacatcattggtaagcacctcaccggtttctgtcgaaaaacgtatatccttccagaccctgccatattgccgtgtaagcgtagtggccaatattgccgaatatgtctcggcggccatattcgacatctgagaagagtcccaatcgggacgccggtgacgaatgaatctagaaaatatcgagaatccctcgcgatgggcagtctcaaacccgagtcgtaactgaataacctcgctcattacacgcataccaactatagatagagattccgataatggctcggaaatcatgctcgggcgtattagtttgcccgattgcccacataacatgatagccagtttctcaccaacagcatggtaacaaagtttatattctattggagttccattggtatccagaaaagtctttgggaacagtatgtcgatatgggaagtggcctccctgagagtgtatatcacgtcacgatcgcccatgatagcagcgacccgtccacaaaggcgcctatgttcatcctctaccaggttccctttcttaaattggttacttcctatagtaattttacatggagtaacagtagatagtttactatattcaccataaagggtaatccttaataaaagcagttctctgtatacagcgtattttttttgaacccatttaggtagggactttgcaatacgatcaaattctcctagaaatacatctagggttgctgggtccacctctggaggaattcgcctaagcagcgcctcaaattttgagatgttagcctccgtggcatgaaaggtatcatagaatttttccagggctgagttcctctctgcatatctagcatctaaggatccagtaagttttgctggaaggcgacggtaattttcattcgtacgtaatcccaagacgatccccattgctgcttgtagggagcgcgcgcggacacccgtggaggtgaatgcctcactggaattggtaatatgagggagttctcgttgaaaattgtcccatgcctcattccaggcagcttccgccgcaattagctgtgatcccaaatcattcaaatcatcacggatagataaaagtttatcaattcgttctccatatggctccattggccctttaccatctatttttgtggtcaatggatgggagtctatgatagaccgactttgttgtaaccactccacagccttaacgtctaattcacgggcgtcctccaccgtttgtaggaaaagggccatctgggtgactagatcctccgcagaagaagctttgtcgatgctattggctattgagggtggtggggggtttagacgcaatagattttttaggttggctaagtgggtactcgcgtctctatcttgagcctgggccgcatctacaacctctcgtagaattttgatggcccgtccggtcgcttccttttttgcgagactcgtctcggcaaatgcatccttggcgcgggtgagatcattttttactaacattaccattatagctgcgtatgcagaccccgtgaaacgggattgatcgaatgtttctaaatattcaatggcatgtctcaaaagtttttcacttggtatatgaagatgaaccccatctgcaataattcttgtggcctcttcaacattccgtacatatgacacttcttccaaagcgtgacctatttcactcgaacgacttcctatcgcctgaagtacatcagcacgtaatttactcaggtggtcccgctcctcctcaaagacaagatatgctttgcgataatattccacatatttaacgagctgggggacttgcattagatcaaaaatcatatgtgttatcagatcattgggtttgagatgaccaccattcacaccggagaaggtaatcataccagacgctatacgcattaaacgtagtaggctatccacatcaataccatgaaacagagttgttagaaccggagcggcaaccgcaaattcgtcccaccatgtaatagcatgtaatagctggatcactggatggatagtaaggtttatgttttctggcgcgtatgggttaaactttaacacctcctctatcacattgagagccctgtgtgtatttgcggcgagggcccgctccgcatgttttcgaaaatcatgtgtattgtacgcatgtccagctgccatgtcacgaagtctgacaagttcgactacgtcgaatgatgattgggtctctatcttatccaaggcggcatgcatgtcgatgcgccatctttcacgtgtcatatcttcttgtctcaccaagcgccttttttcaaggtccgttagccccatctccagatcacatactcgctgcttagctatgtctaccagatgcagtagatcttgacgggctttcatgacgctaggagaatccacaaccatcgattttagaagcaacaggtctccattatgaatcgtcgatgaggccttcgatagaacctgtcgcccctgggttaataccatatcgatatccggggttgtgacatcagcttgggtacttttcaatcgtgtaactgtttctactaaagttcgtgttgtatcaatcaatgtcctcttggtactctctacttgcttaacctctattaatgtttcaaggcgattcttatatttctcggagattacttccaaaaacgtaaattctgtaattatctgtcctcctatactaactgtttgtactgcttcatctaccgcggcgatgaccccccgtatggctgtttctgaatctggggcagcagtagcagatcctatcgctgcggttagttgatccgcacatcgtataaaaaattgatgtgtcgtgtgttgctgtgttggatctaaaatgaattctattacagttgcaaatgccctaccaagacccaatagggaggaggaagtagtcgcgcttggacgttcaatggcctcacgatattggttaaatagtgaccataaatgtgagtggatggtagttagcgttgatttcggtgcatcggctattactagacgaaattcttcaggtgtcttggcatgtgcatcaagcatcaatttctcaatggctccgggtgcggccctcaggccaacaaatcccgccaggggtaacaggagggttttaagttgggagtatatctcatcacgacgagccttgatggtcaatatacgatttctcgccgcctgtgccaatgtttctatctcacgccgctttatctccacctgggccttagactcgcccgtgagacgtcccttggttatatgttcatccagcaccttaacttgccgcataacctcatccgcacctcgaatgactgtgtctatagatgtcagatctgaatccgaggcatctttggcggcctgttcaacagcagtactgagaactcgaaacagctccagctcggtccctagagatgcagctttagaggcccgttcgtttatcatgttaatatctttgattagattgtcaaattctttacgatttatatgtcctccatctagggcatctgatagcaaagctacccaagatacaagtgccccatccgtagatagattctttccagtctgtagtagatctcttaaaagggatgtgttatgcaaggtgttaatacattcaaaaggcgtttgtacaggtgtatttgctgaagcgataatagattggatggttttgtcgatcaacggtatagatgttaccatacgttctaatcccatggtggctgctccagctatttggaaccgacacgcgcctgtcttatccattaaaatggcacgtgcgctatattgcgcccctcgtaaaaaatattcacgtatagcatcattgatcataatcttaaccttgtctacaaccttactcaatctatctgggatcttagatctatctaatgattcggcgagtctagttacctcttcgaccccgctcgcgatcgtcggttgtagaatttctacagcatcgaatctgtcatgggctgtgtgaatagcagattccagcgcatctatgtcacgggccagagctgcgtttgaatgaacggctctggcctctttcgcgcgcgcacgttgaaataatgaatttacacgtttcaccaggggctgggtcctaccgtctatggattttgtgaataatcttgtgctctggtgtgtcagggcatccatgaggcgttcggcgaggtgggtatatagtttaagtgagtttatatccccaacctttaaatcattttctaacttgtcaagttcaagatatatctcctctgtcgatgtctgtatcgccaaggctaccatacgcaatttcgctagaattattattcccatgaaagaccctgtattctgaagcgaactcaaaagtgaatcatccaacgagagattagttagtaccagaccggttgttcgtagaatatctacggactctgtatagacgggtattagatcgagaacccgagtatacaaggattctaatgcacatggtgaatccgatcgggttttggttccattttctatcaagaacccaagtaggcgctccgctataagaactataaaatgttcgaggggatcactatcaccctcatcatgtgttggtgcgttatggatccatactgttgaatctatacattcaattaccagggtatccagatgtgacatctgcacttcaagtccggccatatcagactctaagtcatctatattttggatcagatcatcacttgccgcaccatcaacatctaatattattgggggcttcctgttcctaaagcatgtgattagatctatagggtcggaaggtccacttctggtatttagtgcgtcatcgggttgggagtcctgattagaatgtatagacggatcggtcacagcagagcccgttagagtcgcgggacgccgactccgatatttctttggggccggttttccagaatctgtagatgaatcatcaattgtcacattctcactgcttgtatgtggggtccacgccggccgtcttctttttggtaagctttggcgcttttggcgaggcccaatacccagcgatttggaaaaggggttgacggttgatatatcaggggtggttccattatcccctcctctatctcgggttgttaatgcgtccctccatgattgacccccgtaatgtcggtttgttgtgatggtagattgggtcgactgcccctctccgactaatatggttgtgagtctgggtggattgcgtcctagtggatgggcacttgctaccgatcgctctacataatcttcgtctatgaaataagtctcggagatgccatataaagatgcgacggcggttgtaagttcctcggaatttgttggttgtggtcctgctgtaacaaaaaataccattgctgcagaccattgggactctggatctatttgggcgtaaccggctatataggcatagacatcacttgatgaaagtttgactacaaatgcctgggatatattttcacgaccgtgtggatcgaagatatatgccgtgtcccctgatcggtatattgaaattcctattgcccctattattaccagtgtataggattctacgcgaggtccccatacattatcaaaaaaagaacgtgctgtaatttgagtattcaaaaatccggcacccggtgctgtgtaaaagctacactcgccatatatcctcgaaaatacgcaacatagggccctctccgtatcatcgtaagttatcttattaggtagctccacaatggagcacatagtaggaggatgtcctgggtttttagacgaaagagtccaagtctgggcctctatgagtattccatcgaggctatcggcggtgagtgttgaggtcagaccattggaaaatatcagacgtaggaacgacagagacgaccgtaggcaagatacagatgatccctgccccagctgtgggtcatattggtttcgatagccaaccactactatttcggctcccggtggtaattctcgatccatcacttgatatcaacgataggtctttacacatcaccaccaaaaattagattagcgctctacgtacacacacacccgtatttaataacatggggggacactggaggatctccatgcgggctatggtaggagtgaaatagcaacatcgattatatccccgcctttcaatatttctcatgggcccgaattatatgacataacatagacagacaatgataaaattcattgagtatctttattaaaaccaacattaaagatagaatcaacttcggcgaggaggtcttctggggtaacccaggattttgtaagtagtatttcgtcggcccgcaccacatcgatatcgtttggtgctatcacacctttccgtaatatatccaattcaacggtatcccacgacgctatgtttgacttcccagtgacaggaattccggttgggaccccatcatcaagaacctccgaatattctcccaaaagatttaatacggcatcctcctgcacttggcgcgtgtaagatataatcggggggccatacctttatcatgttgtatcccattccagataccttggaggtctatcaccatatctagaatagcagcttcgggtacgttatcggcggtagaagttacctttgtataacggctacttaaactcctccagcgtcctagaaatgtggtcaggtgtttgagactcgggacttccgatcccaccaaaagcttgccggctctaagtaggagtccggtttgtatcctagttaatttagaatgggctgttagaagattcttgaatgaatccacaaatttagatccttcgatatgcgcgatcatggtctcgcagtcacgaatggcatcacttgtttgtttcatatcactcctgagagtggcaatatacgctctaatgtctgatctagatccttctatgtcacagtaaacccccccgacagcatccactatatcagtcaatctccataccccaaacgccggtgcgctctcaacacccctcgggtatatcgtcagggtagaacgtcccggtgtggccgaaaatcgacaagagaaaaactttttaaggtctagtataccgctagtcatagtcgatattgacgtgattatagaatcacccagttctgctgtatatataattagtcccttgagtctgtgaagaatcgccgatagatacatctcggccaggaatgtactatttttcagggacggtgtttcggggagcattactgacacaccctccgggaccagcatgaccgctttggtaaccacagaatgaagttcgaccattagagatctgagattgtctacatgcgccactgttaatggtttcgaagatgtatcagtgacttgatttaatacattaataatagcagtcagctttgtgcggtacccgtcaacatttgctactacgcctataccataatctcgggcccatgtacataccttaataacatctattagtgttttgcccgaaaatctagaggtgatcagtacgtgtagggcatccaaaactactgttataaatagataaggggtaaaagctggctcagatagaacatgatgtggtactccatcaatagatggatattctatctgtatagccgccaaaagaagatctacttcactaacactgtgttctgacgattctctaccctgaccagttgtataggcatcatattgtacttgaataccccatagatctctcgctttgaaatcgaaccacattaaaagtgattctctcgcagcgttgtgtgtattaccctccacagtaaccactctctgatcaaccaagtttaactccgtctcaaaaaccaccctcgaatttgtgatgtaatctatatatggttttggtgccatacaatggcgtatgatctgttccacactaccattcggtaaaccgagtgaatcgagcaagtctccgtatactagaggtaaatctgcgagagtggtagtataatgtctaatataatctagaccatttccataatgtgtcactaatttaaatatcaaactccccaccatggcaacacagccaattgcctgttgcgcctcatccaagagaggtctttggatagagtgtcggttaatattacttatccgtgccttggaaaatttatcggagatggcacgttctaacatactacaatgaaatgatgtaaatcctgcttgaataagcgcctctatggcttgatgtggattcgtgctcgcctggataacctcccatgtatttgtagtgcattcaaccacataattccatatggtattggatccatcttcataggtgatgtcctttgaatctaatagtctgataattgctgtgacgtggataaggagtgcctctaaatttagatgtcgttcggtaagaatctcggggtccgtgcgtaaacgtacagtcaccgttcctggctgaggattggttatgaaaggattttgatgtgctattgccgaggatattctaacctcataagcagttcgcggtgatagattagcggcctgtaaaggtaccagggctccttcgtcaatctcctgggtgagttccagaaaatctctaacgagcgtagtgtatgtagatgtttgctcgatatcttgaaaatattcgtccactagagccaacatatataacgcacgggtgagtggggttagcgtagaatctctggtcctttgaaccataaccccaaactgtaccaaccaattcgctctgatggctatcacacgtaaaccctcctggacaaaaggcatgccatatagaagttcaaatttcttcgaaacgaggggttctgaaaacttatcaagggatgatataggcggttcgtttaatgcatcagatggggttttatttaacacccctagatatatctccttaaataggactatccattcactaacaagccgatagatgttagatggacccagaaatgaagatactcgtagcccagccgtagttatacaggccatacactcctcgtcgttcatcacatccaataaggttgatatatcagtgagatcattaattgggctctccgtccaatcatgtttaaatctccagaaaattaatcctggcagattttcagccaaaaacgcggtctcggggctccgtgtatgtaaataggcatcacatgctagtttttgtaacttagcccataatctttgcgtatcgcggagttctatagaatccgtagataaaaaaaattcagagatcatagactttatccgcaaaacaccatcctgggttagtctgtcactggctacgcctactagcccttcaagggaggctactagagtcataaggggtccgctattatccatatttgaaaaatcctctacgattaaattaaactctcaatcgctggatatatgaagacatagacggctgtgaaataactgacatatatcccttagaggtgatataagtaacacaccggacccttataccgtggatgtatagtccagtcacgattactatattcgaaaatgttaaaagccgattggcggaatttttcaatcaccggttagctaacacgtgtacaacccacgtccagcacgtataagtatgactttcactcagtgcctaacattaaacgtcgtttcgtgaggcgtggtacgagatcagactgttggtgatatattcgatataaccaataccttatattacgatggatatacgtggaaattcttcgattcaaattgggaacgggcttagaacttatatagtcttaccaccacatggcaaccctcctcggaaaaccggctcggtaaattttaactctacagtgcagcgccataggtcgccatttcacgtgggatatactggtactggtaatacgttggaatggcttcccggatcattacatatcaccccaaattctataattatcaaaaattcaagaggaatccaatttacatctaatgtcatggataccgttatccgagccgaacaaacatgtcctgctactataacctctcaagattcgttaagactcacgcgtcaggtgacacttaccgatttttgtcaccccagtgcagaacgtccgggtcttccaatcctcaccttacgccatccagccgatatagttggagctgctataacatccactccaccggggagggatccacatgagatcgaagaggcttggtccgctttaaccagtaccacaggtggtagggattcaggagaaggcatccgagcatcccttatatcacttaattttttggtggctgcgcgtgtggaagattatccagacagaagcgctgcggatgccgtacgaactcatataattagtaattacaaagaccgtcgcatcgaggcacgcctggacaggcttagagattgtcttgggattatgttacgctgtcgagtgttccctcatcagttttttggtatttttggtgggttactttcctggatatcccaggatcgcctagctagtataactgcgattagtcaaggtgtccaggaagttgggaaaacagatcagaccataatacctagatccacggttcatgtgccagcatgtacctttttagatgccgattgtgaacttagattgaatggcgatggtgtaaagtttgtatatctggtatttgtatataatcagcgcctgggacgtgaaggtgtccgtatccaaattatcactagtaagctcaacagtcaagcattcggtgatatactcagttgtctctttcatagagtatggacagaaaatgcgattagagggactgagggaagcatcgcccccgccacggacccacgagccagatttcctctcgaggaacttacacggacacctaatgcaccaaggtgtaccacgtttcaattacatgacccccaagccaccgcagccctatatcaatggacacctgacctgcgcggccgtgccgataaaaattcctgtctatactctgcctatatgcgcctcggtatcctttcttctgataacacacgacctactcgcaaggtcgaacgctatggaggtgttgaagttcccgtggtatggctcgaaggttttaaatgggatccggaaacctggatagactgctactattaatctaaactgtatggattaacacatcaactctaaatatgtaaattaaactttattttatttacatatgacgtaatgtttgttgctttccccacctgtctgtatatatacatggttgtcgctccattcagccccacatccacatcaggcgactctacattaacataagagagggatttgattccctgtagcatttaattagaccgatcaacaacctgatattctaaccatatggcatcgtcaggctctagtaccccatttatggaaacctgctgcccggaaagtctccagctcgacatacacgactgttataacagactcattggttatgggtattccgggtcgtccatattacaaaacgtatcacgccaagaggctgtggaatatattacaagtgtggtggctaacctcaaaccgcgatgtcgtatcgatgagaggatttacatagaactggggaagatggttcatatcaggattcgtgtaaaaactacaacgttggataaatggctaaattctatggaattggcactcctaccacaagtgatacgggatatctctattaatatgccaggtgtgaaaagggtcttggaacaattcgagatacatgattatgaaccgttatgtgaacttggtctacaatctgcactcaaatatgaagagatgtatttggcaaacctggaaaatggagctttggaatcgatgggccaattcttcctgagattggcagcgaccgccgcaacggcgtccacgcctgagatgagacaggcgcttaacgctaaactaccaacgtggttcagagtattcaaatccttttttttctatctttctaggcagttgatgattccagccacaccaataatgttattcggaggacgtactaagggatctctcgcaagttgttatctgttgagtccgataatgacagacacggaaactgccattaatgccttaattcgagatgtagcgccgattctactacgtagaggggggattggaatatgcctgcagaatttcaacaaaaaactgtctaaggaatgtacaaagggtgtgcttccccttttgaagctactcgattccatgacaatagccatcaacagcgacagcgaacgtcccactggtgtatgtatatacgtagaaccctggcatgccgatatcagatcgatattaaatatgcggggaatgctcgcatcggatgaaaactccagatgtgataatatatttagctgtttatggaccccggacctattcttcgataggtatcaacggcacctaggcggagaggtaaatgtcatttggactctatttgatgatgccgcatcccatctttcgaagctttatggaaaggaatttaatgaggaatatgaacgtctggaggcggctggtatgggtgttgacagcctgcctattcaagagatggcctatcttattgtgagaagtgcaataatgaccgggagtcccttcttaatgttcaaggacgcgtgtaatcgtcactatcacttcgatacacgtggggatgcgctcacaacatcaaacctatgtactgaaatcattcagaaggctacagacactaaacatggcgtttgtaacttgataagtataaatctaccgcaatgtttacgccgcatcgctcatgatcagagcttgtatttcagtatcccattactcattcgcgcagcatataccgctacgatatttgtcaacgcaatgatgcgtgctggaaatttccccacagaagcggccatgcggggtgtagaagaaaatcgctctcttggattgggtatacaggggctccataccacgtttttggccctagagatggatatggtttcttatgaagcccgtcgcttaaaccgccaaattttagagagtctgctcctgggagcaatccacgctagcacatccctatgcaagcttggtatgacaccatttaaaaacttcagagagagtatctatggacgtggtttattaccctttgatgcatacccaaacaccccccttatacattttaaaaaatggcagcaattgagagtagttatgatgaaatacggactttacaattctcaatttgtagcattaatgccaacggtgtcctcgtcccaggtcactgagagtagcgaggggttctctccaatttttactaatctgtttagtaaagtcactagtaccggggagatcttacgaccaaacttacagttgatgcggacgatacgacgcctatttcccagggaatgcgcgcgtctctctgttatatcaaccctggaagctgcccaatggtccatacgtggtgcattcggggatctcggggattatcaccccctagcaaaattcaaaaccgcattcgaatatgatcaacgacagttgatagatatgtgtgcggacagggccccctttgtagatcaaagccagtccatgtctctgtttatctctgaaccggctgatggcaaattacccgcctctaggattatgaacctccttgtacatgcatataaatgtggactgaagaccggtatgtattattgtaagctcaaaaaggctaccaacagtggtgtcttctccggaggcgaactcatttgtactagttgccacctttaaacgattgtatatcatgtctgctaacggatctacccccaataccggtctccactccaataccaaaatgccggtatccatagactctgattgtagcgcctcgcgatacttttacaccctggaatgtccagatataaacatgttgcggtctctcagtatcgcgaataggtggttagaaaccgatttgccaatcggtgatgatataaaggacgttactacactatccgaatcggagttggacttttatcgttttctatttacatttctatctgccgcggacgatctggttaacctgaatctcggcaatctatctgagctcttcacccaaaaagatattttacattattacattgaacaggaatgtatagaggtcgtccattcgcgtgtatatagcgcaatacaactcctcctttttaaatgtgatgcggaggcgcgtacggcctatgtggattctatgattacaaagccggagcttgcgaggaaggttgaatggctccgcacgcgaattggtgaatgtgaatccatagccgagaagtatattctcatgatcttaatagaaggtatcttttttgttgcatccttcgccgctatagcttatctgagaacccacaacatattcatcgtaacttgtcaaaccaacgatcttatcagccgcgatgaggccatacatacaaccgcatcctgctgtatctacaacaactacctcccggctcaaattaaaccatccacggagaggattcactcgttatttcgagaggctgtggaacttgagtgtgagtttatctcaacatgcgctccgcgctgcagtaatctactcaacgtggcggatatttgtaattatgttcggtatagtgcggaccggttgctcggtattatcaaagtggctcctattttcaacgtcccgcctcctcatcccgattttcccttagcctttatggtaattgaaaaacataccaattttttcgagagacatagcactacatacagtggcactgttatcaatgatctataaacaatgtcttaataataaatttaatttaagctaacgtgtatctggattcgtccctttttttcaaaaataactacacatgagtcattggtagcgttaaccggttcttgtttcccggataatccactggttctttatttataaccgccgtcgcgaatcacaatcatcccaataggtaaccagaacaacattatagtcgggcggggttgagatatgcttccagaataagttagttatatgtttggcattggcggcatcccctataaaatgttttagtgtttcgaacaccaggttaaaattagccttctcttggaggatgggaacgcgctttaatattgataagcgaccccttgtctcaggggtcattctagcgataaggtgtttgataaatttccgctcgaggacctcatgttttgtctgtggcgggggtagaaatgagagagtctgtgacgcggttttcattatcggtgggtatcgagatgtgtattttagagtcagactctgctcttctatcatggtcagctgtttagacgatccacgaatttgagatgggctgatcctatatgtgtctgtggacatacattcaatatcccgttcttctgacgatgaagcatcactgctggtatcccgggatatactagtagaggattttagattaattaccttttcttttaccttagttttgctctgatgtcgttgattagatcgtagattttgtacggattttaatataggtgtctggtgtagatctgtatgacagcgaacaaatcgcgccacgaattccgagtatgtcagattaagcgacgcaaggacgtccctacatcgtattgttggtgggaaaagtggaattatatctaatataatatcacaccccattaatataagatcggtatcggttgtatagatctgcgcgaccgtatttgtatgatatagattagcacatacatcatcagcctccatatcactgacatttacatatgggtaccctagatagcggatgaggtttacacataatctataacataaacgtggggtataagctaatgaactccatctcgctgatatacgttcttggatatctactttgcaatctttcgggtttccaccatctggttccgaagtatcttcacacggccctccatgtggaatggaaaattcccccaagcgtccagatccaccctgtaaacacatcgtctgtgtcactatagccttggctccatattttacctgtccatcaccatagatacctctatccgaaacgaagatcggaaagtatgatcgcttctgtaacagtttaagaagcgaaaagaaacactcggcagtcaccgttgcattatccacttgtatatctttctctggaaagaatttctccataagtgtgtacataacattccataaatctatagcgatgggtgtataaataccaggtggtgtagtgatggcatcatgttttaccaaacggttgcagtaggcgtattttaacatcccaaataagcccattctgacactattgattatatctcgtttcctagagcagagtcgtattaattggcgaggtaaacaatcgctccggtgaaggcagttccccaactagattaacccctagttgattatggacattataatgcgctgggtggcggaatcatcgccgcacccaactcaaagcacgaccaaatatgagcgggtctgtggaacctcaaaatcctattggtgatcatgaacaataaaaatgaaaccaaaaatacatggtagataattaatccgtccccccacgctggcgtcatagcgcggcggtgaagcctataaagaatacaggtgcgaggaaattgtcttatttttccctttgtgagttttaatttgtgtgtaaaactagctctctacgatggcatttccaccgtcgagattagaggttggaataaataaagctattaaccatccggcacaagttgtccacgcgggacctcttctcggtggtgccgaatctaacactatcttcggaaacgctgtcctcgaagaagacaagctacgcgaggtaatgaccatattgacaccgatatcgaccagtcttaaaaactcatttttggtttttagtgccgatgggatgttgattcatacgagtgtatgtcacgaacagatatatataccaatatcaaagaatcagttttcatcatatagatggacatatggacagcctgcggtatttttagcgaatatcgacggacgtcgtagcttgttggacgtatttaaaactactgggagaaaaagtgcaaccaagaaggtaattttcgagataactaatgttcatccgggtagaatgttaaaccaagtagtttttaacttagacctcgatggtggactatcttcttcacaacttataaaatcagaatttaataattattgtgttatgttacccacgagagtacccgatttgacgcttgagttttcaaaacctcaactaaacaaaatattggaccttggaaaacgcataaaatctacactagtgtttgaatctacggtgagagaaaccatcaatattatatccgacgtcgggagagtaacatttaccacgactcatgaatcggctgatggaaatcaagatagccgctgtattttacgcagtctcccaaggtcccacatacgtggtaatgtatcatcaaccgttaatttctctggggttttgaaaccctttcgcctagctttggaatcccccgtaaacttttttcaacttcttcgtaaattgaaacttacacataccgacgtcagcctcaatttcttcttcactccaagtactacacccatgttaagtctgactaccagaaaacccgttggtgtaatgatgtttttcttctgtaccacggaatgtctaggatcatccgagtcaattaaaaccggggatatggatgatccctcgacaaccgaggaggaaagtatccccaggttaaagcggcgagtgttagaagagttccgtgattctgaaggacccagtaaaaaactttgtacttttgtttactcatctccactatgcaacccgaatcctggtacacggggagaaaacccatctgatatttagatgtaaatagccaataccacagatgcttcgcctgtatacttgatccccatttatgttaaaataaagtatttttaatgtaatatattgtgtagtttcgtttattcataaacgctagttagatatctccacccacatttttctggtatttgtaataaaaattgagccaggcgaaagaaagtcagtaagtcgccagccagacttcgagtatggccaccgatgactgtacgtctccaactaatgcagctgggagctcaacaaccaacaataacggtctcgctccagaagggatatcggatataacactaccctcatttactgtgaggaactgctcgggatcgaggactggatgtatcgcatgtgtgtacacggcaactaaagcgttatgttatataggggtccaatctggaattttaacagcatcgatcgctctcatttggctcctaacacgtacaacaacatatgcagccggaatccttatatttataagtctaatatccacaatgaggctctctatggtaaaaactgaacgtatcacaactatatgccgctttactcagaccctctgtgtggccatagcggcagttggatgggcgtgtgatgatttgttacaaccagttggatttacccctcttctactcctatgtctagcaggaatcgctgtatgtgctgcgatcatacatgtgttttacttcatctgcacagccaatggatcggggacacattttcgtatggccatcgttaccatgaccctcggtgcgctgttgggagtatcgagtatcgccgtgactgtgaaatctgaaattctcatcggcctcggtattgcatgctcgattattgtctcccagcgagactttggaatgatacttagagacacatgtcattacagattaggtcgttattcgttaatgcgcacttttacggatttggggcgtggtgctaaccataatccagtcgactttatcgtacccaacatcgaggatgtctacgaggacaagattagcagcgttaaaatttttcgagaacaccccactttgattatggccccgttgatagggctaaccctcacccctccgatatggggttattgtcacatcactaaatatggccatgattttcagacgcccttaacagttgtgatttgtgttatcgttggacattgtttggcattttgcctggaacctttgatggtctaccgaagaatgtatatacctgaggtcctcgtgagtttccatggcatggctgaaataaccgggatagtcttggcactgcttggtgtaaattttggcacgccgctggttttgactctggctatatctgagactctaacttgcctactccatctacgaaaaatcatcctcggcgcgaaacgcctggctgctacctacctatgcaggggtctacacacgggcatgtatgttactgctggaatgtgttatttgtacagtcatatgtaatgtaccactcaacacgatatatttatatcgcggttgtgtctaataactgtttttaaataaagagataagtcgaaatcacaggcagtgaaatgccttaaaaatgggtctcctgtctatgttaggaatctcttattttaagtagtcccgcgagacgatttacatcccgggatcaccaacaatctgcgatgagacgatataggatgggacgcggaatctaccttctctatatctgtctgttatatacatatctccagtttggtacttcgtcgacaaccgcggtcagtattgaaaatagtgataatagtactgcggagatgttatcatctaccagcatgtccgctaccaccccgatatcccagccaacatctccattcactactccaactagaagatctacaaatatagctacaagttcgagtaccacccaggcatcccagccaacatctacattaactactctaactagaagctcgacaactatagctacaagtccgagtaccacccaggcagccacattcataggatcatctaccgattccaataccactttactcaaaacaacaaaaaaaccaaagcgtaaaaagaataagaataacggggccagatttaaattagattgtggatataagggggttatctacagaccgtattttagccctcttcagctaaactgtactctacccacagaacctcatattaccaaccctattgacttcgagatctggtttaaaccacgcaccagatttggggattttcttggggataaagaagacttcgtagggaatcatacccgcaccagcatattactatttagcagccgtaatgggagtgttaattccatggatcttggggacgcgacactcgggatcctacaatctaggataccagattacacattatataatattcccatacaacataccgaagcgatgtcattgggaatcaaatctgtggaatctgccacgtccggtgtttatacatggcgggtctatggtggagatggactaaataaaacagtgctaggacaggtaaatgtatctgtagtggcatatcaccccccgagcgtaaatcttacaccacgcgccagtctatttaataagacctttgaggcggtatgtgcagtggcgaattacttccccccgcgatccacgaaactaacatggtatcttgacgggaagccaatagaaaggcaatacatttcagatacggcaagtgtatggatagatggactcatcaccagaagttctgtgttggctattccgacaactgaaacagattccgagaaaccagatatacgatgtgatttggaatggcatgaaagtcctgtgtcctataagagattcacgaaaagtgtagccccggacgtctattacccacctactgtgtctgttaccttcgctgatacacgggctatatgtgatgttaaatgtgtaccacgggacgggatatccttgatgtggaaaattggtaactaccatctaccaaaagcaatgagtgctgatatactgatcacaggtccgtgtatagaacgtccaggtttggtcaacattcagagtatgtgtgatatatcagaaacggatggacccgtgagttatacctgtcagaccatcggatacccaccaattctaccgggattttacgacacacaagtctacgacgcgtcccctgaaatcgtcagtgaatcaatgttggttagtgtcgttgctgtaatactaggagctgttctcatcacagtctttatctttattacggcattatgtttatattattctcatccccggcgattataactcttatagttcgtataaattacttatcataaccgtgtttcagcggttatatttttataacagttaattgtttactaatagtttacaaagtccatcgtttataaaaaacaagcccagtggtattataatcattcgtatggatataaaccgactccaatccgtgatctttggtaacccgcgacgtaattactctcacacattttaactagtctacgatcacccagatataataaaaagattcgcgtggacatgcaaggtatgaggtctacgtcacagccgttggtcgagataccactggtagatatggaaccacagccatctatacactccaacgagcctaacccaccgaataaaatgttgacgacagctatttcatcgcgtaggagtggaatttttttattttctctgggtatgttttttttcggagttatcctaacagctactattatagtatgtacattcatatttacaataccagtggatatgctccagatgccacgctgccctgaggaaacggtgggtatcaaaaactgttgtatccgaccgattagacgccatgttaaatcacaccaagatctagttgccacatgtgccgaatacatggaacaacccgccaccgcatctgctgttggagctcttataccattattggacatcttcaatggagatgggatatctacaaacgactctctttacgattgtattctctctgatgaaaaaaaatcgtgtaatacatcaatggccgtatgtcaatcaacatatcttccaaatcccctaagtgactttattatgcgcgttaggcagatattttctggaatcctaaatcattaatccatttactaaataaataaacaataccgtttaggtaattaaacatgattctagtgtttattgtcgtatgtacgggcgatgggtggataacaactcgacaatgatcaattatattgattaaccttgtaataaattcgtcggattattggatatatcgagatgatatcacattattttctaatagcgtgtgtttgaaagtccaccctactagtgccatgtgcgcgtttgatcgaagaggcatttaatgttgccagagtttcaattccgtatgtatcgtcgagtaatctagaccgtgggcgaaatctttctactacttcttcaatcccaggcgaggatgatcgtctgcgtgggaggtttttctttacatcaccacattcgttatataattcgggataatcacctttaggtcccccgggcttggaacattgacactttttatgacaaatcggtgtctggtaatgctccgtatattggagctgtgaggtagttccagacgcggacgatcctctggactgcgcggtatcttcaggggaaatacaacgagggtgttggtaatgagtctggtatgcatctcgaggttcatctccattactgagattcgaggaattaaaagtttcagtgagaccgtaggacggattattaatatgatcttccgactcttcgggtcggatgtcatctaaactggtatagggtgttccgtcacagtccgaggaatcaaaacgatcatcgagttgttttgtgcgcgcatccgatctcaagggcgttctatggaagcacccctctaccccgtctggggtattagaagggtggtctccaagacctggggaggatatatcccgaggggttagtggggaggctaagagtgatgccatacccatatatgggtttggggggtgatgacagctggtgggtaggtaacatcatgatgagcgtgtggagtgggtggggatggtagtgggaggctctgccgatctatgtgtgtcatcatctgtgatacacaccgcctctcagttttcgccctctcccgggtggatctccgtcttccacgttctatcgaaccaagaattcctaaatagtgctgtgttggggtcacgtggccgataagacttttggccaatgaatatttaaaccaacaggagacactcttttccattccactccaggttaatgcagacattaccgggataacagcaccaatatagtgattaaatcttccctggagacgcagggctgacctgatataagaatcttccatacctccctcagcccagcacatatatcccccaaatacaagattgataaaatactgaagatggagatgtatctgggtaaccatctccaatgccgctacgattgtactcgtaactccatgtgtatgcgtactccatttcccatagctccacattgaacttatacgtaatagcgtcgctagtgcgtttgccgtctcggctgatagtaaaagggcctcacccgttcccgatgaaataccaggtccgctcgttaaatgtctttggatgaaatcgcgtggagactgggattctcttggtccatgtctaataatcggtttcttatccattgcacataatacccgattatctaggaggtccatgaaccataagagccacctataactgacgcttacataggctagaatttcatgaagtcgaagcatagtgtcggtacttttgctcatcgggtcgtccggtcgcatataatacgcatatttttctagagttatcataacacgcttcagttcgctgattaatgcttcatgattatcattttgcttgctatgcttaaagggttgcttggataaccgtttcccactgaatgttgaccataacaacatgaccttcagagaagagggggtcctggaatctaccgtcatatccgttccactcgatgtcttgagataggtccaataatttttaatcataatctgccaaattccggcacgtgtcatgttcaatccttctatcaccctcctggctgccattatatatccacggcggaattcttccccattgtcctttcccatacacgtagtaattatagtctccggtatagtgttatctcgaattctggatagagcggtaacacgttccatacagaagagtccaggtccagatagattttcagctgactccttcagagcactcaccgcggactctaaaatctctgcgggggttggaagcatacaccccttttctacccgccgctcgagtagattctccgcgactccaacccacgaagacacatccctggatgagttagagtttctaaatgatctatatgcagatgagtgacccggtaatggaagggagttagacatactaaaaaataaaagaacgaatagacttcgcgtactgtcaggttaacatgcgaggtttattgatatgcacagagtacttatacatggttaattccccgcctccctcgtctccttgaagctccacctcgcatacgttccagcccttgccgtcccaactgatctgatgacatctccccaaggatcatccgatgataatgttgaagagtatccgccaggtctacctggatatctcccaatgccgtgttagattgtatagctccctggggtgtcaatccctcgagatcctcagatggcatagccagatttataaccacctgttttccttgtgtgggtggacccggcattggtacgagtcccaattggtggcatagtatatctcttgttttccagaaacctgtcaatgtctgttgttgaagcaacggagctagtatatcagcaatcatggtataccgtggtagcgtcgcctgatgtatgggcactcggcgtcctccatgtatagccgccccggccagggtcaacaatattaagttcaagtgccccccaaggcgttggattagcagtacaatacctagatatgcgctcatggccgtccgttcatgacctaacatagaagcgaccgcataatctctaatcagtgccgttagggcagttctggttgtataatatgctaaaaataatgcttgacctaacgcggtggtgtcacgggctaccgacacctcaggacggcgtaaaaggtcatattcagcatccgcgagtttaacacttcccagtggaacaccggacgcgaatactgtactacgtacgctacggtacgctttgatattcgggtgctcactgagaatgcgagccgtggctacgtacatccactgcaccgccacctctaacataagtgtccccagagataagatagtttccataccatctaattcctccctcgtatatccaggatttttagatgtaccacgagagagaagatagacctctgctgtctggatatgatacccggcatatctgatattaatatttccaggatccccaatggccaatctgagttccgaccaatacaaaagcgcaccaaaggaactgcggtatattgaattacgagatcctctaggtgccccaaaaaattccttcagtggaaaatactgagtgcatggtgttggaagtggtcgtatggccgttcttcctaccatccatgcaagagagcctataggggcttcctgaccttccaactgattataggcattatgaattaatcttcccatcatatgacagtttatacacgatcggatacacgcatccaatagaaaattgattgcggaccgatggtgtaagtcattgatggttaccgctctgataaaattaccgaggcaaagtctgtgttggaggactatcccctgtcgaatagtacgtctccatatcccacgtacctcactggtgggtgtagtatacataccccctggtggatatgcgatatccgcctcaaaccaacaaccctgcgcaaaaccgtgtagagatggcatggtagtccatatctgttcttcgttataattatcgctctctgataggtccctaaacataaacagaacattcattttctcgataaatgcctctcgtgtcaacctatccgctaggacagatggtacacgtaacctacgcaatccttccacatcttcatagaaaatagtttctgggtttaatgtgcgtcgccggcgaccatgatcacgtattactaattcccatgatggagttagtgtaactgtgatgggtaccagcctccgccattctccagtttcggcatttggatcatcttcttcctctaaaaagggtccccgaaattgtaaatctgaaatcccaccccggaccagacggtttagacgatcagatatcccagattctacctctgctccacgaccacctggttgaaattgatccatgtatacctccctcgcggttctctctatcaatcttcgtgatgatgtcccaagtattccactttccaaatatcttaggagagatgtaagacgatccctatcagctggaaggtcttcagagtccacatcctcttcccccccatccccactaggtatcgggtcatcagagtcgattgccaaggaggcttcttcatctccccttagattgggataattatcttcctctccatatccaaccatatcgatttcattatcatcttcttcctcatcatcgcttattccgtccatatcagaagtgtcatcatctgttatattattgtcagcctcttcttgatcatccgaatccccttccgcctcgtattgatctccatatagtaacatgtcaccccatgaggcaaattgccgggggtcctcttcatcatcttcatcgcttgagtcttcacccggtggtggttgaaaatcacgtcttgggcgatgcatcacaaccgggttctcatcttcaccaccgaatacccctcgcataaaatgccaaaaaccagacgttgatggtccatcttccctgggatctgcgctagtcccatactgatctctaatatcttgtacttgctgtccacgttcaggattgccaccttcaggtctcaaaaagggatgactacgacgattagaagatcttcgtgggcgtcgtgtgggtccaccgctttcggggacccaatctgccatattctctccttaaaaatataccctttttgatacggagatgtacaactccgatatatgatgatgtaatacccgataatcttgttatcatatggtgataagacactctcagacagaggcgtatagatttcaccaaacgtgttcaaatgacaacgctcacgattatatgggcgtttccagtaccagccacccgtcttttatccatatgtgctccatgtgggtgtcaccaatttgaaccatgatgacgtaaatcctacacccccttgggatgtttcctcaacccacaccgctaaaaaaaccgttgatggacacttatgttcgtcaaaatagtttttaattttttatgacgagaggggttgattatttataatcatagatgaaacattttattaagctcgtgttcaggaatatcatcatatgataatccctgtgccgagattacataacctttggattctagatgatccagagtcacgtgtgaaggcagttggagggattgagttgtattctggatcttcacacctatcttgggatctggatcccctggaagcacggcacttggtgatggtgggtccatcatggcctcggttgtagttccatagtttataggatgctcctgttttgcatacgaatgctccagatgcgggaatctaggtaatgtcgggtgacgatcggaaaaagcttccatcttcgcgcggatatgttgtgtgagaaatccgattgttcttggtaggctcccagaaaattctggctttaacagtaggggttgaccctcctcttcgatcagacccgctcgtatgagcggtaatcccagagcgtgacgtctatagttaatagtttgtaactctctgaatgtaagcggtctatcctccaacataacgactccgtgattaaataatatcgggtggaaaatacactcaagttgtctcctatggatccacgtataagatagggaaacgaataaactctgtgatactacttgactagcatgtagcttccaagatacttctctgactacagagatatatagatggagatataacacacgtgcgattgttgctacttccttataatatctattgcgaatagtggccctaagcctctgcctcagagactcatccgttacccccttacctttatcccttgggcctcgccgcgcaccggcatatagataacgtaatatagcccgacaatactcagttaataaaattgaatattggacttccctggcccgtagttccgctataaaaaacccctgaatggctgttatataatcctcgagatcatcgtcaaccatgggtacttccggaaatggttctaatccatggacctggagatcacagatcatttcagtcttacgggggttctcaagatccccaataacctcatcgacagatgtcgataataaaacattttctgcatagagctctgtaaatctcggtaaacaagagaataaatcctcgttccatttctccatagctgataataagccaggcccgtctgaaaaattaagctcattacagagtttttcgtaaagacttgccgggctagcaggttttggtggatgtgtgaagattaatcgattattacccaaattagaattatacccaggagtacccaatagcgattcatcgaagtctgcgatgatatcatctggatagtctggttctagaggtacaaccgaatcttcgtaatgggtaaactcacgagccatgatacagggtattcctacatccaaccaccacacgctcaatgaaaaaagagtaggactagagagctttatatatgcaaaacattatactttatttgtaaatataatatgggtgtggaattcaaatttaacacttcagtcactatcaccgtcgaaatcacccagtctaggctttccaatagaacgtcttcttgacttggaggaggatctggtcttaggtctgactacagcatcagatggtactggagtgggcatagttttagacacacacggcgaggcctgtaccgcagaagattgtacattattagctgtgtgtaataggttaagcccctcacagacggtgacacggattacagcctttttcaatagcctctccaactcatcattatttgaggggggttccatgtcccagatcgaggctgcagcatccgccgcatgtctggctgcaaccttcgccatggccccacagaaaatgttcttgttaaacaaatgcgttgcccctctccatggcgtggatgaggttcttggagtcccgctaaatgctagggtttttccagcaattactttattggtacccggcgcggctcttggtttaactggtgtgggtgagggaggtgtagatctcggcgatccacaacccatagaagaacatgccgcgagtttagtatcagagcctgtcttacatcgttccttaggatctgccaccggaagctgcgttggatgtcgcatgaccatggctggtcgtctgaccaaagtagtcttatctgtagaaggtctatcctttttcttatggtgattagttgacgatccacaaatagcatagtcgtagaggggatcctcttctacctcgtaaatatgttctgtattatatgggttgtccagattctcataaacaacttcataatccttgttattcacgtttgatggggcttcatatatatcttcatcaaagaacctagtatcgtacaatgggttctccataataagtcgattttgtacctgtttcaagggtacagttggtttattcttggggactcgcggaggggtttctgtggtacgccccgctggttgtcgacgaccggccatcgcgaagacaacgcttctcgggcgatgaatgtgaataccaatgtccactaatcccgggtcttttatagggtgtccggcattcgacatgatgctgaatcgtttccggggaaaacccccaaatttccgagacgttttagtgtggcatgcgtcgaagttcgctgcttaccatgcgtagacacatccggtaggcgtaacaaatcatagccacacccacggcaatcaaggacatgtaaaataaaacagcggatgcagtgggggtgttgatattcacaccccttgccgaacacgaggcgctccaaaattgctttagccgctccatactctcaaaatcttccgcgttgcggggcatctgaatcgctgtagtagaagacatggcgaccaataggataaccagcactgatgttacggataaacgatccatctccgttttggtagaaaagacttcagaggcgtggagtttattggtcagtgagaatgctctgttggtgacaaataatcaaccaatcgagcttgggttgatgagtagagagccggataatgcggacatcttttataccagaccggtagatattgggcttagattagccacgcctgatggatatgcaattttaattacgcaaacgtgctcctcccaagatcccagtcaagcagtgtcaatcatcaatggggttatcgattctgggtatcgcggaattctcaaagcgctaatctaccacagaccgtgtatcgagactattaaggagtatggtctaaagttacaactacctctactcaagcttagtaaagctacgattacattagcaccatgtcctgctacgattaggcataaacaaggggtcccaatgggtgctcgactgtgtgatttctatgaaatttttaaacaaaaacgggacgaagatgctggttatgatatatccgcacctgaaacgttccagatttatccgggatttaatcactatgtagagatcccagtagttcaccttccgggtgacaacccccccatagcatgtatctttgggaggtcttctcttaacgtgagtggtatcgtagttctccctactgtatggaaacctgagacaaaatgtgggttcttcataaaaaatatgcgtcgcgatccagtaatcattagagctgggcaaaggattgcccagttattacttctggaagaattacctatggaatggctacctacggaaacgaataatcacgatccatttccagaaaccccagaacctgcacctggaacaatcatggctcacgcggatttatggacctttactgaaaacttcgatcgtgatgccccatcgagccttcggggggataaaggatttgggtcaaccggggtataataaaataaacgatatgataaataacataacaacgaaatctgtttttattccatagtcaattctgtttgtcgatgtgatttgcgcttggattttttagtatgtcttatagaatctccattatcaaagatatggtcgttaaccagaaccggttcacgggctacgacgtttggagttatgtccatagtagtaacagatgtgacattcgggtgatttgatattttgcactcctccgatgcggatggttgattagtaactagatgtctgttccccgagcttgcagcaccgataccaaaagtcttctctaatacagctacatcggccatcacaatgttattttcggaactcatgcgtatggcttggtcgactatagtgtcggtggtggtatctacggaccccacagatagatatagatgtacgatggcagctagacaggagtccgagaggcgccgtgcattagctatatgtgattcgactatggaagtgagacttggattatctccagttacttggtgcttggaaagacattctaggtttcgttgacatgcttgataggttctagccaaggcatgcgccttgatcagtttcttggtttgttctgccgatgaaattacatccgcaaccgtcaagggattgggaagtagcgtgtttataacggcaagcgcctccctgaggcggagcgtggtatcattgcctggcccattatgaattggttcataagtttcggcgttatccaccggatttccacacagacctgccaggaaactttttatcatggcgcagcctttcgatgggtcgtatatacaaatattatatgcaacggacgggtgtgcgataacagactccttgatgatactgtcaggacagaagactttccgcgagatatatgttgtcagttatgcctggagtgaagacctaaacgatgtattcaatatgacacctactgggaatctcccaatgatggcagatgaagatttagacttatcagccggtgctggtgatggtgcgcaattttcacaccaccatctagccgccgtccttttagagcatggaacacaccttagtttctgcctattgggtggtttgccattcaccatggaacgtgaggggtgtaaaccacgcacacatcctgtgtttttctgtaggctccaaacacccatggaaagtatagcgctggcacgggctatactttatggagaccccttgtctacgggattgttattagattgtttagatgaggaacgaacttttgcactccacgacgatctgattttggctctggcgataaatacaaaacaattagttccaaggcttggatgcacaactgactcagctcaatatgatccaaaccccgagacctccattaagacgctttctggttacatgaatggccgaagggggttaacttcgatgtacatccaccacgaacaggccgtcttggcaacatttcgtcgtatttatggtattaatcctacccccttttggttcgtggcaaaatttggacctggagaaaagaccctcgtgttagctacgcgctattatctattacaggcaacccaattatcacacaccggagcttgttatgatctgcaggccgttaaagattttatctgtacctacaacgtaaatgttaccgataataccagtggccttgacgtctccaacctagcctcatttgcgacattttcaagattttgttgtcagagtggttatgccagagggtcagcggcggcacacatctgcgattatatacattatcgtataacagcagatatttcggagattagcacactcggggaatatatagccgcagatagacagagtttgcgtatctccgatggagagtttattacctatatctatctagcccatttcgagagctttaatcataggcaggtccgcgatcatctacggtccgtaactctgacagacatagcgctaatcaaagacgttgtatcagcctcatcacttaaacggagtactatagaaaacttcttttctcatatacgagctcagctacatataagtaattatatatcacagtgcgttaacccgcatgtcgtggtcatcaatgagaaactcggacgtgtttatgctgcctccaaaacctatccctctttgacagcacaatctggtgggtacattggtatgtgtgatgtcgcgggggctttcataaagctcatcgatggtgtggagaactcaatagcccgtcggggatggccctataatattacttcacaaacccaagggatatattcacaacatgattacccctccggagatactacaacagtatcgagtagtttatccagaagacttctacatttagtggcttccacgtcgccatcggaacaactcatctcaccagttaatgttttattggggcgtcgtgatctgcacagacctatcccggtatatagaatatccctaccagagcataaacaggcattcgctgtcattcaccaagatgactggagtatcgttacacgccgtattcaatcagttaatagcttaaatcttctcccggaatcactgaaaaacataactgatgaacacacagctgcattaagggatgcttatttgacgcatgtaatgcaccaacgtctagacggttcaacatctggtctggcatcgagcatcccatcatcaaccatgtatattaatagaaatgaaatatttaacggggggttattaattagtaatatcattctggatattgatttttgtctcaaacaacctatcccacttccagtcctacatacagctatgcgtggtttcagagcgggggtgattgaagccctatcacttttattatctgaggctgtaacagactggacttcccatccttgttatttttataaaacctcatgcccccctcaaccgcgacatactatatgtgatatagaatcacaagatattaatgaaatccaggattttatgtgggatgcagatatcatcgaacaagatctcactttaaatgaattacattacgagggtgcctatgatctcaactttaaagttagtgaggatgttgtgaacatggaatccaaagaacaactaggcaagctagataccgcgtatgtatgtggttgtacagaaaaattggggtttcgtgtggcgattccaataccacctccatacctcttttttggatcggataccctaaagggaatggctaggataattcagcaagccgtgatgatagaacgaaccttcgccgagacaatgtacccatatttaaaagacttttcctttattgatactggagtatatggacatggtcatagtttacggttaccattttttagtaaggttggtgatggtgggctatttaacggacttctcctacccttttacgttgtccccataggatgtaaggacttggagaccttcttatatgcccacacagatccgaacaattttcatttccatgctatgccggcgtatgaaacaccaacacacatcctaactggtctaggtggtgaatatctcagcttcttcgaacgaaaggcggccagtcatagaaccgcggctctatccggaaagacctctctcagagcggcgctccagaaactccacatacaacccgattcaccagaatctatagagacctttattacagatattgtcttggataccattaccggctatatcagagtccactttccagactatgatactgactatcaagctattaccgtacgcaccgtagttctaaaacccgagtggattctactccaagttgttcgtaccggttgctattccactcgtatgcaaggtttccaatgtttacgttataaacatatgcgtggatcacgcaattctgctcgcacctttgtagccctcagcgtggacacctataaccgcgtatgcgcgtctctgagtcaacaatgcttcgctaccaaatgtgggaataacaagttttcgactatatttactgttgaggtggcatcaacgtgttgatcgctagccatgtttatcggagggaagccatctcacatcatcatcctcactctgattacaacatatgcagtattcacggtttggtattcgatacatgcaaataccattaataagtgcgtatacgcaacggtccctcttgatggaccgagtataaaaaatttttcttggaccgtctacaacacatcactcatttatgttttatcaagttctgggaactacgctaattacgatggactttctggttatgactacatatgcaggaaggatttggccctggagcctaaaattcgtgccatcgagggtgatgaaaatgtaaagcggagaatgcgtatagtgatggggacacgtaactgccaggcatatctgtggtgtgtacatctcaaattaataactatcacctggatcttttacatcacattcatgttcttgcgtcacgagagacgtatatttggtccatttagggatcgttgtgaatttattcatcctgcgggatatacattgaattatacgacgcgtgttatatcacagaccctccttcattgtcactatacaaagtttgccagactcatgtgtgaggtctccatacagagacaattcctgagtcagcagtttaaaatagatccagtcacatttctctataaacatccatacattgccatcttcatcgcggttgaaaccgcgatgcatctaacggcgaaggtggtcttgatggggaccgtagctctggtatataccccctgcgctcaaacatatcctctgtatgtgaaaatattagcttggatatttgtttctgcggtcttcctagtagatgtaatcgcaattttttacaatagaccacagaagcctaaagaggggtctgaagacgccgtgtcgagatcgcgtggtggtacaaatgggatattcacgaattgctgttcaaccatattggcggggttaatggttaaaatcttttacgtgtctataatcataggggccgtagttgttctgctgcgctacgaacaacgtattcaaattaatctgttcggtagtggtgatacgaaataattatcatatatgggcgggattaatggtcctataaagtctcacaagctacctgggctcgatactgccttggcgtaactgattcactactcggtatcagatctaaaatcctaccacagagagatggcttccgatacaatcgccacagactctcatcacccggaggatgagatgagtattatggggtccgatacggaggaaggagaagatgacagcgtttctatacccgatgagacctccccgacacctccagaacctgagcgtacagtcccattgagtcgcaatgggcgaagatctccttcgtcatcatcgagtcgttatgtatctaccaactatagccaatcatctaaaaggatccgcggtcatatgtggtcttcacctcagcgatccgttgtgagtagagttattcgctcaacttatggaaactcttcaccaactcgattcagatctcgaagaccaccaatccacatgcgccttgggcgaagggttttcgatagaaaccagaaccctcgttttgaccgactgagagccccgtttcaccgtttcgatcatcgacggcacagacgagatctcgttacgtcctccaattccaatagatcgatccggaggagcgagcaaggcggacatgacaagatctgtacaagagccttcaatgaaatatacgaactaaaagatagaataaacgctcaacgcgtaagtgatatgtttggagcggctcaccaaacattgaccctacccataccaaatgggggtttcagaccacctgcagactctgcatggatggcggcgctcgaatttggaaaagatcacttcacccaagaaggtcgtcgtgtaacctgggaaactctgatgttacatggtcacgaactttacagactgttcgagattagagcacatgcgtcagagtctgcacgtgcattgaggagcttggttctacgtggagagggacttctagaggcattagcttctgcagatgagaccatcacgtggtgtaagatgtgcgcggaaaaaaatttacggttgcgcccgcaagaccctatcttagcaacaacgagtgccgtcttggaaaatctgaagcttaagttggcacctgtgatgtgttgttatctgcgctcaagtgggtcaccctctttggaagagctatgtgcgaccccgagattgagcgatgtcacctgtgtacctacctttatgttcatcactctagcgcgactcgctagagccgtagaactcaatgaggagtgtattccatatgatgttattagtggaagcagtcgtgtattggctgaatacacccccggaacctgtttagctggggtgttagaggctatagatctccatcgagggaaatgtgataatacaacatgtcgtttgacctgcggttataccaccaccccgctatatatgcatggcaaatatttttattgtaatagtctgttttgaacctttcgataaattccccatactatatgcttatcaataaacccttatgtaacataatttccgtctgagtctgtttttagtgggtgtgttcctcggacaacatttaaaacatggtttctatatataaatgtttcagagcatccattccgagcttttactcaagatctcccaagacatctattgcttgataatgtctacaagaacccgtccatctgaacaggtagatagaccagaagcggatgatactatggatgattgtaaaatagaacttcttgcgaatataagtcatgtagttaccccccaagctctcgatatcacctggggatcatggctcacgtctaatggtcaggaacggagtatatcgttcatgggaacgcggacatatacgctacgagccaaatgtaacaccatattacgcctacacgcatatctgataggcgtttacgagaagcatcactgtggtcaacagcttcctcttgctgatctccgtatattttgtcgccttctaaataatcctcgtttactaggtgatctgtcatcaaaacatcagatttgttcggccccgtttagcgcggctacccaacatcatgaagataatgaagtacactctactattaacgccattgggtttcattgtcattgtaaaaatccattttcgcttgagtgttggattactgcccatgctgcatccactaaaatattaagtgtggctagaggtatcaccgccgtaaaatgtaaaaagaatatccgcgaataaaaactcattaacaatttatggtgtttttctttattaaaaaagtacagatttgatagaatcgtcaccttgtgcgtgtgggtacccgctgatgcttctggatctgctttttatatctgatggtatcataccttcatgagcatcatcgttcgattctagggtaatcgatcgtgatttgctctgatatcgtctcccaagtctggttgatagcgattgtttctcgtgtaatagtggttgctgctcacctgataaaggtaagtctgtgtgaccggaacctcgaagtgtcggctgttggttagtgtatattcccacttcgatgagatcattgtttgtaggtttgggaaaaattaaaggtcccatagatgctccatataactgtctatccatcactttgaaatctcttttactcaacccatatacggttaaaaactcctgtaatgattttgctaaaaaatgcaaccctgatccctgctcatatcccaacacactcatattctttgataaatagatatgaaattttctattactggctatggcgtatgctctgccgatgaactgtaaagtttttaggtgattcgttggcgtccgtacatacaccaacgtatctggtgtttttcctgtatcagcagagacgcgaaattcatctccatttggaagatctataatacgcccggcgatcccatccagtaggatctgggtcgttgagatggctaactctaacctagattgtcgcgaggaatcagtcgtcgatgcagttcctcccatattatcgactatgttagaaaaggagacgcgtggatatcccgagagataatttaattgaagtgtgtatatatatcgattcacggtttttaaaggcccacggtctgacatcccatacgtaacgttgtgattacattattttcaatactcgtcatcgtcttaggccatacccatttcccaataacctttaaaaccccatacaaattgatatattcatgcctaaactctgtgatcacacatctccgtgtctagatctatagaaatagaagagggtggcattatgaataaacctaatgtccacactaacccatggctcgactctacaactatgaaattaccccgatcctctacacagtggtatacagatgaactgggatctccttatctacattcaaccccacagggacgtgtaaaatttgggcatccagacgagaaaatcttaatgatgaaacgggatttctcagatagcactgtggagatcccactaacccctcaaccattaaagtcgtctaatcgtacctggcatcaaccacgtggtatgtctgagataccatcttatatcagcccatctccattttcaggtggaaggacctacggacccaagaattgtatgtgtagacttatgcttctcaatatgggactcactctcctattatttatcgcattacttattgtcatcgtatatcatgggggaaattcatgtaataaaggataagaattttcaataatatattactccatttttttatttctcgcgaaatcatttatgatgatcacaactacagttaatattgatactacaaaaatgacgagaccgatgaaggctgctgtgagccatccatccatagatccgaatcttaggtgggaggtaaatgatgggggtggttcttccctattattcatctcatctgtgatttcaggatcggaattgaaaggtaataatactccatacgctggtggtgccgagagtaaagcatcatgatatgatgggggtgatgttaaatgtggatctagctcgcctgagatgacggtcccgatgaatggttggtggcgtccacaatatatatcgtcgcatagaatactagtgtaagacggtggtcttgatggtaaatcattcagggtttgatctgatgtgtagatctgcgagattctggttgacgtcaagtgtgtctcatcacgagacagttcgtgatgaggggtttgtagggtaacatcacctccgcacattccaagctcagtcagagacgaaactgctcgcataggggccatgggagaacacctctcgcttctcctctgacttccctgacgacgtgggtagcgtgggttatgaaagatacgagccatctggtcgtccctcccagtagtgtgaagatatagcaaatccccgataaaaggttgaggtttagagatctgtttgccccatgtgtggactaatctgtagatcgggtggcctggatatatgggccgaggagggcagttcacatagctggtgatttaaccgttatcgtgggggtgtgtcatgtggtgtgcttaaaaatccgtgttatgttaacaccacatttcatatacgacatttatcggttgtaatcgtccccacagcccctcgtccccacagcccctcgtccccacagcccctcgtccccacagcccctcgtccccacagcccctcgtccccacagcccctcgtccccacagcccctcgtccccacagcccctcgtccccacagcccctcgtccccacagcccctcgtccccacagcccctcgtccccacagccccacatatgagactttaattaaatttaatcagtttaataacttcgtataatgtatgctatacgaagttatgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcgactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacacgccttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctagagtcgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggatcccgggcccgcggtaccgtcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactacgtacgataacttcgtataatgtatgctatacgaagttattatctctggtgatcttgaagagacacatcaactctcgggtccaataaacgtaattcacatccactggaatgttcaacatatccccactttggtgcggggtagtggatcgttatattcacttgtttataattaggatgtcctatgggttgtggtgatgtatctctggggtcggcgtaacagagatccattggggcattcctagcagccgcgatggttatgtagaatgctgcaatgcatattaaaatatgtatacgatttcccatttttcttccaagttgagtgattgaagttcttactcgtagaagggtaagaaggatgaatgctattgtggagtcccaaatactttatactgtcttggtggaaagtcccacccatagataatgatgtaactatgtatcacgaaacattggatagtttagaatttcagcggccgatggtcggaactaacacacacacacaattttatatcctcatgaatatgtccataaatagtacagcattacaagaaacatgcatggatatgtcgcacccctgtgaggagggaggaagagggaaggggaggtgggggagagctctatctatccatctataactatacacacacatttacgtaggtagggtttatatatacttacatataaacacacatttttatatatacattatagtacttatatatatagctatatatacacacatacacatataggacttaattctcaaaaagcattctttaaatttttttttttcaacgtttatttattttggggacagagagagacacagcatgaacgggggaggggcagagagagagggagacacagaatcggaaacaggctccaggctctgagccgtcagcccagagcccgacgcggggctcaaactcacggaccgcgagatcgtgacctggctgaagtcggacgcttaactgactgcgccacccaggcgcccctcaaaaagcattcttaaaataagagatctaaagccaattggcaaaatgttaacatttaatctggtagtgggtacatatgtgtctgtcctatcattgttgtaagttttatttatcttgtaagaattattttgttcaagtttatttatttatttatttatttagagtgtgtgagtgggaaaagggcggggggttatcatgctagctgcatattaataacctgatggatttgatgatgcattttaagttgaaatattagagatttttcttttgtttgtctgattctatgttttctcatgtgtccattctatttccttttctttaagacagaggtttctctaatttattcctaaaaacacacctcttatcttttttattgaagtatgcaggtagctatgcatagtaaaccatatttctcattaattttcaaactttgctttctgactgaagaggtttggagtccatgatgggataggacttcattaaaatcctggtttttattctcaaatttaaattttcaaatgcttttcagctacatctcagttatgtctattaaatctggatacttcttttcacccttaatagtgctgagatatttattatacttatataaagtatatgaaaactggagacactccatggtattgaaaactgggaattctgacccaatatgcatgatgagtcttagatttgacctggcttatttggaaagctcctctgtcagcaaaatttgtctgccaagtgagaacaagaggcctggcattgttttcagaggaattcggcctgttttcagaatgtgaaaacattctcatctagaggctagagattttaatgactaaggtatttgcccaaatataaatgaattgagtgcaatggagaaggaagttgtgtaaatattttagatttgcaacatcctgtatttatctgttaaatttttattgttacttgtcaccctcataaaagcactgcattttattcatttgaaattaaaatcatagtggctgtctctagtaaaaaaagattgagtgagatgggcacctagtaggtcttagagattgagcttacaatatccattcagctctcaaaagccaatcatgggatcagagtcaccttgtcaatgattggtttagaaatagaaaaatgatctattcttggtcaatgagacatgaaggagaccattacgagagggcctcagaaatattttcttgcttttaagtagtaaacatggaggaagacacttcttttctacttgccctggacattgtagtatctgcacacaatggccagaatcactggagcccgcttgctgtcagcctgaggttgaagcacacatagaaatgaaggaagagctaaggtgttggagaagcagaaaccaagttttcacacctcccttctgatgttattctatctcaaggtttgcaatgtgagctattcatttttgttctgtttaagctggggttaattttctgttccttacaaatgtgaaaacatctctgattcaaatagatccaagatcttgatcctcaattaactgtctactattacctgtaactatcaacggagagatgcaaaaagaaaagaagaccaggtagatgtatgctagataatctaaagagaagtatatggcagattccttttgtccagacaaggagcttacataaacagcctcattttcctatacatcagtcgttcttatgtgtgttctaaagacccttggcagtctggtaataccaacagaccactttaagataaatatttttaaattattttttaaaaagcatgtaaccacaaatattatattgcaatagttatcaaaatatgtttaaaaaactaaatttgtggtgtatattacacgtgcttcttcatcaatgtttttttatgaaagacttattgccaagtcacatagctactataatttcaaagtagtaaataaaaatatttcaagatatctgcaaatattaagtgatataaaaaatcaacattttctattttatcatggtcacagtttttgttaatacttctgtggattattaccttcaattcataactgaagaaaatacttaattggagtctaggtgtgggtggagtctaggtgtgggtggagtctaggtgtgggtggagtctaggtgtgggtggagtctaggtgtgggtggagtct

SEQ ID NO: 2 - DNA, 5787 bp, Feline herpesvirus 1
cgagttatcacatcagtaaatattcaaatctactattcatatgtagattagatgtgcaaccggaacaacatggcttccaaccttcccactttcatgtagattagatgtgcaaccggaacaacatggcttccaaccttcccactttcatgtagattagatgtgcaaccggaacaacatggcttccaaccttcccactttcatgtagattagatgtgcaaccggaacaacatggcttccaacctaattaaatatgtagatgataatcagtaaaattcaaattatatcaacactcccagataaaatggaaattagatgtgcattattccaggaagttatgactcacaaattctccaattggtccccatacaacacctgtcccatcactccacccagatatacaggtaatgccaaggtaaaccaatcggagataatctcgtgggtggacaatctcctcccatgcttattgtccaatcccaggtctgtaaggtggagtcactccccatcgtagtagtatatatacctgaaccactgtcatcgtagcatctcgctccctggggagctgatgatcgtactcagcgttcttccattcagctgtacccagtctcagcatcgagatacccggcacccatcttatctggcagtcgctgtactcaccgtctcggagtcgggagccgatccaccatcagacggcctcggaaggggtgaggagatagactaggtggaagggtggtaatggaggtgggctctattttagacgaccctttccaaaatttcataaaatgggatatcactcaaaaatgtatacaggcattatttcagtagttagtgtaaaccggctatctatgtgggtgctagcgaacaaactattaatatggcatttctaattattgctatagaaaatgcacctgagggagtgaaaagtttaagagtttactcgcgtagcaattaacccacaagatcgccgctcaccttcgctcctcccctcgtaccttcgctcctcccctcgtaccttcgctcctcccctcgtaccttcgctcctcccctcgtaccttcgctcctcccctcgtaccttcgctcctcccctcgtacgaattggatagggatgtataggcttgtctggtcttttccaatcacaggcaagtctctataacatccccgcatccaacacgccgggaaatgcgttagacccccacgagtgacacatcgttatatgcgcttgtaaaaaaaatagggaacagtgtaaataatatctagttagagtctttttaacattgtgggatccggaacttaccgcatctgtctccatctttttttataaattttataggccgatatatcaggttgtaccaataggcctatcctcgacatcattttcctgaggatcccgtgactcaaacccccaacgcatataaaaacacttccaagagccctgtgtagtgatgttcagcccacatcacccacgccccgatatggacggtgatgagaggttccctcaaatagacctcttcgacctcatcgaagacttcacggatggtagagaggagtttgaattcttgggggataccaactctgatgtagagtcgaggggacgacctcaagacttactctacgggagtcagggtttcctcgtatcgccaccgggggataaagatgaggtattttttgattttgggaagacccccgatgatgtcaacagggtggggaatcccacatccggagggactaaaactctgacgtctccgcgacccacacgtgaaacaattacctcgggacccaccactaaaccaccaccacccccgacgatacattcggcagcgtctcctgagaccccagtacccgacatcattgagatgttgcgtagggtccaagaggatctcggggtctctgaatcacacgggcgttccactagcgatacggaaacctgttcccatcccagcaacgccctctccatcaccgattcatccgcccatagcccctcatcggtgggtgtctgtgacgtcacagaatgtgattccttgggagcccgcgattattccagggggcgtcgatcccccatctcggtaggatcgatgagtcctggaccacgaaggggtcgacggagtcgaagcggtagcagcagtagtagcagtagcagtagcagttcgggaagcagtagtaccggcagcagaagtagtagttcttcgtcttccagcacctcacggtccccatcgccaccagcaccaccgaatcgagaattgaagccggggaccccgtctccccctcggcctgccggagaaggtagtccaatcacaaatgacgtcattgcctctataaccccccaatcaaacgttgtggggtctgcctctacgacccctggagcgacgctcactgatggagcgacgctcactgatgggcctggggatacaacaccaaagaggcgccgtcgacggggtccgctatcccgccccccaggttccagacttccgcgggccgttcggggattgctccccagggccgagaggatctcggaaaggacctccgtttttgggatacaacgacccccaaccacaggaccgctattgacacccaccggagaggtctggccggactgtgacccaccatgtgaggggcgtgttagatatggggggatcggggactcgagggaaggtctgtgggacgctccggagatcaaaagtgccgcggagcggtatgccgcggaccccggcccggttcccgtctacgttccggagatgggggactcgaaaaagcagtatctggccctcgtgcgtctcatctatgagagtggtagagacgcgatggcgtggctacaaaactcaaagatgtccgggccggatcagatattagcacagttttgtcagagggccatccacggcccccggggacatggatctttcattaccggctcagtggccaatcccctcccccacgtcggggacgccatggcgaccatggaccctctcctggctctcccccacgtcacggcggccgtggccatgagcagacgctacgatcgcacgcaaaaaacctacatgctccagagcctccggcgtgcatactgggacatagcgcgtccctatctcctcggggatcataggacagactgtattaggtcttcaccggggatcgctgacgcaattcccgacgcccccccaaaaacccccacatcctcggatagggtgctcatggtctctgaagacatactagcgttactccagtccacattggcgggggggacgaccaggggaacgggggatcccacccagataccaactcccggaatgggtggagaaaatccgagggatctggcggagacttgtacattggcatgtaccgccgtctttgacagcctcatatttcattgcgggggcgtagaccgcatgccgggattgttcgaacccccctcaatggctcggtctttcccctcggtggccaagtacccctcgccggtctgccccccggatgcactctgtgcccacccggatgtgatagaggcggtgactgcacgcctctatagtttaagagacctggtggaactttctagaattcggggcggtgaggcgccgcccgagagactctcccgggatgatcttcagctcgtcgtacacagtctactcttggtagccaggaccctggcccccctcgtcagatacaacatcgacgtggccaggaagcgcgcacccggatggaacctcacgcgggccgtctttgccatcccttcgcgcgtagcctctctcatggaggaggtcgccggtacccccgacccccatactcccaccccccgggcgaccgcttcgggaaaccgagccgggggagtcacggtggaggagatctcgcctccacggtcgtctaacaaaatgcaacagaggcgggatgttcacaaccccgtagcggggggttgtgatccactccaacagagcccctcgaatttcagacccctttcaccccacctcacccgcccccaatcctctatacggtgcaagcgtaaatcggagccgcttccccggggccacgcatccggcaacctccccatcgatgccgttcggggggaggagatgcgagcccaccccccgacggacgcgccaccacccaccaaaaaacgcgcgggtccccctaggggtccccttggacccatgccacttggggggccctcacccaagggtggttttagacgtatcccaagtggagactgtcatacccccagacccaccgatgcggtgagctccgtttactgccccccggaaattgtcgcggagctggtagatcaccccctctttcccgaagcgtggagaagcgctatcacgtttgacccgggggcactcgctaccatagccgccagatgtacgggacctcgaaggcaggagggtacaggtctcggggggctggtggttagcgccccattgcggcgcgcttccgcctggatgacccagatagcagaccccgaggacgtgaaagtagtggtgttgtataaccccctaccacacgaacatctggcccaccaaaatccaaacagggactctcggcacaccccggcgtggtctgaaactaaggggggtctatcgaccctcttagcggccttgggaaaccgcctcctctcccccgaaagtcactcctgggcagggaattggagtggtcccccggatatccgcagactcaacgcccagggggtgttattactctccaccggggatctagccttcgccggatgcgttgaatatctatgtctgcgtctggggtccgccagacgacggctcatagtatttgataccgtggatcccgaagactggccggcgacgggcccggccgtgagccattaccacctatacatccgcgccacggtcaccccgaccgcttactgcgcggtgcgctggccggccctagaggacctctccagggcagttctgacctcgagcgccatttttggaccggggagtttcgcccgggtggaagcggcctttgagcgcctgtacccaaactgtgacgggttgcgactctgcgcttccggtaatgtgggctacaccgtccgcacggacgcgggggaccgcacacccgttcccatgttaccccgagagtatcgccagcgggtacttcctgcctacgacgggtgtaaggatatggaggctcagagtgcgggcctcaagctcggagagcctgatttcgtagacggtgccgcccacagccatcgtgcctgtaatcgctggggtctaggggcgcccctacgtcccgtgtatctggcaaccgggaggcgtgcgttggcgtctactgaccccacccggattccagcggtcgtcagagccttctgtaggacggccctcctcgcacctgacactgaggctccacccctgattctaactccggcggatccccagtccgctatcatcgagaacataccgggtgtgacgtgggactcgggatttggaaaacaggaaactgtgatacgtatcggggggcgaagcgagactctgtctcctccaaactatcatacgggcgattcacaggctgtggagattgtctcggtcgagaccggtatcaacggcccggggtcgctcgcctctgccgagccggttaaagtggaggttgtctccgacgacgaggacgcggagagtggatggttccataacagcaacccttacatctgacggggtccggtgtcccagacaaggtcacccccctccgcgggttctaaaacttgtaaacacaattgccagcagctgtccatctgtgccaaagaaagaaaataaaacctaccgttcagcacttccataacaattaccagtgtgttgtctgtag

SEQ ID NO: 3 - DNA, 15327 bp, Feline herpesvirus 1
aataaaccagatgttcttatgtatcactttattgagacgtgtcttatttgtggtgtcgtttaccggggtgatattgatatttacaataggtattacatataccaaacttttggatacacttacattcgtatagatatcaatgatgtatggatataaagttaaatcttattaatatgtctatataacccctcccccccatgtatgatggggggaaccaaaattttaaactgaactcaattttcccccaggtttatattgtccatttttgagagggggggttgaaaactacagtttatttgagacataagaaactggtcacgatatactaatttgggggatctgattaacccattaagactgggatatcctaacccccaaattattatcctgttaatatacgatccatatcctatatgccagattcgagttttttagaatcattaagacatggtcttatgtgggggaaaaatcaaccggtaattactggacacataacccgcgttaggcgaaaatatgcgcgtgcacgcggctgtcatcaccgagacctacaggcatatatgggcgtattttttaagaaagaagtgcgaacacgcagtgttcgcacttcgacttcaataatatataatataacttattgggaatttagtgcggacgaggttctagatcattggctggaactcggtggatatgtaacacacccacaaaaatgacgttaaatttgtgggtgtgttatattttctcattgataaataagagcctggagcttggacgcaccactgtcgcccaacaccgtatcactgcgcatctggatccccgggagtggaacgacgatacagcgatttctggggtggtcgcacgtctcaacggtaacccgatcgagtcgtccacgcaccggctgccgccatccataaccagggagtcccatggcgaagggaaggaagcatcgcggtaggccacatacgacccccgccgctgtaaaacccccagaactcaggagcggtcgattgggtcatggacaaccctgtggtctatgcgacggatcgtgtagactgcgaccccgtggggggttgaatatagatcccagtcccatcctcccctcgttaacaccatcgaccggttcatcgaccttcgcatcgcaaacaccaggaggatcaaagatacccgtttcttgcgtagaaactgatgatacgagtccggtgcgcgctcccgcgacacttcccgatggggatgatcatcccgagtatggaagcgtggtcgcgagtcgtgacctcagagcttatttgacacacggacccggggctttttgttccccaccgtggcgatgtgatctaaaacggctgatgaaagatgtcaacatcatgttcagatgtatagccacgtcgtccgtaaatgtcagctcggattcccgcgcgcttcgtcgggcgttgttagacttttatatcatgggttatacgaaccaacgtccgaccagagcctgttgggagaccctcttacagttgtcttcggaacaggctcgtcccctgcgagccaccttacgccacctcaataccactgaaccatgcgatcggcggttccttgagcctcccacacaggtccccccgattttatttggacaggaatgtgatgttagtggtagcgaagactctgaagatggtgagggggtggatatagaaagtagcgattcggattgtggagagacatcgttgtccgaatttagttatgtggggggtgatggggacgagactagtaccagtgactcagattctgggacggatgacgactcggatgaaacggaggatcggagctctagttcgtcagagtcggatagttctgacgttgattatggtacccgtgtctgtggtaaaaaacgcagatgtaatcccgtcagaaggagcgcgcgccacgcggccaaacggaagaggatgtagatatcaaattaaaaaggtatataggaaaccacatgtctcacaaccgcctcctgattcctttccaccttacctaggtgcaatagatttattacccacaccccaacccctgtctctcatgatgagtacatagcccggataatcacggaccatatagttggtgtaataccccccgatatatacattcctacacgacgcccgactcctatgggagtccactagactccagcggacgcgtatatcatgaaccgtcgctgggaagacactaacatcgagtcctttaacatgactggtgtcgcggagatggagatgtaccccctgaggggggactccgcggatcacgcggaaacgctacctcgttctgtcagggctctgttcgacgctctccgggtcgcgtcgtgtgaggccttctgcctcatgcgcctcggtggtcccccacccgcggacatatggcccggggtatatcggcagtatcgtgaggtatttcgaagctactcacgctccatggagggtagcgggggtagcccatttcacgtcgccgatcccatacggcacctggtaggccgttatctcatgggtctgggacccgcaaaaccagaatcccatccggagctacacacccgcctcctctactgcgcctattggtgttgtctgggtcacgcggctacatgcacccactcccacatctatgaggatgcctgtcggcggtttttcgaagagggtttcggggccggggaaatcccccctgcggatgccgtagctcactggaacgcgctatacgagatggtgctcgacgaaccggagctcctggtgaaacacgcggcagccgcggtttatctacagcggagaaactatggggggtgtataccgaatattgagaggtagaagacccccgttttcctcccatctctccccactaatctaaataaactcgccttcataagccatctaaggtctcaaaattctttatcacacgatcgatcccagcaacaagatttatgtactcgtaccctatcccctccccgtggtgatctacgacccaaaacctagggttcgtggagactcccctgtacaatattgtacgcgcctcgctgaaaacccaatctaaaacaatcgtccccatataagacgcggtagaccatgtcataacgtatgctatgtttattgaacgagcccgtcaccggcgcgtcccgggggcatccatcagccgccacagcagcgcacctaataatcccgatagcacgcatagtgtcgccacccctgagacggccagtgccaccagacaccgcctccgtcgccgtctcttggccgtctcgcgtttgcctattctatttatgaacagcgtcgccgtctcgttgtcactctcgctataataacagccggtatcggagtcgtaatttgtcctattggcccctcgtacgtcgtggcttggggactgccggtccgcattcccacgcgggatctcgatggtggcgcatagcggtatagcgaccggggagcccttgtcggatagagtttcttatgtacaaatgggatggtagttgtacaagcggacattaatagttctctctaagtaatatttggatggtattcgataaacccaatatcaataatttaaaaatattgggtttccatgggtcacctggccattaataatatatatttaacataatctcttatagctcaacatttctataaatatgagtccatataaatctgtggccccaaaagattcatacatacgacagataaacccgtttacaaatttcagattatacttagtcgttagagagttcattttgacgttaataccgaggtcatatttatatagaacccatcagaatccacggtctgatgtaactgggatatcgtcaatagattcttacaatgggtcccaatatcaaagatgggcacaacataagatggacaaaattataggcttagaagactattatcacattaggcactcagtgggccaaagtaaaatatgttacatcatcatgctttagaaatcaaatatcgtagggtagatgttatataatcacaccaggagtgtctcatatttgtcaacaaaagtaacaatagacattataaccacacccatatcaacatcatccaataaaagtctgatatagaacccaaaatatcacagacctttccgtcaggtggagacgtagggcaggtttctgttatttaggaagtgtatacgtttggctaatatggatagatttccacgtgttggactttcttgttgtatccctacgagcaaaggtgatattgatactggggataattacaaattacaatcgaccatggaggatgtctctgttacacccaacgaagctcctcgtggtatgctggacgatgatctctactctgatattagcgacggtgatttcgaggatatatgtgatgatactgataccgaaactagtgatagtgatgatgaaagtcttaaacctactaaacaggaagcattggaggccgtccatgcattgaattatacgatcgtgaaaacgcttactccgggatctgaaggtagagtatttgttgctaaacctccagggagtgagacacccgttgtattaaaaattggtatgaaaggggcaacgttgattgaggcacttctactacaaaatatcaaccacgaatctattataaaactattagacactctattttataaggaacttacttgtatagttttaccgcgatataaaagtgatctttatacctatctatcaaacaatatgcgaccattgtctttagcttcagctttcacaattcaaaagcagatactcaagggattgaattatctacatcaacataaaattatccatcgggatatcaaaatagaaaacatcctcatagacgatgaatccagcgtatgtatcggagaccttggagcggcacaatttccagtgaatgccccagatttcctcggtgttgccggtacagtggaaactaattcaccggaagttttagcaaaggatgcctacaattctaaagctgatatatggagtgctgggataatccttttcgagatgatcgcatacccccattctatatttgatgatgaagactccagcgattcgtcaagaagctcccgcaaccatcttcgtaaagttataaccactctaaaggttcacccagaggaattcccaggcgacccgacttctaaattaactatagactttattcattatgcctcatgtgtaagacagccttatacacgatacgattgtatgtcaaaatacgatttgccgctagatggggagtttgtagtgcataagatgttgactttcgatgccaagttccgaccatcggccgctgaaattctaaactatccaatgtttcgtgatacatagttacatcattatctatgggtgggactttccaccaagacagtataaagtatttgggactccacaatagcattcatccttcttacccttctacgagtaagaacttcaatcactcaacttggaagaaaaatgggaaatcgtatacatattttaatatgcattgcagcattctacataaccatcgcggctgctaggaatgccccaatggatctctgttacgccgaccccagagatacatcaccacaacccataggacatcctaattataaacaagtgaatatcaacgatccactacccgcaccaaagtggggatatgttgaacattccagtggatgtgaattacgtttattggacccgagagttgatgtctcttcaagatcaccagagaaggcagacgctacgattgcttggacttttgatctcggaacatgtcaaatacctatcgcgtatagagaatattataactgtactgggaatttaataccctccccagaaacttgcgaagggtattccgcgacctccatacgcttcgaaggtctaaccatctataccttggtaaatataagtctactccttcaaccaggaatattcgattccgggagtttcctgtattcatttatatatggtcaaaatagatacaatggacgtattatagttcatgtagaaaaaaatactgattatccctgcaaaatgtatcatggactcatggctccatttgaccatcatccccaaagccacgttgaaactccgaatgataagaatcatcgtagagggcggggatgttttcccgaattggtggaacctgttctatgggttaatatcagcagtgatcttattggtggtccacctttcgactataatcatgaagatgaggctgatattgagagtgatgagctcccggaggaggatatacataactactcagattgtcgtgcgactaatatgtttgttccgagagagcccctcagtcaagttcttggttctcaaagtctactggttggtagtttaggtttccagataattactcaaccctggcaactgaagcagaatgaaagttatgatggactaagaaatgcctctcttgaaccccgacaccttgactccagtaacgatcgtgatctactagatgaaactgaaatgattggatcgattattacgactccaccaccaacccatccaaaaggtgtcaatgggggtttcctccaagatctaccaattatcgagcctacgaccgaaccatgcttagtacatacaaagatcattgggatcggaacagtagtcgttgtatttttgttatttattctcatatccctatgtgtttatacttgcgttctacgatcccgcatcggtatggtagatcgcgcctatgtgaaacaagtacgatttaattccaatccatcatatcaacagttgacaagatacccccaaccataataaactgattaaatttaattaaagtctcatatgtggggctgtggggacgaggggctgtggggacgaggggctgtggggacgaggggctgtggggacgaggggctgtggggacgaggggctgtggggacgaggggctgtggggacgaggggctgtggggacgaggggctgtggggacgaggggctgtggggacgaggggctgtggggacgaggggctgtggggacgaggggctgtgggacgattacaaccgataaatgtcgtatatgaaatgtggtgttaacataacacggattttttaagcacaccacatgacacacccccacgataacggtttaaatcaccagctatgtgaactgccctccattctactcaaatgagtggtggtgtgtggcatattagaaccatttcgtctaatgatgacacgtctacatttttggtggtgtggaatctttgcggtcctgaaatatctggtatgtacttcaagccttacgaccacgccaaaaacaactacggtttatgtgaagggatttaatatacctccactacgctacaattatactcaagccagaatcgtgccaaaaattccccaggcgatggatccgaagataacagctgaagtacgttatgtaacatcaatggattcatgtgggatggtggcattgatatcagagccggatatagacgctactattcgaaccatacaactatctcaaaaaaaaacatataacgcgactataagttggtttaaggtaacccagggttgtgaataccctatgtttcttatggatatgagactttgtgatcctaaacgggaatttggaatatgtgctttacggtcgccttcatattggttggaacctttaacaaagtatatgttcctaacagacgatgaactgggtttgattatgatggccccggcccaatttaatcaaggacaatatcgaagagttataaccatcgatggttccatgttttatacagattttatggtacaactatctccaacgccatgttggttcgcaaaacccgatagatacgaagagattctacatgaatggtgtcgaaatgttaaaactattggccttgatggagctcgtgattaccactattattgggtaccctataacccacaacctcaccataaagccgtactcttatattggtatcggactcatggccgagaacccccagtaagattccaagaggccattcgatatgatcgtcccgccataccgtctgggagtgaggattcgaaacggtccaacgactctagaggagaatcgagtggacccaattggatagacattgaaaattacactcctaaaaataatgtgcctattataatatctgacgatgacgttcctacagcccctcccaagggcatgaataatcagtcagtagtgatacccgcaatcgtactaagttgtcttataatagcactgattctaggagtgatatattatattttgagggtaaagaggtctcgatcaactgcatatcaacaacttcctataatacatacaactcaccatccttaagtccacattccaatcgagttggtagggaagatatgaagtgggcggtaccaaccatcataaaataggttggagtctggaccaacgttcactcttttgagtgtaaaggaccacagcataatacttaatatgtcgtcgatagccttcatctatatattgatggcgattggaacagtttatgggattgtgtatcgtggagatcatgtaagtcttcatgttgatacaagctccggctttgtaatatatccaacactggagaattttacgatctacggccatctaatctttctcgacgaccaaccattaccagtaaacaattataatggaaccctcgagattatacattacaaccatcactcttcttgctataaaatcgttcaagtaatagaatattcatcatgtccacgtgtacgcaataatgctttccggtcctgtctccacaagacctctatgcaccaatacgatcagctttccataaacacatccgttgaaacggggatgttattgacaataacatctccgaaaatggaagatggtggaatctacgcactgcgggtaagatttaaccataataacaaagctgatgtatttggcctttcggtgtttgtttactcattcgatatgcgtggtcatcgacatcatgcggacgaaaatttgaatggtgaaattcttactactccatcaccgatggaaacatatgttaaagttaacacaccaatatatgatcatatggtgacaactcaaacaacttctaataaatcgatggagtctgaaccatcaaatacatcaatatcatgccatacatttcaaaatgacccgaatgagggtgagactttatatacacacttattgaacatcgctggaaatataacatatgatgacatggttatggatggcaccacattgaaacccagattaatcgatatgggacttaacttgtctgttacatcttcctttaaaaatgaaaccacgcaaaaatggacaccagacagaaaggtgggttttgttatagtaatctcaatcgcagttttactactccttgcggtcatcggatccatcatcaatagtgcaatacgcaagcatataatggtctgtgctgggcggcggatctatataccaaacaacgatgggcgaccatcaacggaaatgacacggtttactcgccagactaaaccatcgaattcgagttccaagtccctactcgatgtccccagatcttcgaattccaccccaaccgatggcgtctctagaagtcagttaaccgtaattaacgaagaaacctaatatatttataaacaaataaaatacttttaaaaatggatatctggtcatgtgtaatgttgacgcatagtgggtggtgacctaagattatattaaaatgtagaaggttttatgcccagttcacagtatctactgtgacctaccccggggtggtaataacaatactatcgaatagccaacaatgggactgcttgttaccatcctcgtgatattattgattgttacttcatcaagttctactattcatcaagtaacgatgacagaaggtgccgcacttttagtcgatggggatgggatcgacccacctttaaacaaaacttcacattttttgcgaggttggacatttctagagactccgaaaggatgtacaggagaggtgagtgttctaaaagtatgtatagatcgtggggtatgtccggatgatatcgttataaataagagatgtggtcacaaaatgcttgaaaccccactagcgttggcggaatttggaatttctaatagttctctcatcagaaccaaagacgtatatttcgtgaataagaccgtgtttccaattctcacacccgaaaaaagtggccttggtattcagggggccactacgaatatatccgggatatataccctgcatgagcacggtgataatggatggagtcatcaatctacattttttgtgaccgtaaaggcaaaacatcccggaccatcgttaaccccagcaccggttcacttaataacaccacatcgccatggggcacatttccacgtaagaaactatcattcgcatgtctacattccgggagataagttcttattagaaatgcacctcaaatcagatatctatgatccagaattttcagcaacaatagactggtattttatggagactgatataaaatgcccagtttttagaatttatgaaacttgtatatttcacccccatgccgcatcctgtctacatccggaagatccctcatgcagttttacatcaccacttcgagcggtatctttaattaatagattttatccaaaatgcgatcacagatatgccgattggacatccagatgtatcaacactccaagtataaatcatatgccatatatcgaacagccggccaataacgtggatctaaagtttatcaatgtacccaccaacgcttctgggttgtacgtattcatacttcgttataatggacatccggaagaatggacctatacactcatatcaacaggagctaaatttttgaatgtgattagggatctgacacgcccacgtcttggtagtcatcaaatagagaccgatattagcacatcttcgcagtcgcctaccacggagacaccacgaaacatacatataacgtgggcgagacgttatctaaaggttatcataggaataatttgcgtagctggtatccttttgattgtaatctctatcacatgttatattcgatttcgtcatatgcgatataaaccatatgaagtgatcaacccattccctgcggtatataccagcattcctagtaacgatcccgacgaactctactttgaacgtatcgcatcgaacgacgaagaatcggcagatgattcttttgatgaatcagatgaggaggagccattgaataatcatcatatttcaacaacccaacatactgatattaatccagaaaaatccggatctgggtacagtgtatggtttcgtgatacagaagatacatcacctcagcccctacacgctcctccagattacagtcgcgtagttaaaagattaaagtctattttaaaatgacccgtcgacgagttctagcaccacgggaattggaagctgctcgtaaactccgtgagattttcaacgcagagtacgtcgcacctacgttcacactagtcgatccgggggatacgtcaaacgcgtatattgtatgtaggaccccggtgaccgaagtagtctcttccatatcaagaggtatcgacaatagaaaatcggtagattcttcatttattcgaatcgtcagtaaattaatcattaggaatgctattcacatgggactatccgtcctatgtgcatttatatcctataataaaccatgataaattttatgtggatattttattaatcctccaaaccgtatgggggagcacttttatagaaatctaccataaagagtatatccgttaaatacccgggtttgattatatgtttgtcagttggtagttgaacttccatcgccatctaagatggaccatcaaacatcacttattaatgccacagatgataattgcttagacacggattctagtataaacttgccaagcatagataaatgcgaaattgatgacaattcaattgcggatgaaactctatccgacaagggctccccggtcgctataccgctatgcgccaccatcgagatcccgcgtgggaatgcggaccggcagtccccaagccacgacgtacgaggggccaataggacaaattacgactccgataccggctgttattatagcgagagtgacaacgagacggcgacgctgttcataaatagaataggcaaacgcgagacggccaagagacggcgacggaggcggtgtctggtggcactggccgtctcaggggtggcgacactatgcgtgctatcgggattattaggtgcgctgctgtggcggctgatggatgcccccgggacgcgccggtgacgggctcgttcaataaacatagcatacgttatgacatggtctaccgcgtcttatatggggacgattgttttagattgggttttcagcgaggcgcgtacaatattgtacaggggagtctccacgaaccctaggttttgggtcgtagatcaccacggggaggggatagggtacgagtacataaatcttgttgctgggatcgatcgtgtgataaagaattttgagaccttagatggcttatgaaggcgagtttatttagattagtggggagagatgggaggaaaacgggggtcttctacctctcaatattcggtatacaccccccatagtttctccgctgtagataaaccgcggctgccgcgtgtttcaccaggagctccggttcgtcgagcaccatctcgtatagcgcgttccagtgagctacggcatccgcaggggggatttccccggccccgaaaccctcttcgaaaaaccgccgacaggcatcctcatagatgtgggagtgggtgcatgtagccgcgtgacccagacaacaccaataggcgcagtagaggaggcgggtgtgtagctccggatgggattctggttttgcgggtcccagacccatgagataacggcctaccaggtgccgtatgggatcggcgacgtgaaatgggctacccccgctaccctccatggagcgtgagtagcttcgaaatacctcacgatactgccgatataccccgggccatatgtccgcgggtgggggaccaccgaggcgcatgaggcagaaggcctcacacgacgcgacccggagagcgtcgaacagagccctgacagaacgaggtagcgtttccgcgtgatccgcggagtcccccctcagggggtacatctccatctccgcgacaccagtcatgttaaaggactcgatgttagtgtcttcccagcgacggttcatgatatacgcgtccgctggagtctagtggactcccataggagtcgggcgtcgtgtaggaatgtatatatcggggggtattacaccaactatatggtccgtgattatccgggctatgtactcatcatgagagacaggggttggggtgtgggtaataaatctattgcacctaggtaaggtggaaaggaatcaggaggcggttgtgagacatgtggtttcctatatacctttttaatttgatatctacatcctcttccgtttggccgcgtggcgcgcgctccttctgacgggattacatctgcgttttttaccacagacacgggtaccataatcaacgtcagaactatccgactctgacgaactagagctccgatcctccgtttcatccgagtcgtcatccgtcccagaatctgagtcactggtactagtctcgtccccatcaccccccacataactaaattcggacaacgatgtctctccacaatccgaatcgctactttctatatccaccccctcaccatcttcagagtcttcgctaccactaacatcacattcctgtccaaataaaatcggggggacctgtgtgggaggctcaaggaaccgccgatcgcatggttcagtggtattgaggtggcgtaaggtggctcgcaggggacgagcctgttccgaagacaactgtaagagggtctcccaacaggctctggtcggacgttggttcgtataacccatgatataaaagtctaacaacgcccgacgaagcgcgcgggaatccgagctgacatttacggacgacgtggctatacatctgaacatgatgttgacatctttcatcagccgttttagatcacatcgccacggtggggaacaaaaagccccgggtccgtgtgtcaaataagctctgaggtcacgactcgcgaccacgcttccatactcgggatgatcatccccatcgggaagtgtcgcgggagcgcgcaccggactcgtatcatcagtttctacgcaagaaacgggtatctttgatcctcctggtgtttgcgatgcgaaggtcgatgaaccggtcgatggtgttaacgaggggaggatgggactgggatctatattcaaccccccacggggtcgcagtctacacgatccgtcgcatagaccacagggttgtccatgacccaatcgaccgctcctgagttctgggggttttacagcggcgggggtcgtatgtggcctaccgcgatgcttccttcccttcgccatgggactccctggttatggatggcggcagccggtgcgtggacgactcgatcgggttaccgttgagacgtgcgaccaccccagaaatcgctgtatcgtcgttccactcccggggatccagatgcgcagtgatacggtgttgggcgacagtggtgcgtccaagctccaggctcttatttatcaatgagaaaatataacacacccacaaatttaacgtcatttttgtgggtgtgttacatatccaccgagttccagccaatgatctagaacctcgtccgcactaaattcccaataagttatattatatattattgaagtcgaagtgcgaacactgcgtgttcgcacttctttcttaaaaaatacgcccatatatgcctgtaggtctcggtgatgacagccgcgtgcacgcgcatattttcgcctaacgcgggttatgtgtccagtaattaccggttgatttttcccccacataagaccatgtcttaatgattctaaaaaactcgaatctggcatataggatatggatcgtatattaacaggataataatttgggggttaggatatcccagtcttaatgggttaatcagatcccccaaattagtatatcgtgaccagtttcttatgtctcaaataaactgtagttttcaaccccccctctcaaaaatggacaatataaacctgggggaaaattgagttcagtttaaaattttggttccccccatcatacatgggggggaggggttatatagacatattaataagatttaactttatatccatacatcattgatatctatacgaatgtaagtgtatccaaaagtttggtatatgtaatacctattgtaaatatcaatatcaccccggtaaacgacaccacaaataagacacgtctcaataaagtgatacataagaacatctggtttatt

SEQ ID NO: 4 - DNA, 147238 bp, Feline herpesvirus 1
tctggcggtttgtgggttggcatattcaaatctggcggtttgtgggttggcatattcaaatctggcggtttgtgggttggcatattcaaatctggcggtttgtgtgttgtctgtagttctggcggtttgtgggttggcatattcaaatctggcggtttgtgggttggcatattcaaatctggcggtttgtgggttggcatattcaaatctggcggtttgtgggttggcatattcaaatctggcggtttgtgggttggcatattcaaatctggcggtttgtgggttggcatattcaaatctggcggtttgtgggttggcatattcaaatctggcctaataaggaaggggaggggcctaataaggaaggggaggggcctaataaggaaggggaggggcctaataaggaaggggaggggcctaataaggaaggggaggggcctaataaggaaggggaggggcctaataaggaaggggaggggcctaataaggaaggggaggggcctaataaggaaggggaggggcctaataaggaaggggaggggcctaataaggaaggggaggccccctagccacgccccccaaaaaaagaccccgccccctttttggaccccctcggaaaagtggctaggggggccgagatctcccgcggcggccggagtgggggaaaaaaattgagttggagctcagtctccatatacggaggtggacagtgtagggggatggtattgaccctcatccaggtcatgtggtgccacccacacctagactccacccacacctagactccacccacacctagactccacccacacctagactccacccacacctagactccacccacacctagactccacccacacctagactccacccacacctagactccacccacacctagactccacccacacctagactccacccacacctagactccacccacacctagactccacccacacctagactccacccacacctagactccacccacacctagactccacccacacctagactccacccacacctagactccacccacacctagactccacccacacctagactccacccacacctagactccacccacacctagactccacccacacctagactccaattaagtattttcttcagttatgaattgaaggtaataatccacagaagtattaacaaaaactgtgaccatgataaaatagaaaatgttgattttttatatcacttaatatttgcagatatcttgaaatatttttatttactactttgaaattatagtagctatgtgacttggcaataagtctttcataaaaaaacattgatgaagaagcacgtgtaatatacaccacaaatttagttttttaaacatattttgataactattgcaatataatatttgtggttacatgctttttaaaaaataatttaaaaatatttatcttaaagtggtctgttggtattaccagactgccaagggtctttagaacacacataagaacgactgatgtataggaaaatgaggctgtttatgtaagctccttgtctggacaaaaggaatctgccatatacttctctttagattatctagcatacatctacctggtcttcttttctttttgcatctctccgttgatagttacaggtaatagtagacagttaattgaggatcaagatcttggatctatttgaatcagagatgttttcacatttgtaaggaacagaaaattaaccccagcttaaacagaacaaaaatgaatagctcacattgcaaaccttgagatagaataacatcagaagggaggtgtgaaaacttggtttctgcttctccaacaccttagctcttccttcatttctatgtgtgcttcaacctcaggctgacagcaagcgggctccagtgattctggccattgtgtgcagatactacaatgtccagggcaagtagaaaagaagtgtcttcctccatgtttactacttaaaagcaagaaaatatttctgaggccctctcgtaatggtctccttcatgtctcattgaccaagaatagatcatttttctatttctaaaccaatcattgacaaggtgactctgatcccatgattggcttttgagagctgaatggatattgtaagctcaatctctaagacctactaggtgcccatctcactcaatctttttttactagagacagccactatgattttaatttcaaatgaataaaatgcagtgcttttatgagggtgacaagtaacaataaaaatttaacagataaatacaggatgttgcaaatctaaaatatttacacaacttccttctccattgcactcaattcatttatatttgggcaaataccttagtcattaaaatctctagcctctagatgagaatgttttcacattctgaaaacaggccgaattcctctgaaaacaatgccaggcctcttgttctcacttggcagacaaattttgctgacagaggagctttccaaataagccaggtcaaatctaagactcatcatgcatattgggtcagaattcccagttttcaataccatggagtgtctccagttttcatatactttatataagtataataaatatctcagcactattaagggtgaaaagaagtatccagatttaatagacataactgagatgtagctgaaaagcatttgaaaatttaaatttgagaataaaaaccaggattttaatgaagtcctatcccatcatggactccaaacctcttcagtcagaaagcaaagtttgaaaattaatgagaaatatggtttactatgcatagctacctgcatacttcaataaaaaagataagaggtgtgtttttaggaataaattagagaaacctctgtcttaaagaaaaggaaatagaatggacacatgagaaaacatagaatcagacaaacaaaagaaaaatctctaatatttcaacttaaaatgcatcatcaaatccatcaggttattaatatgcagctagcatgataaccccccgcccttttcccactcacacactctaaataaataaataaataaataaacttgaacaaaataattcttacaagataaataaaacttacaacaatgataggacagacacatatgtacccactaccagattaaatgttaacattttgccaattggctttagatctcttattttaagaatgctttttgaggggcgcctgggtggcgcagtcagttaagcgtccgacttcagccaggtcacgatctcgcggtccgtgagtttgagccccgcgtcgggctctgggctgacggctcagagcctggagcctgtttccgattctgtgtctccctctctctctgcccctcccccgttcatgctgtgtctctctctgtccccaaaataaataaacgttgaaaaaaaaaaatttaaagaatgctttttgagaattaagtcctatatgtgtatgtgtgtatatatagctatatatataagtactataatgtatatataaaaatgtgtgtttatatgtaagtatatataaaccctacctacgtaaatgtgtgtgtatagttatagatggatagatagagctctcccccacctccccttccctcttcctccctcctcacaggggtgcgacatatccatgcatgtttcttgtaatgctgtactatttatggacatattcatgaggatataaaattgtgtgtgtgtgttagttccgaccatcggccgctgaaattctaaactatccaatgtttcgtgatacatagttacatcattatctatgggtgggactttccaccaagacagtataaagtatttgggactccacaatagcattcatccttcttacccttctacgagtaagaacttcaatcactcaacttggaagaaaaatgggaaatcgtatacatattttaatatgcattgcagcattctacataaccatcgcggctgctaggaatgccccaatggatctctgttacgccgaccccagagatacatcaccacaacccataggacatcctaattataaacaagtgaatataacgatccactaccccgcaccaaagtggggatatgttgaacattccagtggatgtgaattacgtttattggacccgagagttgatgtgtctcttcaagatcaccagagataataacttcgtatagcatacattatacgaagttatcgtacgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacataacttcgtatagcatacattatacgaagttattaaactgattaaatttaattaaagtctcatatgtggggctgtggggacgaggggctgtggggacgaggggctgtggggacgaggggctgtggggacgaggggctgtggggacgaggggctgtggggacgaggggctgtggggacgaggggctgtggggacgaggggctgtggggacgaggggctgtggggacgaggggctgtggggacgaggggctgtggggacgattacaaccgataaatgtcgtatatgaaatgtggtgttaacataacacggatttttaagcacaccacatgacacacccccacgataacggttaaatcaccagctatgtgaactgccctcctcggcccatatatccaggccacccgatctacagattagtccacacatggggcaaacagatctctaaacctcaaccttttatcggggatttgctatatcttcacactactgggagggacgaccagatggctcgtatctttcataacccacgctacccacgtcgtcagggaagtcagaggagaagcgagaggtgttctcccatggcccctatgcgagcagtttcgtctctgactgagcttggaatgtgcggaggtgatgttaccctacaaacccctcatcacgaactgtctcgtgatgagacacacttgacgtcaaccagaatctcgcagatctacacatcagatcaaaccctgaatgatttaccatcaagaccaccgtcttacactagtattctatgcgacgatatatattgtggacgccaccaaccattcatcgggaccgtcatctcaggcgagctagatccacatttaacatcacccccatcatatcatgatgctttactctcggcaccaccagcgtatggagtattattacctttcaattccgatcctgaaatcacagatgagatgaataatagggaagaaccacccccatcatttacctcccacctaagattcggatctatggatggatggctcacagcagccttcatcggtctcgtcatttttgtagtatcaatattaactgtagttgtgatcatcataaatgatttcgcgagaaataaaaaaatggagtaatatattattgaaaattcttatcctttattacatgaatttcccccatgatatacgatgacaataagtaatgcgataaataataggagagtgagtcccatattgagaagcataagtctacacatacaattcttgggtccgtaggtccttccacctgaaaatggagatgggctgatataagatggtatctcagacataccacgtggttgatgccaggtacgattagacgactttaatggttgaggggttagtgggatctccacagtgctatctgagaaatcccgtttcatcattaagattttctcgtctggatgcccaaattttacacgtccctgtggggttgaatgtagataaggagatcccagttcatctgtataccactgtgtagaggatcggggtaatttcatagttgtagagtcgagccatgggttagtgtggacattaggtttattcataatgccaccctcttctatttctatagatctagacacggagatgtgtgatcacagagtttaggcatgaatatatcaatttgtatggggttttaaaggttattgggaaatgggtatggcctaagacgatgacgagtattgaaaataatgtaatcacaacgttacgtatgggatgtcagaccgtgggcctttaaaaaccgtgaatcgatatatatacacacttcaattaaattatctctcgggatatccacgcgtctccttttctaacatagtcgataatatgggaggaactgcatcgacgactgattcctcgcgacaatctaggttagagttagccatctcaacgacccagatcctactggatgggatcgccgggcgtattatagatcttccaaatggagatgaatttcgcgtctctgctgatacaggaaaaacaccagatacgttggtgtatgtacggacgccaacgaatcacctaaaaactttacagttcatcggcagagcatacgccatagccagtaatagaaaatttcatatctatttatcaaagaatatgagtgtgttgggatatgagcagggatcagggttgcattttttagcaaaatcattacaggagtttttaaccgtatatgggttgagtaaaagagatttcaaagtgatggatagacagttatatggagcatctatgggacctttaatttttcccaaacctacaaacaatgatctcatcgaagtgggaatatacactaaccaacagccgacacttcgaggttccggtcacacagacttacctttatcaggtgagcagcaaccactattacacgagaaacaatcgctatcaaccagacttgggagacgatatcagagcaaatcacgatcgattaccctagaatcgaacgatgatgctcatgaaggtatgataccatcagatataaaaagcagatccagaagcatcagcgggtacccacacgcacaaggtgacgattctatcaaatctgtacttttttaataaagaaaaacaccataaattgttaatgagtttttattcgcggatattctttttacattttacggcggtgatacctctagccacacttaatattttagtggatgcagcatgggcagtaatccaacactcaagcgaaaatggatttttacaatgacaatgaaacccaatggcgttaatagtagagtgtacttcattatcttcatgatgttgggtagccgcgctaaacggggccgaacaaatctgatgttttgatgacagatcacctagtaaacgaggattatttagaaggcgacaaaatatacggagatcagcaagaggaagctgttgaccacagtgatgcttctcgtaaacgcctatcagatatgcgtgtaggcgtaatatggtgttacatttggctcgtagcgtatatgtccgcgttcccatgaacgatatactccgttcctgaccattagacgtgagccatgatccccaggtgatatcgagagcttggggggtaactacatgacttatattcgcaagaagttctattttacaatcatccatagtatcatccgcttctggtctatctacctgttcagatggacgggttcttgtagacattatcaagcaatagatgtcttgggagatcttgagtaaaagctcggaatggatgctctgaaacatttatatatagaaaccatgttttaaatgttgtccgaggaacacacccactaaaaacagactcagacggaaattatgttacataagggtttattgataagcatatagtatggggaatttatcgaaaggttcaaaacagactattacaataaaaatatttgccatgcatatatagcggggtggtggtataaccgcaggtcaaacgacatgttgtattatcacatttccctcgatggagatctatagcctctaacaccccagctaaacaggttccgggggtgtattcagccaatacacgactgcttccactaataacatcatatggaatacactcctcattgagttctacggctctagcgagtcgcgctagagtgatgaacataaaggtaggtacacaggtgacatcgctcaatctcggggtcgcacatagctcttccaaagagggtgacccacttgagcgcagataacaacacatcacaggtgccaacttaagcttcagattttccaagacggcactcgttgttgctaagatagggtcttgcgggcgcaaccgtaaatttttttccgcgcacatcttacaccacgtgatggtctcatctgcagaagctaatgcctctagaagtccctctccacgtagaaccaagctcctcaatgcacgtgcagactctgacgcatgtgctctaatctcgaacagtctgtaaagttcgtgaccatgtaacatcagagtttcccaggttacacgacgaccttcttgggtgaagtgatcttttccaaattcgagcgccgccatccatgcagagtctgcaggtggtctgaaacccccatttggtatgggtagggtcaatgtttggtgagccgctccaaacatatcacttacgcgttgagcgtttattctatcttttagttcgtatatttcattgaaggctcttgtacagatcttgtcatgtccgccttgctcgctcctccggatcgatctattggaattggaggacgtaacgagatctcgtctgtgccgtcgatgatcgaaacggtgaaacggggctctcagtcggtcaaaacgagggttctggtttctatcgaaaacccttcgcccaaggcgcatgtggattggtggtcttcgagatctgaatcgagttggtgaagagtttccataagttgagcgaataactctactcacaacggatcgctgaggtgaagaccacatatgaccgcggatccttttagatgattggctatagttggtagatacataacgactcgatgatgacgaaggagatcttcgcccattgcgactcaatgggactgtacgctcaggttctggaggtgtcggggaggtctcatcgggtatagaaacgctgtcatcttctccttcctccgtatcggaccccataatactcatctcatcctccgggtgatgagagtctgtggcgattgtatcggaagccatctctctgtggtaggattttagatctgataccgagtagtgaatcagttacgccaaggcagtatcgagcccaggtagcttgtgagactttataggaccattaatcccgcccatatatgataattatttcgtatcaccactaccgaacagattaatttgaatacgttgttcgtagcgcagcagaacaactacggcccctatgattatagacacgtaaaagattttaaccattaaccccgccaatatggttgaacagcaattcgtgaatatcccatttgtaccaccacgcgatctcgacacggcgtcttcagacccctctttaggcttctgtggtctattgtaaaaaattgcgattacatctactaggaagaccgcagaaacaaatatccaagctaatattttcacatacagaggatatgtttgagcgcagggggtatataccagagctacggtccccatcaagaccaccttcgccgttagatgcatcgcggtttcaaccgcgatgaagatggcaatgtatggatgtttatagagaaatgtgactggatctattttaaactgctgactcaggaattgtctctgtatggagacctcacacatgagtctggcaaactttgtatagtgacaatgaaggagggtctgtgatataacacgcgtcgtataattcaatgtatatcccgcaggatgaataaattcacaacgatccctaaatggaccaaatatacgtctctcgtgacgcaagaacatgaatgtgatgtaaaagatccaggtgatagttattaatttgagatgtacacaccacagatatgcctggcagttacgtgtccccatcactatacgcattctccgctttacattttcatcaccctcgatggcacgaattttaggctccagggccaaatccttcctgcatatgtagtcataaccagaaagtccatcgtaattagcgtagttcccagaacttgataaaacataaatgagtgatgtgttgtagacggtccaagaaaaattttttatactcggtccatcaagagggaccgttgcgtatacgcacttattaatggtatttgcatgtatcgaataccaaaccgtgaatactgcatatgttgtaatcagagtgaggatgatgatgtgagatggcttccctccgataaacatggctagcgatcaacacgttgatgccacctcaacagtaaatatagtcgaaaacttgttattcccacatttggtagcgaagcattgttgactcagagacgcgcatacgcggttataggtgtccacgctgagggctacaaaggtgcgagcagaattgcgtgatccacgcatatgtttataacgtaaacattggaaaccttgcatacgagtggaatagcaaccggtacgaacaacttggagtagaatccactcgggttttagaactacggtgcgtacggtaatagcttgatagtcagtatcatagtctggaaagtggactctgatatagccggtaatggtatccaagacaatatctgtaataaaggtctctatagattctggtgaatcgggttgtatgtggagtttctggagcgccgctctgagagaggtctttccggatagagccgcggttctatgactggccgcctttcgttcgaagaagctgagatattcaccacctagaccagttaggatgtgtgttggtgtttcatacgccggcatagcatggaaatgaaaattgttcggatctgtgtgggcatataagaaggtctccaagtccttacatcctatggggacaacgtaaaagggtaggagaagtccgttaaatagcccaccatcaccaaccttactaaaaaatggtaaccgtaaactatgaccatgtccatatactccagtatcaataaaggaaaagtcttttaaatatgggtacattgtctcggcgaaggttcgttctatcatcacggcttgctgaattatcctagccattccctttagggtatccgatccaaaaaagaggtatggaggtggtattggaatcgccacacgaaaccccaatttttctgtacaaccacatacatacgcggtatctagcttgcctagttgttctttggattccatgttcacaacatcctcactaactttaaagttgagatcataggcaccctcgtaatgtaattcatttaaagtgagatcttgttcgatgatatctgcatcccacataaaatcctggatttcattaatatcttgtgattctatatcacatatagtatgtcgcggttgaggggggcatgaggttttataaaaataacaaggatgggaagtccagtctgttacagcctcagataataaaagtgatagggcttcaatcacccccgctctgaaaccacgcatagctgtatgtaggactggaagtgggataggttgtttgagacaaaaatcaatatccagaatgatattactaattaataaccccccgttaaatatttcatttctattaatatacatggttgatgatgggatgctcgatgccagaccagatgttgaaccgtctagacgttggtgcattacatgcgtcaaataagcatcccttaatgcagctgtgtgttcatcagttatgtttttcagtgattccgggagaagatttaagctattaactgattgaatacggcgtgtaacgatactccagtcatcttggtgaatgacagcgaatgcctgtttatgctctggtagggatattctatataccgggataggtctgtgcagatcacgacgccccaataaaacattaactggtgagatgagttgttccgatggcgacgtggaagccactaaatgtagaagtcttctggataaactactcgatactgttgtagtatctccggaggggtaatcatgttgtgaatatatcccttgggtttgtgaagtaatattatagggccatccccgacgggctattgagttctccacaccatcgatgagctttatgaaagcccccgcgacatcacacataccaatgtacccaccagattgtgctgtcaaagagggataggttttggaggcagcataaacacgtccgagtttctcattgatgaccacgacatgcgggttaacgcactgtgatatataattacttatatgtagctgagctcgtatatgagaaaagaagttttctatagtactccgtttaagtgatgaggctgatacaacgtctttgattagcgctatgtctgtcagagttacggaccgtagatgatcgcggacctgcctatgattaaagctctcgaaatgggctagatagatataggtaataaactctccatcggagatacgcaaactctgtctatctgcggctatatattccccgagtgtgctaatctccgaaatatctgctgttatacgataatgtatataatcgcagatgtgtgccgccgctgaccctctggcataaccactctgacaacaaaatcttgaaaatgtcgcaaatgaggctaggttggagacgtcaaggccactggtattatcggtaacatttacgttgtaggtacagataaaatctttaacggcctgcagatcataacaagctccggtgtgtgataattgggttgcctgtaatagataatagcgcgtagctaacacgagggtcttttctccaggtccaaattttgccacgaaccaaaagggggtaggattaataccataaatacgacgaaatgttgccaagacggcctgttcgtggtggatgtacatcgaagttaacccccttcggccattcatgtaaccagaaagcgtcttaatggaggtctcggggtttggatcatattgagctgagtcagttgtgcatccaagccttggaactaattgttttgtatttatcgccagagccaaaatcagatcgtcgtggagtgcaaaagttcgttcctcatctaaacaatctaataacaatcccgtagacaaggggtctccataaagtatagcccgtgccagcgctatactttccatgggtgtttggagcctacagaaaaacacaggatgtgtgcgtggtttacacccctcacgttccatggtgaatggcaaaccacccaataggcagaaactaaggtgtgttccatgctctaaaaggacggcggctagatggtggtgtgaaaattgcgcaccatcaccagcaccggctgataagtctaaatcttcatctgccatcattgggagattcccagtaggtgtcatattgaatacatcgtttaggtcttcactccaggcataactgacaacatatatctcgcggaaagtcttctgtcctgacagtatcatcaaggagtctgttatcgcacacccgtccgttgcatataatatttgtatatacgacccatcgaaaggctgcgccatgataaaaagtttcctggcaggtctgtgtggaaatccggtggataacgccgaaacttatgaaccaattcataatgggccaggcaatgataccacgctccgcctcagggaggcgcttgccgttataaacacgctacttcccaatcccttgacggttgcggatgtaatttcatcggcagaacaaaccaagaaactgatcaaggcgcatgccttggctagaacctatcaagcatgtcaacgaaacctagaatgtctttccaagcaccaagtaactggagataatccaagtctcacttccatagtcgaatcacatatagctaatgcacggcgcctctcggactcctgtctagctgccatcgtacatctatatctatctgtggggtccgtagataccaccaccgacactatagtcgaccaagccatacgcatgagttccgaaaataacattgtgatggccgatgtagctgtattagagaagacttttggtatcggtgctgcaagctcggggaacagacatctagttactaatcaaccatccgcatcggaggagtgcaaaatatcaaatcacccgaatgtcacatctgttactactatggacataactccaaacgtcgtagcccgtgaaccggttctggttaacgaccatatctttgataatggagattctataagacatactaaaaaatccaagcgcaaatcacatcgacaaacagaattgactatggaataaaaacagatttcgttgttatgttatttatcatatcgtttattttattataccccggttgacccaaatcctttatccccccgaaggctcgatggggcatcacgatcgaagttttcagtaaaggtccataaatccgcgtgagccatgattgttccaggtgcaggttctggggtttctggaaatggatcgtgattattcgtttccgtaggtagccattccataggtaattcttccagaagtaataactgggcaatcctttgcccagctctaatgattactggatcgcgacgcatattttttatgaagaacccacattttgtctcaggtttccatacagtagggagaactacgataccactcacgttaagagaagacctcccaaagatacatgctatgggggggttgtcacccggaaggtgaactactgggatctctacatagtgattaaatcccggataaatctggaacgtttcaggtgcggatatatcataaccagcatcttcgtcccgtttttgtttaaaaatttcatagaaatcacacagtcgagcacccattgggaccccttgtttatgcctaatcgtagcaggacatggtgctaatgtaatcgtagctttactaagcttgagtagaggtagttgtaactttagaccatactccttaatagtctcgatacacggtctgtggtagattagcgctttgagaattccgcgatacccagaatcgataaccccattgatgattgacactgcttgactgggatcttgggaggagcacgtttgcgtaattaaaattgcatatccatcaggcgtggctaatctaagcccaatatctaccggtctggtataaaagatgtccgcattatccggctctctactcatcaacccaagctcgattggttgattatttgtcaccaacagagcattctcactgaccaataaactccacgcctctgaagtcttttctaccaaaacggagatggatcgtttatccgtaacatcagtgctggttatcctattggtcgccatgtcttctactacagcgattcagatgccccgcaacgcggaagattttgagagtatggagcggctaaagcaattttggagcgcctcgtgttcggcaaggggtgtgaatatcaacacccccactgcatccgctgttttattttacatgtccttgattgccgtgggtgtggctatgatttgttacgcctaccggatgtgtctacgcatggtaagcagcgaacttcgacgcatgccacactaaaacgtctcggaaatttgggggttttccccggaaacgattcagcatcatgtcgaatgccggacaccctataaaagacccgggattagtggacattggtattcacattcatcgcccgagaagcgttgtcttcgcgatggccggtcgtcgacaaccagcggggcgtaccacagaaacccctccgcgagtccccaagaataaaccaactgtacccttgaaacaggtacaaaatcgacttattatggagaacccattgtacgatactaggttctttgatgaagatatatatgaagccccatcaaacgtgaataacaaggattatgaagttgtttatgagaatctggacaacccatataatacagaacatatttacgaggtagaagaggatcccctctacgactatgctatttgtggatcgtcaactaatcaccataagaaaaaggatagaccttctacagataagactactttggtcagacgaccagccatggtcatgcgacatccaacgcagcttccggtggcagatcctaaggaacgatgtaagacaggctctgatactaaactcgcggcatgttcttctatgggttgtggatcgccgagatctacacctccctcacccacaccagttaaaccaagagccgcgccgggtaccaataaagtaattgctggaaaaaccctagcatttagcgggactccaagaacctcatccacgccatggagaggggcaacgcatttgtttaacaagaacattttctgtggggccatggcgaaggttgcagccagacatgcggcggatgctgcagcctcgatctgggacatggaacccccctcaaataatgatgagttggagaggctattgaaaaaggctgtaatccgtgtcaccgtctgtgaggggcttaacctattacacacagctaataatgtacaatcttctgcggtacaggcctcgccgtgtgtgtctaaaactatgcccactccagtaccatctgatgctgtagtcagacctaagaccagatcctcctccaagtcaagaagacgttctattggaaagcctagactgggtgatttcgacggtgatagtgactgaagtgttaaatttgaattccacacccatattatatttacaaataaagtataatgttttgcatatataaagctctctagtcctactcttttttcattgagcgtgtggtggttggatgtaggaataccctgtatcatggctcgtgagtttacccattacgaagattcggttgtacctctagaaccagactatccagatgatatcatcgcagacttcgatgaatcgctattgggtactcctgggtataattctaatttgggtaataatcgattaatcttcacacatccaccaaaacctgctagcccggcaagtctttacgaaaaactctgtaatgagcttaatttttcagacgggcctggcttattatcagctatggagaaatggaacgaggatttattctcttgtttaccgagatttacagagctctatgcagaaaatgttttattatcgacatctgtcgatgaggttattggggatcttgagaacccccgtaagactgaaatgatctgtgatctccaggtccatggattagaaccatttccggaagtacccatggttgacgatgatctcgaggattatataacagccattcaggggttttttatagcggaactacgggccagggaagtccaatattcaattttattaactgagtattgtcgggctatattacgttatctatatgccggtgcgcggcgaggcccaagggataaaggtaagggggtaacggatgagtctctgaggcagaggcttagggccactattcgcaatagatattataaggaagtagcaacaatcgcacgtgtgttatatctccatctatatatctctgtagtcagagaagtatcttggaagctacatgctagtcaagtagtatcacagagtttattcgtttccctatcttatacgtggatccataggagacaacttgagtgtattttccacccgatattatttaatcacggagtcgttatgttggaggatagaccgcttacattcagagagttacaaactattaactatagacgtcacgctctgggattaccgctcatacgagcgggtctgatcgaagaggagggtcaacccctactgttaaagccagaattttctgggagcctaccaagaacaatcggatttctcacacaacatatccgcgcgaagatggaagctttttccgatcgtcacccgacattacctagattcccgcatctggagcattcgtatgcaaaacaggagcatcctataaactatggaactacaaccgaggccatgatggacccaccatcaccaagtgccgtgcttccaggggatccagatcccaagataggtgtgaagatccagaatacaactcaatccctccaactgccttcacacgtgactctggatcatctagaatccaaaggttatgtaatctcggcacagggattatcatatgatgatattcctgaacacgagcttaataaaatgtttcatctatgattataaataatcaacccctctcgtcataaaaaattaaaaactattttgacgaacataagtgtccatcaacggtttttttagcggtgtgggttgaggaaacatcccaagggggtgtaggatttacgtcatcatggttcaaattggtgacacccacatggagcacatatggataaaagacgggtggctggtactggaaacgcccatataatcgtgagcgttgtcatttgaacacgtttggtgaaatctatacgcctctgtctgagagtgtcttatcaccatatgataacaagattatcgggtattacatcatcatatatcggagttgtacatctccgtatcaaaaagggtatatttttaaggagagaatatggcagattgggtccccgaaagcggtggacccacacgacgcccacgaagatcttctaatcgtcgtagtcatccctttttgagacctgaaggtggcaatcctgaacgtggacagcaagtacaagatattagagatcagtatgggactagcgcagatcccagggaagatggaccatcaacgtctggtttttggcattttatgcgaggggtattcggtggtgaagatgagaacccggttgtgatgcatcgcccaagacgtgattttcaaccaccaccgggtgaagactcaagcgatgaagatgatgaagaggacccccggcaatttgcctcatggggtgacatgttactatatggagatcaatacgaggcggaaggggattcggatgatcaagaagaggctgacaataatataacagatgatgacacttctgatatggacggaataagcgatgatgaggaagaagatgataatgaaatcgatatggttggatatggagaggaagataattatcccaatctaaggggagatgaagaagcctccttggcaatcgactctgatgacccgatacctagtggggatgggggggaagaggatgtggactctgaagaccttccagctgatagggatcgtcttacatctctcctaagatatttggaaagtggaatacttgggacatcatcacgaagattgatagagagaaccgcgagggaggtatacatggatcaatttcaaccaggtggtcgtggagcagaggtagaatctgggatatctgatcgtctaaaccgtctggtccggggtgggatttcagatttacaatttcggggaccctttttagaggaagaagatgatccaaatgccgaaactggagaatggcggaggctggtacccatcacagttacactaactccatcatgggaattagtaatacgtgatcatggtcgccggcgacgcacattaaacccagaaactattttctatgaagatgtggaaggattgcgtaggttacgtgtaccatctgtcctagcggataggttgacacgagaggcatttatcgagaaaatgaatgttctgtttatgtttagggacctatcagagagcgataattataacgaagaacagatatggactaccatgccatctctacacggttttgcgcagggttgttggtttgaggcggatatcgcatatccaccagggggtatgtatactacacccaccagtgaggtacgtgggatatggagacgtactattcgacaggggatagtcctccaacacagactttgcctcggtaattttatcagagcggtaaccatcaatgacttacaccatcggtccgcaatcaattttctattggatgcgtgtatccgatcgtgtataaactgtcatatgatgggaagattaattcataatgcctataatcagttggaaggtcaggaagcccctataggctctcttgcatggatggtaggaagaacggccatacgaccacttccaacaccatgcactcagtattttccactgaaggaattttttggggcacctagaggatctcgtaattcaatataccgcagttcctttggtgcgcttttgtattggtcggaactcagattggccattggggatcctggaaatattaatatcagatatgccgggtatcatatccagacagcagaggtctatcttctctctcgtggtacatctaaaaatcctggatatacgagggaggaattagatggtatggaaactatcttatctctggggacacttatgttagaggtggcggtgcagtggatgtacgtagccacggctcgcattctcagtgagcacccgaatatcaaagcgtaccgtagcgtacgtagtacagtattcgcgtccggtgttccactgggaagtgttaaactcgcggatgctgaatatgaccttttacgccgtcctgaggtgtcggtagcccgtgacaccaccgcgttaggtcaagcattatttttagcatattatacaaccagaactgccctaacggcactgattagagattatgcggtcgcttctatgttaggtcatgaacggacggccatgagcgcatatctaggtattgtactgctaatccaacgccttggggggcacttgaacttaatattgttgaccctggccggggcggctatacatggaggacgccgagtgcccatacatcaggcgacgctaccacggtataccatgattgctgatatactagctccgttgcttcaacaacagacattgacaggtttctggaaaacaagagatatactatgccaccaattgggactcgtaccaatgccgggtccacccacacaaggaaaacaggtggttataaatctggctatgccatctgaggatctcgagggattgacaccccagggagctatacaatctaacacggcattgggagatatccaggtagacctggcggatactcttcaacattatcatcggatgatccttggggagatgtcatcagatcagttgggacggcaagggctggaacgtatgcgaggtggagcttcaaggagacgagggaggcggggaattaaccatgtataagtactctgtgcatatcaataaacctcgcatgttaacctgacagtacgcgaagtctattcgttcttttattttttagtatgtctaactcccttccattaccgggtcactcatctgcatatagatcatttagaaactctaactcatccagggatgtgtcttcgtgggttggagtcgcggagaatctactcgagcggcgggtagaaaaggggtgtatgcttccaacccccgcagagattttagagtccgcggtgagtgctctgaaggagtcagctgaaaatctatctggacctggactcttctgtatggaacgtgttaccgctctatccagaattcgagataacactataccggagactataattactacgtgtatgggaaaggacaatggggaagaattccgccgtggatatataatggcagccaggagggtgatagaaggattgaacatgacacgtgccggaatttggcagattatgattaaaaattattggacctatctcaagacatcgagtggaacggatatgacggtagattccaggaccccctcttctctgaaggtcatgttgttatggtcaacattcagtgggaaacggttatccaagcaaccctttaagcatagcaagcaaaatgataatcatgaagcattaatcagcgaactgaagcgtgttatgataactctagaaaaatatgcgtattatatgcgaccggacgacccgatgagcaaaagtaccgacactatgcttcgacttcatgaaattctagcctatgtaagcgtcagttataggtggctcttatggttcatggacctcctagataatcgggtattatgtgcaatggataagaaaccgattattagacatggaccaagagaatcccagtctccacgcgatttcatccaaagacatttaacgagcggacctggtatttcatcgggaacgggtgaggcccttttactatcagccgagacggcaaacgcactagcgacgctattacgtataagttcaatgtggagctatgggaaatggagtacgcatacacatggagttacgagtacaatcgtagcggcattggagatggttacccagatacatctccatcttcagtattttatcaatcttgtatttgggggatatatgtgctgggctgagggaggtatggaagattcttatatcaggtcagccctgcgtctccagggaagatttaatcactatattggtgctgttatcccggtaatgtctgcattaacctggagtggaatggaaaagagtgtctcctgttggtttaaatattcattggccaaaagtcttatcggccacgtgaccccaacacagcactatttaggaattcttggttcgatagaacgtggaagacggagatccacccgggagagggcgaaaactgagaggcggtgtgtatcacagatgatgacacacatagatcggcagagcctcccactaccatccccacccactccacacgctcatcatgatgttacctacccaccagctgtcatcaccccccaaacccatatatgggtatggcatcactcttagcctccccactaacccctcgggatatatcctccccaggtcttggagaccacccttctaataccccagacggggtagaggggtgcttccatagaacgcccttgagatcggatgcgcgcacaaaacaactcgatgatcgttttgattcctcggactgtgacggaacaccctataccagtttagatgacatccgacccgaagagtcggaagatcatattaataatccgtcctacggtctcactgaaacttttaattcctcgaatctcagtaatggagatgaacctcgagatgcataccagactcattaccaacaccctcgttgtatttcccctgaagataccgcgcagtccagaggatcgtccgcgtctggaactacctcacagctccaatatacggagcattaccagacaccgatttgtcataaaaagtgtcaatgttccaagcccgggggacctaaaggtgattatcccgaattatataacgaatgtggtgatgtaaagaaaaacctcccacgcagacgatcatcctcgcctgggattgaagaagtagtagaaagatttcgcccacggtctagattactcgacgatacatacggaattgaaactctggcaacattaaatgcctcttcgatcaaacgcgcacatggcactagtagggtggactttcaaacacacgctattagaaaataatgtgatatcatctcgatatatccaataatccgacgaatttattacaaggttaatcaatataattgatcattgtcgagttgttatccacccatcgcccgtacatacgacaataaacactagaatcatgtttaattacctaaacggtattgtttatttatttagtaaatggattaatgatttaggattccagaaaatatctgcctaacgcgcataataaagtcacttaggggatttggaagatatgttgattgacatacggccattgatgtattacacgattttttttcatcagagagaatacaatcgtaaagagagtcgtttgtagatatcccatctccattgaagatgtccaataatggtataagagctccaacagcagatgcggtggcgggttgttccatgtattcggcacatgtggcaactagatcttggtgtgatttaacatggcgtctaatcggtcggatacaacagtttttgatacccaccgtttcctcagggcagcgtggcatctggagcatatccactggtattgtaaatatgaatgtacatactataatagtagctgttaggataactccgaaaaaaaacatacccagagaaaataaaaaaattccactcctacgcgatgaaatagctgtcgtcaacattttattcggtgggttaggctcgttggagtgtatagatggctgtggttccatatctaccagtggtatctcgaccaacggctgtgacgtagacctcataccttgcatgtccacgcgaatctttttattatatctgggtgatcgtagactagttaaaatgtgtgagagtaattacgtcgcgggttaccaaagatcacggattggagtcggtttatatccatacgaatgattataataccactgggcttgttttttataaacgatggactttgtaaactattagtaaacaattaactgttataaaaatataaccgctgaaacacggttatgataagtaatttatacgaactataagagttataatcgccggggatgagaataatataaacataatgccgtaataaagataaagactgtgatgagaacagctcctagtattacagcaacgacactaaccaacattgattcactgacgatttcaggggacgcgtcgtagacttgtgtgtcgtaaaatcccggtagaattggtgggtatccgatggtctgacaggtataactcacgggtccatccgtttctgatatatcacacatactctgaatgttgaccaaacctggacgttctatacacggacctgtgatcagtatatcagcactcattgcttttggtagatggtagttaccaattttccacatcaaggatatcccgtcccgtggtacacatttaacatcacatatagcccgtgtatcagcgaaggtaacagacacagtaggtgggtaatagacgtccggggctacacttttcgtgaatctcttataggacacaggactttcatgccattccaaatcacatcgtatatctggtttctcggaatctgtttcagttgtcggaatagccaacacagaacttctggtgatgagtccatctatccatacacttgccgtatctgaaatgtattgcctttctattggcttcccgtcaagataccatgttagtttcgtggatcgcggggggaagtaattcgccactgcacataccgcctcaaaggtcttattaaatagactggcgcgtggtgtaagatttacgctcggggggtgatatgccactacagatacatttacctgtcctagcactgttttatttagtccatctccaccatagacccgccatgtataaacaccggacgtggcagattccacagatttgattcccaatgacatcgcttcggtatgttgtatgggaatattatataatgtgtaatctggtatcctagattgtaggatcccgagtgtcgcgtccccaagatccatggaattaacactcccattacggctgctaaatagtaatatgctggtgcgggtatgattccctacgaagtcttctttatccccaagaaaatccccaaatctggtgcgtggtttaaaccagatctcgaagtcaatagggttggtaatatgaggttctgtgggtagagtacagtttagctgaagagggctaaaatacggtctgtagataacccccttatatccacaatctaatttaaatctggccccgttattcttattctttttacgctttggtttttttgttgttttgagtaaagtggtattggaatcggtagatgatcctatgaatgtggctgcctgggtggtactcggacttgtagctatagttgtcgagcttctagttagagtagttaatgtagatgttggctgggatgcctgggtggtactcgaacttgtagctatatttgtagatcttctagttggagtagtgaatggagatgttggctgggatatcggggtggtagcggacatgctggtagatgataacatctccgcagtactattatcactattttcaatactgaccgcggttgtcgacgaagtaccaaactggagatatgtatataacagacagatatagagaaggtagattccgcgtcccatcctatatcgtctcatcgcagattgttggtgatcccgggatgtaaatcgtctcgcgggactacttaaaataagagattcctaacatagacaggagacccatttttaaggcatttcactgcctgtgatttcgacttatctctttatttaaaaacagttattagacacaaccgcgatataaatatatcgtgttgagtggtacattacatatgactgtacaaataacacattccagcagtaacatacatgcccgtgtgtagacccctgcataggtaggtagcagccaggcgtttcgcgccgaggatgatttttcgtagatggagtaggcaagttagagtctcagatatagccagagtcaaaaccagcggcgtgccaaaatttacaccaagcagtgccaagactatcccggttatttcagccatgccatggaaactcacgaggacctcaggtatatacattcttcggtagaccatcaaaggttccaggcaaaatgccaaacaatgtccaacgataacacaaatcacaactgttaagggcgtctgaaaatcatggccatatttagtgatgtgacaataaccccatatcggaggggtgagggttagccctatcaacggggccataatcaaagtggggtgttctcgaaaaattttaacgctgctaatcttgtcctcgtagacatcctcgatgttgggtacgataaagtcgactggattatggttagcaccacgccccaaatccgtaaaagtgcgcattaacgaataacgacctaatctgtaatgacatgtgtctctaagtatcattccaaagtctcgctgggagacaataatcgagcatgcaataccgaggccgatgagaatttcagatttcacagtcacggcgatactcgatactcccaacagcgcaccgagggtcatggtaacgatggccatacgaaaatgtgtccccgatccattggctgtgcagatgaagtaaaacacatgtatgatcgcagcacatacagcgattcctgctagacataggagtagaagaggggtaaatccaactggttgtaacaaatcatcacacgcccatccaactgccgctatggccacacagagggtctgagtaaagcggcatatagttgtgatacgttcagtttttaccatagagagcctcattgtggatattagacttataaatataaggattccggctgcatatgttgttgtacgtgttaggagccaaatgagagcgatcgatgctgttaaaattccagattggacccctatataacataacgctttagttgccgtgtacacacatgcgatacatccagtcctcgatcccgagcagttcctcacagtaaatgagggtagtgttatatccgatatcccttctggagcgagaccgttattgttggttgttgagctcccagctgcattagttggagacgtacagtcatcggtggccatactcgaagtctggctggcgacttactgactttctttcgcctggctcaatttttattacaaataccagaaaaatgtgggtggagatatctaactagcgtttatgaataaacgaaactacacaatatattacattaaaaatactttattttaacataaatggggatcaagtatacaggcgaagcatctgtggtattggctatttacatctaaatatcagatgggttttctccccgtgtaccaggattcgggttgcatagtggagatgagtaaacaaaagtacaaagttttttactgggtccttcagaatcacggaactcttctaacactcgccgctttaacctggggatactttcctcctcggttgtcgagggatcatccatatccccggttttaattgactcggatgatcctagacattccgtggtacagaagaaaaacatcattacaccaacgggttttctggtagtcagacttaacatgggtgtagtacttggagtgaagaagaaattgaggctgacgtcggtatgtgtaagtttcaatttacgaagaagttgaaaaaagtttacgggggattccaaagctaggcgaaagggtttcaaaaccccagagaaattaacggttgatgatacattaccacgtatgtgggaccttgggagactgcgtaaaatacagcggctatcttgatttccatcagccgattcatgagtcgtggtaaatgttactctcccgacgtcggatataatattgatggtttctctcaccgtagattcaaacactagtgtagattttatgcgttttccaaggtccaatattttgtttagttgaggttttgaaaactcaagcgtcaaatcgggtactctcgtgggtaacataacacaataattattaaattctgattttataagttgtgaagaagatagtccaccatcgaggtctaagttaaaaactacttggtttaacattctacccggatgaacattagttatctcgaaaattaccttcttggttgcactttttctcccagtagttttaaatacgtccaacaagctacgacgtccgtcgatattcgctaaaaataccgcaggctgtccatatgtccatctatatgatgaaaactgattctttgatattggtatatatatctgttcgtgacatacactcgtatgaatcaacatcccatcggcactaaaaaccaaaaatgagtttttaagactggtcgatatcggtgtcaatatggtcattacctcgcgtagcttgtcttcttcgaggacagcgtttccgaagatagtgttagattcggcaccaccgagaagaggtcccgcgtggacaacttgtgccggatggttaatagctttatttattccaacctctaatctcgacggtggaaatgccatcgtagagagctagttttacacacaaattaaaactcacaaagggaaaaataagacaatttcctcgcacctgtattctttataggcttcaccgccgcgctatgacgccagcgtggggggacggattaattatctaccatgtatttttggtttcatttttattgttcatgatcaccaataggattttgaggttccacagacccgctcatatttggtcgtgctttgagttgggtgcggcgatgattccgccacccagcgcattataatgtccataatcaactaggggttaatctagttggggaactgccttcaccggagcgattgtttacctcgccaattaatacgactctgctctaggaaacgagatataatcaatagtgtcagaatgggcttatttgggatgttaaaatacgcctactgcaaccgtttggtaaaacatgatgccatcactacaccacctggtatttatacacccatcgctatagatttatggaatgttatgtacacacttatggagaaattctttccagagaaagatatacaagtggataatgcaacggtgactgccgagtgtttcttttcgcttcttaaactgttacagaagcgatcatactttccgatcttcgtttcggatagaggtatctatggtgatggacaggtaaaatatggagccaaggctatagtgacacagacgatgtgtttacagggtggatctggacgcttgggggaattttccattccacatggagggccgtgtgaagatacttcggaaccagatggtggaaacccgaaagattgcaaagtagatatccaagaacgtatatcagcgagatggagttcattagcttataccccacgtttatgttatagattatgtgtaaacctcatccgctatctagggtacccatatgtaaatgtcagtgatatggaggctgatgatgtatgtgctaatctatatcatacaaatacggtcgcgcagatctatacaaccgataccgatcttatattaatggggtgtgatattatattagatataattccacttttcccaccaacaatacgatgtagggacgtccttgcgtcgcttaatctgacatactcggaattcgtggcgcgatttgttcgctgtcatacagatctacaccagacacctatattaaaatccgtacaaaatctacgatctaatcaacgacatcagagcaaaactaaggtaaaagaaaaggtaattaatctaaaatcctctactagtatatcccgggataccagcagtgatgcttcatcgtcagaagaacgggatattgaatgtatgtccacagacacatataggatcagcccatctcaaattcgtggatcgtctaaacagctgaccatgatagaagagcagagtctgactctaaaatacacatctcgatacccaccgataatgaaaaccgcgtcacagactctctcatttctacccccgccacagacaaaacatgaggtcctcgagcggaaatttatcaaacaccttatcgctagaatgacccctgagacaaggggtcgcttatcaatattaaagcgcgttcccatcctccaagagaaggctaattttaacctggtgttcgaaacactaaaacattttataggggatgccgccaatgccaaacatataactaacttattctggaagcatatctcaaccccgcccgactataatgttgttctggttacctattgggatgattgtgattcgcgacggcggttataaataaagaaccagtggattatccgggaaacaagaaccggttaacgctaccaatgactcatgtgtagttatttttgaaaaaaagggacgaatccagatacacgttagcttaaattaaatttattattaagacattgtttatagatcattgataacagtgccactgtatgtagtgctatgtctctcgaaaaaattggtatgtttttcaattaccataaaggctaagggaaaatcgggatgaggaggcgggacgttgaaaataggagccactttgataataccgagcaaccggtccgcactataccgaacataattacaaatatccgccacgttgagtagattactgcagcgcggagcgcatgttgagataaactcacactcaagttccacagcctctcgaaataacgagtgaatcctctccgtggatggtttaatttgagccgggaggtagttgttgtagatacagcaggatgcggttgtatgtatggcctcatcgcggctgataagatcgttggtttgacaagttacgatgaatatgttgtgggttctcagataagctatagcggcgaaggatgcaacaaaaaagataccttctattaagatcatgagaatatacttctcggctatggattcacattcaccaattcgcgtgcggagccattcaaccttcctcgcaagctccggctttgtaatcatagaatccacataggccgtacgcgcctccgcatcacatttaaaaaggaggagttgtattgcgctatatacacgcgaatggacgacctctatacattcctgttcaatgtaataatgtaaaatatctttttgggtgaagagctcagatagattgccgagattcaggttaaccagatcgtccgcggcagatagaaatgtaaatagaaaacgataaaagtccaactccgattcggatagtgtagtaacgtcctttatatcatcaccgattggcaaatcggtttctaaccacctattcgcgatactgagagaccgcaacatgtttatatctggacattccagggtgtaaaagtatcgcgaggcgctacaatcagagtctatggataccggcattttggtattggagtggagaccggtattgggggtagatccgttagcagacatgatatacaatcgtttaaaggtggcaactagtacaaatgagttcgcctccggagaagacaccactgttggtagcctttttgagcttacaataatacataccggtcttcagtccacatttatatgcatgtacaaggaggttcataatcctagaggcgggtaatttgccatcagccggttcagagataaacagagacatggactggctttgatctacaaagggggccctgtccgcacacatatctatcaactgtcgttgatcatattcgaatgcggttttgaattttgctagggggtgataatccccgagatccccgaatgcaccacgtatggaccattgggcagcttccagggttgatataacagagagacgcgcgcattccctgggaaataggcgtcgtatcgtccgcatcaactgtaagtttggtcgtaagatctccccggtactagtgactttactaaacagattagtaaaaattggagagaacccctcgctactctcagtgacctgggacgaggacaccgttggcattaatgctacaaattgagaattgtaaagtccgtatttcatcataactactctcaattgctgccattttttaaaatgtataaggggggtgtttgggtatgcatcaaagggtaataaaccacgtccatagatactctctctgaagtttttaaatggtgtcataccaagcttgcatagggatgtgctagcgtggattgctcccaggagcagactctctaaaatttggcggtttaagcgacgggcttcataagaaaccatatccatctctagggccaaaaacgtggtatggagcccctgtatacccaatccaagagagcgattttcttctacaccccgcatggccgcttctgtggggaaatttccagcacgcatcattgcgttgacaaatatcgtagcggtatatgctgcgcgaatgagtaatgggatactgaaatacaagctctgatcatgagcgatgcggcgtaaacattgcggtagatttatacttatcaagttacaaacgccatgtttagtgtctgtagccttctgaatgatttcagtacataggtttgatgttgtgagcgcatccccacgtgtatcgaagtgatagtgacgattacacgcgtccttgaacattaagaagggactcccggtcattattgcacttctcacaataagataggccatctcttgaataggcaggctgtcaacacccataccagccgcctccagacgttcatattcctcattaaattcctttccataaagcttcgaaagatgggatgcggcatcatcaaatagagtccaaatgacatttacctctccgcctaggtgccgttgatacctatcgaagaataggtccggggtccataaacagctaaatatattatcacatctggagttttcatccgatgcgagcattccccgcatatttaatatcgatctgatatcggcatgccagggttctacgtatatacatacaccagtgggacgttcgctgtcgctgttgatggctattgtcatggaatcgagtagcttcaaaaggggaagcacaccctttgtacattccttagacagttttttgttgaaattctgcaggcatattccaatcccccctctacgtagtagaatcggcgctacatctcgaattaaggcattaatggcagtttccgtgtctgtcattatcggactcaacagataacaacttgcgagagatcccttagtacgtcctccgaataacattattggtgtggctggaatcatcaactgcctagaaagatagaaaaaaaaggatttgaatactctgaaccacgttggtagtttagcgttaagcgcctgtctcatctcaggcgtggacgccgttgcggcggtcgctgccaatctcaggaagaattggcccatcgattccaaagctccattttccaggtttgccaaatacatctcttcatatttgagtgcagattgtagaccaagttcacataacggttcataatcatgtatctcgaattgttccaagacccttttcacacctggcatattaatagagatatcccgtatcacttgtggtaggagtgccaattccatagaatttagccatttatccaacgttgtagtttttacacgaatcctgatatgaaccatcttccccagttctatgtaaatcctctcatcgatacgacatcgcggtttgaggttagccaccacacttgtaatatattccacagcctcttggcgtgatacgttttgtaatatggacgacccggaatacccataaccaatgagtctgttataacagtcgtgtatgtcgagctggagactttccgggcagcaggtttccataaatggggtactagagcctgacgatgccatatggttagaatatcaggttgttgatcggtctaattaaatgctacagggaatcaaatccctctcttatgttaatgtagagtcgcctgatgtggatgtggggctgaatggagcgacaaccatgtatatatacagacaggtggggaaagcaacaaacattacgtcatatgtaaataaaataaagtttaatttacatatttagagttgatgtgttaatccatacagtttagattaatagtagcagtctatccaggtttccggatcccatttaaaaccttcgagccataccacgggaacttcaacacctccatagcgttcgaccttgcgagtaggtcgtgtgttatcagaagaaaggataccgaggcgcatataggcagagtatagacaggaatttttatcggcacggccgcgcaggtcaggtgtccattgatatagggctgcggtggcttgggggtcatgtaattgaaacgtggtacaccttggtgcattaggtgtccgtgtaagttcctcgagaggaaatctggctcgtgggtccgtggcgggggcgatgcttccctcagtccctctaatcgcattttctgtccatactctatgaaagagacaactgagtatatcaccgaatgcttgactgttgagcttactagtgataatttggatacggacaccttcacgtcccaggcgctgattatatacaaataccagatatacaaactttacaccatcgccattcaatctaagttcacaatcggcatctaaaaaggtacatgctggcacatgaaccgtggatctaggtattatggtctgatctgttttcccaacttcctggacaccttgactaatcgcagttatactagctaggcgatcctgggatatccaggaaagtaacccaccaaaaataccaaaaaactgatgagggaacactcgacagcgtaacataatcccaagacaatctctaagcctgtccaggcgtgcctcgatgcgacggtctttgtaattactaattatatgagttcgtacggcatccgcagcgcttctgtctggataatcttccacacgcgcagccaccaaaaaattaagtgatataagggatgctcggatgccttctcctgaatccctaccacctgtggtactggttaaagcggaccaagcctcttcgatctcatgtggatccctccccggtggagtggatgttatagcagctccaactatatcggctggatggcgtaaggtgaggattggaagacccggacgttctgcactggggtgacaaaaatcggtaagtgtcacctgacgcgtgagtcttaacgaatcttgagaggttatagtagcaggacatgtttgttcggctcggataacggtatccatgacattagatgtaaattggattcctcttgaatttttgataattatagaatttggggtgatatgtaatgatccgggaagccattccaacgtattaccagtaccagtatatcccacgtgaaatggcgacctatggcgctgcactgtagagttaaaatttaccgagccggttttccgaggagggttgccatgtggtggtaagactatataagttctaagcccgttcccaatttgaatcgaagaatttccacgtatatccatcgtaatataaggtattggttatatcgaatatatcaccaacagtctgatctcgtaccacgcctcacgaaacgacgtttaatgttaggcactgagtgaaagtcatacttatacgtgctggacgtgggttgtacacgtgttagctaaccggtgattgaaaaattccgccaatcggcttttaacattttcgaatatagtaatcgtgactggactatacatccacggtataagggtccggtgtgttacttatatcacctctaagggatatatgtcagttatttcacagccgtctatgtcttcatatatccagcgattgagagtttaatttaatcgtagaggatttttcaaatatggataatagcggaccccttatgactctagtagcctcccttgaagggctagtaggcgtagccagtgacagactaacccaggatggtgttttgcggataaagtctatgatctctgaattttttttatctacggattctatagaactccgcgatacgcaaagattatgggctaagttacaaaaactagcatgtgatgcctatttacatacacggagccccgagaccgcgtttttggctgaaaatctgccaggattaattttctggagatttaaacatgattggacggagagcccaattaatgatctcactgatatatcaaccttattggatgtgatgaacgacgaggagtgtatggcctgtataactacggctgggctacgagtatcttcatttctgggtccatctaacatctatcggcttgttagtgaatggatagtcctatttaaggagatatatctaggggtgttaaataaaaccccatctgatgcattaaacgaaccgcctatatcatcccttgataagttttcagaacccctcgtttcgaagaaatttgaacttctatatggcatgccttttgtccaggagggtttacgtgtgatagccatcagagcgaattggttggtacagtttggggttatggttcaaaggaccagagattctacgctaaccccactcacccgtgcgttatatatgttggctctagtggacgaatattttcaagatatcgagcaaacatctacatacactacgctcgttagagattttctggaactcacccaggagattgacgaaggagccctggtacctttacaggccgctaatctatcaccgcgaactgcttatgaggttagaatatcctcggcaatagcacatcaaaatcctttcataaccaatcctcagccaggaacggtgactgtacgtttacgcacggaccccgagattcttaccgaacgacatctaaatttagaggcactccttatccacgtcacagcaattatcagactattagattcaaaggacatcacctatgaagatggatccaataccatatggaattatgtggttgaatgcactacaaatacatgggaggttatccaggcgagcacgaatccacatcaagccatagaggcgcttattcaagcaggatttacatcatttcattgtagtatgttagaacgtgccatctccgataaattttccaaggcacggataagtaatattaaccgacactctatccaaagacctctcttggatgaggcgcaacaggcaattggctgtgttgccatggtggggagtttgatatttaaattagtgacacattatggaaatggtctagattatattagacattatactaccactctcgcagatttacctctagtatacggagacttgctcgattcactcggtttaccgaatggtagtgtggaacagatcatacgccattgtatggcaccaaaaccatatatagattacatcacaaattcgagggtggtttttgagacggagttaaacttggttgatcagagagtggttactgtggagggtaatacacacaacgctgcgagagaatcacttttaatgtggttcgatttcaaagcgagagatctatggggtattcaagtacaatatgatgcctatacaactggtcagggtagagaatcgtcagaacacagtgttagtgaagtagatcttcttttggcggctatacagatagaatatccatctattgatggagtaccacatcatgttctatctgagccagcttttaccccttatctatttataacagtagttttggatgccctacacgtactgatcacctctagattttcgggcaaaacactaatagatgttattaaggtatgtacatgggcccgagattatggtataggcgtagtagcaaatgttgacgggtaccgcacaaagctgactgctattattaatgtattaaatcaagtcactgatacatcttcgaaaccattaacagtggcgcatgtagacaatctcagatctctaatggtcgaacttcattctgtggttaccaaagcggtcatgctggtcccggagggtgtgtcagtaatgctccccgaaacaccgtccctgaaaaatagtacattcctggccgagatgtatctatcggcgattcttcacagactcaagggactaattatatatacagcagaactgggtgattctataatcacgtcaatatcgactatgactagcggtatactagaccttaaaaagtttttctcttgtcgattttcggccacaccgggacgttctaccctgacgatatacccgaggggtgttgagagcgcaccggcgtttggggtatggagattgactgatatagtggatgctgtcgggggggtttactgtgacatagaaggatctagatcagacattagagcgtatattgccactctcaggagtgatatgaaacaaacaagtgatgccattcgtgactgcgagaccatgatcgcgcatatcgaaggatctaaatttgtggattcattcaagaatcttctaacagcccattctaaattaactaggatacaaaccggactcctacttagagccggcaagcttttggtgggatcggaagtcccgagtctcaaacacctgaccacatttctaggacgctggaggagtttaagtagccgttatacaaaggtaacttctaccgccgataacgtacccgaagctgctattctagatatggtgatagacctccaaggtatctggaatgggatacaacatgataaaggtatggccccccgattatatcttacacgcgccaagtgcaggaggatgccgtattaaatcttttgggagaatattcggaggttcttgatgatggggtcccaaccggaattcctgtcactgggaagtcaaacatagcgtcgtgggataccgttgaattggatatattacggaaaggtgtgatagcaccaaacgatatcgatgtggtgcgggccgacgaaatactacttacaaaatcctgggttaccccagaagacctcctcgccgaagttgattctatctttaatgttggttttaataaagatactcaatgaattttatcattgtctgtctatgttatgtcatataattcgggcccatgagaaatattgaaaggcggggatataatcgatgttgctatttcactcctaccatagcccgcatggagatcctccagtgtccccccatgttattaaatacgggtgtgtgtgtacgtagagcgctaatctaatttttggtggtgatgtgtaaagacctatcgttgatatcaagtgatggatcgagaattaccaccgggagccgaaatagtagtggttggctatcgaaaccaatatgacccacagctggggcagggatcatctgtatcttgcctacggtcgtctctgtcgttcctacgtctgatattttccaatggtctgacctcaacactcaccgccgatagcctcgatggaatactcatagaggcccagacttggactctttcgtctaaaaacccaggacatcctcctactatgtgctccattgtggagctacctaataagataacttacgatgatacggagagggccctatgttgcgtattttcgaggatatatggcgagtgtagcttttacacagcaccgggtgccggatttttgaatactcaaattacagcacgttctttttttgataatgtatggggacctcgcgtagaatcctatacactggtaataataggggcaataggaatttcaatataccgatcaggggacacggcatatatcttcgatccacacggtcgtgaaaatatatcccaggcatttgtagtcaaactttcatcaagtgatgtctatgcctatatagccggttacgcccaaatagatccagagtcccaatggtctgcagcaatggtattttttgttacagcaggaccacaaccaacaaattccgaggaacttacaaccgccgtcgcatctttatatggcatctccgagacttatttcatagacgaagattatgtagagcgatcggtagcaagtgcccatccactaggacgcaatccacccagactcacaaccatattagtcggagaggggcagtcgacccaatctaccatcacaacaaaccgacattacgggggtcaatcatggagggacgcattaacaacccgagatagaggaggggataatggaaccacccctgatatatcaaccgtcaaccccttttccaaatcgctgggtattgggcctcgccaaaagcgccaaagcttaccaaaaagaagacggccggcgtggaccccacatacaagcagtgagaatgtgacaattgatgattcatctacagattctggaaaaccggccccaaagaaatatcggagtcggcgtcccgcgactctaacgggctctgctgtgaccgatccgtctatacattctaatcaggactcccaacccgatgacgcactaaataccagaagtggaccttccgaccctatagatctaatcacatgctttaggaacaggaagcccccaataatattagatgttgatggtgcggcaagtgatgatctgatccaaaatatagatgacttagagtctgatatggccggacttgaagtgcagatgtcacatctggataccctggtaattgaatgtatagattcaacagtatggatccataacgcaccaacacatgatgagggtgatagtgatcccctcgaacattttatagttcttatagcggagcgcctacttgggttcttgatagaaaatggaaccaaaacccgatcggattcaccatgtgcattagaatccttgtatactcgggttctcgatctaatacccgtctatacagagtccgtagatattctacgaacaaccggtctggtactaactaatctctcgttggatgattcacttttgagttcgcttcagaatacagggtctttcatgggaataataattctagcgaaattgcgtatggtagccttggcgatacagacatcgacagaggagatatatcttgaacttgacaagttagaaaatgatttaaaggttggggatataaactcacttaaactatatacccacctcgccgaacgcctcatggatgccctgacacaccagagcacaagattattcacaaaatccatagacggtaggacccagcccctggtgaaacgtgtaaattcattatttcaacgtgcgcgcgcgaaagaggccagagccgttcattcaaacgcagctctggcccgtgacatagatgcgctggaatctgctattcacacagcccatgacagattcgatgctgtagaaattctacaaccgacgatcgcgagcggggtcgaagaggtaactagactcgccgaatcattagatagatctaagatcccagatagattgagtaaggttgtagacaaggttaagattatgatcaatgatgctatacgtgaatattttttacgaggggcgcaatatagcgcacgtgccattttaatggataagacaggcgcgtgtcggttccaaatagctggagcagccaccatgggattagaacgtatggtaacatctataccgttgatcgacaaaaccatccaatctattatcgcttcagcaaatacacctgtacaaacgccttttgaatgtattaacaccttgcataacacatcccttttaagagatctactacagactggaaagaatctatctacggatggggcacttgtatcttgggtagctttgctatcagatgccctagatggaggacatataaatcgtaaagaatttgacaatctaatcaaagatattaacatgataaacgaacgggcctctaaagctgcatctctagggaccgagctggagctgtttcgagttctcagtactgctgttgaacaggccgccaaagatgcctcggattcagatctgacatctatagacacagtcattcgaggtgcggatgaggttatgcggcaagttaaggtgctggatgaacatataaccaagggacgtctcacgggcgagtctaaggcccaggtggagataaagcggcgtgagatagaaacattggcacaggcggcgagaaatcgtatattgaccatcaaggctcgtcgtgatgagatatactcccaacttaaaaccctcctgttacccctggcgggatttgttggcctgagggccgcacccggagccattgagaaattgatgcttgatgcacatgccaagacacctgaagaatttcgtctagtaatagccgatgcaccgaaatcaacgctaactaccatccactcacatttatggtcactatttaaccaatatcgtgaggccattgaacgtccaagcgcgactacttcctcctccctattgggtcttggtagggcatttgcaactgtaatagaattcattttagatccaacacagcaacacacgacacatcaattttttatacgatgtgcggatcaactaaccgcagcgataggatctgctactgctgccccagattcagaaacagccatacggggggtcatcgccgcggtagatgaagcagtacaaacagttagtataggaggacagataattacagaatttacgtttttggaagtaatctccgagaaatataagaatcgccttgaaacattaatagaggttaagcaagtagagagtaccaagaggacattgattgatacaacacgaactttagtagaaacagttacacgattgaaaagtacccaagctgatgtcacaaccccggatatcgatatggtattaacccaggggcgacaggttctatcgaaggcctcatcgacgattcataatggagacctgttgcttctaaaatcgatggttgtggattctcctagcgtcatgaaagcccgtcaagatctactgcatctggtagacatagctaagcagcgagtatgtgatctggagatggggctaacggaccttgaaaaaaggcgcttggtgagacaagaagatatgacacgtgaaagatggcgcatcgacatgcatgccgccttggataagatagagacccaatcatcattcgacgtagtcgaacttgtcagacttcgtgacatggcagctggacatgcgtacaatacacatgattttcgaaaacatgcggagcgggccctcgccgcaaatacacacagggctctcaatgtgatagaggaggtgttaaagtttaacccatacgcgccagaaaacataaaccttactatccatccagtgatccagctattacatgctattacatggtgggacgaatttgcggttgccgctccggttctaacaactctgtttcatggtattgatgtggatagcctactacgtttaatgcgtatagcgtctggtatgattaccttctccggtgtgaatggtggtcatctcaaacccaatgatctgataacacatatgatttttgatctaatgcaagtcccccagctcgttaaatatgtggaatattatcgcaaagcatatcttgtctttgaggaggagcgggaccacctgagtaaattacgtgctgatgtacttcaggcgataggaagtcgttcgagtgaaataggtcacgctttggaagaagtgtcatatgtacggaatgttgaagaggccacaagaattattgcagatggggttcatcttcatataccaagtgaaaaacttttgagacatgccattgaatatttagaaacattcgatcaatcccgtttcacggggtctgcatacgcagctataatggtaatgttagtaaaaaatgatctcacccgcgccaaggatgcatttgccgagacgagtctcgcaaaaaaggaagcgaccggacgggccatcaaaattctacgagaggttgtagatgcggcccaggctcaagatagagacgcgagtacccacttagccaacctaaaaaatctattgcgtctaaaccccccaccaccctcaatagccaatagcatcgacaaagcttcttctgcggaggatctagtcacccagatggcccttttcctacaaacggtggaggacgcccgtgaattagacgttaaggctgtggagtggttacaacaaagtcggtctatcatagactcccatccattgaccacaaaaatagatggtaaagggccaatggagccatatggagaacgaattgataaacttttatctatccgtgatgatttgaatgatttgggatcacagctaattgcggcggaagctgcctggaatgaggcatgggacaattttcaacgagaactccctcatattaccaattccagtgaggcattcacctccacgggtgtccgcgcgcgctccctacaagcagcaatggggatcgtcttgggattacgtacgaatgaaaattaccgtcgccttccagcaaaacttactggatccttagatgctagatatgcagagaggaactcagccctggaaaaattctatgatacctttcatgccacggaggctaacatctcaaaatttgaggcgctgcttaggcgaattcctccagaggtggacccagcaaccctagatgtatttctaggagaatttgatcgtattgcaaagtccctacctaaatgggttcaaaaaaaatacgctgtatacagagaactgcttttattaaggattaccctttatggtgaatatagtaaactatctactgttactccatgtaaaattactataggaagtaaccaatttaagaaagggaacctggtagaggatgaacataggcgcctttgtggacgggtcgctgctatcatgggcgatcgtgacgtgatatacactctcagggaggccacttcccatatcgacatactgttcccaaagacttttctggataccaatggaactccaatagaatataaactttgttaccatgctgttggtgagaaactggctatcatgttatgtgggcaatcgggcaaactaatacgcccgagcatgatttccgagccattatcggaatctctatctatagttggtatgcgtgtaatgagcgaggttattcagttacgactcgggtttgagactgcccatcgcgagggattctcgatattttctagattcattcgtcaccggcgtcccgattgggactcttctcagatgtcgaatatggccgccgagacatattcggcaatattggccactacgcttacacggcaatatggcagggtctggaaggatatacgtttttcgacagaaaccggtgaggtgcttaccaatgatgtgataaaaaatactaatgtagatcgaccggcaaaatctggtagtcgcgataatataacattcaccctctcggatatgatgataagccttgtgatgacatttccgttgcatttggtaaattttctcagattggatctaatccgccaacacgaatacatggcaaaaaccctaactctggctataaccgaagctttgacatccaggattctaatcaacactattgaagttaaccctaaacaagaacatggatcatcacatccctggcgcccattaccagtggatgtacatccagaagatatatcacatgggtcactctttgctttaagagcctctgactggaaagctatcaagacttctcttactggcttgttggatgtctggctaagttcccccgatgaaaaaagtagaaccatcgccggggatgttaaaagactcatccctggagacctacttaaaacatttactgtgatggcacgtatgtgtataccggtcaaggtattatcatccctctggacggcgctaaggccagatgtattggatcaacaaaatatgacctatgacgatatgatagatagccgcctagatacagttgctaatatacaaagtacagtctcaacgatagacccggctgtagtcttcgcgacgaccgatcaggagagagataacctatataccccagtcggcaagcttacaacattcacattaacgggatcgccacctggcacgattaagaatgtgacagcaatggatatagttacatgcgccatcttattgggtgcacccctagtggtggccgtggagaatgcagatgtgttttcaacaacatcagggttgaccatgtgtatgaagctcttcgactctagaccaggggggactgatcatgatatagggatggccgtgtcatctgatctgagttcttggggaaatagactcttagcattggatcccaataccatagaaaatgtgtgtctgtccggccaattagatagattatctgctttagtggccagtaaaccactcgctgatgctagaccatgtctgataacattggatgtgggatcgaatataacacatatattttggtcaaacccggtacccccaaaaccacctataatctcactcgcggcagacgaattaattgcagagattccttttattgagaccgatgacgatctattaccaaatagggacgtctccgatccattcttcacaaaactgataagtggtgagaatctaccaaatttagcggaatgtggaagtttttatgcaggcccccctacctataaattctcggatgaaattccatttccatatgcagctccaaatgcccaattgccaagtttttcagcagaatccgttgtagacagtgaaggtatcggagcggatattacgcgtaattacgatcccatagatccacttactgatttggtagtttctacatataatacaaaccttgaaaatgatgatgtaacaaccctctctatagaagtctcgagcatcacgtccggctctacgtcactagacgctttgatgaatgatgttaataacacgaccccaattataggcgactgggaagactggttatctgatggactcccagatgtgaatgcgaatgtggctaacgcagattatctatctgaagccgaaatctcgagtactgttccacactcaccagatgatgtaacctcgggggctttatcggatacctgggagactgtgaagcctatatctaagatattatttacaccggaatctaagtatactcacgattatgacacgggtgcgctcagatctcccatcgaactgagtctacacagacacacccctttaattatcacctcccagacagtagataaaacacagagtcggtcatgtggagactcattaacccacgacgcacctggcctccctgtgacaatttctacatctgctaccaatcaagattcattcgataacgaaacagatgaacacgatcatggaatttgtaccccagaatctataccctgggacggtgacgacggacctgatggacatgtccagatttcgactacatcgagtaatgatcgatacacgccttccgggctcacttcgatggccatatccgtaccccccatcgttctacactccgaaacgcgctcgtcgtttggccctgacggagatcgccaggagaaacccaatgataacatatccgaatattttagaacaaccgaaccacttgccactcagagggatatatcgaccaaaaagacacccggtgagaaggcgattcggggatcgcataagaaaaaaggtgatccatcgcctaagaagaatatcactagcggtcgtctgagacctataacagttacatcaaatgtgacaaccgaagccaatcaaagtgaatttgagcggcttgctacgatttatagagcagcactcgttaccatctctaccggcgtttcttcaaaagaagacccatctgaggaaaccgcgcatgtttatagcggtattggggataaaggcgaaacgatgagcgtgaatatagtgcatagtcccagaacacaagaccttgtttccatttcgagattatctcctcgagcaccccaaatcatcaatccacccaagagacctttaaagagtaagactaaaagggttaataaatataatagcgatccatggacagtattagatactgtggaaccgcctattaaagatacactaattatcaccagggccccggccctggttaattcttgggagtcgatggataacctatcatccgagattgatacaatggaaaagagtccccggttaacgtataatttagagaacttatcactgaatcagacttcattacccacatcagattcaaccacattgattgatgacagttcgggatatgtgaatgtgaatgtggacagcggagggacgagtcccacactacctgtcaccattgataatatcggaactgacagtggggagtcgccccaatattctgctgacacaaccttcacgagagtggtagcagcggacgatctacttcaaggcaggcgatatttcagaaacacaggtataggagtcctagcgctcctgacaactgcgtgtcgaatgatcgccgaacgcctacgtcgcacgagaaagcttcttacggataaaaaacactgcctattaactgacttacacaagattcgtcttatactcggatgaccaccccaaaaacatttttgatatattaaatacataataacaaaatattcgaaatcctgttttattaattttatatctgaagtttttgaagggttcaatcctcgacggtctcgacctgtattagtcttggagagaatgttcgtttcagaccaatagtaggtcgtgcccaatatgacccatcatactcagcgaacatggggaggcggttgattgtatgttgggcatggcgttgacgtagagcgaccattgataatgctgtaccgacgaatagagtcctttttgcatctaacatggccgcttgcgatgcgccctctggacgccccgccctattaagactttgaattatatcaaccgggagtaacccgtttagatttgcagcggtgatggtctgtagatcagcggggttaaactcacttgccattattaactaaacgatggctggttatcgcctatacacgatgggggagttgtggttccgaggggttatacggacgctgtgagaggttaataaacactggcaatttacagatatatacatgggctttttattggtttctcccagacaacaaaattccaagggtgatggctatcacgaatacaaaacatgatatactaccgaaaacgattggtctagcggaggatactagtgatgttttcatagaactaggtcttacaggagaaattatatttgagatgatatcggctgcgtttgctaaaagccgtgatggatccgcggatctctgcagcggttgtggagcatctatagatcctatacgaccgtcatttattccagtcgatacattagtagattttatgttacgatgtctacgtgtgagattgaagtttgaaatcctctcaaataagcgttggctccgcgagacgtcctccgggcccatgaacctaaaagagagttgaacccttggcatgcgtacgaaacaatgtaattgcatcattacccgcagtggacgcaggtccattccactctcacgttttgccacatccaacgtagaaagtgagatcccagtactctgagtagatcctaggattacattagtattttcggagttgatcggactctccgcggccatggccggtttaaaaaagaaacctttaaataaaactgacacaccggtgttttgaatctcgatatatgggtcacagtgtacatccgcccaatcactcataagacgtaatacatattccacaggaaatgtcacgtcattatcatcttgaccactgaactggaatatacatcgagcgggtggattttttgggttggatagatgtatatctccatcgccgtaacgaagattacccgacaccactaggcgtatacgctgcagtagttgtgacttccccataaatatatcacgcgatttgttggccgatcactataattagtagagaatgagagttatgatgtggacttccaagtcctaatgtagaaattaaaacataggatgacaagcgacttggtatatatagagcatgggggggccttacaccaccccacctgccatcaaatcacacgtcgcgtataatttgatgtagatcgagaaaacggtttataacagccgagaaacgtgatttttgcgtggccagagctgtaccatggacacacccattagaggatttcaaatcgtgtatgatggtagatgcgagtcgctgggtatatgataaatagtttagctttagatccgtggtaggtaaaaagagttcaagttctggtggggttaaatcctcaaaccagatatctatgtcatcaaaaccatttagataaatagactcgagactgtcgagatttaaagcattaaggtcgatagaagggatcctatctttaagcctcctggtctgatcgttgctagatacggtcttttgagacataggcctggacatgttccctattgtaataccggatgacgagagtacctggaggaacgacgcgttcgaggccccttatgcagcattcaacccgaggttactcaccacaaacgacactctctgtaacgagctgttatttgctgctcatttaatacagatcccagtatccccttcatcggaagatgacacgttaaatcatggaaatcctataggtgaatctcaaccactacccaacgacgtgtcacagtttgtggaggtgattgctgattcactcgcattggatagaccgtgtatcgtgtgccgaataattgatctttataggcggaagtttggattggatcccaagtggatatctgattatgctatgctatgttccaaatgtctatccataccactctgtgccacctccacgttcgtggccgcctttgaatttgtatatctaatggataaacactatctaagacatgaaaatgttactttggtgggggcgttcgctaggcgaaccgtcacaccgatagatattcaacgacatttttttctacatggctgctttcggaccgacgggggcatgtcccagataaatacaccaaattcgttatcactacaaaatctaaaggcgggtagattggagcacgacaaggttcagttttcaaactattcatttctaatccagtcttcgacgagagccatgttatgtaccacgcgtatccaaaacacttcaaccacccaccgtgtgagcgatggaactcaaccagccaattgtaacataaatgcaccaccccaatcactaacaaccgccttggttagttggaaggattgtgcgaaactcctcgactgttccaataatgaacggagacgacgcgggtgcctgacgtgttgtgagcgagccaaatcccaagataatgaatatgaggaactattattgaaagaggctcaaaattaccgaccccggaataatgaacgagattatgcggacctcgttcttttgttgttaagtggtataaccacctgggaggtggatgatcgtataactcaagcggcgagaatgcgtagggtatgtgtggagaattactggtcaatacacagggagaagttagtaagcgataccgcccctagatttactaaatttactggtgtggatgcctctccaaatttggaatttggaccaatactcttaacgacactaaaacacgcccaggttagggggagaactgtctcagaatgtatcttatgcaatctcctgatcattagagattattgggtggcattacgtcgattcaagagggaggtcgtcacatattcagcaaataatgcaggactgtttgattgtatacctcctattttggaagcatggaaggatgaagtcaccagtggtgaagcgtattcctttagtggggatggtgggcggtttataactttactttccgcggtgggaactgaagctatatataaacacttcttttgcgacccaatgtgtataatctccgagcttcaaacaaaccctcgtgttctatttgaccatccaacttcatcggagaaggaggatctagaattatataaagcgcagttggcgagtgataatagatttgaggggcgtgtatgcgtgggtctattgacattggcgtatacctttaagacctatcaggtctttcctcccaagccaacggccggggcagcctttattaaggacgccggagcatttttccgccgccatagtatctctttggtttctctggagcatacgttatacaattatgtctgagaaacggtccgatcggagttcgcacgagcgcgacgctgaagtgcgatctgcattgtgttctcttttgaacgttcgtcaacaccataggaaatcctttcatcattcacgtaaaaatactcgcaaggatctccattccacgacaggagtacgaagaactaggggggattccacaacaaaacaggcacgtcttctagaaaaaactccaatgattacctgtttcaaagaaagagaacgttatagtctatattttgattatgtatcggtttctccatcagatgaactcgcggccgtacgagacttggttgttcctatagtaaaaactacgccaatatcattaccgttcgatctcaatcagacagttgccgataactgtgtatcgctatctgccatgggatatcatctagggataggtggctattgtccaacatgtgccgtctccggagaacctcgtctatatcacactagccgggcggctttagttttagcctatgtccaacaattaaataacatatacgaatatagagtatttttggcatcaatcatctccctcgctaaccacatcggtggtagtacatccacacaaatacagggttcagcggagagatttttagaagatgttctcagtcaaccagaattgttttttgtatatcatgttctaagagatgggggaccacaaaactttaaagttctattctatcgcgaccttgaattttcggggtatatgatgtatgtggtatttccaggaaagaccgtacaccttcaccatcgtttattggacagactattatctgcttgtcacgggtataggattatcgcacatgtgtggcaaactatgtttatattggtggttcgtcgagatggtgaaagacagacggatatggagataccaaccgtgagtgcaactgatatttattgtaagatgagggaccttagctttgatggagagttgctcttggaatacagaaaactttattcaatattcgacgagtttcctccaccccgctagtttcgatcaatgcctcaaacccatattcagataacttggtttgtatccgtctgtctactattgatgtttccggaataaacctcttgagaagtatttcagttgttttgacatcatttccaaacaaagctttaaatgtaacgctaatcgtacttaaaagatgtgaaaagtaatattctgtattcaggtgtatgccattagctattacataggatgggtcttcagcaagatccgacacgagcaattttctttttttggtattgttgagggccttattgattatagtcccgcgcaacccatcgactccaagcaggtcactgtccgacacctgcgctggtgatataattacatatggtatacgttctttcacactaggcacttcctcgttacgcataaccaatttatagtacaccgtgagatgggcaatccttttattgatatatgcatcaggtggacggctgagttccgcggtcataacaaaatcgttcacatcgagatctggagcggttatatgagtatatgcttcagccagtacccttccaaattcccaaaagttggcaggcaacgcacgtcccatccattctctgggatgcacgcttgcgatttctgcggctgcctcggaaactgtatcattatataataatatatccactaatcggcgagcgtatgtattgataaaattacagttattcttacgaacaaggtcgacacccttcataagcattttccctccgttgatcacaccgatgtatttcttctttgttatcaatagtaatttacagaaagttttttcacattcgagtttgattggagctctgaacagggcttgtgaaatttgcttggccatgagatcacctaatctcgttactccatcatacgtgaagcctctaaacttaataaatacggaatctgtatcaccgtagattacctgtacattatagggtttatccggaatagtaacggtcatagcttctggaaaatcactttcgactccttctctagttgcccatctagactccacgtaatgtttagtattgagtagcatatctcttcctatcgtagttacagtggcggctatctgtagacatgggagaagaccattagccaccccggtaaatccatatacggagttacatactacctttatggcagcttgttgtttatccagtaaaatcgcctcgtctgatgtgctatcgggaatacgggcccggatggcctttctcatggcaagccaatcgcgaagtaaaacaccaagcaggctttctcgaacatgcgaatgtacaaaatataatttatgtccaccgacatcaaattccgaaaaatctattcctggtcggagggtttcgagagattgacgttctgtggtaagagtcgtgaaacatagattatgggcctggataatacttgggtagaggctggcaaaatccaatacaactactgggtctatatgaaatccggagattgggtctagaaccttggccccttgatatccaacagaacgcgatgtcgcttgagatggggtaggagccacacactcggctacggtagatgactgctgatcgaattcctctgtgtaatcaccttccatgctgtcacatgtgtttatattttctatgtcctctgagggtatattcgggtctccacagaaacggttgcggttatcaggtaggatgaaatttctgtctctggctaattttaggagacacgtgtatacccggatttgttgcccgtcgaagatggcacgtgtcagagttatatttgctaatttcgctatagcagatagctccagatgtgggagatatttgaaaaacaacttccccacgagtagagaatcttggatacaatattcacctattatcccccgttgcctggcaccgctcgcgtaatagcgtggaatttctttataggggaggtctattttatactcccctaaggcctcctccgcgacggcgtttaatttatagctagataaacgaagtttctctgtggcgacggtgtacatatcaagtgtcacaagaccattaatcttgaccttgctccgtttttgaaaatttactcggcccatatcccagactttgaataatccacctcgattaaattttccatatccatccaagcgaatattgtataccgattttagtttctcgatgatataggcccaatcgaaattgacaatattataacctgtagcaaactcaggggagtattgttttataaaggttaggaatgctataagcatttcaaactcgctatcgaattctaaaatgatgggcgtcggctcccccctttcagtacttccgagcagacagctgtctggtagatcacaggaacccagagaaaatagtagcgtgagttcgtgtttacgagtggccaatgaatatatcaaacatgaaatttgaatgacaacgtcatcttggtttgtagcgacaggaaatgctaattcagtaccagttccggctttacactctatatcaaaacatagtaatttgtagtctggccatgtatcatcgtttggtaatgcctctaaattgtcaactgtacaatttatctcaacatcacttgacgtcacgtatttatcgattgatcgtaattgaacccgctccccgtttgctccagggcgtaacctataccatccaaaggttataaatccagcattatctaatatgaacctggatattacatcaacgttcccttcatattttgtaatttcaggatgaaaattatcacaaatatatccacataatcgactgctaccggtagtaattttataatataacttgggtcgtgagtgaaaaaaataaatattggtcgcataaaccacgtctatcttgaaacattcttgggaaattttcatattcaatccacgatcccccgaatgtctaccccccaccccattatttatatatgaatttttaatggactcgaccatgagcgacaccaattgttgattattagaaatattacacaccctatccactagatccttatccatgtaaaaatatggattcactccatatacatgcacggctacccttttaccacacgcactaagacccaataacgttataacacttccattgggacgtataaagtttgcaaaacgcgaagaatcatcccctgtagctatttcaatattttcgataatatcgtatacatgaaaacgatcaaatctaaaatcgaagttgggtgggagtaaggatttttgctcccatgtatatatgcgtctaggccatggtagttctgtgctatcattaaaatcaaatatgttatattcttgtccctcgcgatataccttcggttgtctggagagggttcctatatgcatccccttgcgttggtcaggatgtagattttcatcaagacagcggggagctataaacttaaattcagccagatctgtataatacgtaggtctatatccattatatcgcatcgattcatttttaggtagtctggaggagtctccacaggcccctctgaataggaatgggttgaaaaaaccacctcgattagatagcatgtttcgatcctccccatttctcatagctcgacgccgttgatcggaggtaaattatagacgagcccagataagaaaaaagtttcgatgtgtatctagatgggaaattaatatccttttacacaaaacctactacctacatgtgacctgtgtgggcggggacattcacacacttaagtataactcatacccaggtatttggtatatccactcattcgtcttagtttacaccaggtacaaattacggtgtagagatagcttctcatcgagacttaataggataatacctacaatggaatctgccccaaagaccatctcacttcaagccggtccactcggttacatctatgcatgcagaagtgattccctgcagattccagaattggggattctatcggctcggagttcagactctgatttagcggttgttcctctgattcaaggtctcactgtagagactggatttattcataacgtagccgtgttgtctggatcgaaaacctctggtctcagtggacatgggatatcttacaagcttacccccagtcactaccatccaaatgcctttatatttcatggaggggagaccatcccagcatgttcaatggcgccaaatctccagcgagcatgtgaaaccgcaagaaagcgctttggttttagtgggtatacaagccccccggtcgatggagctatcgagactaccgcggaagatatatgcaagtctttgagacttgatccagaaaacaccatactctatttggttattactgaaacttttaaggaagctgtatattcctgtaatacatttttacattatggtggcgtgagtactgtagttatcaattctcaagacgtactcaggattccgctttatccgatccagttatacatgccggatgtaaatagggtatcacatgagccgtttaagaccaagcagaggtctatatgtgatgaatttatctactgtcaaccattcttcaatgatcgtctctgccgtcttatccacgggtatatcctaggtccagctgcagttagtctgcgagtgcgtaacctcgacgcacttgcacggggagcggcacatttggcctttgacgaaggccatgaaggaggtgtattaccacaggatgtaactttcacactgtttgattctggttcaagtgctactaaaggaggcagtcgcaccggtgcatcgcgcgggggagatggagcctctgggaaaaatatatctacaaacggaatagaaagaagattggcatctgtaatggccgctgacactgcattatccatagactctctaataggtgctggtatgtatggagactccgttccagatattaatcaatggcctatgttagggggtctttctggtccagatcgattggatgcccttggagcctacgtctcaagactggctggattggtaggagctatgatattttcctctaattctgtattgtatatgaccgaggtagacgatggtggggccgtggatggtaaagatggtcccaatccctcttatcaccgtttttacctaattccagcaccacacgtggccggtaatccacaaaccgacaaggacggccgtgttttacccactatggcgaaccaaccaatgatccccattaatggttccggacaagaattttctgtggattatctggccctgatctgtggattctgcccccagataatggcgagattgttattttacctcgaacgcaatgatgctggggcgtttgtagggagaggagaacccgatgccgtgaaatatttaaccaatacattcgacctggatgtcccatgtaatctctgcactaaacatacacgtcatttttgtgtacatactaccctctatcgattacggcaacgcctacctaaatttggcatcccaacaagagccccgatgggtatttttggtacaatgaacagcagctatagcgattgtgatgtacttgggaattatgcttcatatggggcactgcggagaccaaacgacaacgaacctcctaggagcatcatgaatgatacctacaaggcaactatagacaaactactcggtgatatggagcagcaagggttactcgagagagactcgctgatgttacccaatgcatcccatgctctctcgcgcatcatcgtcgaccacgctagctttcggaacgtcttaacctcagtaaagactggtgtggagcaggcgattgagcaatttatgcgacatctagtggaagttagagattttaaaatcagggatgcattggctgatgcaaatcacaccatgtcaatttcactcgagccatatgcaagcgtcctatgccccgtcacgtcttttttgagaagacgcacacttttggctattatccaagatttaatacttagtcaatgtcattgcgttttttacggacaggccgttgaaggtcgaaactttcgggtacaattccaaccagttctaagaaaaaggtttatggatctgcttaatggtggatttattacatcgaagaccgttacagttaccttggcggattctggcatcgtagccccggatctaacaaaattatcaacagaacctcctattaaagactgtgatggtgatatagcgcgggtgagtatagaagtcctaagggagatcagaatcaagaaccgtgttttgttcgctaccggcattggtggaaatctatcagatctcgcccgttcacgcatagcagggatgaccagcgcatatcaaagacccgagcgtcattcaaatatattgacaggggccgtgggttttttgttaaaacaattccacgggcttctattccccaatggacatccacccgggagccagactccaaacccccaatggtattggactctattacagaggaaccagatgccggcccgtctcctgagtaaggaggagattgagaccatcacaacggtcaagcgattctcagatgaatatgccgcaataaattttataaatctaaccccaaataacatgggggagctgatacaattttatgcagcaaacctaattctgaagtattgcgatcatagtcaatttttcatcaacgggataacagcactgctatctcaatcaaaacgtccaaaagaccccgcatcagttatatcctggatctcaggtactttacgcagcgtctccgatatagaaccagctatacaagacgtgttatcggaagtgccacgacgcccagaattatggactggtacatatacatcgacaaatatgatgcgagttgtgatgggaacacgcccaatggttgtattgggtctaagcgtcagtaaatacaatggcagtgctgggaataaccgtgtattccaatctggaaattggagtgggttgaatggaggaaaaaacgtttgtccactaatgtcatttgatcgcacacggaaatttcttatagcatgcccacgggttgggtatacctgtgagattattgggggtggtgggggggtccgagatcattctctaggagaagttatcaaaactattatttcagaaggtggacccatggttcaaacggctgtattttcggcaatactcaattccatgggttcccgtgttcagcacttggagcttgaagattggagggctgtagttgatgatgagtttttggcaattaatatgagagatattaatacccggatctcctcacaccccgatgggtggagtatagagaccgcacaaatcgtcattaaagaaatggaaactcaaactagcaattatgttggaatcgatggggacggggtcgcgtttgattttggcacatgtggagaaaatatgacgggtcatgcccccttagttggaattaagcggcctcacccagaagatgatctattcgacatgagtaatgttatagaaaagaaggctggactcacattcgatatgttataaatcatgtctacgtaaacctacgtaattttgacgtattttaataaacaaaatctatatgttgagttgtgttcttgtattttctttcaatatgaattcaggtgaaatcaagcatatcggtactgctgccgcgcgccaacgcctgcttgtgattttaggccagattcaaacgtacatattccagatagaattgttgaaacgctgcgatccagaggtgctacgacgttatatgaacccacttaaacttaatgctcttacaatcagatttctatcacggaagctgtttccggcactggggaatcaggcgcgacatatacagaccccattaacctatgctgtgtggatcgcacttagatatgcatccattgagggggagagacttattgatgtagctaccacatttatagatcgatcattccttgagttttttaaggagacgatgaaactcgaagcaccgtgtgagtatcacaaaacattaaacctgacaacctatggtggaaatgttattacagagatgcgatttttacatgacctggaaaattttctaaagcaactaaattattgccatctcataacctcgttcgggtcggcgctacgagtaatggaggacattgatatgtttatggtaaagactcttggtggtgggtttatagttcctccggaattattcgatcccatgcaaccatgttctatatgttttgaggagttatgtgttacagccaatcaaggcgaggctgtacatcgccgtctcttagggtgtatatgtgatcatcttacacggcagatgagcctgaaaatagaggaagattatctcgttaagcatctcccatacattcctgggttggtggacgagaagcggaatgcggcccaagcagctatagagaaaatagaggctggtaggcgaaaagctattgataatcattcaaaaaacgaccttaccgataacctagcagatggcctcctcgacactcataacgtttttaagtctgcaccaaagagcttatatgccgtaagtgagctgaaattctggctggcctcagcgggattggacggagtagaacgccggactatcgactcgttcactgacaatctacagacgttgagtttacgcgaacaacacgttagacttcagatagttacagcagaattggctctatttggaagatctatgaatcatttcgaacgtgttgtgggagagggtttagtggattttgatataatcgataatctcatgatcgggagttatgtaatctcaccagacgatcaattaggggctctcatccgcgcgtgttatgatcatcacttatcaacaccactactccgccaactgaccaacccagataagagcgataaagaggctttaaaacaattattggaaagaataggaaataaccaagaaagcagtaatgaagaagagtgtctcgatgaccacatcgagacacagtcccaatcggaccgggccatagactcccccgaagggtgtaatgtgctacctcaacggtcgtgggataagttattggagcgcgccgcttccgatgccgcgcatagacgccgtctctatgcagagcgtctatctaaacggtcccttgcaagcctgggtcgctgtgtcagagagcaacggcgtgaactcgagaagactctacgggttaatgtatatggggacacgttgatacacacgtatgtgtcggtatacaacggtttttgcgccagacgagcattttgtgacactgctgcccgtgccggtatggtaatagacaaccgctataatgaatcggtgtttgattcacatagttttatgaaggcttctcttttacgtcactcgattgacccatcattacttccatcgctaactcataagttttttgagctggtgaacgggcctctattcgaccacgacagtcataactttgcccaaccccccaacacagcgttttatttcagtgttgagaacgttggtctgcttccacatttaaaagaagaattggcgggatttatgttaacgtccacccggggtgggtggacggtgagtaaatttcaaagattttactatttcggtgatgatacgtctggcgtcacaacaactcagcggttggcttggaaatatatccgtgagctcattctagcatctgccatattttcctccgtgtttcactgcggtgaggtgaagttgctacgctctgatcgcacacgaccggctaatacaggtacccagatctgcccacccggcatttatctaacatacgaagaatcatgtccactcgtggcgatcttgggaagcggcgacgagggagtcgttggcagggacacagtggctattttcgacagagatgttttttcccttctctactcggtattgcagcgactggctccagacatggtaacggatcgtcgggattaaccagactagctagatatgtttcatttatctggatcgtactattcttagtcggtccccgtccagtagagggtcaatctggaagcacatcggaacaaccccggcggactgtagctacccctgaggtagggggtacaccaccaaaaccaactacagatcccaccgatatgtcggatatgagggaagctctccgtgcgtcccaaatagaggctaacggaccatcgactttttatatgtgtccaccaccttcaggatctactgtcgtgcgtttagagccaccacgggcctgtccagattataaactagggaaaattttaccgagggtatagctgtaatatttaaagaaaatatagcgccatataaattcaaggcaaatatatactataaaaacattattatgacaacggtatggtctgggagttcctatgccgttacaaccaaccgatatacagacagggttcccgtgaaagttcaagagattacagatctcatagatagacggggtatgtgcctctcgaaagctgattacgttcgtaacaattatcaatttacggcctttgatcgagacgaggatcccagagaactgcctctgaaaccctccaagttcaacactccagagtcccgtggatggcacaccaccaatgaaacatacacaaagatcggtgctgctggatttcaccactctgggacctctgtaaattgcatcgtagaggaagtggatgcaagatctgtatatccatatgactcatttgctatctccactggtgacgtgattcacatgtctccattctttgggctgagggatggagcccatgtagaacatactagttattcttcagacagatttcaacaaatcgagggatactatccaatagacttggatacgcgattacaactgggggcaccagtttctcgcaattttttggaaactccgcatgtgacagtggcctggaactggaccccaaagtctggtcgggtatgtaccttagccaaatggagggaaatagatgaaatgctacgcgatgaatatcagggctcctatagatttacagccaagaccatatccgctactttcatctccaatacttcacaatttgaaatcaatcgtatccgtttgggggactgtgccaccaaggaggcagccgaagccatagaccggatttataagagtaaatatagtaaaactcatattcagactggaaccctggagacctacctagcccgtgggggatttctaatagctttccgtcccatgatcagcaacgaactagcaaagttatatatcaatgaattagcacgttccaatcgcacggtagatctcagtgcactcctcaatccatctggggaaacagtacaacgaactagaagatcggtcccatctaatcaacatcataggtcgcggcgcagcacaatagaggggggtatagaaaccgtgaacaatgcatcactcctcaagaccacctcatctgtggaattcgcaatgctacaatttgcctatgactacatacaagcccatgtaaatgaaatgttgagtcggatagccactgcctggtgtacacttcagaaccgcgaacatgtgctgtggacagagaccctaaaactcaatcccggtggggtggtctcgatggccctagaacgtcgtgtatccgcgcgcctacttggagatgccgtcgccgtaacacaatgtgttaacatttctagcggacatgtctatatccaaaattctatgcgggtgacgggttcatcaacgacatgttacagccgccctcttgtttccttccgtgccctcaatgactccgaatacatagaaggacaactaggggaaaacaatgaacttctcgtggaacgaaaactaattgagccttgcactgtcaataataagcggtattttaagtttggggcagattatgtatattttgaggattatgcgtatgtccgtaaagtcccgctatcggagatagaactgataagtgcgtatgtggatttaaatcttactctcctagaggatcgtgaatttctcccactcgaagtttatacacgagctgagctggaagataccggccttttggactacagcgagattcaacgccgcaaccaactccacgccttaaaattttatgatatagacagcatagtcagagtggataataatcttgtcatcatgcgtggtatggcaaatttttttcagggactcggggatgtgggggctggtttcggcaaggtggtcttaggggctgcgagtgcggtaatctcaacagtatcaggcgtatcatcatttctaaacaacccatttggagcattggccgtgggactgttaatattagctggcatcgtcgcagcattcctggcatatcgctatatatctagattacgtgcaaatccaatgaaagccttatatcctgtgacgactaggaatttgaaacagacggctaagagccccgcctcaacggctggtggggatagcgacccgggagtcgatgacttcgatgaggaaaagctaatgcaggcaagggagatgataaaatatatgtccctcgtatcggctatggagcaacaagaacataaggcgatgaaaaagaataagggcccagcgatcctaacgagtcatctcactaacatggccctccgtcgccgtggacctaaataccaacgcctcaataatcttgatagcggtgatgatactgaaacaaatcttgtctaaccaaccagaccatctctaaatttttatccacaaaaaaagttagagataataaattttgaactcaaaaatatcctgtaatgtcatcattctccgcccattcacgtcacggtctctttaaaaataacccggttttgagggttaggtacacatttctctcgcgccgatcaatccaacacagaagctaaacactttttttccatcgataactatacatggagctcaatcgtacacccgagaacatgaccactgaaaagcctaatcctattgtacaagaccctggaagtgatccttcaccaacgcagcatgaagatgtgaccacagcggagactaaatcgaccgacggggtgaagtttttcggatcatcgtttgtgatgagggggaatttcgtatgccaccgcgatccagattgtcgggcgtgtcgccatttaccatttcgtcattcataccgcgatggactcgattcacctcacctatcggatgcccttgatatcattggatcgaattgggttaagatccctcgagtattcccagatactcccgtggagccctggatgcgggattatgtgatccctgacttggatgagtgtgtagatagtatggaataaaacacccgacaactgtttatcatgcgtgctattgtttattattaatattttaataaaccgatgggtgggatgtatcagcgagcgttcatcatctgagatacaaagaaatccgtatcatcacgtactccgagacctgtcacggtactggcatccaccgttgctgcgatattttgggagtgattggttccagcaatagccacatctattttcggtgtgatatcgtttgtttgaatatttgatgtaatggggacattctgactgttaatatttgtaggaggttgtgaaacacagactggtggcgttcccacacagggttgtaatgctccgcttgggtattgctgattctgattattcggatgtagatcctgtctgggttgttgataaacaccaatcgaacgcaatgctctcagttgtgtattttcttgttgtaatgaggacacaacccctgccagttctgctataatggcattatttgtcccactacgctgtgtttgttgtggtgggctttctcccggataataacactgatgatcgtattcatcacgttggccagaagaatcccaatcatgtctcctcttgcgagcgtatctacgttcttgggacggggaggtggccagcgcctgtgtatatccagaatccattggctccatgcgctgggaaattctacggtctgatgttaatgccccgagcaatgcgctaatttgcgcttcgagaccactcgacgatggagtaaatccatatggatggaaatggtttggagggaggggtggatggtagatagcagtagagggaaatatattaggtgggaatgtagaggccggggggtattggataggaacattcgccgcctgatgttgattaaccacgagttgattatactgtgctgttgggaccaagatataatctccagtgtttgggggttgtggatggcttatggtagaattcccctgggatgccatctctggagtattactgtacttctccgtagcagactctgcgttgggtacagatggaacgtctaaactggtatgagctgagtcgcaggcaggctcccccgttttaccttgcttataagcggtatttccatcggactccaccccatttaaaacatcacgtatgaaatttgatataccaactctggtactagcctgtaggtacaaatgaccggatattcccgcttccttgcggcgctttgccactagatcccatcgatcttttaataacatgctatttacggctgtcgatagaagggcccgtgtcagggcttgttcggtgagacaccaccgtctaccatcaagattgacttccgtttcacgtgcacgcacgagtaattcacgggttgattctggggtcagatgtctaaatggtgcgataacggcctcgggtgtaacgtcatacgtaactatcgtcccgaccctacgacctattgcacataacgcaacatgtgcaaagagagtgtcgtcggggtcttcatccggtgataatctccgagatgatagcgatgcagatggaagataattactgacaagatagagtaattgctccacgcgtgtcaatttaccctcctttactccaaaaaaccccggacttgcggccgatgcgagtaccacccccaattgcggacaatttagtaaccctataaaaaatggtcctttaggatcgtcgacaatagatagtacggaaccgatgacacatttacgattatgatctatattaatctgtagtggtgatgctgggggcaatgctgcacgcaccacatcacgagttaaagtcaattccccgccatctcccatatcatagagggctagataaccagccacgtagatatggagatcggatgtggtatcctcgataatatcgtgtgtatccattgcatgtgaagaaactggtgcttcctactggagagccggttgaaaataaaaagacccacacgcaccaggtgtgttatatttaatatgtaatgtttatttcgtatatgatctacttagcaaaacatttcagttatatagatgctgtgaattgaggtatataccccaaacacagaaagtatattagatcgtaatcactgccaatgttaaattgccataagcgatgattttcctcaagagaggctctaagtcgtggttgttgcatgagaagacccaagccatgttcagtttgaagagatatagcgtcgtgtgccgtgatcacctcacctatgggtactgttcctgtccggcgtctattttcgagttcgagaccaaccaaccgaattaagttgtcctgcaaacgtccagttttcacttgcaccacgcgcctcgcggtgcgggtttgtgctatgagggcttgcgcatctaaacatagcgctgtaagacccggaaataattgcgtgatctcgacatctggcatagatgtatagatcggtaatatatatctagaacataaaaactctagattgtgactctcgctttttatggtgagtggtgctcccaaatcccccccggttgccccgcgagtcaatgtacccagcatctgtatagaggctgtctggggtatgagtgttccaagctggaaattattacgtaatttatccgtatagatgtttccattccatagtaagcgtcggagtagtaatagtgaggttatagtgtttatagtagctctgagaagcgtctgatcctccgtgagaaatagattatgtgccctccctaggaaagctgccgcggctctattaaccgggtctacgaaagcagtctggtctatatctatggctatagccgggggaccacctttaatatgtccaggaatcggaggtaaaaccttaaattggagaagtagatttaatacgctatggtcttcaagggcatgatgttcataccgtccaccattaggcgcctgaaattgatgtctaggtagtttatctatattatatatataaattactctaccagacggcatagtacctagggtgttagcaataatatccaatgatgtcggtaggtgctctattagatcggaaaatgtagtcggtgggtgcgtcatgattcccttatcgagttcccgatgtaaaagatgaagacataaaactgcacattcaaaagccgagtaggctctattcccgatatagagtcgggcacaggattggagagacattattgttgctgtcatgaaagttttagacattcgcccatctcgataatctatacttctggtggttaacggacgatctgtgataagacgatcttgtaacgctctataccatgaaccaaatgatataccatttgatcccgcggcacgccctaaatataccatcgttaaaaaatctatggctaggtttgtttcatatgccaatggagtgtcatttttggtaatctgaacctcaagccctccaggtgtctcatccgatctgcttccaccattagactggagaggtctagtatccttatccatctcagcgtccgccgtttccgcttcccgtgctacctcctctagagaggccaatgcgttagccacatttagtacctgttgctccaagggatatagacgatcctctatatccgtttcgcccaatagtttttgatgggttaaattatccagggccgcggcagcagcctttcggcggacatctagactttcaggagtagttttttcatttaaataaaaggttggctcacgccaataatccactggaaatgtgggagtaataaaatttctagtgtccgataaaatcagatcatccccgatgttagttattcgcatcgagtcgaatacgtattcgactcgtccagtcatgatgacccgtcttcgacaacttctcctctccaacaaatgataccagtgattaatctctttgatcctaaataaggttttgatatatgatctatatagactcattacccatcacatatagggcatattaatactcacgtggttgcgtagcggagtgatcgggaaagagagatgctacccattttaaagggttactttcgccgcttcctattggagttaaagtgttttttttatccttcacagatacacacgatgacatcggtacgtggttcgtatgtttatagaccccagacccagaccttgggtattttgattttttcttggagcggattacaccaccactctcgattggattgtattcggcgagcttggtaatattagcagccagaacagaaatgttactcgtaagcatctgcgggggtaaactagtaactcccaaaattctcagaccgcgctgcgcgataaatgccaatatgggtattagagtgagcttctccccccctggtggtagaatcttggttattaaacccacagaatctgttaattgtttcaaaccctcacgacgttgaatgtctttactagttgtatccatattttttgaaaaacgacacgttttcagctcaattagaaaacatataccacccccttctccctcaaattgtatagtacatacacaatcaggtcggcgacgacccaagttaacctcacatgctaggtacacgcccttagcctttttaagagactctgcggatacagagccgcccaataaacactcgagtcggtcggtatatactccactcgcagaggtcgaggatatatcgcgcttgaggacagcataaaagcgattgtggcatcgaattccagcccggagcctcaatccgacactgcgtcgttgttcacgtttcatcatacacagatcagacgatggcgagtggaaccatccccgttcagaatgaagagattattaaatcacaggtgaatactgtccgcatttacatagatggtgcctatggaataggtaagagtttaacggcgaagtacctggtcagagcggatgaaaatcgaccgggatatacttactacttcccagaaccaatgctatactggcgtagtctctttgaaactgatgttgtcggtggtatctatgccgtccaggaccggaaacgacgtggtgaattatcagctgaagatgctgcctatatcaccgcccactatcaagcaagatttgccgcaccataccttcttttacattccagactatccacaataacaggatatcagaaagttgtatgtgaggaacaccccgacgtgaccctaatcatagatagacaccctctcgcctctctggtctgtttcccactcgcaagatattttgtgggtgatatgactcttgggtctgtacttagtctaatggcaacacttccacgagaacctcctggtggaaatctagttgtaacaaccttgaatatcgaggaacatttgaagcgtctcaggggacgctcaagaaccggagaacagatagacatgaagctaattcacgcactacgcaatgtatatatgatgttggtacatactaagaaatttttaacaaaaaatactagttggcgtgatgggtgggggaagcttaaaattttctcccactatgaacggaataggctcgtggaaactacaatagtttccgattcgacggagtcagatttatgtgacacattattcagtgttttcaaagcccgggagctctccgaccaaaatggagatctacttgacatgcatgcatgggtcctcgatggacttatggaaaccctccaaaatttacagatctttactttaaatctggaaggaacccctgatgaatgtgccgccgccttgggagcactgagacaagatatggatatgacatttatagccgcatgtgatatgcaccgtataagtgaagccttgacgatataccattaaacattagtggtgttccctattacccccctgtggtgaatgtgtggaggtcaggggataattgtataatgaccatcgtttcatgaataaaataaccgtgtgtgatgtggatgtattcattaattgaatttctcttccggttttagatctttataagcgtaaaactggtgttttaaatccaagagccgggttctttggaggttggtcacatcatcgccacagcccgtggattcaagcaatctatgatgtgtttgataatatacctatcgatattcctgatcattgtatcgaggatgttgactggtttaccgatgatggatagacctgatgaaggtgggctggctcgacgaacagttggtgaagtagaaggggagttttcttatagggacgatgttgatgtagcagacgtgagaaacttatttatcatgttaccaaaaaatgggagcgatatatttctattcatattcgatagacgcagtcaacgtcaacgcggtactatgtttttattccccaaggctgggtttgtacaaccaacacccgcgaaggttcgcgatgaagcggcggccgccccatttgggtttatatcccctgtatatccactatcgagtcttttatttaatccatacaatgggagatatctgacgacacgccatctgattgcctttgaggtaaccccggaatcctctcttcatgattggtattttgcacgatcaccaacaactgctactcagacacagccattaggacatataactaaccccccccgacgatcgccaaaagacaaaccgaccacctccggccatacagatttaattatacgctattgcgcattggagttggattttttccaggacacaagacgacagcgtgatggaatatatttacctaattacgaggccgtatggccattggcaatgaattttttggaggggatgtggatatggagtaatcgtactttagtcaatgtaacgatcggtgttggctttatggggttttctttaacctccatctcttatccacccttggagattatcgtcacacctcactacaccaatgcaagaatgataacacgatttaaatctagtctagtattagatccaccgggaccttcggaaggcccattgtataaagtatatgttttaggctatggtaacaataggatcaatgggagcttttataagaccatgcgtacgatagcatcatacccagaacaaagcctagattatcgttaccacctttccatggcacatatggaaacggccttatttttatcacacgctacaccacaagacatggacggaaccacagcttatatttcaaaaatttcaactaggttggcaactgctcttttttctctttctgaagtacggagattgagtggatatgtggccattgatgagctaatagacttggattttaacacccgtcttctcgctaatacattactggccgatggaatgcaaaatttccaggatccaatcaacattacatattattataattcggatgttggtaggacacatcttcgcgatgcattggacactatcgatcatcaacacgtttcacatgggagccttataactcgcgcgagatacctccagttaatttatacgtatacatctatggaaaagcaatcccaattatcactcaaactctccggtgatatagtcaaggacctatacctagaaaccctttacagtgatgttgtcagatggaacacaacggccaagcaggcattatttctgagttcgatgctgatctatatagctggaaatatacagagttctgtggagcaagaggcgattaacgcaggtcgtatgttatttctacaatgtacgtcaatgtgtacgacagaacacgcttctaccgttaggtggaacccacaacaaattctctatgatctaaccaaatcatcgacaagatttaatatttttgatggtttttcaccgtgtatggcatctaatagatatgatataatatcaacttatggaatcctggatctgttctcggcatttcccatttcatcgtatcggtccatagaaaagccggcagttgattctaatacccataacataatatttaatctgcgaaacctttacacgttcattccggagctattttcatgtccgggtgtttcatctaatcatcagagaccgatagctgttttacctattggtattaactgtacttatcttataacgagacgtgacccaagacgcggaacattatacatagttgatggtatagatgtatcaaatccgataataatctcatatctacgcagcggtgaatgtggtatagagcgtgggataatattacccggcaatcttaataacccggagaacacagaccagtgtctatactgcgggtgtgtttttatgcgttataaatcatccggagaaattgtggatctgctcttgatcaacgataaggctgttgaactggagttggtggctggtgaaaattctacaatatcagcattcaatcccaccaaatactcatccccgtctagtgttttattattatttccaaatggaacgattgttacccttatggccttcacctcacacgaagtgattgtattctcaagtaactttatctgggcgtctataggtggagttttcgcggcctgtcttataatatatataattataaaaatgctctgtagtttcacaccagatgtacgctatacgctattaaataattaacagtgggtaattaggtctcctgtctcttctttcaccgccacacacttttaaatatgacccaaagaaacccatataatacatgaattggatataaaagattagtttattgaggcagtacatatttatttatcaaaaccgctacatcccccgagtctaagacgacgagtcaccagtttatatataaactgtgccccgcgcgcagcatcttcgtggatatcaaacgccgtgatagatttcatagccattgcaaggtataccgggacagcatttcggatagaacgacatgcagccagggcacagccatctattccgccaccagaaaagagcgcgtctaggagtttggcgatttcaaagtccacacgtctcagctcatctaccattagaaactgtgtatttttcttctcgtgttcagctgtgaatagggtcagcatttcggcctccttctcgacagtcgggctgaactgtttatatcctatcatgggatgtaactgcatgtaaaaaattaacacacttaatttacccaagatggccccttctccaagtaaatcgtttaccgcgtccaatgtagtatgtacatcaccaggtttgggttctggagtagaaaaggccctagaatagctgtcggaaagtcgttcgagtgtagcgtagcaattgaacaaattccatcgcataaaaataaattggcatagcacgaacctctgcgctggggtaattttattaccagcggaggacctagcgcctaaaaatggtttctctccagtctccccgaataacgcagatattgctgtgttgattgtactacgggattcgttgagctcatcatccaacccgtcccaacagtcatctatacttagtatctcatcaaccctctgtatcagggcacataaaacttgaatagaattaactccagatgcccgatccctaggatcccagatattagatggttttgggatatagcgcacctgcacaaaattgctaataatcatatcacgtcgcaacgcgcgtgtggcataatgagttggacgaaccgccatggcaggtactgctcttttagatgtcactatcacatctgtagttatgggtggttcatctccattgataggtagccggggacagggaatcccatcaaaaagtccatcgattaattcctctagagctttcggcaattctgataaatatgcttgtatcagtctaaaacaagatctatttggggtatatacaaatccagacgatgcgtttgtaatagtgggtacctcaaataatctcagcgttccatcttggggtagatctcgtcccgtagtcagtactatacctgatgatactttaagggataccatgcactctgtcaggtattgatcgtagatctcaagctcgaatcctccgcacaggtctctatcggaaacatctggtccgtgtgataataggtccactttatctagaaaaacatcctcatcggtttgtggaacagcccatccccgaccggtattattaagttcactacgcacataatttgcaactaaacgatccccttcccccacaccgcgtacagtgagaccaaatttagcgatctccggtcgatttttactttgtgggcgttggaccgatattagacagccaccatgtgtgaagtagatgcgattcccattatctgcaatatagaaaagtactccccgggataccatagtgtcatggtatttgaactccatggctgtagacggtataaaccaacggacttgatcgtggaatggctttaaaacgcagaatgctctagactaccacacagattggaagagtttaatggtctaaagagccttttgattgggagaaaaaacataaatgaatttgagctcgttgatgacgtccatagacatgttacctaagacatgacatatcttattaataactcctcaattagataatacatataaccgaggaggtggcctactactccacacacagcggagctgagtaaacttacatgaaatagataagggggggtgaattgttatatgattgactgtacctgacggggacgagagctttcatcggccgccaataaacatctgaacattatttaccatcatggctacactatgtcgtgatgatgatcaagcgggtataataattcatgaccaagagctagatagcgacgatgaaagcgtttgtgagtacatctctatgtccgcatatgggggggatgcggattttttgttaagctcagcttataccatcgagccgggtaaacaccaacccacctttacaatatctgtagttttttttgccatttcagcatttgtaattaaaccattgtgctgtctggcatttttaaactattacagagcgacaggtaatagtatattcataacatctgggggtataattaccttagtatattacatacaatcaatgttaatgatattttttatctataataatatacgtgtagatttgttgccattgcgatggtggcaacaagccgtgatcgggtctatgtccgttggtagacttatcgcctttatagtgatatttcactccgccatttttgtagactctgaactatttttccgcgtctcatcatcggattctcctatggattatataacacctcttatagttcatactctttttccttcgatcagtgtcagaacagtagcttttatgcttataatcgcggccgctgtctatgtcgcggatgctatatgtgacgccgtggaattcgtcgccccgcgtatgtgggtctgtattctgatgcggacacgcctctcattttagaggtattacgggtcgcgaatataaaaagctaccacgcaccgcgagtgctattcactctttcatcaagctccacaaactactaccacgtctccattctcccaatatactgttgaatagacacgatggatagaccaaatgaaccctttcgggtgccaatagccgatttcaccaccccaacaggacatattttatctacaatagaggtggcatcacataggactcttttcgatttttttaaaagaatacgctctgacgataacggcttatacgctgcacaatttgatgtcttattgggcgcgtattgcaataccctaacctttgttcgttttctcgaactcggcctctccgttgcatgtgtctgtaccaaattcccagagctgaactatgtaaatgaaggaaccatccaatttgaggttcaacaacctatgatagcacgcgatggaccccaccctgtaaaccaacctatacacaattatatggcaaaacgtatcgatagacggtcccttagtgccgcattttccatagcggctgaggccctagggctcatcaccgagacttccattgatggtacacatatctcgaccgcattacgcacaagagccattcaacaactagccagaaatattcaagcggccctggatgcctttgaacgtggaactgccgatcaactcctccgtgttttactggaaaaggcaccaccgctatctctattggcccctcttagactatatcgtgatggtgggcatagtatgggagctgtggccaatgctacactattatcagaacttaaacgccgggtgatcgatgagatgttttttatgaccaaatatgaccgtcatcgtgatgttatactcacccacctgaatgatctggttaactgcacagccccatcggttgcaatcgcgcgcttaacacattccgatactaaagggagacctgtagacggggtcttgataacgacctctggcatacggcaacggctcttaaggggaattgtaaatgttgaagacagtgctgcagacgtaccggttacatatggagaaatgatgattaccggtggtaatctcgtcacagctctagtgatgggaaaggccgtgagaaatttagatgatgttgctcgtaatctcctcgggtttcaaaaggagcaagtaaaaacaaatgttcacaatctagaagattatgaagataatccacgatctgcgcgtgtgcgagcagatctcgtgcaaattggtgatagactagtcttcctggaagccttagaaaaaaaggtatatcaagccacgcaagttccctatcctcttataggaaatcttgatcttaccttcattatgccattgggagtttttaaacccccttcggaaagatacgctcgtcatgccggaagcattgtaccagctcccggacaacccgattcccgtgttttcccaccacgtggtctttatttttttaataaggatggggtcctccaatatctatcattcgagagtgctataggcaccgtctgtcatagctcttttctagacatcgaggctacgatattagcgctcagacagattataccagaactaaattgtgtatttggagcatatgtagcacaaccacaacgggatgatctaccgacacagatgcgtcattttggaaatcaatggcgtgttatgatgcccaatagaccaagatgggttctcgaagcacttatgacaacagatcaatttctctcccctggtaatcagaatctgatttttgaattacatccagcgtttgatttctttgtaattcccggtgatgttaaccagccaggtccaccagaaccacctcaggtcatgcctggacttggcgtcagtctacgtataatcaatggaaacataccactaccactctgtcctatcgattttagagacacccgtggcttagaattgagtgtcgatcgccacagactcagcccagccaccgttgccgccgttcgtggcgccttccgagaccagaattatcccatggtattgtatataatagaggcggttattcatgggagcgagcggatattttgcggcctcgcgcgtcttatcatccaatgcatcaagagttattggcagaattcacacaatgtcgcctttgtaaataatttctatatggtaatgtacataaacatctatctgtgtaatggggagctgccagaagagtgtacaactatctatagagaactcctagaacatacacgtgccctctggcgcctggttggagatcatacattaccagccgagcccatggctggtcgggatcacgaggagctaaacaacattctcatggaccatgcccttctccccccactcatctgggattgtgatccgctaatctaccgtcaggatttagcacaaaatcgtgtcatagaattatgggttggtgatggtgattacaacccggtaccgtgggtcgagatggcccaggtaaatttcagaaataattctggtgatctgatccacaataaacctgtacgcggggaaaacgccagggccccaatcatcccacaccacggtccggaatggtcaactctatcgaagatttattattatacgatagttccagccttctctaggggcaactgttgtaccatgggcatccgatatgaccgagtctaccagctcgtacaatccctagttatcccggatctgggaagggatgaggaggcacctgcatcacccgatgacccccgccaccctctaaacccgcgacatttagtggctaactcctttaatgttatgtttcataatggccgcataatggtagatactgatgcactattagtactacaggacgtagtgactaacatggccgaacggacaactcccattctcaccgccgccggacccgatgtcggtgcgaccacggccaccacccgagaaatgcgtacgtttgatggcactctacatcatggactcttgatgatggcctaccaacggaacgatgaaaccctcttcgatgggacgtttttctatcctgccccggtaaatgccttatttgcatgtccggatcatttagcggctcttcccggtatggcgggtgaagccttgagattggcacgggacgtaccacccgtaccgcctttcctgggagcaaattattacgccacggtacggcagccagtcgctcaacacgccgctctgagtaaggtagatgagaacaccctcacatatgcgcttatggctggatactttaaaatgacacctttggcctttatacaccaatttcgcacaggattacatcctggatttgcgttaaccgttatccgtcaggacagattctcgacggaaaatatactgttcgcggagaaggcatccgaatcttattttatgggccaacttcaggtgagtcgccacgaggcggtaggtggtgtcaatttcacactcacacaacccagggccaacgtggatctgggtgtaggctttactgctgtatacagcgcggctgcgctacgcacccctattaccgacatgggcaatcttccacaaaacctctacctaactcgtggcagtgttccattgttggatgttgatacggataactttttgagacgtgctgtcaattctggcaacagactagacccacaggagccagcaccgttatttggccagataatgccacgtaccccagtcggtattacccacggtcaagccgccatatgtgagtttatagtgacccctgtttctgccgatttgaattactttcggcgaccgtgtaacccacgggggcgcaccacaggtatgatctacacgggggatggacgacaaaattccgagagtatcatgtttgaccatagtcaaggagacccggcatatccaaatagggcgacgataaacccatgggcctcccaacgccattcgtacggagatcgtctatacaacggggcatacaacctcagtggagcctcacccgtctacagtccatgttttaaattttttactccaaccgaggttgaaaataaacatcgaagtcttgcccaactgcttgtagaaaccgggtccagtttagccccaagcaccgccagtacagaaattcaattcaaacgaccacccggtgcatccgagttagtagaagacccttgcggcctctttcaagaggcatatccaccgttgagctccagtgatatagctctactgcgaacatcaaacaataacgagcgtggtacggatgagacacacttggcacaatacctcattcgcgatgggtcgccattgaaaggctgcatcgccaatgcgtcataaaaaggacttccatatttttgagaacatataagaaccaataaagtatcttttatcatacacttgcgtattcgtccgcgggtgaactagtacatacgctcttctctcataacctcccggtgacggtacctgtctatagtcgtttatcatcgacaatattcacaaaatactgggatggcatcacaatcatttgagatagaaattctcctccccggtgatatttcacaggcggacatgacagctttacagaaatgtgaaggaaagatagtatttttaaatatacttaaacgcagggcgactttgggggatatagcgttatcatcctattatattaatgggtcccctccggacaccttagctctcatggcggcctacagaagaagatttccagccattattatgcgtgtattacctggaaagatgacagccgtggcgatcggcgtggcaccacttcctcctggtgcctttatccagaacactggaccattcgaactttgtaatggtgatgcaatttgccttgtacccccattaataggcaacgaggatcgactgcgattaacttcttgcggtttagatataatttttccatccactgtaccagtagcccaggcgagggaaatgttagcacgagtggttgctagggccgctgagatgttaggtgccggtgggagacgggggatggatataatgttctacaacggccgcagatatcaactaactccggatatacaacgaagggatggtatagatgcagcgacgaggaccctcatcctgaatatgatatttctcctcaacgagggctgtttattattattgaccttgataccgaacctactcacccttggtgcgcaagacggttatgtgaatgccataatccaactgggaagtgctactcgagaagtggcccaactaatccatcgaccaccagtccaccctccccaggacggcacgcggcgattttgtgtgtacgaggccttagctacatgggtaacctcagcatcacgacttggtgatataatcggtgcaaagccgatattaagaatctgtactttcgatgcgtctccagtgatccccgtgggagagcgggcatctattgtacattcattcacatagacaataaacacggtgtatatttttattgatgttgtattgggtttaaaatgactaatatctccgcggtcgctcgtctatgactctcataatgatctagaaagcggggcaaatgaaccatccgttggggggagacttccaatgtataccgccattaccagagctaccatcatatcgtcagatgcgccgttttttttaccggtatacgtccttgtatcggctgggcctgttaccacttcagtgagatttttcatttgggaatacagatattctaccgggtccgtctggagacgcactgtaacagatacgagttcttgtgatgctagaacatgtccagagttaaattgtctaataaagtggtcaaacgcccccgtcttctgtttctgtaatagaaaaaatggatatgcgaccagactacctggaggtgaacaatgataaaataagatggtcggaggaatgggtacgatcgagattgaagagagctcctcaatttctctttgtatacatctagctatagcaacagctgaatcttggctgctgttcccctctacggctatcttcacggtggtgaatggacgagaatgcagcgcaaaaacttgagccaggctctgcgcggcacatctccctatatcatccgcggaatttccagtaagagccctcagaaaaaaatgttccaggccaaaaatgatccaatctgagttatagcgaccgacaattgctatgccggtgcccgatgcgcgtgagtttgttgtgaatgctgggtctatgtacatatacaaatcggtggataataccgactgattcgccacagtggaaggcctgtatatcataaacctctccccggccgtctttgtaaaaactaactcggtattggttttatgagagggtcccccacctataatttcctgcaagaaagagtcttgtaggaagagttcggcggtattacgcatggcgctatccatagttataaacacgggcttatttaaaatataacacgaacaagccgtggcgttggtgtgtgttaacactctctgcatgtgttcatcgcatatatacgtaacaacatttaataattcatcagccgctcctcttaagttatataaaaaactcgtactggattttcccgtgttggtcgaggatacaaatatgatcttacagttcgtctgatttaaaaacccgatgattgtctgtacagcttccggtcgaataaaatttgcttcgtcgacaaaaagcaagttaaaatcttggcctcggataccctggagggaatataaaagagggtagaatcaccggttagtgcagctcaaaaagctttaaacttcattatatacacgattatttttgtacccattcgatatatatttactaccgtagcagtttctcatctccataacctatacatgcattatggatgctcatatagccaatgagacgaagtttcaactaaattatggtgtgtcgcctagagaccgaaccttggttcatctcatcatcccaacagcctgcttggtgaaatctggaattgtgcccacaaagttaataaaaccaacggatggtgaggatctccccacctcatctttcaaggtgataacacagacaaggtatcatgcaagtggtaaatgtacgccatgggagtcgacatttggtggttttatcccaaatggtgcaattaccaatacccttgtgccaacatgtccgcgtgatcacccttatctatttagggtatctcctgatcccggaggattatttatcactcttgaaatctattgtgatgtcaatagacaattcgacccgtttaatattatagccatacgactggagattccgaatgatagaaaaacatatgaactactattcacgtacgatgaattattaccacctgggacgagatatggtgcggatgcttctagactaacattcctatgcaagaatttttccgcatatactaaacgtcatcccggtctacctgatagagcggtagtggccggggccaacatagaagcctgtatgggggatgatacaatatccaaccacacccaatcccttacacaagatggaactatcgatgctctacaccaactggtgaatggagggggttttgacactcctacggctatatcacggttggaggatacagacagagagataatgtcattgatcaggcgtgcctcggaggccataaccaccagacacccagtaaggagggtgccacagcatcagaaggatggttgtggagtggcatctggattacgacagggtgcaatcgggatatcaaatcataaaaacagacccatggctaccggggatgttagacaggagaccgacgctttattatcaggccttgagccacgggggtccggacgttttattcaaactagtcgtccctatacacacagggaggaaaatccggagaatcttcttataatctctcaggaacaacccaagcctctcacacccctagactggctcgatattggacatgtagccctccttggtggagataccccaagtgatgtgtggcgacgacgcccgatctctctagtagcacgtcgccactatatcaccggagaaacattcgtagtggtatcatatgaaaattctatagcctggggtgggaggagggccaaagaatcaaacctagatggatcgctgattaagataatctctgctgagtgtctagcgcatggtataaaccatccaagggatctccaatatgacgtacgaaagcgaataatagaacaatatcctatgctagaagttccattaggagacctgccaccacctatgttggcattcgatgtcgcggcagaggttgtattactagagagatttaagaacgcatgcatacaggccctgatcgcgtccatgacagaaacgataaccacacagccacgcatgtcccaattattggaatatgaactccatgatacacagcgggattatatacataaagtggtaaatcggataccggaattattaaaggccctcgtggggtccatcacccagatatctgttcgtgattttcgagacagctcattaatgcttgctgccctgggacacttgaatgcaatttcaccagcctcgggaggtgtgggacgtctaccttaccaccaggcttggataccaaatatatcagggggggatgccatgtacttatttgactacttcagttcttctggtagcgttttgagactatcggcggaacctttggcggtattattaacaaaatgtgtgggtgggcaatatcgttgtagattcgctagagttagtcagggctctggatcaagatcatatgaacgctatctacccggcgaatgctatgcttacatatgcattgggttcaacagggaattcaggggaattattgttttccccgggggattcgcttttcacgtgacaccagcggaccatttaatttggcccgcccatcttttcgaggccattttaagtcgcttctgttggtctatcccacccactccctagctcgtgttcgtcgtgtaaacatcatcaatcatggagagagcttcccatcctatatggacagctacaggaccggaacgacggttgacacaagatgccgttgagcttctaatagaagctctttcggacgatatctgttctcttaagccgatccgcagtgactcgcggattaaaatctacaagggggtaggtctgctaacaccgaggctcgctccatttactccagctccatctcaactttcatccatcaaaaactattgtgtatccgtgacattgtacataactcgaccaaaggcgctcaagttatctaagggtcaatttcatatcctagctttattcggtatatccacagcatatgcgtttatatccaatgtcatcctgaaacctatgcttcccgacagtacgattctaggggcttcgttttacgacgctagtgccatccccctcccaaaggaggttccagaccccaccgcggaactgattatatgtgatgatccattacatctagatgtgagtatgtttgcgacgcccgcacagcctccagatcaacgctatgattgttgtgttctagctccgggggtatggtggtcatatcgagaatgttctatatactatctctgtatggataccacgctcctcgccctatgcccctcgggatggagatgtagaagcttatgcggtatattggccagattacttaatcatcgggaaggttgtggattgtgtcaattcgagggacatgtcgacgcgcttaatgcatcatccgaaccatcgaacataccaccagagtcatgtctatgctgggcgccctgtttatggcgcaaggccggtcaacgtgatcttcccgtggagggggatcacgtcttattcagagtgttgtttatggatgcagtggaccgtattagattaccaggatcgccgcggaatccaaaaattaccaacaacatctctgatgttatcagtggaattggctcagatggacgacagatcccagccaatggggctggttggaacctagttgtcctagacgagaatatcactaaggctctaatatgcagctgttttcacctaaggagattatgtatggattccccaccctcggacaatatatctaccacggattgaatgttattagaccagcgtctgtattataaattcctatcaatagataatacccaataaaatatatattatactcacgtttgtgttatgactcgatgcgaacacgatggtactcttagctccatccggaaacgagaatgaaatagtttcccctttaacgtggtccactctagacaccccgaaccattgacgcaacctcgctccaatttcctcaaacaccggttcggtagcttttcgtatatgggccgtatatcccactttaataccttttatcgttgccaacgtaacagcaatcaatggaactaaaaaccaggtttttccatgacgtcttggaacgagaaatactgtagcgcgttgtctgaaatggttaatacaggcgtctgaaaatgtgggggtgttgaaaaccatttttaagaaaatgcttatgcgttccgcatgatcacctaataacacagacgctataaaatatgtggcgtgcattaagatcattttctgaaataactctagagtccctctagttttaccataacatgccacatcaacttttgctcgttttggtgggtgtcgcgttaaaccaccatcaaaattagcatccctgaaagatgtatctacgagatgattgaatctcctcacaaacgctaatatctgttcaaaagcatccgacgtacgtaaggcctcaaatgtattcattacactataatatgcatttcgatatgcgctacttgaggtattatcatttttggtgaacgtgagtccctgaatcgtctgtaacatatccgctgtcaaaaccgaaccggacaccgcctcctccccagtgtcgtcgttgctgattcgcttccggaccatgctagtaaaaagcaactgccgtgctatgcaagagaaaataggtgcgctttcacagcagtcgtgtagagctcccaccgcgggtacaacggtctggtggcgctggggtgtcgatatcgagaaattaaaaaagggtacacgggtatcgtcgtctacatccgtcgatccctccgtgatttcacggagggaacctagacgggcttgacgttccctttttaaagattcaaaatatccgacggtttgctcattaagtgatctaccgaacatattaatagtcttcaagatacccgataactctaaaggatgaattcaaaacgaactttccgcaggcgaagattggaactagaagaagcttaccaacgtgaatcaatatttaaggcacgtacgctagacatgatccgtggagggatagataggcgtgatcctatttttgttcacacatttacgtcagcaaaacgggccgggcaggcccttgacagacaactacacacatactctagggtggaggcgatagaacaaaagacacaatcactgcgactgcgcattgaggcacagacggccgtcgaagaagtactgcatagccatcgacggtatctccagtcagacttcactgataaaattgatgctgtggatgataagatatttgaaaaggaaaatcaattaaacgaaactattctagaagtagatccagaccccgaaactctggaacgtggatggctagacgaggcggacgaagcgctactgacgagatggatgttggcggaagctcccaaggcgaccgactgtcacatggaccaatactctcacctatcacaagaccatcatcgggggtccgggaaggggggggacattgcaacacggccgatccccactctcaaggaaaccatatcaaacggggtatatgtaaaccaggagtctctggatccggtaacacagccgatagcgctcacaaacacctcaccatgtccccaaggaggctgcgcccattaccacacagagaagggatcttacgtcaccggattaaagaagaatgtcaagactttcaggctggtaacggagaaggtaaaatcagggcaaacaccgccatcgacagatattttacacgagcgagacgtatcttcaaatatacacccagacgcatgtctagtagacgaggcgggagaaccactcccccatgtatggctgggtgggcttccccctctggtggacgatatgacgggctcattcgaggggactccaacaatggacggaccgatataccaaataccctgactcgaattcctatacatgaggtatgtaccccattaacaacaaatcccggcaacaggtcatctattttgaaaattaggaaaattaagcgtgttacgatccctgtgttctcagtgtcagcagaaatgcattactctaaggtggcactaggagaaccaccgaagttcgggggggctggtgggtatggagaagtacagatttatcgacaaacaggtctggccatcaaaacatcatcaagtccatcgtgttttgaacatgaattattagtcactttattagccggggagagctctctacgcgctagatcatccataggcataactgggataatttaccccgttgcattttcattaaccgaacaccaaatggtattcaaagcgtatgatatggatctgaatgtatattgtaataaactatcatccgctggacccccaacatcaaatatacttaatgcgatggaacatgcgttcatcgggttgggtaaggctgtggcatacctgaacaccaaatgcggcttaacgcatttggatatcaaatgtggaaatatattcgtcaacacaaaaaattttgttataaaagattatgtcatagccgattttagtctgatgactctaaacacaaattctaccgtaatgcgggcggagtttgaaattcccactggggatgcgtcaaataaggtcctacgcctttcacgaggggcggcgacaactatatttagtctggtattgggtcatggacataaccaacccacggagatactggttgactttattaataacagtggactggctcgacaccgcggcccattagacagtgacgttggtgtagctgttgacttgtatgctcttggacaggtgctattggaactgcttttgactggatgcctttcccctcggttaccggtccccattcttagaaatacgacatattactactacctacaccaggtgaccgtggaatatgccttggatctcttagcatatcgctgcgcactatacccccatatatttccttcttcacctattacaacaattcatggtgttccataccctgcagtagaagatatcgcctctaagatcaccgcagtagatcatcgcagtgcattccacgctcaccacgagagatatcagaaaacccatcgccatatctttgaaagtgtgcgcgtaccggacgaatatatccatctatttgaattggtggctatattttgtcattctgacccgatcgcccgtgcttctataccagtgttatggacttccaattaaccaacccacgccattcatacgcgctacgaagtcaacgcaatttagatgggactgttttaagaggggataccacggttccattccccatcgaagacgctgtaaattctacgagttccatgcaaacattcctaccatctgtacttcctaagagcttttcaacattaacgttttggtcacatttcttaaaaagctctccattaccaaaaaatgataaacggttgaacccaattttttaccgtcttgcttatatacatgatctagtgggacagatggaactagagggtctagttagcggttacctggcaacaaccacatatggtatcggtaagccgatacgatttgtggaaggaacctatacacacgccaaaaccctcgctgattcattaccctctacacgtatacaagatgtaatactacgtatcgaagcggagactcgtgatcagagtgaaagtgatctatggcgacttttacgcagaggactcgcgaccgcatctacaatgcgatgggggccctatggacctgtatacccaccccaatggtgtgagcttagtaccaatgcacgccccattcccgacaacccggcaatggcgtttgggagaacaaatgaacctatggtacgagcacttatcgaaacgttgtatatacctcctatagaggtccctaagaacgaggagactcgaacagaagatcccatgttctacattttcgaggaaagtcccaaacgtgagagaggtgaggcgtactcatgcggtctattgatagatattcgaacaggggttgtgggggcgtcccttgatatgctagtctgtgcaagagatgatctcgggagacttaaaccctctaattcacaaacaaccctggatttttttgaaattaaatgtcgtgccaagtacctttttagtcccgaatcctcttccccgccgtctatagcatatgagaatcttctaaaatatcgcagtgccgcgtctttgcgacggttcatcagatcaatcaagaatccatgtgtggaatacttcgaacctaataaagctcccagttccacagaagcactcaccacatgcaattccgcatggagaccacgcctagtagcagagactaacagacgatgtggggatttcgataaacaacatctatgtttgaatttagatacgtcatcggatgtctggttatttagtgaacctgatctagagaggggtgtcattactcccgtctgctgggataccggcgatctcacattatccacgcccatcttcgccaatccgagacacgcaaactttaaacagatattggttcaatcgtatgtactctcgggtcattttccacacagacgtcttagagcattcctcgtgacgtatataggcagacatcgccgcatcggtgaggctggcaaaactttcactatcctcgatcgtccgggatcacgaagttataatctacacgagaccgtcccatctacttgtgctatacccgtactcttgatcgttactcctgtgatagtggatcacgaagagacgtgggcagatattgaaacgtgtagtttagaagcgttcaacactacagccgatacgatatgggacagaaattctcctgttcatggtgtcggaaaaatcatctcttgatgcataatggagagaccatgtccctcgatcccagtttttttgaggacttcagtttggatgaagattgggactcccttcataatataattcaccaacaaccgcctgtaaaagttcagcccgtacaaaaaccatccagtcgttcccataaaaaatctaaacgaccaagatatgacccctattaaaatatgttgaactgaatatcgcgtgttgtgattaatatatgactattttttatttaacataggatgaataacaagacacaaggaacgttatttttaaaatggttttatttatagaaactatcgcggtattcgtcgggggtgacgctatcatagatcacctcgtcatcatgtgcgtcggagagcagtagatctttatccctgtacagcttgctttgcttggatcgccccctcttgcggccgatataatttttcatatttgctttgacatactgtacgttgtcatagaaattcgtctgcttgcctcgatgatataaacacgctcgtacacatcgtattaccaacataatcactgctattgaagcgactatggcaagtgctagttttgttccaagaatgatactttgatgctgactcgcaacctcgacgtaaaatctgtcaaaataatcattagcggtaactcccatggccccacaggcgatgataattgagaaggctggcgcgggcagtacatgtatgtagtaagatagcattacctccccaaaaattacatatataatcgctaggaaaacaaatataccgatcgccaccgatgtcgtttgccacagagtgatatgaaagctattagctattatcatcccgaccataagggatgtagtggcgatggagaggcccattatacccaatacaatattaaccataacagcacgggtggggcctaatattccatggagccttggattcgcagtctttatcgcacgtaaattgaggctatattgtgagctcgtgatacccaagcatgtgaagtagatacaaaaaagcgatagaatgaaaaaatgggcgatatagatgatagctgcgagaactatgagtttatgggcgagaagtactatggatgcctgtaagagccactgtgccaaagttccgagtaaaattgtagccgggggggataccattgaagatactctggaagcactaccacgcatattcgaagatgatcttaattctcgtcgaatcataagagctgccgtgatgaggtagcatgccaaaattaaaatgccgagtgtcatcatataaaaaaacgccaccgttgtactggtctcaaaaaacagcgttgggcttatatccccatatcgctgtgagataaaaacaccatttattaactttgaagtattgacagaattgtaatcgacgatggtcccatagaaacaaggaaatcctacctctggaaaggaggcgagaataagggttataaaaagtgtcagtgaagccagaacaaatgtggccatttgaatcaaccaggatctccagctgatactgtcaaacacgctcccgatcataatcgttgctggtgttgtggctatgccgggctccatgatggattccccacaagatctctctaatgacgactcacccacacggtcgacgacggacgagtgttacacgtcgagcgtgtctctagcaaagatgctctatggaggtgatttgggagagtgggtcactaaaacccatccaaatgtgcgattggagcgtcaagacggacacccggttatttttccatcaccagtctccaccaccagcagacgtgttactattgtgagggcaccgatgggatccggaaaaactacggcgcttctgaaatggctacagacagccctcgatggctccgacctgagcgctttggtaatctcttgtagaagaagcttcacccacactctggcaggtagatttaacgacgcgtgcctcgagggattcgttacgtattttacgtcctctacttacataatgactggagaccccttccgcagacttttggttcaggtcgagagcttacatagagtggatgatagtctgttaaataattatgatatattggtcctagatgaggttatgtctacgttgggtcagttatattcacctactatgacaaaactaaatcgcgttgacgcgttacttcttaagctactgagaacttgtcctacgattatcactatggatgcgactacaaatgcaccatttgttgactttttagccacattacgcggtggaaggaatatacaccttattgtcaatacttatgcggcaccggggttttctaaacgggaagctgtaattttacgctcgcttggtgtagatatggccactgccgcacttggatttatacccatcgaaagtcgcgagggggtgatggaaatagatcgatcccccttagatgaaagactcgcccgcgtcggtactttcgaaagtttttttggaaaactcgctacttgtctgatggacgggttaaatgtatgcattttttcctccacagtcatcttttctgaactcgtagccagattctgtcaccaatttaccaacgccgtgttggttcttaattccacgcgcccgacggaggaagttggggtctgggcaactacaagagtattagtatatacgaccgtagtaaccgtcgggataagtttcgagcataaatatttccataaaatgttcgcatatattaagcccatgaaatacggccccgatatggtatcggtttatcagtctctggggcgtattcgcgaacttctacataacgagctcataatttatgtagatgcatttggggcacgaagtgaaccaattttcacaccaatgcttctaaatcatgtaatagggatgggatggccttccaattttacgcaaataagcaatctcttgtgttgccaatttagagatagttgcaagaaggcctataggacaatacgcggtctacatctattttcaaagttccgatacaagcatttttttgaacgctgcacattagcaagcgttaatgatagtataaatatattacatacgcttctcgaggctaattgtattagagtccactttgatggatgtccagcccagttatctacagagagtttctgcaaatttttacgaaatatacacgccgaggcgttatcaaatcagcgagtacttcgacagatacgcgagactgtgaatgtagctatcctaccagggtctgaaacatacgacacggacgaggttgcgggttttatctctaaatatttaataataactagcccttctggttttatgaaaaccctccttcaaagactatcaagtccgagcatacggaaccaattcgttaacatagccgttcttggggcctgtatgcgtttacccgagagcttagaaagtgtcgaggtgttcaccgcgttatatcaccattacgcttccggaattatcccggtgattaatgacactgggatgttacttacggagactgtgaccccaaaccacaacatcgaggctctctggagcctatataaaacatgtgcgtatcttaccagacaaatagattgggatccaaatcgtggcggttctacatccgatctaactcccgaggtcatcctacagctgctaaaacctaaatattctatctgtatcaattcattgcttgaagtggctaaatgtcatatcaccccagctgacctcttagctaaacaacctatatcatcggtaagcgccatattaaagggaaagtcttgctctggcaatatataccccgtctcgcaactagaacacgccgtgggaacattcaagttgctctggggtgagttattcggtgctaaattagttaagagtacacaaacttttccgggtatttcacgtgtaaaaaacctccataaaaccgaaataattgcattattggaatcagtgaatattgatcattccgagactaaaacctataaacaattatattccctgttaatgcagcatcgcaaactatttgatactcctagatataaactccgagcaccaaggtggagccgatacatttgttttttgtccaccggagaagccttgtcatgtacagatgagctggacagcgcactggcttgcataccccccgatcaatggccgtcatatcagggtgctttggatttttctcgcctatgataaaccattaggtgatgacagccataggcttatctaattctccgccagtgatcccgattgaagatatcatggatgaccatagaacacctattgatgatattaaatggacacatggctacgtttgcgcggcaagtctttattctctatggatagaccatgataaactaaacgctctcgtgtatctgatatgtaacgaggatggggaatataccgctcgcttcaccgtggtcagcgtaagtgaaaatcaactaatctggggttcgcgtgagtgtccatatatcacgatccgcaaagcagcggcagcggctggagaggcagccgccccgaatatatggccccttggtgccctcggacagactgttttatggaaggcaatttatagtagtgttatggctgtattgaaacgatcatttaagggtttttctttttattcacgtgtggccttctgtatgaatataaaaactggaatgactgtggacatacaacccatcttgaatgataaaaggaccaattccctcgaacccgaggcaaatccggtgccggtcccagccattttgaatgtcacggcaaaagtagatctggatccaggagccataaaagctaattctatgaatactgaggggggaggattggcaagggctcgcctctgcaccctccgggatggctacttttcccaatccatggaagaggatatatccatggacgttgaggtaatcacgaaaagacatacattccgtagaagatatgattctatacagcgcccagctgttaaacaaacgggggactttaaggacctatttaaaattatagattctagactcgttctccaatccaagaccgtaatggtacgagttttaattccaagatattttgattgctttctagtatgctcttctacattttcaataatttctatcatgaccctttataaacaatggcgcgaaactatgttttcgaacaccggagaactgggatccatacctattttattgccgatatttgcatatataggcccggagttgagccctactggggaggatcgcgattacagttgtatgttgggcttcccgggtataccccttatcaaggtagcatctccagatgtagaggctgtacgggaagctatagacgtatacgtgtctaccgatggtatgtggccggcaatgggaatgttagcctttcatctattagctccgtgggatccaactactgatgatgtaaagcgatcacataccaccagactcattcatctcgagtcaaaactaaccaaggaccaggacgataaatggccagctggaagaatttcgagtatactcgaccatccgattgccatgaaggggctatggcttgccaaatttgatttctctgcattctttccgtctctctatcttgccatattcccggagcatacccgtctagctcagataatatcggcccgtattcgtcgggaaaagggagggctgaaggcagcattggtatctttattcggtgggctacaacatatccaacccctagcctatagagtgataattggcctcgccaatggaatcagtagacatctggaacgattattgaataatgaacaattcgggatatgtacgtacatcaaagatgggttttggggtgtgtttggctctttaacacaggagaccacatcactagcggatgttgaaattgccgcagaaaatataagaaatacctgtcagatggccgctatcaaatatctagaggatatgggtttggtgcttcccgaggggacaaatctgcacttacgtctcgaggggatatacacggacgtcatgtcttggacaacgaatagctattggttatggaatagagagacggatatggaagctttcgttggattccctacacgaagtgctatatgtagagcgatgaaaaatcgcttatctatgttactacgcatgtcagcgagaatgggttgctatgccgatttagaggcgcttctgagtaaggcccgggaaatgtgcgatgaactccttgaattgtcgtatatgaaacggtgggattttgagttttggagcgttccaatgaacatagagaatcgacataataaattaccaaatgccatatattgtggtgggagcgtcttggacagagaccatcgcccacgtgaatgcgttcccgtaaaaaatcatgatcatgaagttattcagataccgtggaaattatatccacatccggccattattccccaaatagactgttattccaagttggaacccttgtttggaaagttctctaccatgctaaaccgttccatagaggcatattatgatacaagtgaaacgaagcccgtattctcgtatccaatctccgaatatacctttttattctcttaaaaataaatgattcgaatatatacaattaaatatatggcttttaatcattagactcaactcattcatacattatgaacatagaagcgggtccgatgtttttttctgggttgagccaccatttgtagagcgcggcttgaatttcataacgttgtagtgccacgttcagaagggcgggtcgtgttcttaccaatccactgggacaccgcctctccgatccaatgatcttgctcccggaactagagcctactgtgaaaatcgccgtaagtatgcttgggggttgttttcccccacccaacaggagtttggcgaatatatgatgcgggagaatcctgttgttatcgaaaggattgtgatccatagtatacataagagtgttaagtgtccatgtaacaccagtgaacaatagatttcgtagtcgagatagaggagatagcgtcttctcaagtctctgtaataaggtgtgtacctgtacaaacatagacgatgttgcttcttgggctgtgcagttttctaggaacatctgtaatatacacaatgtgaaatctgatgtagcgtctggatgtaacaggaccgtcctatgttcaagtatcatttcccctactgctaaagacttgacacggtcctctatgggagttatcacaaaaagggaaaatcctttgaactctggttgatttttacactcctccatataggtagtacatgaaatttcttgtgatcgcccctccaaggttaacataagttcatatgttccgtttacaacccttatacgctctacagatgtcgtggaaaatagcggtgggcgtccagagacctcacgcacttcacaaatcaacctaggtagagcgtgccacgtaagatcgtgcacggtatccgggatgtatgtcggatttatgagacatgtctcgtatacggcgtctacggctaactcgttgtatgcgtctgttgccatggttttcagtagaccgtctccacgatcgtgtcttaaatggtaatgataaatatttctcgatgtcagggtctttaggttcggacacgtttgctatatacgctactttgtaggtgacggcagcttttacgtttgctgtaaagagcgttagcaattctgtgaggtatacttccaggcgggtttttttaaagatacttgcacataactcctcctctgaacgatagacttcctgatgggtaatcaggaaacccagtctgcgaactttcaaaatagaaaagaaatatggagctagaatgagagaaatggaactatttgaataggacactgacatctcctggccttgattattggccgttctattcatcttgaaacatcgaaccagttcctgatcccaaagtctagataaacgatccaagtccgctgagtagggggggatgtaccgcgcttgaaaactatttgccacgtatgagtcttctcccatagacccgctcacatcgataacctcatgcgcaaattcacctattgccatcgatggggtagcggcatacccacttcgttgcacatcgaccgccgcttgggtggcacgcagggcacgttcgctcacatttttaagttcgcgttcggtgctttgtagacgatccaacagaccgttcttatcagcctttaagccttctatggttttaaaaagattatttatataaccttcgagcataccattaatactgttgagtacggaggttctaaaggcatcctgaactggctgtgccccctgaacggaagatccacgtttcgttgtctgaccaaaccctggctgtgatgtgtctacctggggtccatttaaaatatggttatttgtctcgtccaagtaagacctaactgtttcggtgatatcaccaatatgacgcatccccttcatatttactattagcttcactagacgagcggcactcgatcctgcagcagcctccccaccatcgcttaggatcttatctaccgctttcgatgcccatgcccccctttcttcttccgaacgttttcctatcaacaccttgaccggtgatgtgtttaatatttgacataaacgagcatgttctcttagggtgtgacaattcaacacttcgctgtggagccgctgtaaaggcgaatcgaataaaactacaccgtcataccatatgggttgccagagaactaaacactccccttctatacccgttacaggatctacagccctaacttctctacgcttgtaattatagaatagatgtactctatcgtaatccataatagatactccatgagcacatgttgtcagctctgtaagtgaagcgactccttccctcaaaatattatgggctacgtgaagtctcatcgacaggtatctctcatctacacatcctattgattggtgcgatcttcctatatcattcccacgttgagggggtctcatcgggagtctctgtagtggaaccataccaagacaaacgatccaatctacatacttggcaaagctagctgtaccgttagattcactggctgaaaaacaagctgctataccacgcagaaagtccaggagggacatatgaagggtcttataccatgtattaaaaagttgctcggctacccttaccgcttccgcctcaccgtggttaccgtatatcgtagctatggatgttgcactgacaccccgttcatctaaatgtgctcgccaatcgcgcatgagatcatcatatttcgcggcatttagggtatttgttaaaatcgttgcttgaatctgccgaattacagcctcggttgatcgaactgcgttgtatactccttgtccttcagtatagccgaaatttcccataagtatctccttgaatatcattgtttttggggtaggatggataagcttccaatcgtccccagcctcaacttgtaaattatcgagagatccgactttatcgatgcagggttccattttcagcccagctcgcgcatccattaacaatccagacttgacacctgtcgggcgcatgcactctacattgacgtcacgcatataggcaaatatgtattggtctgtagattgttaaagttcgggcataaagagacgtcacctcctgatagaggagaaatggcaagtaccgatctcaccggagactattataatgaccaagctgatttttcgaattcgatatatcttaattttacttcaatgcatggcattcggaatatagtcacgcgtataaaggacctagcgggattatcagtttcaaaggaaaagatcccacccgtaagttggtttaaggacttgacgtccctcgaaacaccgcttgatataaatccacgtgaattaccattttccgtatatctcattagcggtaatgctggttccggaaaaagtacctgtatccagactctcaacgaaaccctggaatgtgttataacaggcgccacccgcgtcgcttcccagaatgtatatcaaaaactcgcgtcttcatactcgagcagacccgtcaatactatttttcaagagttcggtttccgcggaaatcatgtgcaggcacaactcggtaaatatcaatatgtatgtccttctaacccaccaccgatagaagagctacagaagagagatcttatttactactgggaagtacttatagacatcactaaacggttcctggagtcaaacgcaacccgtgtggtgtttgacaccataagaagtatagaactcatcaggggacgtgcttccgggacactggctagtcttgcattctgtgttcaaggatctcttccggcatttacaaaaagtaacgttatagttatagatgaagcggggcttttgggacgacacttattgaccgtggttgtatattgctggtggatgataaatgctatttacaacaccccccaatatctagccggtcgcgttccagtgattgtctgcattggctcccctactcagacagattccctcgaatccacattcgaacaccatcaactaaaatgtcgtattcgttccagtgaaaatattctcacatatctcatcaccaataagacattacgaaattatacttcattgtctagtaattgggccatattcatcaataataaacgttgtctcgagcccgaatttggtgaactgatgaaagtactagaatatggtttacccctcacggaagaccatatgcgcctggtagatcagttcgtcgtcccagatgcctttataaacaacccagctaatctacctggttggacacgattatattcttcacataaggaggtgagtgcatatatgaccaaattacatgcccatttacgagtatccggcgagcgacaatttgtagtatttactctacccgcctatacttttgttcgcatagaagcttttgatgaatatcgtcgcgtgacgggacaaccgggactcgtaattgacaaatggctcgcggctaatgccggccgggtaactaattactctcaaagcagggaccaagatacaggccaggtacgatgtgagctacactcaaagcatggggtcgttgctgctcgagcggatatcacctatgtactcaacagccaagttactgttactacacgcatgaaaaaattagtattcggtttcagtggtaccttcaagtcattcgatgcagtcttgaaagatgatgggtttatgaaagcacatggagagacatcggcagagtatacctatagatttctatctgcactccttttcagcggcatgatcaacttttataacttcctacagaacagagagttggatggaatactggtctcggaggcctatagacgactagctgaattaaccacaaacctattacctgtcggaacgatatcttgtaatgataaagaagcagcgggtgccctgggtcctccattaacttccagccaacccttcgatttttctacgctaggcgatacaacaaagatccatcacaagaactgtgatgatgacgacctcatcttcgccgccctcaatgagggagaaatagacctgctctattgtaattatgaatttgtacgtcctgagagcgcccccgaagtttattcccaattcttgatgcttaagaacatgtttcttggtcgctatgcaatttttagagaactctttggaccatgcatagattccgctcaatttaatacctacgtggataatttgagctttaagggttgtgaagtgtttactggtagtgtacgtggagggttactttcaacggctctccaaacagatagttacaccctaatgggatatacacatgttccagtgcatcaattcaatgatgaccttaatcgccgccgaatccacgaggggacagcggaattacttgggtatatgcatataccacgtgtggtcctgaaggaccaccacggttttatgtcgatattaaataccaatatcagtgaatttgtcgagtctatagatgatctagagctcagcatggccacgactgtagactatggtctaagttctaaattggcaatgactatagccagatcccaaggcctcagtctggaaagggtggctatatgctttacccgtggtaatttgaagctcaacagtgtttacgtggccatgtcccgtacagtctcctcgaagtttttacgtatgaatctaaaccctttacgtgaacgtcacgaacgcgataatgtgattagcgagcatatattagccgcccttcgagacggtggggtgcaaattgtatattaaccatccagccagacgggaggtctttcactacaacatccatccctataaccctttcaactcattctctatggatctccaccagacaaccttccacccactagcaacactcatcacctattctcttattggtgtaaagccgtctccggtctggatacttccaagctacgaacaggtgttatgtagttgtgagggtggcacacgatcgatcgccgttggatcgtcttcgcgatatgaccatttacaaccaggaaatatggttattcaacatggaccatcggtaacaattatactcgtggactgtggtagtgaattttgtgcttattcttttactaggctcggtgctgttcgcgcaccacttaccacttcagcgggatctgtgtttatgatgccatttattggttggtccggtgttgggatggatcgttgtatccagagcaattccgggggggttgtgataatcacatggggtttaaacaacactgttcatatatccataactatataccgtctggatagcccgaccaccccaacatcactagcggtaccaacacgggacatgaacgggcatatatccgatatggaggatttactaacgcacacgccaccctccagagtagaagaggctgaaacttatgatttcttggaagcgattttaaaagaatcggaccttcagatatctccgtttggggatattgctgagaaccacacaacgaatcttgatctcaataaccatcatcttattacacccattattccttatacatgtgaggcggaataaattggcaaataaaaagtatgtttggtgatgtttttatttagattacgttataatatacacgtgtatcagtgtctagacgatttctggcgagttctgaatttattactctgcttaatacctccattttctccgattgtacgttgtggtcttgtgcagcatattccagataatgttctcctgcgaggggggtctgttagacccgatgtagaaatagatgcgttgacatgagatccagtgcgtacctcggcatttatggattgccgagtttgatatcccttacatgtcctcatagaggacggctttgatgtagagacgggctgacgttgggagccctcaatgccgccattggctctactaggaggttgtgatttagatccgtgaattcctaacagagcgaggctaccgctaactgtcgactccactgcctcactggtgttggcagacccgcgacgttttaaggtcttatctgattttcccggatcccctgtagcgctggctatcttacgtgccgcgacagggttgtttaccagaaatttccgataatcttccatggaagccgccctcccaacaccaaacaggttcacgacctggctacgggatcgcttatccatgtgctattcttgtggagttgtctcgataacgttatctttatgtagaaagactctatctactgccgaaaactcgtagatatatacaggaacaactgggtgcgttcgaccatccgacgatcgcgtgtaatattttcttggtttcctcgcctgaatcacggattgaatttgggcgcggatctgttttgaatgagcttttcgacaaaggacgaacatttgtaggcttttattactacgaaattcccgcccacctcgagaattcgcgtgtcttttgcggcgtgtggcacatgcgctggagaacgatgaagtcttcgtacaagctattgaaaatttagcgagggtcatgcgcagatctttccgtattgtatcagtgagctgccgcctacccagttcgtctatagatgataccataaatagtgtatcgaatcctacatagtcagggtctgtgtgaacttgacaatttagttgatcattacttacgtcgactctacaatcttgtagattttgggttgtaggtggtgtgttgtcgattggaggtccaccaacagtgacggtctctaccggccatccccattccccgagaatcgtcaaaataggtctgatatcgtccatctacgttaggttggagaagcaccataaacgacgtagttttgctgtgggttttaagtataccgaaggcctccttatcgcgtaagttgttttcgtcatatacttcactcattcaacactccaatcgatggctcccttacccctctttacaagaaactcatttgccattctaaaatgcgtacactcgctaaatgggatacgtgacagtggagatggatgggtatatgtcagtatcaaatgccgatgtggatctgggttacaagctttttgggcgtgggatccccataacataaataccagcccatcagaaatcgatgccagatgatctaatatagccttaactaatttttcccaaccaatcattctatgagatccaggactcccccgtctaactgtgagtgtagtgttaattaataatactcctctttccacccatttaagaagattaccatgcttcccgattcggatatcggggtaattcttccgtagtgccgcgtagatgtttctgagactcggaggaatggttacgttctcatgaacgctaaacgccaggccattggcttgaccgggtccgtggtatggatcttgtcccaagatgactactttaatgtccatgggttgtatcgcacgtgtccatgaaaatatatcttgtattttaggtaggacctcttcatagcgtagacgacgttcgtattccagtaaaatgcgttgggtatacggtttcttaaattcaggttcgagaatagatgcccatgaatgtgatatattaaactttcgttggatagtcatccacgaaatcggtttctctatattggaaaccacttgtacatctggatctaaaattactccagatgggagaccggtgggtctcaatttttttcttggtggctctgtagatgtagtatctgatggagcattggtagtgcaataggtatggtcgagagctacaacagctgaattatgattcacaagctcattcatagttgacatagatataaggttagtatatctcagcgatatatactaaccttttttgtttgtaggttaaggaagctttatactatcttgtagagatggagagtcacgtggaggcggtggaagagtatctgcatccggggcaagttgatctcgtcggaaagctgtgtccagaacatcagtgaatctggattttaagtgtattgggacggacatccgtctgacatcttcagccaatccctgtagtgcaacaaagggattaacccaatatgctcgtttatcccctttaaaccacagtattacctccggtgggttacaatctgctcgcacgataattccagccaaatcttctttgatattttcatcacctagtccatataattttttttctactcggtgatcacggcatggtcctaaaagaattacatcaatcccatcgtattggacttgctcctccgaggcaccaaaatctggaatataaatcttcaagagtaataaaacgaagacccacatcgtggaaagaccagcggtcgatatacctgtgaaaatcacacaacctaatatgtagaacggtctcaaaaaccaactaacccaacccaatagtgagtgccatttatatagtatggtatacaccctgaaatacagtcaaatgtcatatacatttatgtgattcaaatatttattatgcccgtctattagacagtttacagtattcgattatgtagtactcctaatatccggtagatagcgtatttgtctgcaaaacattcttctgtagtggactctggtaataactcctatcgatctataaacaaaaagccatttgtttaagccattaagggggaacttaagattctggaaaaagctattccagccattaaatgggaacacagagtttggaaacagcacactacatccttttagtaagaattcatgaataaaatataattccacatttattatataggtccgtatagctgcgtgcgggtgggagcatctgaggctattacatcccggggcaagatgtacgaggacatcgtgataaagataagtaacaatttcctaccgggggtttatatccagaatattaaatgctgggggcccaccaatctcggcggtctttattttgtataaactgacaacactgagtttcatgtgtattactatctttattaatgatatatccaacaataattacattgttctactacaatctggtattcggtatatgtacgatcggcccagataccctcctacgacccctgcgggttgtctgtaaccgtgtatgagaaatcgcagtggtcgaagagtctcgttgagtcatatttgacgtcgatggggttgatactaaattaaaggtttctgggctccccctcaatctagcagacctccgctcgttccatcgagacgggggtcgtttgcgctttctatcggttggggttacgcttatgggagtattatgactaccagaatcggtgttgggaggtccatcggcatcatgaacacctctgatatcatcctcatttttttgatacctggatgaaactgtagtatttactataggatcggctctgccgtcatcataggaaactgcagtattttctgtagtattggttctgtcgttatttgcgccatctagagctattacagtagtactgttttcattagaactttctgtgtcatgagccagagtaaggtctaaaggttccatattggtctccggatagttctgttgtatcgattcagccggagccgcgaaattggtagccgatgacccgtgtggaagtgtagataactcaggctgtgtatttagtgaatgacgtgtctcatcatctatccctgtagtgataccaacagataagtctgtgctaattgtggtctctgtatcgtcagaatcatcggtatcatcatatgcagtgtcctcggggtctgtgagatctgctgtggcgagactagacgattcatcgtcggaggaggtatcgtcttctgtatcatctaggaattggaccccggttgaaacgcgtaaagtacgggccgtagaaggaaatacgcactgtgatgcttcggcgagcaaccgtatcacgaagagatccgtctgtgtgtggagcaggggtctgagaaggcgagtaagcgcagcgctattgaatgcatgctcgcacagctcatccataacaataccacacatgacactggctgttgtctcatcgttgtaataattcaaaagccagctgttcatccacgttactagagggcgcagatgtggggctccggcacccccgtctggggtcaggggcacaaatcccagtcctgtagatgtagaatcccgtggtcttgttgaagttggattacctgccccatcgctgggtcgtccagaggtatcaaccgttgttgaattttcaagacgaacactgttcagagatatattagcccatcgtcgggcttcagtccatataaacggatcccaatcgagatactcctcctcctctctatgtgggctatatagtttcgtttcttcaaagattttatcgtcctctatagagtggatgagcgatgtcacactagttttacagaggggacatttgttatttaatcctacccacctactgagacaggaatagcaaaatttatgcaaacatggcatggtgaaagttaaatcattcatgggatcaaggcatattggacagtggtctcccatatcagccatgtcgaggtcctctacccagaccaaaaagagttatgtcaggtccttacaaatcaatcagacccaagtcggactcacgactttaatatatacccataacaggaagtgagctcttgggatatatatgttgtcagtgttcatcaaaattctcattcggtagacacatgtgatagggtttcgtttctagaaatacctcccatactaaaatgtaatgttctaatatatcgttctgtgatctatgtaaataatagcagcgcatcgagtatctgctaggcgggctttcttctggatcttatccatcggaaatccacaccggcccaaagcttcaagttactttcactttcagtgatttataaactacttagaatcccataagacctcttacttgtattgattttatgatatcttggggctttcccctgactaaaaattcaccgggtcttgtttcatgttaccacagtccaaaacatttaatataatagttatttttatctcacatgcaaaaaatataataggtggataagtatcaatgttcttttccaaaaaaatcacattcccattcgtatctcggaatggctacctgcatgtctggccttagtactaaatcgcatgtttctatacgatgtatctgatttgcatgctcgtgtatcatgtatcggtcatggggatcccactacgttccactaaaaaccatttctagacatcccgaggttcggatggaatgttggttacttcccccactccggccgccgcgggagatctcggcccccctagccacttttccgagggggtccaaaaagggggcggggtctttttttggggggcgtggctagggggcctccccttccttattaggcccctccccttccttattaggcccctccccttccttattaggcccctccccttccttattaggcccctccccttccttattaggcccctccccttccttattaggcccctccccttccttattaggcccctccccttccttattaggcccctccccttccttattaggcccctccccttccttattaggcccctccccttccttattaggccagatttgaatatgccaacccacaaaccgccagatttgaatatgccaacccacaaaccgccagatttgaatatgccaacccacaaaccgccagatttgaatatgccaacccacaaaccgccagatttgaatatgccaacccacaaaccgccagatttgaatatgccaacccacaaaccgccagatttgaatatgccaacccacaaaccgccagaactacagacaacacacaaaccgccagatttgaatatgccaacccacaaaccgccagatttgaatatgccaacccacaaaccgccagatttgaatatgccaacccacaaaccgccagaactacagacaacacactggtaattgttatggaagtgctgaacggtaggttttattttctttctttggcacagatggacagctgctggcaattgtgtttacaagttttagaacccgcggaggggggtgaccttgtctgggacaccggaccccgtcagatgtaagggttgctgttatggaaccatccactctccgcgtcctcgtcgtcggagacaacctccactttaaccggctcggcagaggcgagcgaccccgggccgttgataccggtctcgaccgagacaatctccacagcctgtgaatcgcccgtatgatagtttggaggagacagagtctcgcttcgccccccgatacgtatcacagtttcctgttttccaaatcccgagtcccacgtcacacccggtatgttctcgatgatagcggactggggatccgccggagttagaatcaggggtggagcctcagtgtcaggtgcgaggagggccgtcctacagaaggctctgacgaccgctggaatccgggtggggtcagtagacgccaacgcacgcctcccggttgccagatacacgggacgtaggggcgcccctagaccccagcgattacaggcacgatggctgtgggcggcaccgtctacgaaatcaggctctccgagcttgaggcccgcactctgagcctccatatccttacacccgtcgtaggcaggaagtacccgctggcgatactctcggggtaacatgggaacgggtgtgcggtcccccgcgtccgtgcggacggtgtagcccacattaccggaagcgcagagtcgcaacccgtcacagtttgggtacaggcgctcaaaggccgcttccacccgggcgaaactccccggtccaaaaatggcgctcgaggtcagaactgccctggagaggtcctctagggccggccagcgcaccgcgcagtaagcggtcggggtgaccgtggcgcggatgtataggtggtaatggctcacggccgggcccgtcgccggccagtcttcgggatccacggtatcaaatactatgagccgtcgtctggcggaccccagacgcagacatagatattcaacgcatccggcgaaggctagatccccggtggagagtaataacaccccctgggcgttgagtctgcggatatccgggggaccactccaattccctgcccaggagtgactttcgggggagaggaggcggtttcccaaggccgctaagagggtcgatagaccccccttagtttcagaccacgccggggtgtgccgagagtccctgtttggattttggtgggccagatgttcgtgtggtagggggttatacaacaccactactttcacgtcctcggggtctgctatctgggtcatccaggcggaagcgcgccgcaatggggcgctaaccaccagccccccgagacctgtaccctcctgccttcgaggtcccgtacatctggcggctatggtagcgagtgcccccgggtcaaacgtgatagcgcttctccacgcttcgggaaagagggggtgatctaccagctccgcgacaatttccggggggcagtaaacggagctcaccgcatcggtgggtctgggggtatgacagtctccacttgggatacgtctaaaaccacccttgggtgagggccccccaagtggcatgggtccaaggggacccctagggggacccgcgcgttttttggtgggtggtggcgcgtccgtcggggggtgggctcgcatctcctccccccgaacggcatcgatggggaggttgccggatgcgtggccccggggaagcggctccgatttacgcttgcaccgtatagaggattgggggcgggtgaggtggggtgaaaggggtctgaaattcgaggggctctgttggagtggatcacaaccccccgctacggggttgtgaacatcccgcctctgttgcattttgttagacgaccgtggaggcgagatctcctccaccgtgactcccccggctcggtttcccgaagcggtcgcccggggggtgggagtatgggggtcgggggtaccggcgacctcctccatgagagaggctacgcgcgaagggatggcaaagacggcccgcgtgaggttccatccgggtgcgcgcttcctggccacgtcgatgttgtatctgacgaggggggccagggtcctggctaccaagagtagactgtgtacgacgagctgaagatcatcccgggagagtctctcgggcggcgcctcaccgccccgaattctagaaagttccaccaggtctcttaaactatagaggcgtgcagtcaccgcctctatcacatccgggtgggcacagagtgcatccggggggcagaccggcgaggggtacttggccaccgaggggaaagaccgagccattgaggggggttcgaacaatcccggcatgcggtctacgcccccgcaatgaaatatgaggctgtcaaagacggcggtacatgccaatgtacaagtctccgccagatccctcggattttctccacccattccgggagttggtatctgggtgggatcccccgttcccctggtcgtcccccccgccaatgtggactggagtaacgctagtatgtcttcagagaccatgagcaccctatccgaggatgtgggggtttttgggggggcgtcgggaattgcgtcagcgatccccggtgaagacctaatacagtctgtcctatgatccccgaggagatagggacgcgctatgtcccagtatgcacgccggaggctctggagcatgtaggttttttgcgtgcgatcgtagcgtctgctcatggccacggccgccgtgacgtgggggagagccaggagagggtccatggtcgccatggcgtccccgacgtgggggaggggattggccactgagccggtaatgaaagatccatgtccccgggggccgtggatggccctctgacaaaactgtgctaatatctgatccggcccggacatctttgagttttgtagccacgccatcgcgtctctaccactctcatagatgagacgcacgagggccagatactgctttttcgagtcccccatctccggaacgtagacgggaaccgggccggggtccgcggcataccgctccgcggcacttttgatctccggagcgtcccacagaccttccctcgagtccccgatccccccatatctaacacgcccctcacatggtgggtcacagtccggccagacctctccggtgggtgtcaatagcggtcctgtggttgggggtcgttgtatcccaaaaacggaggtcctttccgagatcctctcggccctggggagcaatccccgaacggcccgcggaagtctggaacctggggggcgggatagcggaccccgtcgacggcgcctctttggtgttgtatccccaggcccatcagtgagcgtcgctccatcagtgagcgtcgctccaggggtcgtagaggcagaccccacaacgtttgattggggggttatagaggcaatgacgtcatttgtgattggactaccttctccggcaggccgagggggagacggggtccccggcttcaattctcgattcggtggtgctggtggcgatggggaccgtgaggtgctggaagacgaagaactactacttctgctgccggtactactgcttcccgaactgctactgctactgctactactgctgctaccgcttcgactccgtcgaccccttcgtggtccaggactcatcgatcctaccgagatgggggatcgacgccccctggaataatcgcgggctcccaaggaatcacattctgtgacgtcacagacacccaccgatgaggggctatgggcggatgaatcggtgatggagagggcgttgctgggatgggaacaggtttccgtatcgctagtggaacgcccgtgtgattcagagaccccgagatcctcttggaccctacgcaacatctcaatgatgtcgggtactggggtctcaggagacgctgccgaatgtatcgtcgggggtggtggtggtttagtggtgggtcccgaggtaattgtttcacgtgtgggtcgcggagacgtcagagttttagtccctccggatgtgggattccccaccctgttgacatcatcgggggtcttcccaaaatcaaaaaatacctcatctttatcccccggtggcgatacgaggaaaccctgactcccgtagagtaagtcttgaggtcgtcccctcgactctacatcagagttggtatcccccaagaattcaaactcctctctaccatccgtgaagtcttcgatgaggtcgaagaggtctatttgagggaacctctcatcaccgtccatatcggggcgtgggtgatgtgggctgaacatcactacacagggctcttggaagtgtttttatatgcgttgggggtttgagtcacgggatcctcaggaaaatgatgtcgaggataggcctattggtacaacctgatatatcggcctataaaatttataaaaaaagatggagacagatgcggtaagttccggatcccacaatgttaaaaagactctaactagatattatttacactgttccctattttttttacaagcgcatataacgatgtgtcactcgtgggggtctaacgcatttcccggcgtgttggatgcggggatgttatagagacttgcctgtgattggaaaagaccagacaagcctatacatccctatccaattcgtacgaggggaggagcgaaggtacgaggggaggagcgaaggtacgaggggaggagcgaaggtacgaggggaggagcgaaggtacgaggggaggagcgaaggtacgaggggaggagcgaaggtgagcggcgatcttgtgggttaattgctacgcgagtaaactcttaaacttttcactccctcaggtgcattttctatagcaataattagaaatgccatattaatagtttgttcgctagcacccacatagatagccggtttacactaactactgaaataatgcctgtatacatttttgagtgatatcccattttatgaaattttggaaagggtcgtctaaaatagagcccacctccattaccacccttccacctagtctatctcctcaccccttccgaggccgtctgatggtggatcggctcccgactccgagacggtgagtacagcgactgccagataagatgggtgccgggtatctcgatgctgagactgggtacagctgaatggaagaacgctgagtacgatcatcagctccccagggagcgagatgctacgatgacagtggttcaggtatatatactactacgatggggagtgactccaccttacagacctgggattggacaataagcatgggaggagattgtccacccacgagattatctccgattggtttaccttggcattacctgtatatctgggtggagtgatgggacaggtgttgtatggggaccaattggagaatttgtgagtcataacttcctggaataatgcacatctaatttccattttatctgggagtgttgatataatttgaattttactgattatcatctacatatttaattaggttggaagccatgttgttccggttgcacatctaatctacatgaaagtgggaaggttggaagccatgttgttccggttgcacatctaatctacatgaaagtgggaaggttggaagccatgttgttccggttgcacatctaatctacatgaaagtgggaaggttggaagccatgttgttccggttgcacatctaatctacatatgaatagtagatttgaatatttactgatgtgataactcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccctcccctctgccccccctcgatccccccaataaaccagatgttcttatgtatcactttattgagacgtgtcttatttgtggtgtcgtttaccggggtgatattgatatttacaataggtattacatataccaaacttttggatacacttacattcgtatagatatcaatgatgtatggatataaagttaaatcttattaatatgtctatataacccctcccccccatgtatgatggggggaaccaaaattttaaactgaactcaattttcccccaggtttatattgtccatttttgagagggggggttgaaaactacagtttatttgagacataagaaactggtcacgatatactaatttgggggatctgattaacccattaagactgggatatcctaacccccaaattattatcctgttaatatacgatccatatcctatatgccagattcgagttttttagaatcattaagacatggtcttatgtgggggaaaaatcaaccggtaattactggacacataacccgcgttaggcgaaaatatgcgcgtgcacgcggctgtcatcaccgagacctacaggcatatatgggcgtattttttaagaaagaagtgcgaacacgcagtgttcgcacttcgacttcaataatatataatataacttattgggaatttagtgcggacgaggttctagatcattggctggaactcggtggatatgtaacacacccacaaaaatgacgttaaatttgtgggtgtgttatattttctcattgataaataagagcctggagcttggacgcaccactgtcgcccaacaccgtatcactgcgcatctggatccccgggagtggaacgacgatacagcgatttctggggtggtcgcacgtctcaacggtaacccgatcgagtcgtccacgcaccggctgccgccatccataaccagggagtcccatggcgaagggaaggaagcatcgcggtaggccacatacgacccccgccgctgtaaaacccccagaactcaggagcggtcgattgggtcatggacaaccctgtggtctatgcgacggatcgtgtagactgcgaccccgtggggggttgaatatagatcccagtcccatcctcccctcgttaacaccatcgaccggttcatcgaccttcgcatcgcaaacaccaggaggatcaaagatacccgtttcttgcgtagaaactgatgatacgagtccggtgcgcgctcccgcgacacttcccgatggggatgatcatcccgagtatggaagcgtggtcgcgagtcgtgacctcagagcttatttgacacacggacccggggctttttgttccccaccgtggcgatgtgatctaaaacggctgatgaaagatgtcaacatcatgttcagatgtatagccacgtcgtccgtaaatgtcagctcggattcccgcgcgcttcgtcgggcgttgttagacttttatatcatgggttatacgaaccaacgtccgaccagagcctgttgggagaccctcttacagttgtcttcggaacaggctcgtcccctgcgagccaccttacgccacctcaataccactgaaccatgcgatcggcggttccttgagcctcccacacaggtccccccgattttatttggacaggaatgtgatgttagtggtagcgaagactctgaagatggtgagggggtggatatagaaagtagcgattcggattgtggagagacatcgttgtccgaatttagttatgtggggggtgatggggacgagactagtaccagtgactcagattctgggacggatgacgactcggatgaaacggaggatcggagctctagttcgtcagagtcggatagttctgacgttgattatggtacccgtgtctgtggtaaaaaacgcagatgtaatcccgtcagaaggagcgcgcgccacgcggccaaacggaagaggatgtagatatcaaattaaaaaggtatataggaaaccacatgtctcacaaccgcctcctgattcctttccaccttacctaggtgcaatagatttattacccacaccccaacccctgtctctcatgatgagtacatagcccggataatcacggaccatatagttggtgtaataccccccgatatatacattcctacacgacgcccgactcctatgggagtccactagactccagcggacgcgtatatcatgaaccgtcgctgggaagacactaacatcgagtcctttaacatgactggtgtcgcggagatggagatgtaccccctgaggggggactccgcggatcacgcggaaacgctacctcgttctgtcagggctctgttcgacgctctccgggtcgcgtcgtgtgaggccttctgcctcatgcgcctcggtggtcccccacccgcggacatatggcccggggtatatcggcagtatcgtgaggtatttcgaagctactcacgctccatggagggtagcgggggtagcccatttcacgtcgccgatcccatacggcacctggtaggccgttatctcatgggtctgggacccgcaaaaccagaatcccatccggagctacacacccgcctcctctactgcgcctattggtgttgtctgggtcacgcggctacatgcacccactcccacatctatgaggatgcctgtcggcggtttttcgaagagggtttcggggccggggaaatcccccctgcggatgccgtagctcactggaacgcgctatacgagatggtgctcgacgaaccggagctcctggtgaaacacgcggcagccgcggtttatctacagcggagaaactatggggggtgtataccgaatattgagaggtagaagacccccgttttcctcccatctctccccactaatctaaataaactcgccttcataagccatctaaggtctcaaaattctttatcacacgatcgatcccagcaacaagatttatgtactcgtaccctatcccctccccgtggtgatctacgacccaaaacctagggttcgtggagactcccctgtacaatattgtacgcgcctcgctgaaaacccaatctaaaacaatcgtccccatataagacgcggtagaccatgtcataacgtatgctatgtttattgaacgagcccgtcaccggcgcgtcccgggggcatccatcagccgccacagcagcgcacctaataatcccgatagcacgcatagtgtcgccacccctgagacggccagtgccaccagacaccgcctccgtcgccgtctcttggccgtctcgcgtttgcctattctatttatgaacagcgtcgccgtctcgttgtcactctcgctataataacagccggtatcggagtcgtaatttgtcctattggcccctcgtacgtcgtggcttggggactgccggtccgcattcccacgcgggatctcgatggtggcgcatagcggtatagcgaccggggagcccttgtcggatagagtttcttatgtacaaatgggatggtagttgtacaagcggacattaatagttctctctaagtaatatttggatggtattcgataaacccaatatcaataatttaaaaatattgggtttccatgggtcacctggccattaataatatatatttaacataatctcttatagctcaacatttctataaatatgagtccatataaatctgtggccccaaaagattcatacatacgacagataaacccgtttacaaatttcagattatacttagtcgttagagagttcattttgacgttaataccgaggtcatatttatatagaacccatcagaatccacggtctgatgtaactgggatatcgtcaatagattcttacaatgggtcccaatatcaaagatgggcacaacataagatggacaaaattataggcttagaagactattatcacattaggcactcagtgggccaaagtaaaatatgttacatcatcatgctttagaaatcaaatatcgtagggtagatgttatataatcacaccaggagtgtctcatatttgtcaacaaaagtaacaatagacattataaccacacccatatcaacatcatccaataaaagtctgatatagaacccaaaatatcacagacctttccgtcaggtggagacgtagggcaggtttctgttatttaggaagtgtatacgtttggctaatatggatagatttccacgtgttggactttcttgttgtatccctacgagcaaaggtgatattgatactggggataattacaaattacaatcgaccatggaggatgtctctgttacacccaacgaagctcctcgtggtatgctggacgatgatctctactctgatattagcgacggtgatttcgaggatatatgtgatgatactgataccgaaactagtgatagtgatgatgaaagtcttaaacctactaaacaggaagcattggaggccgtccatgcattgaattatacgatcgtgaaaacgcttactccgggatctgaaggtagagtatttgttgctaaacctccagggagtgagacacccgttgtattaaaaattggtatgaaaggggcaacgttgattgaggcacttctactacaaaatatcaaccacgaatctattataaaactattagacactctattttataaggaacttacttgtatagttttaccgcgatataaaagtgatctttatacctatctatcaaacaatatgcgaccattgtctttagcttcagctttcacaattcaaaagcagatactcaagggattgaattatctacatcaacataaaattatccatcgggatatcaaaatagaaaacatcctcatagacgatgaatccagcgtatgtatcggagaccttggagcggcacaatttccagtgaatgccccagatttcctcggtgttgccggtacagtggaaactaattcaccggaagttttagcaaaggatgcctacaattctaaagctgatatatggagtgctgggataatccttttcgagatgatcgcatacccccattctatatttgatgatgaagactccagcgattcgtcaagaagctcccgcaaccatcttcgtaaagttataaccactctaaaggttcacccagaggaattcccaggcgacccgacttctaaattaactatagactttattcattatgcctcatgtgtaagacagccttatacacgatacgattgtatgtcaaaatacgatttgccgctagatggggagtttgtagtgcataagatgttgactttcgatgccaagttccgaccatcggccgctgaaattctaaactatccaatgtttcgtgatacatagttacatcattatctatgggtgggactttccaccaagacagtataaagtatttgggactccacaatagcattcatccttcttacccttctacgagtaagaacttcaatcactcaacttggaagaaaaatgggaaatcgtatacatattttaatatgcattgcagcattctacataaccatcgcggctgctaggaatgccccaatggatctctgttacgccgaccccagagatacatcaccacaacccataggacatcctaattataaacaagtgaatatcaacgatccactacccgcaccaaagtggggatatgttgaacattccagtggatgtgaattacgtttattggacccgagagttgatgtctcttcaagatcaccagagaaggcagacgctacgattgcttggacttttgatctcggaacatgtcaaatacctatcgcgtatagagaatattataactgtactgggaatttaataccctccccagaaacttgcgaagggtattccgcgacctccatacgcttcgaaggtctaaccatctataccttggtaaatataagtctactccttcaaccaggaatattcgattccgggagtttcctgtattcatttatatatggtcaaaatagatacaatggacgtattatagttcatgtagaaaaaaatactgattatccctgcaaaatgtatcatggactcatggctccatttgaccatcatccccaaagccacgttgaaactccgaatgataagaatcatcgtagagggcggggatgttttcccgaattggtggaacctgttctatgggttaatatcagcagtgatcttattggtggtccacctttcgactataatcatgaagatgaggctgatattgagagtgatgagctcccggaggaggatatacataactactcagattgtcgtgcgactaatatgtttgttccgagagagcccctcagtcaagttcttggttctcaaagtctactggttggtagtttaggtttccagataattactcaaccctggcaactgaagcagaatgaaagttatgatggactaagaaatgcctctcttgaaccccgacaccttgactccagtaacgatcgtgatctactagatgaaactgaaatgattggatcgattattacgactccaccaccaacccatccaaaaggtgtcaatgggggtttcctccaagatctaccaattatcgagcctacgaccgaaccatgcttagtacatacaaagatcattgggatcggaacagtagtcgttgtatttttgttatttattctcatatccctatgtgtttatacttgcgttctacgatcccgcatcggtatggtagatcgcgcctatgtgaaacaagtacgatttaattccaatccatcatatcaacagttgacaagatacccccaaccataataaactgattaaatttaattaaagtctcatatgtggggctgtggggacgaggggctgtggggacgaggggctgtggggacgaggggctgtggggacgaggggctgtggggacgaggggctgtggggacgaggggctgtggggacgaggggctgtggggacgaggggctgtggggacgaggggctgtggggacgaggggctgtggggacgaggggctgtggggacgattacaaccgataaatgtcgtatatgaaatgtggtgttaacataacacggattttttaagcacaccacatgacacacccccacgataacggtttaaatcaccagctatgtgaactgccctccattctactcaaatgagtggtggtgtgtggcatattagaaccatttcgtctaatgatgacacgtctacatttttggtggtgtggaatctttgcggtcctgaaatatctggtatgtacttcaagccttacgaccacgccaaaaacaactacggtttatgtgaagggatttaatatacctccactacgctacaattatactcaagccagaatcgtgccaaaaattccccaggcgatggatccgaagataacagctgaagtacgttatgtaacatcaatggattcatgtgggatggtggcattgatatcagagccggatatagacgctactattcgaaccatacaactatctcaaaaaaaaacatataacgcgactataagttggtttaaggtaacccagggttgtgaataccctatgtttcttatggatatgagactttgtgatcctaaacgggaatttggaatatgtgctttacggtcgccttcatattggttggaacctttaacaaagtatatgttcctaacagacgatgaactgggtttgattatgatggccccggcccaatttaatcaaggacaatatcgaagagttataaccatcgatggttccatgttttatacagattttatggtacaactatctccaacgccatgttggttcgcaaaacccgatagatacgaagagattctacatgaatggtgtcgaaatgttaaaactattggccttgatggagctcgtgattaccactattattgggtaccctataacccacaacctcaccataaagccgtactcttatattggtatcggactcatggccgagaacccccagtaagattccaagaggccattcgatatgatcgtcccgccataccgtctgggagtgaggattcgaaacggtccaacgactctagaggagaatcgagtggacccaattggatagacattgaaaattacactcctaaaaataatgtgcctattataatatctgacgatgacgttcctacagcccctcccaagggcatgaataatcagtcagtagtgatacccgcaatcgtactaagttgtcttataatagcactgattctaggagtgatatattatattttgagggtaaagaggtctcgatcaactgcatatcaacaacttcctataatacatacaactcaccatccttaagtccacattccaatcgagttggtagggaagatatgaagtgggcggtaccaaccatcataaaataggttggagtctggaccaacgttcactcttttgagtgtaaaggaccacagcataatacttaatatgtcgtcgatagccttcatctatatattgatggcgattggaacagtttatgggattgtgtatcgtggagatcatgtaagtcttcatgttgatacaagctccggctttgtaatatatccaacactggagaattttacgatctacggccatctaatctttctcgacgaccaaccattaccagtaaacaattataatggaaccctcgagattatacattacaaccatcactcttcttgctataaaatcgttcaagtaatagaatattcatcatgtccacgtgtacgcaataatgctttccggtcctgtctccacaagacctctatgcaccaatacgatcagctttccataaacacatccgttgaaacggggatgttattgacaataacatctccgaaaatggaagatggtggaatctacgcactgcgggtaagatttaaccataataacaaagctgatgtatttggcctttcggtgtttgtttactcattcgatatgcgtggtcatcgacatcatgcggacgaaaatttgaatggtgaaattcttactactccatcaccgatggaaacatatgttaaagttaacacaccaatatatgatcatatggtgacaactcaaacaacttctaataaatcgatggagtctgaaccatcaaatacatcaatatcatgccatacatttcaaaatgacccgaatgagggtgagactttatatacacacttattgaacatcgctggaaatataacatatgatgacatggttatggatggcaccacattgaaacccagattaatcgatatgggacttaacttgtctgttacatcttcctttaaaaatgaaaccacgcaaaaatggacaccagacagaaaggtgggttttgttatagtaatctcaatcgcagttttactactccttgcggtcatcggatccatcatcaatagtgcaatacgcaagcatataatggtctgtgctgggcggcggatctatataccaaacaacgatgggcgaccatcaacggaaatgacacggtttactcgccagactaaaccatcgaattcgagttccaagtccctactcgatgtccccagatcttcgaattccaccccaaccgatggcgtctctagaagtcagttaaccgtaattaacgaagaaacctaatatatttataaacaaataaaatacttttaaaaatggatatctggtcatgtgtaatgttgacgcatagtgggtggtgacctaagattatattaaaatgtagaaggttttatgcccagttcacagtatctactgtgacctaccccggggtggtaataacaatactatcgaatagccaacaatgggactgcttgttaccatcctcgtgatattattgattgttacttcatcaagttctactattcatcaagtaacgatgacagaaggtgccgcacttttagtcgatggggatgggatcgacccacctttaaacaaaacttcacattttttgcgaggttggacatttctagagactccgaaaggatgtacaggagaggtgagtgttctaaaagtatgtatagatcgtggggtatgtccggatgatatcgttataaataagagatgtggtcacaaaatgcttgaaaccccactagcgttggcggaatttggaatttctaatagttctctcatcagaaccaaagacgtatatttcgtgaataagaccgtgtttccaattctcacacccgaaaaaagtggccttggtattcagggggccactacgaatatatccgggatatataccctgcatgagcacggtgataatggatggagtcatcaatctacattttttgtgaccgtaaaggcaaaacatcccggaccatcgttaaccccagcaccggttcacttaataacaccacatcgccatggggcacatttccacgtaagaaactatcattcgcatgtctacattccgggagataagttcttattagaaatgcacctcaaatcagatatctatgatccagaattttcagcaacaatagactggtattttatggagactgatataaaatgcccagtttttagaatttatgaaacttgtatatttcacccccatgccgcatcctgtctacatccggaagatccctcatgcagttttacatcaccacttcgagcggtatctttaattaatagattttatccaaaatgcgatcacagatatgccgattggacatccagatgtatcaacactccaagtataaatcatatgccatatatcgaacagccggccaataacgtggatctaaagtttatcaatgtacccaccaacgcttctgggttgtacgtattcatacttcgttataatggacatccggaagaatggacctatacactcatatcaacaggagctaaatttttgaatgtgattagggatctgacacgcccacgtcttggtagtcatcaaatagagaccgatattagcacatcttcgcagtcgcctaccacggagacaccacgaaacatacatataacgtgggcgagacgttatctaaaggttatcataggaataatttgcgtagctggtatccttttgattgtaatctctatcacatgttatattcgatttcgtcatatgcgatataaaccatatgaagtgatcaacccattccctgcggtatataccagcattcctagtaacgatcccgacgaactctactttgaacgtatcgcatcgaacgacgaagaatcggcagatgattcttttgatgaatcagatgaggaggagccattgaataatcatcatatttcaacaacccaacatactgatattaatccagaaaaatccggatctgggtacagtgtatggtttcgtgatacagaagatacatcacctcagcccctacacgctcctccagattacagtcgcgtagttaaaagattaaagtctattttaaaatgacccgtcgacgagttctagcaccacgggaattggaagctgctcgtaaactccgtgagattttcaacgcagagtacgtcgcacctacgttcacactagtcgatccgggggatacgtcaaacgcgtatattgtatgtaggaccccggtgaccgaagtagtctcttccatatcaagaggtatcgacaatagaaaatcggtagattcttcatttattcgaatcgtcagtaaattaatcattaggaatgctattcacatgggactatccgtcctatgtgcatttatatcctataataaaccatgataaattttatgtggatattttattaatcctccaaaccgtatgggggagcacttttatagaaatctaccataaagagtatatccgttaaatacccgggtttgattatatgtttgtcagttggtagttgaacttccatcgccatctaagatggaccatcaaacatcacttattaatgccacagatgataattgcttagacacggattctagtataaacttgccaagcatagataaatgcgaaattgatgacaattcaattgcggatgaaactctatccgacaagggctccccggtcgctataccgctatgcgccaccatcgagatcccgcgtgggaatgcggaccggcagtccccaagccacgacgtacgaggggccaataggacaaattacgactccgataccggctgttattatagcgagagtgacaacgagacggcgacgctgttcataaatagaataggcaaacgcgagacggccaagagacggcgacggaggcggtgtctggtggcactggccgtctcaggggtggcgacactatgcgtgctatcgggattattaggtgcgctgctgtggcggctgatggatgcccccgggacgcgccggtgacgggctcgttcaataaacatagcatacgttatgacatggtctaccgcgtcttatatggggacgattgttttagattgggttttcagcgaggcgcgtacaatattgtacaggggagtctccacgaaccctaggttttgggtcgtagatcaccacggggaggggatagggtacgagtacataaatcttgttgctgggatcgatcgtgtgataaagaattttgagaccttagatggcttatgaaggcgagtttatttagattagtggggagagatgggaggaaaacgggggtcttctacctctcaatattcggtatacaccccccatagtttctccgctgtagataaaccgcggctgccgcgtgtttcaccaggagctccggttcgtcgagcaccatctcgtatagcgcgttccagtgagctacggcatccgcaggggggatttccccggccccgaaaccctcttcgaaaaaccgccgacaggcatcctcatagatgtgggagtgggtgcatgtagccgcgtgacccagacaacaccaataggcgcagtagaggaggcgggtgtgtagctccggatgggattctggttttgcgggtcccagacccatgagataacggcctaccaggtgccgtatgggatcggcgacgtgaaatgggctacccccgctaccctccatggagcgtgagtagcttcgaaatacctcacgatactgccgatataccccgggccatatgtccgcgggtgggggaccaccgaggcgcatgaggcagaaggcctcacacgacgcgacccggagagcgtcgaacagagccctgacagaacgaggtagcgtttccgcgtgatccgcggagtcccccctcagggggtacatctccatctccgcgacaccagtcatgttaaaggactcgatgttagtgtcttcccagcgacggttcatgatatacgcgtccgctggagtctagtggactcccataggagtcgggcgtcgtgtaggaatgtatatatcggggggtattacaccaactatatggtccgtgattatccgggctatgtactcatcatgagagacaggggttggggtgtgggtaataaatctattgcacctaggtaaggtggaaaggaatcaggaggcggttgtgagacatgtggtttcctatatacctttttaatttgatatctacatcctcttccgtttggccgcgtggcgcgcgctccttctgacgggattacatctgcgttttttaccacagacacgggtaccataatcaacgtcagaactatccgactctgacgaactagagctccgatcctccgtttcatccgagtcgtcatccgtcccagaatctgagtcactggtactagtctcgtccccatcaccccccacataactaaattcggacaacgatgtctctccacaatccgaatcgctactttctatatccaccccctcaccatcttcagagtcttcgctaccactaacatcacattcctgtccaaataaaatcggggggacctgtgtgggaggctcaaggaaccgccgatcgcatggttcagtggtattgaggtggcgtaaggtggctcgcaggggacgagcctgttccgaagacaactgtaagagggtctcccaacaggctctggtcggacgttggttcgtataacccatgatataaaagtctaacaacgcccgacgaagcgcgcgggaatccgagctgacatttacggacgacgtggctatacatctgaacatgatgttgacatctttcatcagccgttttagatcacatcgccacggtggggaacaaaaagccccgggtccgtgtgtcaaataagctctgaggtcacgactcgcgaccacgcttccatactcgggatgatcatccccatcgggaagtgtcgcgggagcgcgcaccggactcgtatcatcagtttctacgcaagaaacgggtatctttgatcctcctggtgtttgcgatgcgaaggtcgatgaaccggtcgatggtgttaacgaggggaggatgggactgggatctatattcaaccccccacggggtcgcagtctacacgatccgtcgcatagaccacagggttgtccatgacccaatcgaccgctcctgagttctgggggttttacagcggcgggggtcgtatgtggcctaccgcgatgcttccttcccttcgccatgggactccctggttatggatggcggcagccggtgcgtggacgactcgatcgggttaccgttgagacgtgcgaccaccccagaaatcgctgtatcgtcgttccactcccggggatccagatgcgcagtgatacggtgttgggcgacagtggtgcgtccaagctccaggctcttatttatcaatgagaaaatataacacacccacaaatttaacgtcatttttgtgggtgtgttacatatccaccgagttccagccaatgatctagaacctcgtccgcactaaattcccaataagttatattatatattattgaagtcgaagtgcgaacactgcgtgttcgcacttctttcttaaaaaatacgcccatatatgcctgtaggtctcggtgatgacagccgcgtgcacgcgcatattttcgcctaacgcgggttatgtgtccagtaattaccggttgatttttcccccacataagaccatgtcttaatgattctaaaaaactcgaatctggcatataggatatggatcgtatattaacaggataataatttgggggttaggatatcccagtcttaatgggttaatcagatcccccaaattagtatatcgtgaccagtttcttatgtctcaaataaactgtagttttcaaccccccctctcaaaaatggacaatataaacctgggggaaaattgagttcagtttaaaattttggttccccccatcatacatgggggggaggggttatatagacatattaataagatttaactttatatccatacatcattgatatctatacgaatgtaagtgtatccaaaagtttggtatatgtaatacctattgtaaatatcaatatcaccccggtaaacgacaccacaaataagacacgtctcaataaagtgatacataagaacatctggtttattggggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagggggggcagaggggagggggatcgagttatcacatcagtaaatattcaaatctactattcatatgtagattagatgtgcaaccggaacaacatggcttccaaccttcccactttcatgtagattagatgtgcaaccggaacaacatggcttccaaccttcccactttcatgtagattagatgtgcaaccggaacaacatggcttccaaccttcccactttcatgtagattagatgtgcaaccggaacaacatggcttccaacctaattaaatatgtagatgataatcagtaaaattcaaattatatcaacactcccagataaaatggaaattagatgtgcattattccaggaagttatgactcacaaattctccaattggtccccatacaacacctgtcccatcactccacccagatatacaggtaatgccaaggtaaaccaatcggagataatctcgtgggtggacaatctcctcccatgcttattgtccaatcccaggtctgtaaggtggagtcactccccatcgtagtagtatatatacctgaaccactgtcatcgtagcatctcgctccctggggagctgatgatcgtactcagcgttcttccattcagctgtacccagtctcagcatcgagatacccggcacccatcttatctggcagtcgctgtactcaccgtctcggagtcgggagccgatccaccatcagacggcctcggaaggggtgaggagatagactaggtggaagggtggtaatggaggtgggctctattttagacgaccctttccaaaatttcataaaatgggatatcactcaaaaatgtatacaggcattatttcagtagttagtgtaaaccggctatctatgtgggtgctagcgaacaaactattaatatggcatttctaattattgctatagaaaatgcacctgagggagtgaaaagtttaagagtttactcgcgtagcaattaacccacaagatcgccgctcaccttcgctcctcccctcgtaccttcgctcctcccctcgtaccttcgctcctcccctcgtaccttcgctcctcccctcgtaccttcgctcctcccctcgtaccttcgctcctcccctcgtacgaattggatagggatgtataggcttgtctggtcttttccaatcacaggcaagtctctataacatccccgcatccaacacgccgggaaatgcgttagacccccacgagtgacacatcgttatatgcgcttgtaaaaaaaatagggaacagtgtaaataatatctagttagagtctttttaacattgtgggatccggaacttaccgcatctgtctccatctttttttataaattttataggccgatatatcaggttgtaccaataggcctatcctcgacatcattttcctgaggatcccgtgactcaaacccccaacgcatataaaaacacttccaagagccctgtgtagtgatgttcagcccacatcacccacgccccgatatggacggtgatgagaggttccctcaaatagacctcttcgacctcatcgaagacttcacggatggtagagaggagtttgaattcttgggggataccaactctgatgtagagtcgaggggacgacctcaagacttactctacgggagtcagggtttcctcgtatcgccaccgggggataaagatgaggtattttttgattttgggaagacccccgatgatgtcaacagggtggggaatcccacatccggagggactaaaactctgacgtctccgcgacccacacgtgaaacaattacctcgggacccaccactaaaccaccaccacccccgacgatacattcggcagcgtctcctgagaccccagtacccgacatcattgagatgttgcgtagggtccaagaggatctcggggtctctgaatcacacgggcgttccactagcgatacggaaacctgttcccatcccagcaacgccctctccatcaccgattcatccgcccatagcccctcatcggtgggtgtctgtgacgtcacagaatgtgattccttgggagcccgcgattattccagggggcgtcgatcccccatctcggtaggatcgatgagtcctggaccacgaaggggtcgacggagtcgaagcggtagcagcagtagtagcagtagcagtagcagttcgggaagcagtagtaccggcagcagaagtagtagttcttcgtcttccagcacctcacggtccccatcgccaccagcaccaccgaatcgagaattgaagccggggaccccgtctccccctcggcctgccggagaaggtagtccaatcacaaatgacgtcattgcctctataaccccccaatcaaacgttgtggggtctgcctctacgacccctggagcgacgctcactgatggagcgacgctcactgatgggcctggggatacaacaccaaagaggcgccgtcgacggggtccgctatcccgccccccaggttccagacttccgcgggccgttcggggattgctccccagggccgagaggatctcggaaaggacctccgtttttgggatacaacgacccccaaccacaggaccgctattgacacccaccggagaggtctggccggactgtgacccaccatgtgaggggcgtgttagatatggggggatcggggactcgagggaaggtctgtgggacgctccggagatcaaaagtgccgcggagcggtatgccgcggaccccggcccggttcccgtctacgttccggagatgggggactcgaaaaagcagtatctggccctcgtgcgtctcatctatgagagtggtagagacgcgatggcgtggctacaaaactcaaagatgtccgggccggatcagatattagcacagttttgtcagagggccatccacggcccccggggacatggatctttcattaccggctcagtggccaatcccctcccccacgtcggggacgccatggcgaccatggaccctctcctggctctcccccacgtcacggcggccgtggccatgagcagacgctacgatcgcacgcaaaaaacctacatgctccagagcctccggcgtgcatactgggacatagcgcgtccctatctcctcggggatcataggacagactgtattaggtcttcaccggggatcgctgacgcaattcccgacgcccccccaaaaacccccacatcctcggatagggtgctcatggtctctgaagacatactagcgttactccagtccacattggcgggggggacgaccaggggaacgggggatcccacccagataccaactcccggaatgggtggagaaaatccgagggatctggcggagacttgtacattggcatgtaccgccgtctttgacagcctcatatttcattgcgggggcgtagaccgcatgccgggattgttcgaacccccctcaatggctcggtctttcccctcggtggccaagtacccctcgccggtctgccccccggatgcactctgtgcccacccggatgtgatagaggcggtgactgcacgcctctatagtttaagagacctggtggaactttctagaattcggggcggtgaggcgccgcccgagagactctcccgggatgatcttcagctcgtcgtacacagtctactcttggtagccaggaccctggcccccctcgtcagatacaacatcgacgtggccaggaagcgcgcacccggatggaacctcacgcgggccgtctttgccatcccttcgcgcgtagcctctctcatggaggaggtcgccggtacccccgacccccatactcccaccccccgggcgaccgcttcgggaaaccgagccgggggagtcacggtggaggagatctcgcctccacggtcgtctaacaaaatgcaacagaggcgggatgttcacaaccccgtagcggggggttgtgatccactccaacagagcccctcgaatttcagacccctttcaccccacctcacccgcccccaatcctctatacggtgcaagcgtaaatcggagccgcttccccggggccacgcatccggcaacctccccatcgatgccgttcggggggaggagatgcgagcccaccccccgacggacgcgccaccacccaccaaaaaacgcgcgggtccccctaggggtccccttggacccatgccacttggggggccctcacccaagggtggttttagacgtatcccaagtggagactgtcatacccccagacccaccgatgcggtgagctccgtttactgccccccggaaattgtcgcggagctggtagatcaccccctctttcccgaagcgtggagaagcgctatcacgtttgacccgggggcactcgctaccatagccgccagatgtacgggacctcgaaggcaggagggtacaggtctcggggggctggtggttagcgccccattgcggcgcgcttccgcctggatgacccagatagcagaccccgaggacgtgaaagtagtggtgttgtataaccccctaccacacgaacatctggcccaccaaaatccaaacagggactctcggcacaccccggcgtggtctgaaactaaggggggtctatcgaccctcttagcggccttgggaaaccgcctcctctcccccgaaagtcactcctgggcagggaattggagtggtcccccggatatccgcagactcaacgcccagggggtgttattactctccaccggggatctagccttcgccggatgcgttgaatatctatgtctgcgtctggggtccgccagacgacggctcatagtatttgataccgtggatcccgaagactggccggcgacgggcccggccgtgagccattaccacctatacatccgcgccacggtcaccccgaccgcttactgcgcggtgcgctggccggccctagaggacctctccagggcagttctgacctcgagcgccatttttggaccggggagtttcgcccgggtggaagcggcctttgagcgcctgtacccaaactgtgacgggttgcgactctgcgcttccggtaatgtgggctacaccgtccgcacggacgcgggggaccgcacacccgttcccatgttaccccgagagtatcgccagcgggtacttcctgcctacgacgggtgtaaggatatggaggctcagagtgcgggcctcaagctcggagagcctgatttcgtagacggtgccgcccacagccatcgtgcctgtaatcgctggggtctaggggcgcccctacgtcccgtgtatctggcaaccgggaggcgtgcgttggcgtctactgaccccacccggattccagcggtcgtcagagccttctgtaggacggccctcctcgcacctgacactgaggctccacccctgattctaactccggcggatccccagtccgctatcatcgagaacataccgggtgtgacgtgggactcgggatttggaaaacaggaaactgtgatacgtatcggggggcgaagcgagactctgtctcctccaaactatcatacgggcgattcacaggctgtggagattgtctcggtcgagaccggtatcaacggcccggggtcgctcgcctctgccgagccggttaaagtggaggttgtctccgacgacgaggacgcggagagtggatggttccataacagcaacccttacatctgacggggtccggtgtcccagacaaggtcacccccctccgcgggttctaaaacttgtaaacacaattgccagcagctgtccatctgtgccaaagaaagaaaataaaacctaccgttcagcacttccataacaattaccagtgtgttgtctgtagt

SEQ ID NO: 5 - DNA, 1879 bp, Artificial Sequence
  Synthetic
agtcgaaatcacaggcagtgaaatgccttaaaaatgggtctcctgtctatgttaggaatctcttattttaagtagtcccgcgagacgatttacatcccgggatcaccaacaatctgcgatgagacgatataggatgggacgcggaatctaccttctctatatctgtctgttatatacatatctccagtttggtacttcgtcgacaaccgcggtcagtattgaaaatagtgataatagtactgcggagatgttatcatctaccagcatgtccgctaccaccccgatatcccagccaacatctccattcactactccaactagaagatctacaaatatagctacaagttcgagtaccacccaggcatcccagccaacatctacattaactactctaactagaagctcgacaactatagctacaagtccgagtaccacccaggcagccacattcataggatcatctaccgattccaataccactttactcaaaacaacaaaaaaaccaaagcgtaaaaagaataagaataacggggccagatttaaattagattgtggatataagggggttatctacagaccgtattttagccctcttcagctaaactgtactctacccacagaacctcatattaccaaccctattgacttcgagatctggtttaaaccacgcaccagatttggggattttcttggggataaagaagacttcgtagggaatcatacccgcaccagcatattactatttagcagccgtaatgggagtgttaattccatggatcttggggacgcgacactcgggatcctacaatctaggataccagattacacattatataatattcccatacaacataccgaagcgatgtcattgggaatcaaatctgtggaatctgccacgtccggtgtttatacatggcgggtctatggtggagatggactaaataaaacagtgctaggacaggtaaatgtatctgtagtggcatatcaccccccgagcgtaaatcttacaccacgcgccagtctatttaataagacctttgaggcggtatgtgcagtggcgaattacttccccccgcgatccacgaaactaacatggtatcttgacgggaagccaatagaaaggcaatacatttcagatacggcaagtgtatggatagatggactcatcaccagaagttctgtgttggctattccgacaactgaaacagattccgagaaaccagatatacgatgtgatttggaatggcatgaaagtcctgtgtcctataagagattcacgaaaagtgtagccccggacgtctattacccacctactgtgtctgttaccttcgctgatacacgggctatatgtgatgttaaatgtgtaccacgggacgggatatccttgatgtggaaaattggtaactaccatctaccaaaagcaatgagtgctgatatactgatcacaggtccgtgtatagaacgtccaggtttggtcaacattcagagtatgtgtgatatatcagaaacggatggacccgtgagttatacctgtcagaccatcggatacccaccaattctaccgggattttacgacacacaagtctacgacgcgtcccctgaaatcgtcagtgaatcaatgttggttagtgtcgttgctgtaatactaggagctgttctcatcacagtctttatctttattacggcattatgtttatattattctcatccccggcgattataactcttatagttcgtataaattacttatcataaccgtgtttcagcggttatatttttataacagttaattgtttactaatagtttacaaagtccatcgtttataaaaaacaagcccagtggtattataatcattcgtatggatataaaccgactcca

SEQ ID NO: 6 - DNA, 1319 bp, Artificial Sequence
  Synthetic
agtcgaaatcacaggcagtgaaatgccttaaaaatgggtctcctgtctatgttaggaatctcttattttaagtagtcccgcgagacgatttacatcccgggatcaccaacaatctgcgaggatgacgacgataagtagggataacagggtaatcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaagtagtcccgcgagacgatttacatcccgggatcaccaacaatctgcgctcttatagttcgtataaattacttatcataaccgtgtttcagcggttatatttttataacagttaattgtttactaatagtttacaaagtccatcgtttataaaaaacaagcccagtggtattataatcattcgtatggatataaaccgactcca

SEQ ID NO: 7 - DNA, 274 bp, Artificial Sequence
  Synthetic
agtcgaaatcacaggcagtgaaatgccttaaaaatgggtctcctgtctatgttaggaatctcttattttaagtagtcccgcgagacgatttacatcccgggatcaccaacaatctgcgctcttatagttcgtataaattacttatcataaccgtgtttcagcggttatatttttataacagttaattgtttactaatagtttacaaagtccatcgtttataaaaaacaagcccagtggtattataatcattcgtatggatataaaccgactcca

SEQ ID NO: 8 - DNA, 453 bp, Artificial Sequence
  Synthetic
gcggttgtgtctaataactgtttttaaataaagagataagtcgaaatcacaggcagtgaaatgccttaaaaatgggtctcctgtctatgttaggaatctcttattttaagtagtcccgcgagacgatttacatcccgggatcaccaacaatctgcgaggatgacgacgataagtagggataacagggtaatcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagc

SEQ ID NO: 9 - DNA, 545 bp, Artificial Sequence
  Synthetic
gattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaagtagtcccgcgagacgatttacatcccgggatcaccaacaatctgcgctcttatagttcgtataaattacttatcataaccgtgtttcagcggttatatttttataacagttaattgtttactaatagtttacaaagtccatcgtttataaaaaacaagcccagtggtattataatcattcgtatggatataaaccg

SEQ ID NO: 10 - DNA, 1867 bp, Artificial Sequence
  Synthetic
ggtcatgtgtaatgttgacgcatagtgggtggtgacctaagattatattaaaatgtagaaggttttatgcccagttcacagtatctactgtgacctaccccggggtggtaataacaatactatcgaatagccaacaatgggactgcttgttaccatcctcgtgatattattgattgttacttcatcaagttctactattcatcaagtaacgatgacagaaggtgccgcacttttagtcgatggggatgggatcgacccacctttaaacaaaacttcacattttttgcgaggttggacatttctagagactccgaaaggatgtacaggagaggtgagtgttctaaaagtatgtatagatcgtggggtatgtccggatgatatcgttataaataagagatgtggtcacaaaatgcttgaaaccccactagcgttggcggaatttggaatttctaatagttctctcatcagaaccaaagacgtatatttcgtgaataagaccgtgtttccaattctcacacccgaaaaaagtggccttggtattcagggggccactacgaatatatccgggatatataccctgcatgagcacggtgataatggatggagtcatcaatctacattttttgtgaccgtaaaggcaaaacatcccggaccatcgttaaccccagcaccggttcacttaataacaccacatcgccatggggcacatttccacgtaagaaactatcattcgcatgtctacattccgggagataagttcttattagaaatgcacctcaaatcagatatctatgatccagaattttcagcaacaatagactggtattttatggagactgatataaaatgcccagtttttagaatttatgaaacttgtatatttcacccccatgccgcatcctgtctacatccggaagatccctcatgcagttttacatcaccacttcgagcggtatctttaattaatagattttatccaaaatgcgatcacagatatgccgattggacatccagatgtatcaacactccaagtataaatcatatgccatatatcgaacagccggccaataacgtggatctaaagtttatcaatgtacccaccaacgcttctgggttgtacgtattcatacttcgttataatggacatccggaagaatggacctatacactcatatcaacaggagctaaatttttgaatgtgattagggatctgacacgcccacgtcttggtagtcatcaaatagagaccgatattagcacatcttcgcagtcgcctaccacggagacaccacgaaacatacatataacgtgggcgagacgttatctaaaggttatcataggaataatttgcgtagctggtatccttttgattgtaatctctatcacatgttatattcgatttcgtcatatgcgatataaaccatatgaagtgatcaacccattccctgcggtatataccagcattcctagtaacgatcccgacgaactctactttgaacgtatcgcatcgaacgacgaagaatcggcagatgattcttttgatgaatcagatgaggaggagccattgaataatcatcatatttcaacaacccaacatactgatattaatccagaaaaatccggatctgggtacagtgtatggtttcgtgatacagaagatacatcacctcagcccctacacgctcctccagattacagtcgcgtagttaaaagattaaagtctattttaaaatgacccgtcgacgagttctagcaccacgggaattggaagctgctcgtaaactccgtgagattttcaacgcagagtacgtcgcacctacgttcacactagtcgatccgggggatacgtcaaacgcgtatattgtat

SEQ ID NO: 11 - DNA, 1313 bp, Artificial Sequence
  Synthetic
ggtcatgtgtaatgttgacgcatagtgggtggtgacctaagattatattaaaatgtagaaggttttatgcccagttcacagtatctactgtgacctaccccggggtggtaataacaatactatcgaatagccaacaaggatgacgacgataagtagggataacagggtaatcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgactgtgacctaccccggggtggtaataacaatactatcgaatagccaacacccgtcgacgagttctagcaccacgggaattggaagctgctcgtaaactccgtgagattttcaacgcagagtacgtcgcacctacgttcacactagtcgatccgggggatacgtcaaacgcgtatattgtat

SEQ ID NO: 12 - DNA, 268 bp, Artificial Sequence
  Synthetic
ggtcatgtgtaatgttgacgcatagtgggtggtgacctaagattatattaaaatgtagaaggttttatgcccagttcacagtatctactgtgacctaccccggggtggtaataacaatactatcgaatagccaacacccgtcgacgagttctagcaccacgggaattggaagctgctcgtaaactccgtgagattttcaacgcagagtacgtcgcacctacgttcacactagtcgatccgggggatacgtcaaacgcgtatattgtat

SEQ ID NO: 13 - DNA, 468 bp, Artificial Sequence
  Synthetic
cgcatagtgggtggtgacctaagattatattaaaatgtagaaggttttatgcccagttcacagtatctactgtgacctaccccggggtggtaataacaatactatcgaatagccaacaaggatgacgacgataagtagggataacagggtaatcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgat

SEQ ID NO: 14 - DNA, 438 bp, Artificial Sequence
  Synthetic
ttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgactgtgacctaccccggggtggtaataacaatactatcgaatagccaacacccgtcgacgagttctagcaccacgggaattggaagctgctcgtaaactccgtgagattttcaacgcagagtacgtcgcacctacgttcacactagtcgatccggg

SEQ ID NO: 15 - DNA, 22 bp, Artificial Sequence
  Synthetic
atacgattgtatgtcaaaatac

SEQ ID NO: 16 - DNA, 20 bp, Artificial Sequence
  Synthetic
aacggagtaacctcggtgtg

SEQ ID NO: 17 - DNA, 22 bp, Artificial Sequence
  Synthetic
aatgcttaatgaattacaacag

SEQ ID NO: 18 - DNA, 22 bp, Artificial Sequence
  Synthetic
aaatccgtgttatgttaacacc

SEQ ID NO: 19 - DNA, 20 bp, Artificial Sequence
  Synthetic
gcgttactatgggaacatac

SEQ ID NO: 20 - DNA, 22 bp, Artificial Sequence
  Synthetic
atcagccataccacatttgtag

SEQ ID NO: 21 - DNA, 22 bp, Artificial Sequence
  Synthetic
accaattctcatgtttgacagc

SEQ ID NO: 22 - DNA, 20 bp, Artificial Sequence
  Synthetic
atcggcacgtaagaggttcc

SEQ ID NO: 23 - DNA, 22 bp, Artificial Sequence
  Synthetic
tggacagaacaacctaatgaac

SEQ ID NO: 24 - DNA, 21 bp, Artificial Sequence
  Synthetic
atgacagatccatgtgaagtg

SEQ ID NO: 25 - DNA, 22 bp, Artificial Sequence
  Synthetic
taatcacgagctccatcaaggc

SEQ ID NO: 26 - DNA, 32 bp, Artificial Sequence
  Synthetic
cgtacgtagttattaatagtaatcaattacgg

SEQ ID NO: 27 - DNA, 35 bp, Artificial Sequence
  Synthetic
gtcgacgagctcacgccttaagatacattgatgag

SEQ ID NO: 28 - DNA, 26 bp, Artificial Sequence
  Synthetic
acgcgtgaattcccaggcgacccgac

SEQ ID NO: 29 - DNA, 70 bp, Artificial Sequence
  Synthetic
atttaaatcgtacgataacttcgtataatgtatgctatacgaagttattatctctggtgatcttgaagag

SEQ ID NO: 30 - DNA, 70 bp, Artificial Sequence
  Synthetic
aagcttgtcgacataacttcgtatagcatacattatacgaagttattaaactgattaaatttaattaaag

SEQ ID NO: 31 - DNA, 30 bp, Artificial Sequence
  Synthetic
acgcgtgagatagttgtaccataaaatctg

SEQ ID NO: 32 - DNA, 20 bp, Artificial Sequence
  Synthetic
caatttcacacaggaaacag

SEQ ID NO: 33 - DNA, 20 bp, Artificial Sequence
  Synthetic
acggatttttaagcacacca

SEQ ID NO: 34 - DNA, 21 bp, Artificial Sequence
  Synthetic
tattcgaaccatacaactatc

SEQ ID NO: 35 - DNA, 22 bp, Artificial Sequence
  Synthetic
atacgattgtatgtcaaaatac

SEQ ID NO: 36 - DNA, 19 bp, Artificial Sequence
  Synthetic
aaggcgattaagttgggta

SEQ ID NO: 37 - DNA, 20 bp, Artificial Sequence
  Synthetic
gttggggtctttgctcaggg

SEQ ID NO: 38 - DNA, 21 bp, Artificial Sequence
  Synthetic
aagaagtcgtgctgcttcatg

SEQ ID NO: 39 - DNA, 21 bp, Artificial Sequence
  Synthetic
ggagttgttacgacattttgg

SEQ ID NO: 40 - DNA, 20 bp, Artificial Sequence
  Synthetic
ttactatgggaacatacgtc

SEQ ID NO: 41 - DNA, 20 bp, Artificial Sequence
  Synthetic
aacggagtaacctcggtgtg

SEQ ID NO: 42 - DNA, 22 bp, Artificial Sequence
  Synthetic
aatgcttaatgaattacaacag

SEQ ID NO: 43 - DNA, 22 bp, Artificial Sequence
  Synthetic
aaatccgtgttatgttaacacc

SEQ ID NO: 44 - DNA, 20 bp, Artificial Sequence
  Synthetic
gcgttactatgggaacatac

SEQ ID NO: 45 - DNA, 22 bp, Artificial Sequence
  Synthetic
atcagccataccacatttgtag

SEQ ID NO: 46 - DNA, 22 bp, Artificial Sequence
  Synthetic
accaattctcatgtttgacagc

SEQ ID NO: 47 - DNA, 20 bp, Artificial Sequence
  Synthetic
atcggcacgtaagaggttcc

SEQ ID NO: 48 - DNA, 22 bp, Artificial Sequence
  Synthetic
tggacagaacaacctaatgaac

SEQ ID NO: 49 - DNA, 21 bp, Artificial Sequence
  Synthetic
atgacagatccatgtgaagtg

SEQ ID NO: 50 - DNA, 22 bp, Artificial Sequence
  Synthetic
taatcacgagctccatcaaggc

SEQ ID NO: 51 - DNA, 23 bp, Artificial Sequence
  Synthetic
taattccaatccatcatatcaac

SEQ ID NO: 52 - DNA, 23 bp, Artificial Sequence
  Synthetic
agataatgatgtaactatgtatc

SEQ ID NO: 53 - DNA, 23 bp, Artificial Sequence
  Synthetic
tgctattgtggagtcccaaatac

SEQ ID NO: 54 - DNA, 22 bp, Artificial Sequence
  Synthetic
ataaggctgtcttacacatgag

SEQ ID NO: 55 - DNA, 25 bp, Artificial Sequence
  Synthetic
agataatgatgtaactatgtatcac

SEQ ID NO: 56 - DNA, 21 bp, Artificial Sequence
  Synthetic
attacgtttattggacccgag

SEQ ID NO: 57 - DNA, 25 bp, Artificial Sequence
  Synthetic
tgagactttaattaaatttaatcag

SEQ ID NO: 58 - DNA, 22 bp, Artificial Sequence
  Synthetic
acttcttttctacttgccctgg

SEQ ID NO: 59 - DNA, 21 bp, Artificial Sequence
  Synthetic
taaccccagcttaaacagaac

SEQ ID NO: 60 - DNA, 24 bp, Artificial Sequence
  Synthetic
tgcccctcccccgttcatgctgtg

SEQ ID NO: 61 - DNA, 24 bp, Artificial Sequence
  Synthetic
tggagcctgtttccgattctgtgt

SEQ ID NO: 62 - DNA, 23 bp, Artificial Sequence
  Synthetic
aatatcaccccggtaaacgacac

SEQ ID NO: 63 - DNA, 22 bp, Artificial Sequence
  Synthetic
acttcctggaataatgcacatc

SEQ ID NO: 64 - DNA, 24 bp, Artificial Sequence
  Synthetic
gtttttgttaatacttctgtggat

SEQ ID NO: 65 - DNA, 23 bp, Artificial Sequence
  Synthetic
agtatatataaaccctacctacg

SEQ ID NO: 66 - DNA, 23 bp, Artificial Sequence
  Synthetic
acacctctcgcttctcctctgac

SEQ ID NO: 67 - DNA, 24 bp, Artificial Sequence
  Synthetic
tcagagacgaaactgctcgcatag

SEQ ID NO: 68 - DNA, 24 bp, Artificial Sequence
  Synthetic
aatagttatttttatctcacatgc

SEQ ID NO: 69 - DNA, 22 bp, Artificial Sequence
  Synthetic
cctccgcgggttctaaaacttg

SEQ ID NO: 70 - DNA, 24 bp, Artificial Sequence
  Synthetic
gaggacgcggagagtggatggttc

SEQ ID NO: 71 - DNA, 21 bp, Artificial Sequence
  Synthetic
catattcaaatctggcctaat

SEQ ID NO: 72 - DNA, 24 bp, Artificial Sequence
  Synthetic
agatttgaatatttactgatgtga

SEQ ID NO: 73 - DNA, 25 bp, Artificial Sequence
  Synthetic
tccttattaggccagattgaatatg

SEQ ID NO: 74 - DNA, 33 bp, Artificial Sequence
  Synthetic
tctacatatgaatagtagatttgaatatttact

SEQ ID NO: 75 - DNA, 26 bp, Artificial Sequence
  Synthetic
tatttactgatgtgataactcgatcc

SEQ ID NO: 76 - DNA, 26 bp, Artificial Sequence
  Synthetic
gggggaaaaaaattgagttggagctc

SEQ ID NO: 77 - DNA, 27 bp, Artificial Sequence
  Synthetic
tgagttggagctcagtctccatatacg

SEQ ID NO: 78 - DNA, 24 bp, Artificial Sequence
  Synthetic
catatacggaggtggacagtgtag

SEQ ID NO: 79 - DNA, 72 bp, Artificial Sequence
  Synthetic
taagtagtcccgcgagacgatttacatcccgggatcaccaacaatctgcgaggatgacgacgataagtaggg

SEQ ID NO: 80 - DNA, 125 bp, Artificial Sequence
  Synthetic
ataaccgctgaaacacggttatgataagtaatttatacgaactataagagcgcagattgttggtgatcccgggatgtaaatcgtctcgcgggactacttacaaccaattaaccaattctgattag

SEQ ID NO: 81 - DNA, 72 bp, Artificial Sequence
  Synthetic
actgtgacctaccccggggtggtaataacaatactatcgaatagccaacaaggatgacgacgataagtaggg

SEQ ID NO: 82 - DNA, 125 bp, Artificial Sequence
  Synthetic
gagtttacgagcagcttccaattcccgtggtgctagaactcgtcgacgggtgttggctattcgatagtattgttattaccaccccggggtaggtcacagtcaaccaattaaccaattctgattag

SEQ ID NO: 83 - DNA, 20 bp, Artificial Sequence
  Synthetic
agtcgaaatcacaggcagtg

SEQ ID NO: 84 - DNA, 22 bp, Artificial Sequence
  Synthetic
tggagtcggtttatatccatac

SEQ ID NO: 85 - DNA, 22 bp, Artificial Sequence
  Synthetic
tcggatacccaccaattctacc

SEQ ID NO: 86 - DNA, 20 bp, Artificial Sequence
  Synthetic
ggtcatgtgtaatgttgacg

SEQ ID NO: 87 - DNA, 20 bp, Artificial Sequence
  Synthetic
gtctttggttctgatgagag

SEQ ID NO: 88 - DNA, 21 bp, Artificial Sequence
  Synthetic
atacaatatacgcgtttgacg

SEQ ID NO: 89 - DNA, 20 bp, Artificial Sequence
  Synthetic
gcggttgtgtctaataactg

SEQ ID NO: 90 - DNA, 22 bp, Artificial Sequence
  Synthetic
gcttcccatacaatcgatagat

SEQ ID NO: 91 - DNA, 21 bp, Artificial Sequence
  Synthetic
gattttgatgacgagcgtaat

SEQ ID NO: 92 - DNA, 22 bp, Artificial Sequence
  Synthetic
cggtttatatccatacgaatga

SEQ ID NO: 93 - DNA, 22 bp, Artificial Sequence
  Synthetic
cgcatagtgggtggtgacctaa

SEQ ID NO: 94 - DNA, 21 bp, Artificial Sequence
  Synthetic
atcattggcaacgctaccttt

SEQ ID NO: 95 - DNA, 21 bp, Artificial Sequence
  Synthetic
ttgccattctcaccggattca

SEQ ID NO: 96 - DNA, 21 bp, Artificial Sequence
  Synthetic
cccggatcgactagtgtgaac

SEQ ID NO: 97 - DNA, 17 bp, Artificial Sequence
  Synthetic
tggagtctaggtgtggg

SEQ ID NO: 98 - DNA, 21 bp, Artificial Sequence
  Synthetic
ggcctaataaggaaggggagg

SEQ ID NO: 99 - DNA, 30 bp, Artificial Sequence
  Synthetic
tctggcggtttgtgggttggcatattcaaa

SEQ ID NO: 100 - skipped

SEQ ID NO: 101 - DNA, 18 bp, Artificial Sequence
  Synthetic
tggagcgacgctcactga

SEQ ID NO: 102 - DNA, 20 bp, Artificial Sequence
  Synthetic
accttcgctcctcccctcgt

SEQ ID NO: 103 - DNA, 53 bp, Artificial Sequence
  Synthetic
aggttggaagccatgttgttccggttgcacatctaatctacatgaaagtggga

SEQ ID NO: 104 - DNA, 26 bp, Artificial Sequence
  Synthetic
gggggatcgagggggggcagagggga

SEQ ID NO: 105 - DNA, 16 bp, Artificial Sequence
  Synthetic
ggggctgtggggacga